SEQUENCE LISTING (53 sequences)

SEQ ID NO: 1 - DNA, 24 bp, Artificial
  primer
atgtcaaacgtgcgagtgtctaac

SEQ ID NO: 2 - DNA, 24 bp, Artificial
  primer
ttacgtttgacgtgttgtgaggcc

SEQ ID NO: 3 - DNA, 24 bp, Artificial
  primer
gccatgtcagaaccggctggggat

SEQ ID NO: 4 - DNA, 24 bp, Artificial
  primer
ttagggcttcctcttggagaagat

SEQ ID NO: 5 - DNA, 30 bp, Artificial
  primer
cctcgatcctccctttatccagccctcact

SEQ ID NO: 6 - DNA, 30 bp, Artificial
  primer
gaaccaaaatcactttccccaaggaaggag

SEQ ID NO: 7 - DNA, 27 bp, Artificial
  primer
aatgaggtccccacgtttctcgggtgt

SEQ ID NO: 8 - DNA, 29 bp, Artificial
  primer
gccaataagcaaagtgttgaacacttcac

SEQ ID NO: 9 - DNA, 26 bp, Artificial
  primer
aagtgcttgatggcttgctctgctac

SEQ ID NO: 10 - DNA, 27 bp, Artificial
  primer
acatggacagctttgctgagtcggtcc

SEQ ID NO: 11 - DNA, 28 bp, Artificial
  primer
ttgctagacgactgttttccaactccag

SEQ ID NO: 12 - DNA, 25 bp, Artificial
  primer
gctgaaggaccatttattactggag

SEQ ID NO: 13 - DNA, 25 bp, Artificial
  primer
ttcttgctgtgttctttctcccaca

SEQ ID NO: 14 - DNA, 29 bp, Artificial
  primer
ctaatcaaacaggaaattcaaaggaagag

SEQ ID NO: 15 - DNA, 27 bp, Artificial
  primer
cttgatttctcgtaacagatctgcatc

SEQ ID NO: 16 - DNA, 27 bp, Artificial
  primer
actctcagcaacaacaccagagctcta

SEQ ID NO: 17 - DNA, 27 bp, Artificial
  primer
tagctttcgtgccattgccacaacatc

SEQ ID NO: 18 - DNA, 23 bp, Artificial
  primer
ggcctgatgggtcttatctatgg

SEQ ID NO: 19 - DNA, 23 bp, Artificial
  primer
ttagatggaagctggctcaagag

SEQ ID NO: 20 - DNA, 23 bp, Artificial
  primer
gaactttgcctcattcttttgca

SEQ ID NO: 21 - DNA, 26 bp, Artificial
  primer
ctgagctccagtctcttgtatttctg

SEQ ID NO: 22 - DNA, 29 bp, Artificial
  primer
caaggtaaccacttctaagaccataattc

SEQ ID NO: 23 - DNA, 28 bp, Artificial
  primer
cctgcttgaagattacataactttttgt

SEQ ID NO: 24 - DNA, 29 bp, Artificial
  primer
tcaatggagatagcaaaggtattataacc

SEQ ID NO: 25 - DNA, 30 bp, Artificial
  primer
agatttacttaggagcacattatgagtgtt

SEQ ID NO: 26 - DNA, 21 bp, Artificial
  primer
atgtttatcaacggtccgcct

SEQ ID NO: 27 - DNA, 21 bp, Artificial
  primer
catccccagtggcttttaagg

SEQ ID NO: 28 - DNA, 18 bp, Artificial
  primer
tgcgcggagacagcatag

SEQ ID NO: 29 - DNA, 20 bp, Artificial
  primer
tgggttcaaaatgccaggtt

SEQ ID NO: 30 - DNA, 21 bp, Artificial
  primer
ggcccttcagaaatgcttgtc

SEQ ID NO: 31 - DNA, 25 bp, Artificial
  primer
cccattacacaattcatgtgcttag

SEQ ID NO: 32 - DNA, 25 bp, Artificial
  primer
cagatcatctagaagcagctggttt

SEQ ID NO: 33 - DNA, 26 bp, Artificial
  primer
acagaatctggtgcctactaacaaaa

SEQ ID NO: 34 - DNA, 20 bp, Artificial
  primer
gagcagaaggaagcgtggtt

SEQ ID NO: 35 - DNA, 26 bp, Artificial
  primer
gacacatgaatttccacaacagtaaa

SEQ ID NO: 36 - DNA, 28 bp, Artificial
  primer
acgtaatgacattttggtctgagtaact

SEQ ID NO: 37 - DNA, 23 bp, Artificial
  primer
aaatgctgctaaacatcctggaa

SEQ ID NO: 38 - DNA, 25 bp, Artificial
  primer
tgaatcctgaatactatgcctcctt

SEQ ID NO: 39 - DNA, 28 bp, Artificial
  primer
tcccttcttctcaagactactcagtatg

SEQ ID NO: 40 - DNA, 27 bp, Artificial
  primer
accttcttctcacaagactttttaagc

SEQ ID NO: 41 - DNA, 20 bp, Artificial
  primer
ccttctcagccaccatggaa

SEQ ID NO: 42 - DNA, 19 bp, Artificial
  primer
ccaggtttcccctgcagtt

SEQ ID NO: 43 - DNA, 21 bp, Artificial
  primer
ccccctgacttagtcctcgtt

SEQ ID NO: 44 - DNA, 23 bp, Artificial
  primer
cccactgccttgatctagttgat

SEQ ID NO: 45 - DNA, 23 bp, Artificial
  primer
gtcaagcaatctatggcctacca

SEQ ID NO: 46 - DNA, 23 bp, Artificial
  primer
tgctgtgagaataatgggttgtg

SEQ ID NO: 47 - DNA, 21 bp, Artificial
  primer
ggctggcttaggagcttcact

SEQ ID NO: 48 - DNA, 21 bp, Artificial
  primer
acctgcggttcccactttatt

SEQ ID NO: 49 - DNA, 25 bp, Artificial
  primer
accactccaaatgtctaaggtcact

SEQ ID NO: 50 - DNA, 23 bp, Artificial
  primer
ggcctgatgggtcttatctatgg

SEQ ID NO: 51 - DNA, 23 bp, Artificial
  primer
ttagatggaagctggctcaagag

SEQ ID NO: 52 - DNA, 23 bp, Artificial
  primer
gaactttgcctcattcttttgca

SEQ ID NO: 53 - DNA, 26 bp, Artificial
  primer
ctgagctccagtctcttgtatttctg

(19) United States
(12) Patent Application Publication
(10) Pub. No.: US 2010/0021918 A1
(43) Pub. Date: Jan. 28, 2010

(54) METHOD FOR ASSAYING ACTION OF ANTITUMOR AGENT USING SPLICING DEFECTS AS INDEX

(75) Inventors: Yoshiharu Mizui, Ibaraki-Ken (JP); Naoko Hata, Ibaraki-ken (JP); Masao Iwata, Ibaraki-Ken (JP); Koji Sagane, Ibaraki-Ken (JP); Mai Uesugi, Ibaraki-Ken (JP); Tadashi Kadowaki, Ibaraki-Ken (JP); Taku Yoshida, Ibaraki-Ken (JP)

Correspondence Address: FISH & RICHARDSON P.C., P.O. BOX 1022, MINNEAPOLIS, MN, 55440-1022, US

(73) Assignee: Eisai R&D Management Co., Ltd., Tokyo-To (JP)

(21) Appl. No.: 12/529,959

(22) PCT Filed: Mar. 5, 2008
(86) PCT No.: PCT/JP2008/053977

§ 371 Date: Sep. 4, 2009

Related U.S. Application Data
(60) Provisional application No. 60/904,774, filed on Mar. 5, 2007.
(60) Provisional application No. 60/960,403, filed on Sep. 28, 2007.

(51) Int. Cl. C12Q 1/68; C07H 21/04; G01N 33/53; C07K 16/00

(52) U.S. Cl. 435/6; 536/24.31; 435/7.1; 530/387.1

(57) ABSTRACT

An object of the present invention is to provide a method, a probe, a primer, an antibody, a reagent, and a kit for assaying an action of a pladienolide derivative to a living subject. According to the present invention, there is provided a method for assaying an action of the pladienolide derivative using splicing defect as an index.

### BACKGROUND OF THE INVENTION

1. Field of the Invention

The present invention relates to a method for assaying an action of an antitumor agent using splicing defect as an index, more particularly, a method for assaying an action of an antitumor agent using an increase in the expression level of pre-mRNA or abnormal protein as an index.

2. Background Art

Pladienolide derivatives are derivatives of natural pladienolide. Since pladienolide exhibits an excellent antitumor activity (Mizui et al., 2004, J. Antibiotics 577:188-196), search for a compound with higher antitumor activities has been performed to find the pladienolide derivatives (WO2002/060890 and WO2003/099813).

### SUMMARY OF THE INVENTION

The present inventors have found that pre-mRNA of a certain group of genes and abnormal proteins increase when a pladienolide derivative is contacted with a sample obtained from living cells including cancerous cells, and peripheral blood (PBMC) and whole blood (PBC) of a subject. Without wishing to be bound by any theory, administration of the pladienolide derivative may cause an introduction of a mutation in pre-mRNA of a certain group of genes thereby inducing splicing defect. The present invention was made based on such findings.

It is an object of the present invention to provide a method, a probe, a primer, an antibody, a reagent and a kit for assaying an action of the pladienolide derivative on a living subject.

According to the present invention, there are provided inventions (1) to (19) as follows.

(1) A method for assaying an action of a compound represented by formula (I), a pharmaceutically acceptable salt thereof, or a solvate of them to a mammal, which comprises detecting splicing defect caused by the compound represented by formula (I), the pharmaceutically acceptable salt thereof, or the solvate of them:

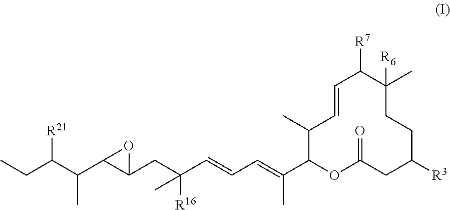

wherein R³, R⁶, R⁷ and R²¹, the same or different, each represents
1) a hydroxyl group or an oxo group formed together with the carbon atom to which it is bound, provided that R⁶ is limited to a hydroxyl group,
2) an optionally substituted C₁₋₂₂ alkoxy group,
3) an optionally substituted unsaturated C₂₋₂₂ alkoxy group,
4) an optionally substituted C₇₋₂₂ aralkyloxy group,
5) an optionally substituted 5- to 14-membered heteroaralkyloxy group,
6) RCO—O— wherein R represents

a) a hydrogen atom,

b) an optionally substituted C₁₋₂₂ alkyl group,

c) an optionally substituted unsaturated C₂₋₂₂ alkyl group,

d) an optionally substituted C₆₋₁₄ aryl group,

e) an optionally substituted 5- to 14-membered heteroaryl group,

f) an optionally substituted C₇₋₂₂ aralkyl group,

g) an optionally substituted 5- to 14-membered heteroaralkyl group,

h) an optionally substituted C₁₋₂₂ alkoxy group,

i) an optionally substituted unsaturated C₂₋₂₂ alkoxy group,

j) an optionally substituted C₆₋₁₄ aryloxy group or

k) an optionally substituted 5- to 14-membered heteroaryloxy group,

7) R^{SI}R^{S2}R^{S3}SiO— wherein R^{SI}, R^{S2}, and R^{S3}, the same or different, each represents

a) a C₁₋₆ alkyl group or

b) a C₆₋₁₄ aryl group,

8) a halogen atom,
9) R^{N1}R^{N2}N—R^{M}— wherein R^{M} represents

a) a single bond,

b) —CO—O—,

c) —SO₂—O—,

d) —CS—O— or

e) —CO—NR^{N3}— wherein R^{N3} represents a hydrogen atom or an optionally substituted C₁₋₆ alkyl group, provided that each of the leftmost bond in b) to e) is bound to the nitrogen atom; and R^{N1} and R^{N2}, the same or different from each other and each represents

a) a hydrogen atom,

b) an optionally substituted C₁₋₂₂ alkyl group,

c) an optionally substituted unsaturated C₂₋₂₂ alkyl group,

d) an optionally substituted aliphatic C₂₋₂₂ acyl group,

e) an optionally substituted aromatic C₇₋₁₅ acyl group,

f) an optionally substituted C₆₋₁₄ aryl group,

g) an optionally substituted 5- to 14-membered heteroaryl group,

h) an optionally substituted C₇₋₂₂ aralkyl group,

i) an optionally substituted C₁₋₂₂ alkylsulfonyl group,

j) an optionally substituted C₆₋₁₄ arylsulfonyl group,

k) an optionally substituted 3- to 14-membered non-aromatic heterocyclic group formed by R^{NI} and R^{N2} together with the nitrogen atom to which R^{NI} and R^{N2} are bound, and the non-aromatic heterocyclic group optionally has substituent(s),

l) an optionally substituted 5- to 14-membered heteroaralkyl group,

m) an optionally substituted C₃₋₁₄ cycloalkyl group or

n) an optionally substituted 3- to 14-membered non-aromatic heterocyclic group,

10) R^{N4}SO₂— wherein R^{N4} represents

a) an optionally substituted C₁₋₂₂ alkyl group,

b) an optionally substituted C₆₋₁₄ aryl group,

c) an optionally substituted C₁₋₂₂ alkoxy group,

d) an optionally substituted unsaturated C₂₋₂₂ alkoxy group,

e) an optionally substituted C₆₋₁₄ aryloxy group,

f) an optionally substituted 5- to 14-membered heteroaryloxy group,

g) an optionally substituted C₇₋₂₂ aralkyloxy group or

h) an optionally substituted 5- to 14-membered heteroaralkyloxy group,

11) (R^{N5}O)₂PO—O— wherein R^{N5} represents

a) an optionally substituted C₁₋₂₂ alkyl group,

b) an optionally substituted unsaturated C₂₋₂₂ alkyl group,

c) an optionally substituted C₆₋₁₄ aryl group,

d) an optionally substituted 5- to 14-membered heteroaryl group,

e) an optionally substituted C₇₋₂₂ aralkyl group or

f) an optionally substituted 5- to 14-membered heteroaralkyl group,

12) (R^{N1}R^{N2}N)₂PO—O— wherein R^{NI} and R^{N2} have the same meanings as defined above or
13) (R^{N1}R^{N2}N)(R^{N5}O)PO—O— wherein R^{N1}, R^{N2} and R^{N5} have the same meanings as defined above, provided that a compound in which R³, R⁶, R⁷ and R²¹ are all hydroxyl groups, and a compound in which R³, R⁶ and R²¹ are all hydroxyl groups and R⁷ is an acetoxy group are excluded,

R¹⁵ represents a hydrogen atom or hydroxyl group.

(2) The method according to (1), wherein the compound represented by formula (I) is selected from the group consisting of:
(8E,12E,14E)-7-(N-(2-(N′,N′-Dimethylamino)ethyl)-N-methylcarbamoyloxy)-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-methylhomopiperazin-1-yl)carbonyl)oxy-18,19-epoxytricosa-8,12,14-trien-11-olide;
(8E,12E,14E)-3,6,16,21-Tetrahydroxy-6,10,12,16,20-pentamethyl-7-((4-methylpiperazin-1-yl)carbonyl)oxy-18,19-epoxytricosa-8,12,14-trien-11-olide;
(8E,12E,14E)-7-((4-Butylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Ethylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-propylpiperazin-1-yl)carbonyl)oxy-18,19-epoxytricosa-8,12,14-trien-11-olide;
(8E,12E,14E)-7-((4-Cyclohexylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-(Cyclopropylmethyl)piperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-propylhomopiperazin-1-yl)carbonyl)oxy-18,19-epoxytricosa-8,12,14-trien-11-olide;
(8E,12E,14E)-7-((4-(Cyclopropylmethyl)homopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Cyclopentylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-7-((4-isopropylpiperazin-1-yl)carbonyl)oxy-6,10,12,16,20-pentamethyl-18,19-epoxytricosa-8,12,14-trien-11-olide;
(8E,12E,14E)-7-((4-Cycloheptylpiperazin-1-yl)carbonyl)oxy-3,6,16,21-Tetrahydroxy-6,10,12,16,20-pentamethyl-18,19-epoxytricosa-8,12,14-trien-11-olide;
(8E,12E,14E)-7-(N-(2-(N′,N′-Diethylamino)ethyl)-N-methylcarbamoyloxy)-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-7-((4-isobutylhomopiperazin-1-yl)carbonyl)oxy-6,10,12,16,20-pentamethyl-18,19-epoxytricosa-8,12,14-trien-11-olide;
(8E,12E,14E)-7-((4-Ethylhomopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Butylhomopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,16,21-Trihydroxy-6-methoxy-6,10,12,16,20-pentamethyl-7-((4-methylpiperazin-1-yl)carbonyl)oxy-18,19-epoxytricosa-8,12,14-trien-11-olide;
(8E,12E,14E)-3,16,21-Trihydroxy-6-methoxy-6,10,12,16,20-pentamethyl-7-((4-(piperidin-1-yl)piperidin-1-yl)carbonyl)oxy-18,19-epoxytricosa-8,12,14-trien-11-olide;
(8E,12E,14E)-3,6,7,21-Tetrahydroxy-6,10,12,16,20-pentamethyl-18,19-epoxytricosa-8,12,14-trien-11-olide;
(8E,12E,14E)-7-((4-(2,2-Dimethylpropyl)homopiperazin-1-yl)carbonyl)oxy-3,6,16,21-tetrahydroxy-6,10,12,16,20-pentamethyl-18,19-epoxytricosa-8,12,14-trien-11-olide; and
(8E,12E,14E)-3,6,16-Trihydroxy-21-methoxy-6,10,12,16,20-pentamethyl-7-((4-methylpiperazin-1-yl)carbonyl)oxy-18,19-epoxytricosa-8,12,14-trien-11-olide.
   (3) The method according to (1), wherein the detection of splicing defect comprises the steps of:

(a) measuring the expression level of pre-mRNA before and after administration of the compound represented by formula (I), the pharmaceutically acceptable salt thereof, or the solvate of them to a mammal;

(b) comparing, based on the expression level measured in (a), the expression level of pre-mRNA before and after administration of the compound represented by formula (I), the pharmaceutically acceptable salt thereof, or the solvate of them, to determine that the compound represented by formula (I), the pharmaceutically acceptable salt thereof, or the solvate of them exerts an action to the mammal when the expression level of pre-mRNA after the administration increases.

(4) The method according to (3), wherein the pre-mRNA of which expression level is measured is pre-mRNA of at least one gene selected from the genes listed in Table 1, Table 2, Table 3, Table 4, and Table 5, or a homologous gene thereof.
(5) The method according to (4), wherein the gene(s) are selected from DNAJB1, BZW1, NUP54, RIOK3, CDKN1B, STK17B, EIF4A1, and ID1.
(6) The method according to (3), wherein in step (a), the expression level of pre-mRNA in samples obtained from a subject before and after administration of the compound represented by formula (I), the pharmaceutically acceptable salt thereof, or the solvate of them, is measured.
(7) The method according to (6), wherein the samples obtained from the subject are selected from hemocytes in peripheral blood, plasma, and serum.
(8) A probe or primer for assaying an action of a compound represented by formula (I), a pharmaceutically acceptable salt thereof, or a solvate of them to a mammal, which consists of a polynucleotide capable of hybridizing with a polynucleotide consisting of a nucleotide sequence of at least one gene selected from the genes listed in Table 1, Table 2, Table 3, Table 4, and Table 5, or a homologous gene thereof, or a complementary sequence thereof.
(9) The probe or primer according to (8), which is capable of detecting a genomic intron region or a part thereof in a gene listed in Table 1, Table 2, Table 3, Table 4 and Table 5, or which is capable of detecting a polynucleotide lacking a part of a genomic exon region in a gene listed in Table 1, Table 2, Table 3, Table 4 and Table 5.
(10) A reagent or kit for assaying an action of a compound represented by formula (I), a pharmaceutically acceptable salt thereof, or a solvate of them to a mammal, which comprises the probe or the primer according to (8).
(11) The method according to (1), wherein the detection of splicing defect comprises the steps of:

(f) measuring the expression level of an abnormal protein before and after administration of the compound represented by formula (I), the pharmaceutically acceptable salt thereof, or the solvate of them to a mammal;

(g) comparing, based on the expression level measured in (f), the expression level of the abnormal protein before and after administration of the compound represented by formula (I), the pharmaceutically acceptable salt thereof or the solvate of them, to determine that the compound represented by formula (I), the pharmaceutically acceptable salt thereof, or the solvate of them exerts an action to the mammal when the expression level of the abnormal protein after administration increases.

(12) The method according to (11), wherein the abnormal protein of which expression level is measured is a protein consisting of amino acids encoded by a polynucleotide of at least one gene selected from the genes listed in Table 1 and Table 3, or a homologous gene thereof where splicing defect has been caused in the polynucleotide, or a protein consisting of amino acids encoded by a polynucleotide of at least one gene selected from the genes listed in Table 2, Table 4 and Table 5 or a homologous gene thereof where splicing defect has been caused in the polynucleotide.
(13) The method according to (11), wherein the abnormal protein of which expression level is measured is a protein consisting of amino acids encoded by a polynucleotide of at least one gene selected from DNAJB1, BZW1, NUP54, RIOK3, CDKN1B, STK17B, EIF4A1 and ID1 where splicing defect has been caused in the polynucleotide.
(14) The method according to (11), wherein in step (f), the expression level of the abnormal protein in the samples obtained from a subject before and after administration of the compound represented by formula (I) or the pharmaceutically acceptable salt thereof or the solvate of them, is measured.
(15) The method according to (14), wherein the samples obtained from the subject are selected from hemocytes in peripheral blood, plasma, and serum.
(16) An antibody against an abnormal protein consisting of amino acids encoded by a polynucleotide of at least one gene selected from the genes listed in Table 1, Table 2, Table 3, Table 4 and Table 5 where splicing defect has been caused in the polynucleotide, or a fragment thereof.
(17) A reagent or kit for assaying an action of a compound represented by formula (I), a pharmaceutically acceptable salt thereof, or a solvate of them to a mammal, comprising the antibody or the fragment thereof according to (16).
(18) Use of a probe or primer consisting of a polynucleotide capable of hybridizing with a polynucleotide consisting of a nucleotide sequence of at least one gene selected from the genes listed in Table 1, Table 2, Table 3, Table 4, and Table 5, and homologous genes thereof, or complementary sequences thereof, for assaying an action of a compound represented by formula (I), a pharmaceutically acceptable salt thereof, or a solvate of them to a mammal.
(19) Use of an antibody against a protein consisting of amino acids encoded by a polynucleotide of at least one gene selected from the genes listed in Table 1, Table 2, Table 3, Table 4 and Table 5, or a fragment of the antibody, for assaying an action of a compound represented by formula (I), a pharmaceutically acceptable salt thereof, or a solvate of them to a mammal.

According to the present invention, an action of the compound represented by formula (I) to a living body can be confirmed using splicing defect in a cancerous or normal tissue of a cancer patient or normal tissue of a healthy individual, more specifically, the expression level of pre-mRNA or an abnormal protein as an index. For instance, when splicing defect is found in the cancerous or normal tissue of the cancer patient, more specifically, the expression level of pre-mRNA or the abnormal protein increases, it can be determined that the compound represented by formula (I) exerts the action in the body and that thus administration of the drug is no longer required or less amount of the drug should be administrated. Further, when splicing defect is not found in the cancerous or normal tissue of the cancer patient, more specifically, the expression level of pre-mRNA or the abnormal protein exhibits the same amount as before the administration, it can be determined that the compound represented by formula (I) does not exert the action and further administration of the drug is required. Hence, according to the invention, by monitoring periodically the expression of pre-mRNA or the abnormal protein, the antitumor agent can be administered more effectively to the patient and a minimally required amount of the drug can be administered to the patient. In particular, since the action of the compound represented by formula (I) can be judged by monitoring the expression level of pre-mRNA or the abnormal protein in samples obtained from normal tissue such as blood of the patient, it is an advantage that the action of the compound represented by formula (I) to the living body can be readily and reliably assessed.

### BRIEF DESCRIPTION OF THE DRAWINGS

FIG. 1 shows accumulation of pre-mRNA of CDKN1B (p27) and CDKN1A (p21) in HeLa cells.

FIG. 2 shows accumulation of pre-mRNA of DNAJB1, BZW1, SPAG5, RIOK3, NUP54, and BRD2 in HeLa cells.

FIG. 3 shows the expression of mRNA in whole blood (PBC) samples obtained from human peripheral blood. A: Results of samples obtained with the Tempus Blood RNA Tube. B: Results of samples obtained with the PAXgene Blood RNA Tube.

FIG. 4 shows expression of a protein translated from the pre-mRNA.

FIG. 5 shows results measured expression of precursor genes (DNAJB1 and EIF4A1) in blood of nude mice to which human colon cancer cells were subcutaneously transplanted and the pladienolide derivative was administered.

FIG. 6 shows results measured expression of precursor genes (DNAJB1 and EIF4A1) in the tumor of nude mice to which human colon cancer cells were subcutaneously transplanted and the pladienolide derivative was administered.

FIG. 7 shows a scatter diagram of the expression level of probe sets and genes of control cells, and standard deviation thereof. “A” represents the expression level and standard deviation of exons. “B” represents the expression level and standard deviation of genes.

### DETAILED DESCRIPTION OF THE INVENTION

All technical terms, scientific terms and terminologies used in the present specification have the same meanings as those that are generally understood by those ordinary skilled in the art in the technical fields to which the present invention belongs, and are used merely for the purpose of explaining a specific embodiment but are not intended to make limitation. The present invention can be carried out in various embodiments as long as not departing from the spirit thereof. All the prior art documents, published publications, patent publications and other patent documents cited in the present specification are incorporated into the present specification as references, and can be used for carrying out the present invention.

The “halogen atom” used in the specification of the present application means a fluorine atom, a chlorine atom, a bromine atom and an iodine atom. Among them, for example, a fluorine atom, a chlorine atom or a bromine atom is preferred, of which a fluorine atom or a chlorine atom is preferred.

The “C₁₋₂₂ alkyl group” used in the specification of the present application indicates a linear or branched alkyl group having 1 to 22 carbon atoms, such as methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, n-hexyl group, 1-ethyl-2-methylpropyl group, 1,1,2-trimethylpropyl group, 1-propylpropyl group, 1-methylbutyl group, 2-methylbutyl group, 1,1-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group, 3-methylpentyl group, n-heptyl group, n-octyl group, n-nonyl group or n-decyl group; preferably a linear or branched alkyl group having 1 to 6 carbon atoms, such as methyl group, ethyl group, n-propyl group, iso-propyl group, n-butyl group, iso-butyl group, sec-butyl group, tert-butyl group or n-pentyl group; and more preferably, for example, methyl group, ethyl group, propyl group, iso-propyl group, n-butyl group, iso-butyl group or tert-butyl group.

The “unsaturated C₂₋₂₂ alkyl group” used in the specification of the present application indicates a linear or branched alkenyl group having 2 to 22 carbon atoms or a linear or branched alkynyl group having 2 to 22 carbon atoms, such as vinyl group, allyl group, 1-propenyl group, isopropenyl group, 2-methyl-1-propenyl group, 2-methyl-2-propenyl group, 1-butenyl group, 2-butenyl group, 3-butenyl group, 1-pentenyl group, 1-hexenyl group, 1,3-hexadienyl group, 1,5-hexadienyl group, ethynyl group, 1-propynyl group, 2-propynyl group, 1-butynyl group, 2-butynyl group, 3-butynyl group, 1-ethynyl-2-propynyl group, 2-methyl-3-butynyl group, 1-pentynyl group, 1-hexynyl group, 1,3-hexanediynyl group or 1,5-hexanediynyl group. It preferably indicates a linear or branched alkenyl group having 2 to 10 carbon atoms or a linear or branched alkynyl group having 2 to 10 carbon atoms, such as vinyl group, allyl group, 1-propenyl group, 2-propenyl group, isopropenyl group, 3-methyl-2-butenyl group, 3,7-dimethyl-2,6-octadienyl group, ethynyl group, 1-propynyl group, 2-propynyl group, 1-butynyl group, 2-butynyl group, 3-butynyl group or 3-methyl-1-propynyl group.

The “C₆₋₁₄ aryl group” used in the specification of the present application means an aromatic cyclic hydrocarbon group having 6 to 14 carbon atoms, and a monocyclic group and condensed rings such as a bicyclic group and a tricyclic group are included. Examples thereof include phenyl group, indenyl group, 1-naphthyl group, 2-naphthyl group, azulenyl group, heptalenyl group, indacenyl group, acenaphthyl group, fluorenyl group, phenalenyl group, phenanthrenyl group and anthracenyl group; and preferred examples include phenyl group, 1-naphthyl group and 2-naphthyl group.

The “5- to 14-membered heteroaryl group” used in the specification of the present application means a monocyclic, bicyclic or tricyclic 5- to 14-membered aromatic heterocyclic group which contains one or more of hetero atoms selected from the group consisting of nitrogen atom, sulfur atom and oxygen atom. Preferred examples thereof include nitrogen-containing aromatic heterocyclic groups such as pyrrolyl group, pyridyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, isoindolyl group, indolizinyl group, purinyl group, indazolyl group, quinolyl group, isoquinolyl group, quinolizinyl group, phthalazinyl group, naphthyridinyl group, quinoxalinyl group, quinazolinyl group, cinnolinyl group, pteridinyl group, imidazotriazinyl group, pyrazinopyridazinyl group, acridinyl group, phenanthridinyl group, carbazolyl group, carbazolinyl group, perimidinyl group, phenanthrolinyl group, phenazinyl group, imidazopyridinyl group, imidazopyrimidinyl group, pyrazolopyridinyl group and pyrazolopyridinyl group; sulfur-containing aromatic heterocyclic groups such as thienyl group and benzothienyl group; and oxygen-containing aromatic heterocyclic groups such as furyl group, pyranyl group, cyclopentapyranyl group, benzofuryl group and isobenzofuryl group; aromatic heterocyclic groups containing two or more different hetero atoms, such as thiazolyl group, isothiazolyl group, benzothiazolyl group, benzthiadiazolyl group, phenothiazinyl group, isoxazolyl group, furazanyl group, phenoxazinyl group, oxazolyl group, isoxazoyl group, benzoxazolyl group, oxadiazolyl group, pyrazolooxazolyl group, imidazothiazolyl group, thienofuranyl group, furopyrrolyl group and pyridoxazinyl group; and preferred examples include thienyl group, furyl group, pyridyl group, pyridazyl group, pyrimidyl group and pyrazyl group.

The “3- to 14-membered non-aromatic heterocyclic group” used in the specification of the present application indicates a monocyclic, bicyclic or tricyclic 3- to 14-membered non-aromatic heterocyclic group which may contain one or more hetero atoms selected from the group consisting of nitrogen atom, sulfur atom and oxygen atom. Preferred examples thereof include aziridinyl group, azetidinyl group, pyrrolidinyl group, pyrrolyl group, piperidinyl group, piperazinyl group, homopiperidinyl group, homopiperazinyl group, imidazolyl group, pyrazolidyl group, imidazolidyl group, morpholyl group, thiomorpholyl group, imidazolinyl group, oxazolinyl group, 2,5-diazabicyclo[2.2.1]heptyl group, 2,5-diazabicyclo[2.2.2]octyl group, 3,8-diazabicyclo[3.2.1]octyl group, 1,4-diazabicyclo[4.3.0]nonyl group, quinuclidyl group, tetrahydrofuran-yl group and tetrahydrothiophen-yl group. The non-aromatic heterocyclic groups also include groups derived from pyridone ring, and non-aromatic fused rings (e.g., a group derived from, for example, phthalimide ring or succinimide ring).

The “C₇₋₂₂ aralkyl group” used in the specification of the present application means a group corresponding to the above-defined “C₁₋₂₂ alkyl group” of which substitutable moiety is replaced by the above-defined “C₆₋₁₄ aryl group”. Specific examples thereof include benzyl group, phenethyl group, 3-phenylpropyl group, 4-phenylbutyl group, 1-naphthylmethyl group and 2-naphthylmethyl group; and preferred examples include aralkyl groups having 7 to 10 carbon atoms such as benzyl group and phenethyl group.

The “5- to 14-membered heteroaralkyl group” used in the specification of the present application means a group corresponding to the above-defined “C₁₋₂₂ alkyl group” of which substitutable moiety is replaced by the above-defined “5- to 14-membered heteroaryl group”. Specific examples thereof include thienylmethyl group, furylmethyl group, pyridylmethyl group, pyridazylmethyl group, pyrimidylmethyl group and pyrazylmethyl group; and preferred examples include thienylmethyl group, furylmethyl group and pyridylmethyl group.

The “C₃₋₁₄ cycloalkyl group” used in the specification of the present application indicates a cycloalkyl group having 3 to 14 carbon atoms, and suitable examples thereof include cyclopropyl group, cyclobutyl group, cyclopentyl group, cyclohexyl group, cycloheptyl group and cyclooctyl group; and preferred examples include cyclopentyl group, cyclohexyl group, cycloheptyl group and cyclooctyl group.

The “C₄₋₉ cycloalkyl alkyl group” used in the specification of the present application means a group corresponding to the above-defined “C₁₋₂₂ alkyl group” of which substitutable moiety is replaced by the above-defined “C₃₋₁₄ cycloalkyl group”. Specific examples thereof include cyclopropylmethyl group, cyclobutylmethyl group, cyclopentylmethyl group, cyclohexylmethyl group, cycloheptylmethyl group and cyclooctylmethyl group; and preferred example include cyclopropylmethyl group, cyclobutylmethyl group and cyclopentylmethyl group.

The “C₁₋₂₂ alkoxy group” used in the specification of the present application means a group corresponding to the above-defined “C₁₋₂₂ alkyl group” to which end an oxygen atom is bonded. Suitable examples thereof include methoxy group, ethoxy group, n-propoxy group, iso-propoxy group, n-butoxy group, iso-butoxy group, sec-butoxy group, tert-butoxy group, n-pentyloxy group, iso-pentyloxy group, sec-pentyloxy group, n-hexoxy group, iso-hexoxy group, 1,1-dimethylpropyloxy group, 1,2-dimethylpropyloxy group, 2,2-dimethylpropoxy group, 1-methyl-2-ethylpropoxy group, 1-ethyl-2-methylpropoxy group, 1,1,2-trimethylpropoxy group, 1,2,2-trimethylpropoxy group, 1,1-dimethylbutoxy group, 1,2-dimethylbutoxy group, 2,2-dimethylbutoxy group, 2,3-dimethylbutyloxy group, 1,3-dimethylbutoxy group, 2-ethylbutoxy group, 2-methylpentoxy group, 3-methylpentyloxy group and hexyloxy group; and preferred examples include methoxy group, ethoxy group, n-propoxy group, iso-propoxy group, iso-butoxy group and 2,2-dimethylpropyloxy group.

The “unsaturated C₂₋₂₂ alkoxy group” used in the specification of the present application means a group corresponding to the above-defined “unsaturated C₂₋₂₂ alkyl group” to which end an oxygen atom is bonded. Suitable examples thereof include vinyloxy group, allyloxy group, 1-propenyloxy group, 2-propenyloxy group, isopropenyloxy group, 2-methyl-1-propenyloxy group, 2-methyl-2-propenyloxy group, 1-butenyloxy group, 2-butenyloxy group, 3-butenyloxy group, 1-pentenyloxy group, 1-hexenyloxy group, 1,3-hexadienyloxy group, 1,5-hexadienyloxy group, propargyloxy group and 2-butynyloxy group; and preferred examples include allyloxy group, propargyloxy group and 2-butynyloxy group.

The “C₆₋₁₄ aryloxy group” used in the specification of the present application means a group corresponding to the above-defined “C₆₋₁₄ aryl group” to which end an oxygen atom is bonded. Specific examples thereof include phenyloxy group, indenyloxy group, 1-naphthyloxy group, 2-naphthyloxy group, azulenyloxy group, heptalenyloxy group, indacenyloxy group, acenaphthyloxy group, fluorenyloxy group, phenalenyloxy group, phenanthrenyloxy group and anthracenyloxy group; and preferred examples include phenyloxy group, 1-naphthyloxy group and 2-naphthyloxy group.

The “C₇₋₂₂ aralkyloxy group” used in the specification of the present application means a group corresponding to the above-defined “C₇₋₂₂ aralkyl group” to which end an oxygen atom is bonded. Specific examples thereof include benzyloxy group, phenethyloxy group, 3-phenylpropyloxy group, 4-phenylbutyloxy group, 1-naphthylmethyloxy group and 2-naphthylmethyloxy group; and preferred examples include benzyloxy group.

The “5- to 14-membered heteroaralkyloxy group” used in the specification of the present application means a group corresponding to the above-defined “5- to 14-membered heteroaralkyl group” to which end an oxygen atom is bonded. Specific examples thereof include thienylmethyloxy group, furylmethyloxy group, pyridylmethyloxy group, pyridazylmethyloxy group, pyrimidylmethyloxy group and pyrazylmethyloxy group; and preferred examples include thienylmethyloxy group, furylmethyloxy group and pyridinylmethyloxy group.

The “5- to 14-membered heteroaryloxy group” used in the specification of the present application means a group corresponding to the above-defined “5- to 14-membered heteroaryl group” to which end an oxygen atom is bonded. Specific examples thereof include pyrrolyloxy group, pyridyloxy group, pyridazinyloxy group, pyrimidinyloxy group, pyrazinyloxy group, triazolyloxy group, tetrazolyloxy group, benzotriazolyloxy group, pyrazolyloxy group, imidazolyloxy group, benzimidazolyloxy group, indolyloxy group, isoindolyloxy group, indolizinyloxy group, purinyloxy group, indazolyloxy group, quinolyloxy group, isoquinolyloxy group, quinolizyloxy group, phthalazyloxy group, naphthyridinyloxy group, quinoxalyloxy group, quinazolinyloxy group, cinnolinyloxy group, pteridinyloxy group, imidazotriazinyloxy group, pyrazinopyridazinyloxy group, acridinyloxy group, phenanthridinyloxy group, carbazolyloxy group, carbazolinyloxy group, perimidinyloxy group, phenanthrolinyloxy group, phenazinyloxy group, imidazopyridinyloxy group, imidazopyrimidinyloxy group, pyrazolopyridinyloxy group, pyrazolopyridinyloxy group, thienyloxy group, benzothienyloxy group, furyloxy group, pyranyloxy group, cyclopentapyranyloxy group, benzofuryloxy group, isobenzofuryloxy group, thiazolyloxy group, isothiazolyloxy group, benzothiazolyloxy group, benzothiadiazolyloxy group, phenothiazinyloxy group, isoxazoyloxy group, furazanyloxy group, phenoxazinyloxy group, oxazolyloxy group, isoxazolyloxy group, benzoxazolyloxy group, oxadiazolyloxy group, pyrazolooxazolyloxy group, imidazothiazolyloxy group, thienofuranyloxy group, furopyrrolyloxy group and pyridoxazinyloxy group; and preferred examples include thienyloxy group, pyridyloxy group, pyrimidyloxy group and pyrazyloxy group.

The “aliphatic C₂₋₂₂ acyl group” used in the specification of the present application means a group corresponding to the above-defined “C₁₋₂₂ alkyl group” or “unsaturated C₂₋₂₂ alkyl group” to which end a carbonyl group is bonded. Examples thereof include acetyl group, propionyl group, butyryl group, iso-butyryl group, valeryl group, iso-valeryl group, pivaloyl group, caproyl group, decanoyl group, lauroyl group, myristoyl group, palmitoyl group, stearoyl group, arachidoyl group, acryloyl group, propiol group, crotonyl group, iso-crotonyl group, olenoyl group and linolenoyl group; and preferred examples include aliphatic acyl groups having 2 to 6 carbon atoms, such as acetyl group, propionyl group, butyryl group, iso-butyryl group and acryloyl group.

The “aromatic C₇₋₁₅ acyl group” used in the specification of the present application means a group corresponding to the above-defined “C₆₋₁₄ aryl group” or “5- to 14-membered heteroaryl group” to which end a carbonyl group is bonded. Examples thereof include benzoyl group, 1-naphthoyl group, 2-naphthoyl group, picolinoyl group, nicotinoyl group, isonicotinoyl group, furoyl group and thiophenecarbonyl group; and preferred examples include benzoyl group, picolinoyl group, nicotinoyl group and isonicotinoyl group.

The “C₁₋₂₂ alkylsulfonyl group” used in the specification of the present application means a group corresponding to the above-defined “C₁₋₂₂ alkyl group” to which a sulfonyl group is bound. Specific examples thereof include methylsulfonyl group, ethylsulfonyl group, n-propylsulfonyl group and iso-propylsulfonyl group; and preferred examples include methylsulfonyl group.

The “C₆₋₁₄ arylsulfonyl group” used in the specification of the present application means a group corresponding to the above-defined “C₆₋₁₄ aryl group” to which a sulfonyl group is bound. Specific examples thereof include benzenesulfonyl group, 1-naphthalenesulfonyl group and 2-naphthalenesulfonyl group; and preferred examples include benzenesulfonyl group.

The “aliphatic C₂₋₂₂ acyloxy group” used in the specification of the present application means a group corresponding to the above-defined “aliphatic C₂₋₂₂ acyl group” to which end an oxygen atom is bonded. Specific examples thereof include acetoxy group, propionyloxy group and acryloxy group; and preferred examples include acetoxy group and propionyloxy group.

The “C₂₋₂₂ alkoxycarbonyl group” used in the specification of the present application means a group corresponding to the above-defined “C₁₋₂₂ alkoxy group” to which end a carbonyl group is bonded. Examples thereof include methoxycarbonyl group, ethoxycarbonyl group, n-propoxycarbonyl group, iso-propoxycarbonyl group, n-butoxycarbonyl group, iso-butoxycarbonyl group, sec-butoxycarbonyl group and tert-butoxycarbonyl group; and preferred examples include ethoxycarbonyl group, iso-propoxycarbonyl group and tert-butoxycarbonyl group.

The “unsaturated C₃₋₂₂ alkoxycarbonyl group” used in the specification of the present application means a group corresponding to the above-defined “unsaturated C₂₋₂₂ alkoxy group” to which end a carbonyl group is bonded. Examples thereof include vinyloxycarbonyl group, allyloxycarbonyl group, 1-propenyloxycarbonyl group, isopropenyloxycarbonyl group, propargyloxycarbonyl group and 2-butynyloxycarbonyl group; and preferred examples include allyloxycarbonyl group.

The “C₁₋₂₂ alkylthio group” used in the specification of the present application means a group corresponding to the above-defined “C₁₋₂₂ alkyl group” to which end a sulfur atom is bonded. Examples thereof include methylthio group, ethylthio group, n-propylthio group and iso-propylthio group; and preferred examples include methylthio group or ethylthio group.

The “C₁₋₂₂ alkylsulfinyl group” used in the specification of the present application means a group corresponding to the above-defined “C₁₋₂₂ alkyl group” to which end a sulfinyl group is bonded. Examples thereof include methylsulfinyl group, ethylsulfinyl group, n-propylsulfinyl group and iso-propylsulfinyl group; and preferred examples include methanesulfinyl group or ethanesulfinyl group.

The “C₁₋₂₂ alkylsulfonyloxy group” used in the specification of the present application means a group corresponding to the above-defined “C₁₋₂₂ alkylsulfonyl group” to which end an oxygen atom is bonded. Examples thereof include methylsulfonyloxy group, ethylsulfonyloxy group, n-propylsulfonyloxy group and iso-propylsulfonyloxy group; and preferred examples include methylsulfonyloxy group.

The substituent of the phrase “an optionally substituted” used in the specification of the present application may be one or more groups selected from:

(1) a halogen atom,
(2) a hydroxyl group,
(3) a thiol group,
(4) a nitro group,
(5) a nitroso group,
(6) a cyano group,
(7) a carboxyl group,
(8) a hydroxysulfonyl group,
(9) a amino group,
(10) a C₁₋₂₂ alkyl group (for example, methyl group, ethyl group, n-propyl group, iso-propyl group, n-butyl group, iso-butyl group, sec-butyl group and tert-butyl group),
(11) an unsaturated C₂₋₂₂ alkyl group (for example, vinyl group, allyl group, 1-propenyl group, 2-propenyl group, isopropenyl group, ethynyl group, 1-propynyl group, 2-propynyl group, 1-butynyl group, 2-butynyl group and 3-butynyl group),
(12) a C₆₋₁₄ aryl group (for example, phenyl group, 1-naphthyl group and 2-naphthyl group),
(13) a 5- to 14-membered heteroaryl group (for example, thienyl group, furyl group, pyridyl group, pyridazyl group, pyrimidyl group and pyrazyl group),
(14) a 3- to 14-membered non-aromatic heterocyclic group (for example, aziridinyl group, azetidyl group, pyrrolidinyl group, pyrrolyl group, piperidinyl group, piperazinyl group, homopiperidinyl group, homopiperazinyl group, imidazolyl group, pyrazolidyl group, imidazolidyl group, morpholyl group, thiomorpholyl group, imidazolinyl group, oxazolinyl group and quinuclidyl group),
(15) a C₃₋₁₄ cycloalkyl group (for example, cyclopropyl group, cyclobutyl group, cyclopentyl group, cyclohexyl group, cycloheptyl group and cyclooctyl group),
(16) a C₁₋₂₂ alkoxy group (for example, methoxy group, ethoxy group, n-propoxy group, iso-propoxy group, sec-propoxy group, n-butoxy group, iso-butoxy group and tert-butoxy group),
(17) an unsaturated C₂₋₂₂ alkoxy group (for example, vinyloxy group, allyloxy group, 1-propenyloxy group, 2-propenyloxy group, isopropenyloxy group, ethynyloxy group, 1-propynyloxy group, 2-propynyloxy group, 1-butynyloxy group and 2-butynyloxy group),
(18) a C₆₋₁₄ aryloxy group (for example, phenyloxy group, 1-naphthyloxy group and 2-naphthyloxy group),
(19) a C₇₋₂₂ aralkyloxy group (for example, benzyloxy group, phenethyloxy group, 3-phenylpropyloxy group, 4-phenylbutyloxy group, 1-naphthylmethyloxy group and 2-naphthylmethyloxy group),
(20) a 5- to 14-membered heteroaralkyloxy group (for example, thienylmethyloxy group, furylmethyloxy group, pyridylmethyloxy group, pyridazylmethyloxy group, pyrimidylmethyloxy group and pyrazylmethyloxy group),
(21) a 5- to 14-membered heteroaryloxy group (for example, thienyloxy group, furyloxy group, pyridyloxy group, pyridazyloxy group, pyrimidyloxy group and pyrazyloxy group),
(22) an aliphatic C₂₋₂₂ acyl group (for example, acetyl group, propionyl group, butyryl group, iso-butyryl group, valeryl group, iso-valeryl group, pivalyl group, caproyl group, decanoyl group, lauroyl group, myristoyl group, palmitoyl group, stearoyl group, arachidoyl group, acryl group, propiol group, crotonyl group, iso-crotonyl group, olenoyl group and linolenoyl group),
(23) an aromatic C₇₋₁₅ acyl group (for example, benzoyl group, 1-naphthoyl group and 2-naphthoyl group),
(24) an aliphatic C₂₋₂₂ acyloxy group (for example, acetoxy group, propionyloxy group and acryloxy group),
(25) a C₂₋₂₂ alkoxycarbonyl group (for example, methoxycarbonyl group, ethoxycarbonyl group, n-propoxycarbonyl group, iso-propoxycarbonyl group, n-butoxycarbonyl group, iso-butoxycarbonyl group, sec-butoxycarbonyl group and tert-butoxycarbonyl group),
(26) an unsaturated C₃₋₂₂ alkoxycarbonyl group (for example, vinyloxycarbonyl group, allyloxycarbonyl group, 1-propenyloxycarbonyl group, 2-propenyloxycarbonyl group, isopropenyloxycarbonyl group, propargyloxycarbonyl group and 2-butynyloxycarbonyl group),
(27) a C₁₋₂₂ alkylthio group (for example, methylthio group, ethylthio group, n-propylthio group and iso-propylthio group),
(28) a C₁₋₂₂ alkylsulfinyl group (for example, methylsulfinyl group, ethylsulfinyl group, n-propylsulfinyl group and iso-propylsulfinyl group),
(29) a C₁₋₂₂ alkylsulfonyl group (for example, methylsulfonyl group, ethylsulfonyl group, n-propanesulfonyl group and iso-propanesulfonyl group),
(30) a C₆₋₁₄ arylsulfonyl group (for example, benzenesulfonyl group, 1-naphthalenesulfonyl group and 2-naphthalenesulfonyl group),
(31) a C₁₋₂₂ alkylsulfonyloxy group (for example, methylsulfonyloxy group, ethylsulfonyloxy group, n-propylsulfonyloxy group and iso-propanesulfonyloxy group),
(32) carbamoyl group, and
(33) formyl group.
Among them, a preferred example is an amino group, a C₁₋₂₂ alkyl group, an unsaturated C₂₋₂₂ alkyl group, a C₆₋₁₄ aryl group, a 5- to 14-membered heteroaryl group, a 3- to 14-membered non-aromatic heterocyclic group and a C₃₋₁₄ cycloalkyl group, and a more preferred example is an amino group, a C₁₋₂₂ alkyl group, a 3- to 14-membered nonaromatic heterocyclic group and a C₃₋₁₄ cycloalkyl group. The above-mentioned (9) amino group and (31) carbamoyl group as the substituent in “an optionally substituted” may each be further substituted with one or two of a C₁₋₂₂ alkyl group, an unsaturated C₂₋₂₂ alkyl group or a C₆₋₁₄ aryl group.

In the present specification, the chemical formula of the compound according to the present invention is illustrated as a planimetric chemical formula for convenience but the compound can include certain isomers drawn from the chemical formula. The present invention can include all isomers and mixtures of the isomers such as a geometric isomer which is generated from the configuration of the compound, an optical isomer based on an asymmetric carbon, a rotamer, a stereoisomer and a tautomer. The present invention is not limited to the expediential description of the chemical formula, and can include either of isomers or a mixture thereof. Accordingly, when the compound according to the present invention has an asymmetric carbon in the molecule, and its optically active substance and racemate exist, any one is included. Further, when polymorphic crystals exist, the crystal form according to the present invention is not specifically limited to one form, and any one of the crystal forms may be single or a mixture of the crystal forms.

The “pharmaceutically acceptable salt” used in the present invention is not particularly restricted as long as it can form a salt with the compound represented by formula (I), and is pharmaceutically acceptable. Preferred examples thereof include halide hydroacid salt such as hydrochloric acid salt, hydrobromic acid salt, hydrolodic acid salt; inorganic acid salt such as sulphic acid salt, nitric acid salt, perchloric acid salt, phosphoric acid salt, carbonic acid salt, bicarbonic acid salt; organic carboxylic acid salt such as acetic acid salt, trifluoroacetic acid salt, maleic acid salt, tartaric acid salt, fumaric acid salt, citric acid salt; organic sulfonic acid salt such as methanesulfonic acid salt, trifluoro methanesulfonic acid salt, ethanesulfonic acid salt, benzenesulfonic acid salt, toluenesulfonic acid salt, camphorsulfonic acid salt; amino acid salt such as aspartic acid salt, glutamic acid salt; quaternary amine salt; alkaline metal salt such as sodium salt, potassium salt; and alkaline earth metal salt such as magnesium salt, calcium salt.

The “solvate” used in the present invention is not particularly restricted as long as it can form a solvate with the compound represented by formula (I) or the salt thereof, and is pharmaceutically acceptable. Preferred examples include hydrate, alcoholate such as ethanolate, and etherate.

The present invention also includes a metabolite generated by degradation of the compound represented by formula (I) within a living body, as well as a prodrug of the compound represented by formula (I) and the salt thereof. The “prodrug” used herein means an inert substance to which “an active moiety of a drug” (meaning “drug” corresponding to a prodrug) has been chemically modified, for the purpose of improvement of bioavailability and reduction of a side effect. After absorbed, it is metabolized into the active moiety in vivo and exerts an action. Accordingly, the term “prodrug” refers to any compound having a lower intrinsic activity than the corresponding “drug”, which is, once administrated to a biological system, converted into the “drug” substance via a spontaneous chemical reaction, enzyme catalyzed reaction or metabolic reaction. Examples of such prodrugs include various prodrugs, for example, compounds produced by acylation, alkylation, phosphorylation, boration, carbonation, esterification, amidation, or urethanation of a functional group such as an amino, hydroxyl, or carboxyl group in the compound represented by formula (I). However, it should be noted that the exemplified prodrugs are not comprehensive but are merely typical, and other conventional various prodrugs can be prepared by a conventional method by a person having ordinary skill in the art from the compound represented by formula (I).

When the compound represented by formula (I) is administrated to a mammal in the method according to the present invention, the compound represented by formula (I) may be formulated by known methods. Conventional carriers are used for formulation, and the pharmaceutical products are prepared by conventional methods. Namely, when a solid formulation for oral use is prepared, a filler is added to the main drug, and if necessary, a binder, a disintegrant, a lubricant, a colorant, a flavoring agent and the like are added thereto, and then tablets, coated tablets, granules, powders, capsules and the like are prepared by conventional methods. It is needless to say that sugar coating, gelatin coating or suitable coating may be conducted on the tablet and granule, if necessary. When the compounds are formulated as an injection, a pH regulator, a buffer, a stabilizer, a solubilizer and the like are added to the main drug, if necessary, to prepare an subcutaneous, intramuscular, intra-articular or intravenous injection according to conventional procedures. When the compound represented by formula (I) is administered as a therapeutic or preventive agent for various diseases, it may be orally administered as tablets, powders, granules, capsules, syrups and the like, and may be parenterally administered as a spray, a suppository, an injection, a topical preparation or an infusion. Although the dose remarkably varies according to the severity of symptom, age, the kind of disease etc., approximately 1 mg to 100 mg per day for an adult is administered in general at one time or several times per day.

When the expression level of pre-mRNA of the genes listed in Table 1, Table 2, Table 3, Table 4, and Table 5 or homologous genes thereof, preferably the genes listed in Table 1 and Table 2 or homologous genes thereof as well as an abnormal protein generated by splicing defect of the gene is monitored in the method according to the present invention, the expression level may be preferably monitored before administration of the compound represented by formula (I), followed by another monitoring at 3, 6, 8, 24, or 48 hours after the administration. In a preferred embodiment, follow-up monitoring of the expression level is carried out at the earliest three hours after the administration of the compound represented by formula (I).

Upon implementing the method according to the present invention the splicing defect can be detected using an increase in the expression level of pre-mRNA or the abnormal protein caused by the splicing defect as an index.

According to a first aspect of the invention, a method for assaying the action of the compound represented by formula (I), using the increase in the expression level of pre-mRNA as an index (invention (3)) is provided.

In step (a), cancer tissue or normal tissue such as hemocytes in peripheral blood, platelets, and serum can be taken from the mammal subjected to the assay and pre-mRNA samples can be prepared from the obtained samples to quantify the expression level of pre-mRNA. Preparation of pre-mRNA is well known (for example, “Molecular Cloning, A Laboratory Manual 3^{d} ed.” (Cold Spring Harbor Press (2001)), and required devices, instruments, and reagents therefor are commercially available. Hence those skilled in the art may prepare pre-mRNA with no difficulties using the devices, apparatuses, and reagents as needed.

Measurement of the expression level of pre-mRNA in step (a) can be performed with any method selected from a Northern blot method, a dot blot method, an RT-PCR method, and a microarray (preferably, Human Exon 1.0 ST Array). The principles of these methods and how to carry out these methods are well-known, and the required devices and apparatuses therefor are commercially available. Moreover, in Examples below, an example in which the expression level of pre-mRNA is measured with these methods will be described. Those skilled in the art can measure the expression level of pre-mRNA with no difficulties using the Northern blot method, the dot blot method, the RT-PCR method, and the microarray. In the measurement of the expression level of pre-mRNA in step (a), preferably, the microarray can be used.

For the measurement of the expression level of pre-mRNA in step (a), a probe and a primer which consist of a polynucleotide capable of hybridizing with nucleotide sequences of genes listed in Table 1, Table 2, Table 3, Table 4, and Table 5 and homologous genes thereof, preferably genes listed in Table 1 and Table 2 or homologous genes thereof, or their complementary sequences can be employed as a detection marker.

Any probe and primer according to the present invention may be employed as long as it can detect the expression of pre-mRNA (including a part thereof) of the genes listed in Table 1, Table 2, Table 3, Table 4, and Table 5 and homologous genes thereof, preferably the genes listed in Table 1 and Table 2 or homologous genes thereof. The probe and the primer according to the present invention refers to a polymer consisting of bases or base pairs such as deoxyribonucleic acid (DNA) or ribonucleic acid (RNA). It has been known that double stranded cDNA can be used in tissue in situ hybridization, and the probe and the primer according to the present invention include such double stranded cDNA. Particularly preferred RNA probe and primer for detecting RNA in tissue include RNA probes (riboprobe).

The probe and the primer according to the present invention includes a probe or a primer which consists of a polynucleotide comprising a nucleotide sequence of at least 10, preferably at least 15, more preferably at least 20, further preferably at least 25 continuous nucleotides of the genes listed in Table 1, Table 2, Table 3, Table 4 and Table 5, and homologous genes thereof, preferably genes listed in Table 1 and Table 2 or homologous genes thereof, or complementary sequences thereof as well as all mutated polynucleotide sequences thereof. The probe and the primer according to the present invention include a probe or a primer which consists of a polynucleotide comprising a nucleotide sequence of 10 to 50 or 10 to 30 continuous nucleotides, 15 to 50 or 15 to 30 continuous nucleotides, 20 to 50 or 20 to 30 continuous nucleotides, 25 to 50 or 25 to 30 continuous nucleotides, of the genes listed in Table 1, Table 2, Table 3, Table 4 and Table 5, and homologous genes thereof, preferably the genes listed in Table 1 and Table 2 or homologous genes thereof or mutated polynucleotide sequences thereof.

The probe and the primer according to the present invention may be at least 10 bases in length, preferably at least 15 bases in length, more preferably at least 20 bases in length, further preferably at least 25 bases in length. The probe and the primer according to the present invention may also be 10 to 50 bases or 10 to 30 bases in length, 15 to 50 bases or 15 to 30 bases in length, 20 to 50 bases or 20 to 30 bases in length, 25 to 50 bases or 25 to 30 bases in length.

According to the preferred embodiment of the probe and the primer according to the present invention, there are provided the probe and the primer having 15 to 30 bases in length for assaying the action of the compound represented by formula (I) to the mammals, which consist of a polynucleotide comprising at least 10, preferably at least 15, more preferably at least 20, further preferably at least 25 continuous nucleotides of a polynucleotide sequence of the genes listed in Table 1, Table 2, Table 3, Table 4, and Table 5 or homologous genes thereof, preferably the genes listed in Table 1 and Table 2 or homologous genes thereof, as well as all mutated sequences thereof, and which are capable of hybridizing with a polynucleotide sequence of the genes listed in Table 1, Table 2, Table 3, Table 4, and Table 5 or homologous genes thereof, preferably the genes listed in Table 1 and Table 2 or homologous genes thereof.

According to a preferred aspect of the probe and the primer according to the present invention, there are provided a probe and a primer which are capable of hybridizing with a more distinct region within a nucleotide sequence of the genes listed in Table 1, Table 2, Table 3, Table 4, and Table 5 or homologous genes thereof, preferably the genes listed in Table 1 and Table 2 or homologous genes thereof. The above probe and primer allow more precise detection of the action of the compound represented by formula (I) to the mammal.

The probe according to the present invention can be chemically synthesized based on the nucleotide sequence of the gene subjected to be detected. Preparation of the probes is well known and can be carried out in accordance with, for example, “Molecular Cloning, A Laboratory Manual 2^{nd} ed.” (Cold Spring Harbor Press (1989)), “Current Protocols in Molecular Biology” (John Wiley & Sons (1987-1997)).

The primers according to the present invention can be used as a primer set comprising of two or more types of the primers.

The primer and the primer set according to the present invention can be used as a primer and a primer set in accordance with a conventional method in a known method for detecting the target gene using a nucleic acid amplification method such as a PCR method, a RT-PCR method, a real-time PCR method, and an in situ PCR method.

The primer set according to the present invention can be selected such that the nucleotide sequence of the target gene can be amplified with the nucleic acid amplification method such as the PCR method. The nucleic acid amplification method is well known and selection of the primer pair therein is obvious for those skilled in the art. For example, in the PCR method, the primers can be selected such that one of two primers (a primer pair) undergoes base pairing with the plus strand of the double stranded DNA of the gene subjected to be detected whereas the other of the primers undergoes base pairing with the minus strand of the double stranded DNA, as well as an extending strand extended with one primer can be paired with the other primer. In a LAMP method (WO00/28082), three regions from the 3′ terminus termed F3c, F2c and F1c, and three regions from the 5′ terminus termed B1, B2 and B3 are respectively defined for the gene subjected to be detected. Four types of primers can be designed using these six regions.

The primer according to the present invention may be chemically synthesized based on the nucleotide sequence of the gene subjected to be detected. Preparation of the primer is well known and can be carried out in accordance with, for example, “Molecular Cloning, A Laboratory Manual 2^{nd} ed.” (Cold Spring Harbor Press (1989)), “Current Protocols in Molecular Biology” (John Wiley & Sons (1987-1997)).

Table 1, Table 2, Table 3, Table 4, and Table 5 describe information specifying the genes and homologous genes thereof listed in these tables. Accordingly those skilled in the art can obtain information on the nucleotide sequence of the subject gene to be detected based on the information described in Table 1, Table 2, Table 3, Table 4, and Table 5 to design the probe and primer based thereon.

In addition, the genes listed in Table 1, Table 2, Table 3, Table 4, and Table 5 are known genes and probes and primers for detecting them are commercially available individually or as a detection kit or detection array.

The term “to hybridize” used in the specification of the present application means to hybridize with a target polynucleotide under stringent conditions. A specific example of the polynucleotide which hybridizes under stringent conditions includes a polynucleotide having at least 70% or more, preferably 80% or more, more preferably 85% or more, further preferably 90% or more, further more preferably 95% or more, particularly preferably 98% or more, and most preferably 99% or more homology to the target polynucleotide when the homology is calculated by a homology search software, such as FASTA, BLAST, Smith-Waterman (Meth. Enzym., 164, 765, (1988)), using default parameters. Further, hybridization “under stringent conditions” can be performed, for example, by a method of carrying out the reaction at a temperature of 40° C. to 70° C., preferably at a temperature of 60° C. to 65° C., in a hybridization buffer solution generally used by those skilled in the art, and carrying out washing in a washing solution at a salt concentration of 15 to 300 mmol/L, preferably at 15 to 60 mmol/L. The temperature and salt concentration can be appropriately adjusted depending on the length of the probe to be used. Further the hybridized product can be washed under conditions in 0.2× or 2×SSC and 0.1% SDS at a temperature of 20° C. to 68° C. Whether stringent (high stringency) or mild (low stringency) conditions is used depends on a difference in salt concentrations and temperatures while the washing process. In cases where the difference in hybridizing depends on the salt concentration, the washing process can be carried out in 0.2×SSC and 0.1% SDS as a stringent washing buffer (high stringency wash buffer) and 2×SSC and 0.1% SDS as a mild washing buffer (low stringency wash buffer). Also, in cases where the difference in hybridizing depends on the temperature, the washing process may be carried out at 68° C. for stringent conditions, at 42° C. for medium (moderate stringency) conditions, or at room temperature (20-25° C.) for mild conditions, but 0.2×SSC and 0.1% SDS are used in all the cases.

When pre-hybridization is carried out, it is carried out under the same condition as in hybridization, but pre (preliminary)-washing is not necessarily carried out under the same condition as in hybridization.

The “homologous gene” used in the specification of the present application means a gene encoding a protein functionally equivalent to a protein encoded by a certain gene. Whether it is “functionally equivalent” or not can be determined by evaluating if the protein has functions equivalent to biological phenomena or functions related to the expression of the gene. Such a gene encoding the functionally equivalent protein includes not only the so-called homologous gene but also a gene with polymorphism and a gene having a mutation without affecting the function.

Examples of the homologous gene include genes which have a nucleotide sequence of a certain gene wherein one or more (preferably one to several, or 1, 2, 3 or 4) nucleotides are inserted, substituted or deleted, or added to one or both termini, and which encode the functionally equivalent protein.

Examples of the homologous gene also include genes which encode an amino acid sequence encoded by a certain gene wherein one or more amino acids are inserted, substituted, or deleted, or added to one or both termini (modified amino acid sequence), and which encode the functionally equivalent protein.

“One or more amino acids are inserted, substituted, or deleted, or added to one or both termini” used in the specification of the present application means that a modification is made by a known technical method such as a site specific mutagenesis method or by substitution of several amino acids as in naturally occurring mutation.

The “modified amino acid sequence” used in the specification of the present application can be an amino acid sequence wherein, for example, 1 to 30 amino acids, preferably 1 to 20 amino acids, more preferably 1 to 9 amino acids, further preferably 1 to 5 amino acids, particularly preferably 1 to 2 amino acids have been inserted, substituted, or deleted, or added to one or both termini. Preferably, the modified amino acid sequence may be an amino acid sequence having one or more (preferably one or several or 1, 2, 3, or 4) conservative substitutions.

The term “conservative substitution” is used herein to mean that one or more amino acid residues are substituted with other chemically similar amino acid residues, so as not to substantially modify the functions of a protein. Examples of such conservative substitution include a case where a certain hydrophobic residue is substituted with another hydrophobic residue and a case where a certain polar residue is substituted with another polar residue having the same electric charge. Such functionally similar amino acids that can be used in such substitution are known as every amino acid types in the present technical field. Specific examples of a nonpolar (hydrophobic) amino acid include alanine, valine, isoleucine, leucine, proline, tryptophan, phenylalanine, and methionine. Examples of a polar (neutral) amino acid include glycine, serine, threonine, tyrosine, glutamine, asparagine, and cysteine. Examples of a (basic) amino acid having a positive charge include arginine, histidine, and lysine. Examples of an (acidic) amino acid having a negative charge include aspartic acid and glutamic acid.

In cases where the first aspect of the present invention is carried out using a cancer tissue and its surrounding tissue as assay samples, the action of the compound represented by formula (I) can be assayed preferably by using the expression level of pre-mRNA of the genes listed in Table 1 and Table 3 or homologous genes thereof as an index. Measurement of the expression level of the pre-mRNA in cases where the cancer tissue and the surrounding tissue are used as the samples, the microarray can be preferably used.

In cases where the first aspect of the present invention is carried out using a normal tissue, in particular peripheral blood and whole blood as the assay samples, the action of the compound represented by formula (I) can be assayed preferably by using the expression level of pre-mRNA of the genes listed in Table 2, Table 4, and Table 5, or homologous genes thereof as an index. Measurement of the expression level of the pre-mRNA in cases where the peripheral blood and whole blood are used as the samples, the microarray can be preferably used.

According to a second aspect of the present invention, a method for assaying the action of the compound represented by formula (I), using an increase in the expression level of an abnormal protein expressed resulting from splicing defect of pre mRNA (invention (11)) is provided.

The abnormal protein measured in step (f) is an abnormal protein resulting from splicing defect of the genes listed Table 1, Table 2, Table 3, Table 4, and Table 5, or homologous genes thereof. Table 1, Table 2, Table 3, Table 4, and Table 5 describe information specifying the genes listed in these tables and homologous genes thereof.

In step (f), samples are taken from a cancer tissue or normal tissue such as hemocytes in peripheral blood, plasma, and serum from a mammal subjected to the assay. Measurement of the expression level of the abnormal protein may be carried out with the collected samples as they are or a protein extracted therefrom. Extraction of the protein is well known (for example, Campa, M. J. et al. Cancer Res. 63, 1652-1656, 2003), and devices, instruments, and reagents necessary for carrying out the extraction are commercially available. Hence those skilled in the art may extract the protein with no difficulties using such the commercially available devices, apparatuses, and reagents as needed.

The measurement of the expression level of the abnormal protein in step (f) can be carried out with a method selected from a fluorescent antibody method, an enzyme immunoassay (ELISA) method, a radioimmunoassay (RIA) method, a Western blot method and an immunostaining (immunohistochemistry) method. The principle and implementation procedures of these methods are well known and devices and instruments necessary for carrying out the methods are commercially available. Furthermore, in Examples below, an example in which the expression level of pre-mRNA is measured with those methods will be described. Those skilled in the art may measure the expression level of pre-mRNA with no difficulties using the fluorescent antibody method, the enzyme immunoassay (ELISA) method, the radioimmunoassay (RIA) method, the Western blot method and the immunostaining (immunohistochemistry) method. In the measurement of the expression level of the abnormal protein in step (f), the enzyme immunoassay (ELISA) method, the Western blot method, the immunostaining (immunohistochemistry) method and a mass spectrometry method can be used.

In the measurement of the abnormal protein in step (f), an antibody against the abnormal protein generated from the splicing defect of the genes listed Table 1, Table 2, Table 3, Table 4, and Table 5 and a fragment thereof can be used as a detection marker.

Any abnormal protein may be employed for obtaining the antibody according to the present invention as long as it has antigenicity. A protein having an amino acid sequence of the abnormal protein wherein one or more amino acids are deleted, inserted, substituted or added can be used as the antigen for the abnormal protein. It is known that such a protein maintains the same biological activity as the original protein (Mark et al. (1984) Proc. Natl. Acad. Sci. USA 81:5662-6; Zoller and Smith (1982) Nucleic Acids Res. 10:6487-500; Wang et al. (1984) Science 224:1431-3; Dalbadie-McFarland et al. (1982) Proc. Natl. Acad. Sci. USA 79:6409-13). A technique to delete, insert, substitute or add one or more amino acids while maintaining the antigenicity of the original protein is known. For example, such a protein may be obtained by preparing and properly expressing a polynucleotide encoding an abnormal protein by site directed mutagenesis technique (Molecular Cloning, A Laboratory Manual 2nd ed., Cold Spring Harbor Press (1989); Current Protocols in Molecular Biology, John Wiley & Sons, (1987-1997), Section 8.1-8.5; Hashimoto-Goto et al. (1995) Gene 152:271-5; Kinkel (1985) Proc. Natl. Acad. Sci. USA 82:488-92; Kramer and Fritz (1987) Method. Enzymol. 154:350-67; Kunkel (1988) Method. Enzymol. 85:2763-6).

The antibody according to the present invention includes an antibody having specificity against a part of the abnormal protein. That is, the abnormal protein for obtaining the antibody according to the present invention includes a polypeptide having the full length amino acid sequence of the abnormal protein as well as a fragment thereof having at least six amino acid residues (for example, not less than 6, 8, 10, 12 or 15 amino acid residues). A preferred fragment is a polypeptide fragment such as an amino terminus and a carboxyl terminus of the abnormal protein. An antigen determination site of the polypeptide can be predicted by a method analyzing the hydrophobicity/hydrophilicity of the amino acid sequence of the protein (Kyte-Doolittle (1982) J. Mol. Biol. 157:105-22), and a method analyzing a secondary structure (Chou-Fasman (1978) Ann. Rev. Biochem. 47:251-76) and further confirmed by a computer program (Anal. Biochem. 151:540-6 (1985)) or a technique such as PEPSCAN analysis (patent application publication JP60500684T) involving the synthesis of a short peptide to confirm the antigenicity.

Table 1, Table 2, Table 3, Table 4 and Table 5 describe information specifying the genes listed in these Tables and homologous genes thereof. Accordingly, those skilled in the art can obtain information on an amino acid encoded by the subject gene to be detected based on the information described in Table 1, Table 2, Table 3, Table 4 and Table 5, and can design and obtain an antibody based thereon.

In addition, the genes listed in Table 1, Table 2, Table 3, Table 4 and Table 5 are known genes and an antibody for detecting a protein encoded thereby is commercially available individually or as a detection kit or detection array.

The antibody according to the present invention may be obtained with a method known those skilled in the art (for example, “Current Protocols in Molecular Biology” (John Wiley & Sons (1987), Antibodies: A Laboratory Manual, Ed. Harlow and David Lane, Cold Spring Harbor Laboratory (1988)).

The antibody according to the present invention includes a polyclonal antibody, a monoclonal antibody, a chimeric antibody, a single chain antibody (scFv), a humanized antibody and a multispecific antibody. Also, the fragment of the antibody according to the present invention includes an antibody fragment such as Fab, Fab′, F(ab′)₂, Fc, and Fv.

For a polyclonal antibody, blood can be taken from a mammal sensitized with an antigen and blood serum can be isolated with known procedures from the blood to yield blood serum containing the polyclonal antibody. As needed, a fraction containing the polyclonal antibody can further be isolated from this blood serum.

For a monoclonal antibody, antibody-producing cells are taken from spleen or lympho node of a mammal sensitized with the above-mentioned antigen, and then undergo cell fusion with myeloma cell. The resultant hybridoma is subjected to cloning and the antibody was recovered from the culture thereof to yield the monoclonal antibody.

A fragment of the abnormal protein can be used as an immunogen. Alternatively, the synthesized one based on the amino acid sequence of the abnormal protein can be used. The antigen can be used as a complex with a carrier protein. A variety of condensing agents can be used for preparation of the complex between the antigen and the carrier protein, which condensing agents include glutaraldehyde, carbodiimide, and maleimide active ester. The carrier protein may be a usually used one such as bovine serum albumin, thyroglobulin, and hemocyanin. A procedure for coupling at a rate (volume) of 1 time to 5 times is usually employed.

Examples of the animal immunized include mice, rats, rabbits, guinea pigs, hamsters. An example of a method of inoculation is subcutaneous, intramuscular or intraperitoneal administration. The administration may be done in combination with Freund's complete adjuvant and Freund's incomplete adjuvant, and usually once every two to five weeks.

The antibody-producing cells obtained from the spleen or lymph node of the animal immunized undergo cell fusion with myeloma cells, and is isolated as hybridoma. As the myeloma cells, cells derived from mouse, rat, Homo sapiens and etc. are used. It is preferred that antibody-producing cell be derived from the same species. Yet there are cases where the cell fusion can be carried out between different species.

Procedures for the cell fusion may be carried out with a known method, in accordance with, for example, Nature, 256, 495, 1975. Examples of fusion accelerator include polyethylene glycols and Sendai virus. The cell fusion can be usually carried out by using about 20 to 50% of concentration of polyethylene glycols (average molecular weight 1000 to 4000); at a temperature of 20 to 40° C., preferably 30 to 37° C.; at a ratio in number of cells between antibody production cells and myeloma of usually about 1:1 to 10:1, and for about 1 to 10 minutes.

Various immunochemical methods can be employed for screening the antibody-producing hybridoma. Examples thereof include ELISA method using a microtiter plate coated with the abnormal protein, EIA method using a microtiter plate coated with an anti-immunoglobulin antibody, immune blot method using a nitrocellulose blotting membrane after electrophoresis of samples containing the abnormal protein.

Using such wells, cloning by, for example, a limiting dilution method can be further carried out to obtain a clone. Selection and breeding of the hybridoma is usually carried out culture medium for mammalian cells (such as RPMI1640) containing 10˜20% bovine fetus serum and supplemented with HAT (hypoxanthine, aminopterin, and thymidine). The clone obtained in such a way is intraperitoneally transplanted into a SCID mouse previously administrated with pristine. Ten to fourteen days later, ascites containing the monoclonal antibody at a high concentration is obtained, which ascites can be used as a raw material for antibody purification. Also the clone may be cultured and the obtained culture may be used as a raw material for antibody purification

Any purification method may be used for purifying the monoclonal antibody as long as it is a known method for purifying an immunoglobulin. The purification can be readily accomplished by, for example, an ammonium sulfate fractionation method, a PEG fractionation method, an ethanol fractionation method, and use of an anion exchanger, as well as means such as affinity chromatography using the abnormal protein.

Purification of the polyclonal antibody from serum can be carried out in the same manner.

In cases where the procedure in the second aspect according to the present invention is carried out by using the cancer tissue and its surrounding tissue as assay samples, the action of the compound represented by formula (I) can preferably be assayed by using the expression level of the abnormal protein(s) expressed resulting from splicing defect of the genes listed in Table 1 and Table 3 or homologous genes thereof as an index. In cases where the cancer tissue and the surrounding tissue are used as the samples, the measurement of the expression level of the abnormal proteins can preferably be employed with the enzyme immunoassay (ELISA) method, the Western blot technique, the immunostaining (immunohistochemistry) method and the mass spectrometry method.

In cases where the procedure in the second aspect of the present invention using peripheral blood or whole blood as assay samples, the action of the compound represented by formula (I) can preferably be assayed by using the expression level of the abnormal protein(s) expressed resulting from splicing defect of the genes listed in Table 2, Table 4, and Table 5 or homologous genes thereof as an index. In cases where peripheral blood or whole blood are used as the samples, the measurement of the expression level of the abnormal proteins can preferably be employed with the enzyme immunoassay (ELISA) method, the Western blot method, the immunostaining (immunohistochemistry) method and the mass spectrometry method.

The samples obtained from a subject refer to tissues, cells, body fluids, and the like which are obtained from the subject. Specific examples include biopsy, blood (including hemocytes, plasma, and serum), urine, tissue samples such as curettage tissue (buccal scrapes) of oral cavity, and tumor cells (cells from tumors of breast, lung, stomach, head and neck, colorectum, kidney, pancreas, uterus, liver, urinary bladder, endometrium, and prostate, as well as hemocytes of leukemia patients or of lymphocytes).

### EXAMPLES

The present invention is described in more detail by the examples below. The followings are illustrative of the invention and by no means intended to limit the invention to the embodiments described herein.

### Example 1

### Analysis with Northern Blotting

Gene expression that affects the cell cycle was examined by Northern blotting analysis using RNA from HeLa cells treated with pladienolide B. As a result, it was discovered that CDKN1B (p27) gene caused pre-mRNA accumulation.

HeLa cells (5×10⁵ cells/mL) were first seeded in a six-well plate and cultured overnight in RPMI1640 medium (containing 10% FCS, penicillin, and streptomycin). The cells were then treated with 10 μM of pladienolide B, 100 μM of DRB, or 1 μg/mL of Actinomycin D for 0, 1, 2, and 4 hours. Subsequently, total RNA was obtained using RNeay mini kit (Qiagen) and absorbance at 260 nm was measured to quantify an amount of RNA. Each total RNA sample (10 μg) was, after combined with RNA SAMPLE LOADING BUFFER (SIGMA), subjected to electrophoresis in 1% denatured agarose gel containing formaldehyde, followed by blotting to nylon membrane and cross-linking with UV to make a Northern membrane.

In order to make a probe for CDKN1B(p27) and CDKN1A(p21), the entire length of gene was amplified by PCR with the following primers using cDNA reverse-transcribed from total RNA prepared from U251 cells as a template.

| | |
|---|---|
| Primers for CDKN1B | |
| P27-1F: | |
| 5′-ATGTCAAACGTGCGAGTGTCTAAC-3′ | (SEQ ID NO: 1) |
| P27-2: | |
| 5′-TTACGTTTGACGTGTTGTGAGGCC-3′ | (SEQ ID NO: 2) |
| Primers for CDKN1A | |
| P21-1F: | |
| 5′-GCCATGTCAGAACCGGCTGGGGAT-3′ | (SEQ ID NO: 3) |
| P21-2: | |
| 5′-TTAGGGCTTCCTCTTGGAGAAGAT-3′ | (SEQ ID NO: 4) |

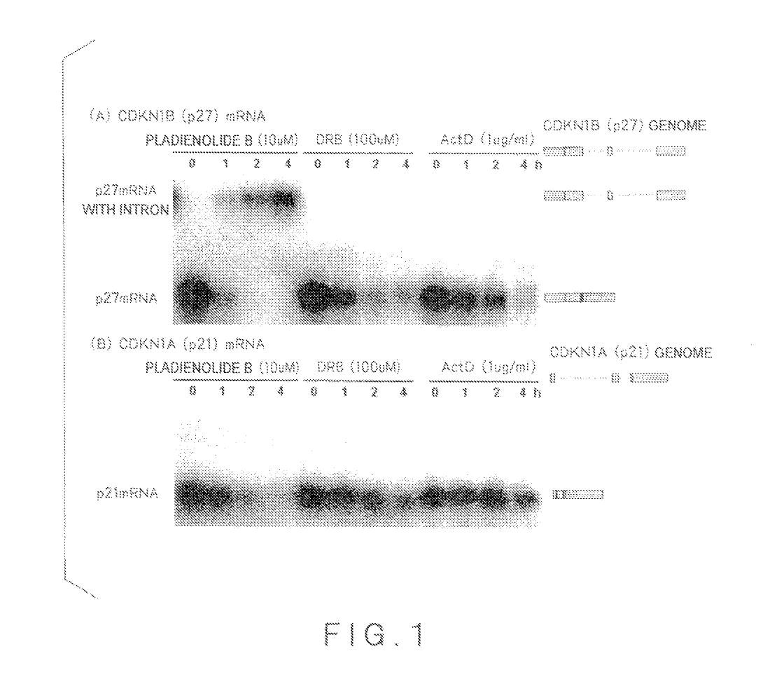

The obtained fragment was separated by electrophoresis in agarose gel and purified. The fragment was labeled using Megaprime DNA labeling kit (Amersham) and Redivue ³²P (Amersham) and purified with ProbeQuant G-50 Micro Columns (Amersham). Using PerfectHyb hybridization solution (TOYOBO), the labeled probe was hybridized onto the blotting membrane. The membrane was washed with a buffer containing 2×SSC and 0.05% SDS, followed by a buffer containing 0.1×SSC and 0.1% SDS. The membrane was exposed to Imaging Plate (FUJIFILM) to determine intensity of photosensitivity with BAS2000 (FUJIFILM). Accumulation of CDKN1B pre-mRNA was observed only when the cells were treated with pladienolide (FIGS. 1A and B).

### Example 2

### Analysis with cDNA Library

In order to check if any other gene accumulated its pre-mRNA, a cDNA library was constructed from HeLa cells treated with pladienolide and by randomly sequencing 42 clones, genes containing an intron were screened. Primers were designed within the introns surrounding an exon of those genes. RT-PCR was performed for the total RNA of HeLa cells treated with pladienolide. DNAJB1, BZW1, SPAG5, RIOK3, NUP54, and BRD2 were identified as pre-mRNA-accumulating genes.

Semi-confluent HeLa cells cultured in a 10 cm dish in DMEM high glucose medium (containing 10% FCS, penicillin, and streptomycin) were treated with 10 nM, 100 nM, and 10 μM of pladienolide B for 4 hours and 6 mL of TRIzol (Invitrogen) was added thereto so that the cells were dissolved. Total RNA was obtained in accordance with the protocol of TRIzol and absorbance at 260 nm was measured to quantify an amount of RNA.

Subsequently, mRNA was purified from total RNA collected from HeLa cells treated with 10 nM, 100 nM, 10μ of pladienolide B and without the treatment, using μMACS mRNA Isolation Kit (Miltenyi Biotec). mRNA was concentrated by ethanol precipitation and absorbance at 260 nm was measured to quantify an amount of RNA. For 1 μg of mRNA, single stranded cDNA synthesis, double stranded cDNA synthesis, adaptor ligation in order was carried out using SuperScript Plasmid System for cDNA Synthesis and Cloning (Invitrogen), and the resultant DNA fragment was ligated to pCLex, which is a retrovirus vector. The resultant vector construct was then transformed into XL10-Gold ultracompetent cells (STRATAGENE). The transformed cells were seeded on LB plates containing ampicillin and cultured overnight. From each plate of the sample treated with 10 nM, 100 nM, and 10 μM of pladienolide B and without the treatment, 42 clones were separately picked up. A total of 42 clones was individually cultured and the plasmid was purified.

A nucleotide sequence of the genes inserted in the plasmid of the 42 clones extracted from the cDNA library of the sample treated with 10 nM, 100 nM, and 10 μM of pladienolide B and without the treatment was obtained using BigDye Terminator (Applied Biosystems) and senseXIY primer (sequence: CCTCGATCCTCCCTTTATCCAGCCCTCACT) (SEQ ID NO: 5) with ABI PRISM3130 (Applied Biosystems). The obtained sequence was then compared with genome information with UCSC/BLAT to collect genes containing a sequence of an intron region. For the genes containing the sequence of the intron region, the following primers were designed within both-sided exon regions surrounding the intron region.

DNAJB1-FW:

(SEQ ID NO: 6)

5′-GAACCAAAATCACTTTCCCCAAGGAAGGAG-3′
DNAJB1-RV:

(SEQ ID NO: 7)

5′-AATGAGGTCCCCACGTTTCTCGGGTGT-3′
BZW1-FW:

(SEQ ID NO: 8)

5′-GCCAATAAGCAAAGTGTTGAACACTTCAC-3′
BZW1-RV:

(SEQ ID NO: 9)

5′-AAGTGCTTGATGGCTTGCTCTGCTAC-3′
SPAG5-FW:

(SEQ ID NO: 10)

5′-ACATGGACAGCTTTGCTGAGTCGGTCC-3′
SPAG5-RV:

(SEQ ID NO: 11)

5′-TTGCTAGACGACTGTTTTCCAACTCCAG-3′
RIOK3-FW:

(SEQ ID NO: 12)

5′-GCTGAAGGACCATTTATTACTGGAG-3′
RIOK3-RV:

(SEQ ID NO: 13)

5′-TTCTTGCTGTGTTCTTTCTCCCACA-3′
NUP54-FW:

(SEQ ID NO: 14)

5′-CTAATCAAACAGGAAATTCAAAGGAAGAG-3′
NUP54-RV:

(SEQ ID NO: 15)

5′-CTTGATTTCTCGTAACAGATCTGCATC-3′
BRD2-FW:

(SEQ ID NO: 16)

5′-ACTCTCAGCAACAACACCAGAGCTCTA-3′
BRD2-RV:

(SEQ ID NO: 17)

5′-TAGCTTTCGTGCCATTGCCACAACATC-3′

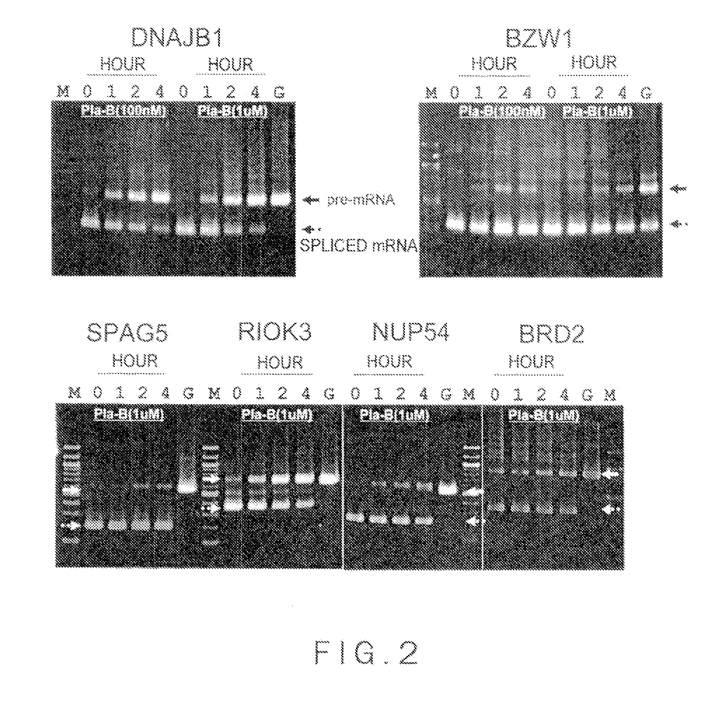

Total RNA was collected from HeLa cells treated with 100 nM or 1 μM of Pladienolide B for 1, 2, or 4 hours or untreated. The total RNA was treated with DNase, and then subjected to reverse transcription reaction to yield cDNA. The obtained cDNA was then subjected to PCR using FastStart HiFi Polymerase (Roche Diagnostics) with the designed primers. Based on an electrophoresis pattern in agarose gel, a decrease in the expression of mature RNA and an increase in the expression of premature mRNA upon the Pladienolide B treatment was confirmed in DNAJB1, BZW1, SPAG5, RIOK3, NUP54, and BRD2 (FIG. 2).

### Example 3

### Analysis with Microarray

Using RNA purified from human colon carcinoma cell strain WiDr treated with 14 nM of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide (also referred to as “E7101”) for six hours, microarray analysis (Human Exon 1.0 ST Array: Affymetrix) was carried out. Not only does this array comprehensively cover the exons for all genes, but it is also designed to have probes for regions estimated as potential exons by a computer prediction program, which enables detection of the expression of the exons and introns of all genes in theory. As a result, genes of which mature mRNA was sufficiently expressed when untreated and of which intron exhibited not less than ten times increase upon the treatment with (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide in at least two probe sets were identified.

WiDr cells were first suspended in RPMI1640 medium (containing 10% FBS, penicillin, and streptomycin) and seeded in a 10 cm dish (2×10⁶ cells/dish). After an overnight culture in an incubator with 5% carbon dioxide gas at 37° C., the medium was changed with medium containing 14 nM of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide or containing vehicle alone. After cultured for additional six hours, TRI reagent (SIGMA) were added to the cells and the cells were harvested. RNA was purified in accordance with the protocol of TRI reagent, followed by further purification with RNeasy (QIAGEN). Absorbance at 260 nm was measured to quantify an amount of RNA. This experiment was repeated three times and RNA samples (n=3) were prepared.

Ribosomal RNA was removed from 1 μg of each total RNA of control cells (n=3) and cells with the treatment (n=3) by using RiboMinus Human/Mouse Transcriptome Isolation Kit (Invitrogen). Single stranded cDNA synthesis, double stranded cDNA synthesis, cRNA synthesis, second single stranded cDNA synthesis, cDNA fragmentation, and cDNA labeling in order were carried out for the total RNA from which ribosomal RNA was removed, by using GeneChip Whole Transcript Sense Target Labeling and Control Reagents (Affymetrix) to make a cDNA probe. The cDNA probe was used for hybridization with Human Exon 1.0 ST Array (Affymetrix). The array was washed and stained, and luminescence intensity was measured by a scanner.

The expression level of the probe set and the gene was quantified by using Expression Console Ver. 1.0 (Affymetrix) with Summarization Method and Normalization Method being set to “Median polish as used in RMA” and “None”, respectively. The expression level of the probe was normalized with that of the gene. Welch's t-test was conducted for the treated group and the control group to determine a p value, which was converted into a q value by controlling False Discovery Ratio to pick out a significant probe set.

Probe sets showing that the q value was less than 1%; the normalized change in the level expression of the probe set in the group with the treatment was more than 10 times; the expression level of the probe set and the gene in the group with the treatment was more than 100; and probe set annotation was “extended, full, free”, were picked out and further arranged by gene. A gene was, when more than one probe sets therefor were found, determined as a candidate gene with increased expression level of the intron regions (Table 1). In Table 1, for the gene evaluated, gene name (Gene Name), abbreviated name (Gene Symbol), accession number (Accession), alias name (Synonym), transcription number in Human Exon 1.0 ST Array (Human Exon 1.0 ST Array), chromosome number (Chromosome), type of strand (Strand), start region (Start), stop region (Stop), and change in expression level (Fold Change) were respectively shown.

TABLE 1

| | | | | Human Exon 1.0 ST | Human Genome hg18 | | | | |
|---|---|---|---|---|---|---|---|---|---|
| Gene Name | Gene Symbol | Accession | Synonym | Array | Chromosome | Strand | Start | Stop | Fold Change |
| sprouty-related, EVH1 | SPRED2 | NM_181784 | FLJ21897 // FLJ31917 // | 2556752 | chr2 | − | 65391279 | 65513247 | 31.56 |
| domain containing 2 | | | MGC163164 // Spred-2 | | | | | | |
| WD repeat domain 73 | WDR73 | NM_032856 | FLJ14888 // HSPC264 | 3636956 | chr15 | − | 82987018 | 82998719 | 30.61 |
| muscleblind-like 2 | MBNL2 | NM_144778 // NM_207304 | DKFZp781H1296 // MBLL | 3497586 | chr13 | + | 96672563 | 96844371 | 28.86 |
| (Drosophila) | | | // MBLL39 // MGC120625 | | | | | | |
| | | | // MGC120626 // | | | | | | |
| | | | MGC120628 // PRO2032 | | | | | | |
| brix domain containing 2 | BXDC2 | NM_018321 | BRIX // FLJ11100 | 2806231 | chr5 | + | 34951248 | 34962845 | 25.94 |
| development and | DDEF2 | NM_003887 | AMAP2 // FLJ42910 // | 2468811 | chr2 | + | 9264345 | 9463243 | 22.92 |
| differentiation enhancing | | | KIAA0400 // PAG3 // PAP | | | | | | |
| factor 2 | | | // Pap-alpha // SHAG1 | | | | | | |
| integrin, beta 1 (fibronectin | ITGB1 | NM_002211 // NM_033667 | CD29 // FNRB // GPIIA // | 3284186 | chr10 | − | 33211051 | 33321367 | 21.48 |
| receptor, beta polypeptide, | | // NM_033668 // | MDF2 // MSK12 // VLAB | | | | | | |
| antigen CD29 includes | | NM_033669 | | | | | | | |
| MDF2, MSK12) | | | | | | | | | |
| eukaryotic translation | EIF2C2 | NM_012154 | AGO2 // MGC3183 // Q10 | 3156193 | chr8 | − | 141599440 | 141726037 | 21.33 |
| initiation factor 2C, 2 | | | | | | | | | |
| chromosome 10 open | C10orf18 | XM_374765 | DKFZp781E1986 // | 3233322 | chr10 | + | 5766851 | 5846629 | 19.86 |
| reading frame 18 | | | FLJ20360 // bA318E3.2 | | | | | | |
| KIAA0174 | KIAA0174 | NM_014761 | MGC117220 | 3667766 | chr16 | + | 70481983 | 70522196 | 19.65 |
| discoidin, CUB and LCCL | DCBLD2 | NM_080927 | CLCP1 // ESDN | 2686023 | chr3 | − | 99997526 | 100124598 | 19.52 |
| domain containing 2 | | | | | | | | | |
| ubiquitin protein ligase E3A | UBE3A | NM_000462 // NM_130838 | ANCR // AS // E8-AP // | 3614087 | chr15 | − | 23006655 | 23355108 | 19.35 |
| (human papilloma virus E8- | | // NM_130839 | EPVE6AP // FLJ26981 // | | | | | | |
| associated protein, | | | HPVE8A | | | | | | |
| Angelman syndrome) | | | | | | | | | |
| isopentenyl-diphosphate | IDI1 | NM_004508 | IPP1 // IPPI1 | 3273601 | chr10 | − | 1075869 | 1092634 | 19.25 |
| delta isomerase 1 | | | | | | | | | |
| chromosome 6 open reading | C6orf166 | NM_018064 | FLJ10342 // dJ486L4.2 | 2963784 | chr6 | − | 88441297 | 88476721 | 19.23 |
| frame 166 | | | | | | | | | |
| pumilio homolog 2 | PUM2 | NM_015317 | FLJ36528 // KIAA0235 // | 2542816 | chr2 | − | 20291440 | 20450197 | 19.01 |
| (Drosophila) | | | MGC138251 // MGC136253 | | | | | | |
| | | | // PUMH2 // PUML2 | | | | | | |
| suppressor of var1, 3-like 1 | SUPV3L1 | NM_003171 | SUV3 | 3250204 | chr10 | + | 70609946 | 70638996 | 18.81 |
| (S. cerevisiae) | | | | | | | | | |
| forkhead box K2 | FOXK2 | NM_004514 | ILF // ILF-1 // ILF1 | 3738969 | chr17 | + | 78070883 | 78195807 | 18.79 |
| WW domain containing | WAC | NM_016628 // NM_100264 | BM-016 // MGC10753 // | 3240340 | chr10 | + | 28828043 | 29005095 | 18.53 |
| adaptor with coiled-coil | | // NM_100486 | PRO1741 // Wwp4 // | | | | | | |
| | | | bA48B24 // bA48B24.1 | | | | | | |
| LTV1 homolog (S. cerevisiae) | LTV1 | NM_032860 | C6orf93 // FLJ14909 // | 2929036 | chr6 | + | 144194149 | 144227146 | 18.37 |
| | | | dJ468K18.4 | | | | | | |
| CDC20 cell division cycle | CDC20 | NM_001255 | CDC20A // MGC102824 // | 2333136 | chr1 | + | 43597180 | 43601431 | 18.22 |
| 20 homolog (S. cerevisiae) | | | bA276H19.3 // p55CDC | | | | | | |
| TAR DNA binding protein | TARDBP | NM_007375 | TDP-43 | 2320048 | chr1 | + | 10995025 | 11013170 | 18.13 |
| fetal Alzheimer antigen | BPTF | NM_004459 // NM_182641 | FAC1 // FALZ // NURF301 | 3732448 | chr17 | + | 63252111 | 63410954 | 17.61 |
| villin 2 (ezrin) | VIL2 | NM_003379 | CVIL // CVL // | 2981912 | chr6 | − | 159106770 | 159160432 | 17.50 |
| | | | DKFZp762H157 // | | | | | | |
| | | | FLJ26216 // MGC1584 | | | | | | |
| sorting nexin 19 | SNX19 | NM_014758 | CHET8 // DKFZp667I205 | 3398482 | chr11 | − | 130250320 | 130291615 | 17.12 |
| | | | // KIAA0254 | | | | | | |
| Cdk5 and Abl enzyme | CABLES1 | NM_138375 | FLJ35924 // HsT2563 | 3781531 | chr18 | + | 18940699 | 19101596 | 16.94 |
| substrate 1 | | | | | | | | | |
| SMAD specific E3 ubiquitin | SMURF2 | NM_022739 | DKFZp686F0270 // | 3766980 | chr17 | − | 59968891 | 60088896 | 16.60 |
| protein ligase 2 | | | MGC138150 | | | | | | |
| solute carrier family 2 | SLC2A1 | NM_006516 | GLUT // GLUT1 // | 2409104 | chr1 | − | 43124794 | 43338329 | 16.44 |
| (facilitated glucose | | | MGC141895 // MGC141896 | | | | | | |
| transporter), member 1 | | | | | | | | | |
| v-raf-1 murine leukemia | RAF1 | NM_022490 | CRAF // Raf-1 // c-Raf | 2663244 | chr3 | − | 12600117 | 12680654 | 16.41 |
| viral oncogene homolog 1 | | | | | | | | | |
| chromosome 16 open | C16orf80 | NM_013242 | EVORF // GTL3 | 3693511 | chr16 | − | 56683698 | 56739201 | 16.25 |
| reading frame 80 | | | | | | | | | |
| eukaryotic translation | EIF4A1 | NM_001416 | DDX2A // EIF-4A // EIF4A | 3708826 | chr17 | + | 7416768 | 7423040 | 16.15 |
| initiation factor 4A, isoform 1 | | | | | | | | | |
| testis derived transcript (3 | TES | NM_015641 // NM_152829 | DKFZP586B2022 // | 3020192 | chr7 | + | 115637811 | 115743800 | 15.95 |
| LIM domains) | | | MGC1146 // TESS // | | | | | | |
| | | | TESS-2 // TESTIN | | | | | | |
| ring finger protein 40 | RNF40 | NM_014771 | BRE1B // DKFZp686K191 | 3856555 | chr16 | + | 30681120 | 30695182 | 15.84 |
| | | | // KIAA0661 // MGC13051 | | | | | | |
| | | | // RBP95 // STARING | | | | | | |
| squamous cell carcinoma | SART3 | NM_014706 | KIAA0156 // MGC138188 | 3470253 | chr12 | − | 107440140 | 107479296 | 15.13 |
| antigen recognised by T | | | // RP11-13G14 // TIP110 | | | | | | |
| cells 3 | | | // p110(nrb) | | | | | | |
| heat shock 105 kDa/110 kDa | HSPH1 | NM_006644 | DKFZp686M05240 // | 3508330 | chr13 | − | 30558258 | 30651695 | 15.12 |
| protein 1 | | | HSP105 // HSP105A // | | | | | | |
| | | | HSP105B // KIAA0201 // | | | | | | |
| | | | NY-CO-25 | | | | | | |
| kelch-like 18 (Drosophila) | KLHL18 | NM_025010 | FLJ13703 // KIAA0795 | 2621275 | chr3 | + | 47298888 | 47367590 | 15.00 |
| myosin 1E | MYO1E | NM_004998 | HuncM-1C // MGC104638 | 3626828 | chr15 | − | 57215720 | 57452363 | 14.49 |
| | | | // MYO1C | | | | | | |
| LIM domain 7 | LMO7 | NM_005358 | FBX20 // FBXO20 // | 3494137 | chr13 | + | 75092571 | 75586974 | 14.34 |
| | | | KIAA0858 // LOMP | | | | | | |
| ring finger and SPRY | RSPRY1 | NM_133368 | KIAA1972 | 3662612 | chr16 | + | 55777703 | 55831882 | 14.32 |
| domain containing 1 | | | | | | | | | |
| KIAA1429 | KIAA1429 | NM_015496 // NM_183009 | DKFZP434I116 // | 3145020 | chr8 | − | 95569109 | 95634851 | 14.19 |
| | | | DKFZp781B2117 // | | | | | | |
| | | | MGC136493 // MGC141940 | | | | | | |
| | | | // MSTP054 | | | | | | |
| centromere protein L | CENPL | NM_033319 | C1orf155 // CENP-L // | 2444451 | chr1 | − | 172035313 | 172104622 | 14.13 |
| | | | FLJ31044 // RP3-383J4.1 | | | | | | |
| | | | // dJ383J4.3 | | | | | | |
| Smcy homolog, X-linked | JARID1C | NM_004187 | DXS1272E // MRXJ // | 4009062 | chrX | − | 53159032 | 53271329 | 14.09 |
| (mouse) | | | MRXSJ // SMCX // XE169 | | | | | | |
| guanine nucleotide binding | GNL3 | NM_014366 // NM_206825 | C77032 // E2IG3 // | 2624074 | chr3 | + | 52694976 | 52705212 | 14.08 |
| protein-like 3 (nucleolar) | | | MGC800 // NS | | | | | | |
| RIO kinase 3 (yeast) | RIOK3 | NM_003831 // NM_145906 | DKFZp779L1370 // SUDD | 3781654 | chr18 | + | 19161035 | 19320545 | 13.67 |
| interleukin enhancer binding | ILF2 | NM_004515 | MGC8391 // NF45 // | 2436132 | chr1 | − | 151900372 | 151910103 | 13.79 |
| factor 2, 45 kDa | | | PRO3063 | | | | | | |
| chromosome 9 open reading | C9orf86 // KIAA1984 | NM_024718 // | FLJ10101 // FLJ13045 // | 3194635 | chr9 | + | 138810077 | 138855460 | 13.66 |
| frame 86 // KIAA1984 | | NM_001039374 | Parf // RP11-216L13.9 // | | | | | | |
| | | | bA216L13.9 // pp8875 // | | | | | | |
| | | | MGC15438 // PARF // | | | | | | |
| | | | RP11-216L13.7 // RP11- | | | | | | |
| | | | 216L13.9 // bA216L13.7 | | | | | | |
| adhesion regulating | ADRM1 | NM_007002 | GP110 // MGC29536 // | 3892607 | chr20 | + | 60305485 | 60317305 | 13.51 |
| molecule 1 | | | Rpn13 | | | | | | |
| keratin 18 | KRT18 | NM_000224 | CYK18 // K18 | 3415576 | chr12 | + | 51596769 | 51632949 | 13.41 |
| heat shock 70 kDa protein | HSPA9 | NM_004134 | CSA // GRP75 // HSPA9B | 2877508 | chr5 | − | 137917364 | 137939144 | 13.33 |
| 9B (mortalin-2) | | | // MGC4500 // MOT // | | | | | | |
| | | | MOT2 // MTHSP75 // | | | | | | |
| | | | PBP74 // mot-2 | | | | | | |
| 3-hydroxy-3- | HMGCS1 | NM_002130 | HMGCS // MGC90332 | 2855501 | chr5 | − | 43324231 | 43349337 | 13.21 |
| methylglutaryl-Coenzyme A | | | | | | | | | |
| synthase 1 (soluble) | | | | | | | | | |
| neuroepithelial cell | NET1 | NM_005863 | ARHGEF8 // NET1A | 3233182 | chr10 | + | 5444535 | 5491001 | 13.02 |
| transforming gene 1 | | | | | | | | | |
| SMAD, mothers against | SMAD3 | NM_005902 | DKFZP586N0721 // | 3598959 | chr15 | + | 65145043 | 65296818 | 12.88 |
| DPP homolog 3 (Drosophila) | | | DKFZp686J10186 // | | | | | | |
| | | | HSPC193 // HsT17436 // | | | | | | |
| | | | JV15-2 // MADH3 // | | | | | | |
| | | | MGC60396 // Smad 3 | | | | | | |
| cyclin L2 | CCNL2 | NM_030937 | ANIA-6B // | 4041923 | chr1_random | + | 359569 | 372958 | 12.84 |
| | | | DKFZp761A1210 // | | | | | | |
| | | | DKFZp762O195 // HCLA- | | | | | | |
| | | | ISO // HLA-ISO // PCEE | | | | | | |
| | | | // SB138 | | | | | | |
| ST3 beta-galactoside | ST3GAL1 | NM_003033 | Gal-NAc6S // MGC9183 // | 3154398 | chr8 | − | 134535811 | 134653449 | 12.77 |
| alpha-2,3-sialyltransferase 1 | | | SIAT4A // SIATFL // | | | | | | |
| | | | ST3GalA // ST3GalA.1 // | | | | | | |
| | | | ST3GalIA.1 // ST3O | | | | | | |
| WD repeat domain 74 | WDR74 | NM_018093 | FLJ10439 // FLJ21730 | 3376235 | chr11 | − | 62358627 | 62365857 | 12.70 |
| cyclin D1 | CCND1 | NM_053058 | BCL1 // D11S287E // | 3338192 | chr11 | + | 69161881 | 69178408 | 12.66 |
| | | | PRAD1 // U21B31 | | | | | | |
| nucleoporin like 1 | NUPL1 | NM_014089 // | KIAA0410 // PRO2463 | 3482219 | chr13 | + | 24773258 | 24822202 | 12.63 |
| | | NM_001008564 // | | | | | | | |
| | | NM_001008565 | | | | | | | |
| WDR45-like | WDR45L | NM_019613 | WIPI-3 // WIPI3 | 3775157 | chr17 | − | 78185748 | 78199803 | 12.59 |
| ubiquitin specific peptidase | USP47 | NM_017944 | DKFZp686C13257 // | 3320604 | chr11 | + | 11819556 | 11937446 | 12.32 |
| 47 | | | FLJ20727 // TRFP | | | | | | |
| eukaryotic translation | EIF5 | NM_001969 | EIF-5A | 3553607 | chr14 | + | 102743652 | 102881108 | 12.30 |
| initiation factor 5 | | | | | | | | | |
| methionine | MAT2A | NM_005911 | MATA2 // MATII // | 2491615 | chr2 | + | 85597809 | 85625908 | 12.29 |
| adenosyltransferase II, alpha | | | SAMS2 | | | | | | |
| WEE1 homolog (S. pombe) | WEE1 | NM_003390 | DKFZp686I18166 // | 3319937 | chr11 | + | 9550916 | 9579143 | 12.26 |
| | | | FLJ16446 // WEE1hu | | | | | | |
| GTP binding protein 4 | GTPBP4 | NM_012341 | CRFG // FLJ10686 // | 3231774 | chr10 | + | 942450 | 1055862 | 12.24 |
| | | | FLJ10690 // FLJ39774 // | | | | | | |
| | | | NGB | | | | | | |
| zinc finger protein 384 | ZNF384 | NM_133476 | CAGH1 // CAGH1A // CIZ | 3442205 | chr12 | − | 6645924 | 6668961 | 12.13 |
| | | | // ERDA2 // NMP4 // NP | | | | | | |
| | | | // TNRC1 | | | | | | |
| ATPase, Na+/K+ | ATP1B1 | NM_001677 // | ATP1B // MGC1798 | 2366422 | chr1 | + | 167342204 | 167368946 | 12.11 |
| transporting, beta 1 | | NM_001001787 | | | | | | | |
| polypeptide | | | | | | | | | |
| ADP-ribosylation factor | ARFIP2 | NM_012402 | POR1 | 3360941 | chr11 | − | 6453496 | 6459171 | 12.04 |
| interacting protein 2 | | | | | | | | | |
| (arfaptin 2) | | | | | | | | | |
| zinc finger protein 410 | ZNF410 | NM_021188 | APA-1 // APA1 | 3543884 | chr14 | + | 73423093 | 73468718 | 12.01 |
| DnaJ (Hsp40) homolog, | DNAJB1 | NM_006145 | HSPF1 // Hdj1 // Hsp40 | 3852783 | chr19 | − | 14485622 | 14501114 | 11.95 |
| subfamily B, member 1 | | | | | | | | | |
| guanine nucleotide binding | GNB2L1 | NM_006098 | Gnb2-rs1 // H12.3 // HLC- | 2891052 | chr5 | − | 180596592 | 180612024 | 11.87 |
| protein (G protein), beta | | | 7 // PIG21 // RACK1 | | | | | | |
| polypeptide 2-like 1 | | | | | | | | | |
| Rho GTPase activating | ARHGAP12 | NM_018287 | DKFZp779N2050 // | 3283920 | chr10 | − | 32134245 | 32261226 | 11.60 |
| protein 12 | | | FLJ10971 // FLJ20737 // | | | | | | |
| | | | FLJ21785 // FLJ45709 | | | | | | |
| tight junction protein 1 | TJP1 | NM_003257 // NM_175610 | DKFZp686M05161 // | 3615579 | chr15 | − | 27703228 | 28048334 | 11.55 |
| (zona occludens 1) | | | MGC133289 // ZO-1 | | | | | | |
| dyskeratosis congenita 1, | DKC1 | NM_001363 | DKC // NAP57 // NOLA4 | 3996667 | chrX | + | 153644243 | 153659150 | 11.53 |
| dyskerin | | | // XAP101 // dyskerin | | | | | | |
| BAT2 domain containing 1 | BAT2D1 | NM_015172 | BAT2-iso // XTP2 | 2367154 | chr1 | + | 169716078 | 169829282 | 11.26 |
| TERF1 (TRF1)-interacting | TINF2 | NM_012461 | TIN2 | 3558012 | chr14 | − | 23776465 | 23781950 | 11.05 |
| nuclear factor 2 | | | | | | | | | |
| YTH domain containing 1 | YTHDC1 | NM_001031732 | KIAA1966 // YT521 // | 2772017 | chr4 | − | 68858700 | 68934802 | 10.66 |
| | | | YT521-B | | | | | | |
| adenylate kinase 2 | AK2 | NM_001625 // NM_013411 | ADK2 | 2405364 | chr1 | − | 33245959 | 33275155 | 10.05 |

### Example 4

### Analysis in PBMC

Since it is not easy to obtain a cancer tissue clinically, the measurement of the marker in hemocytes readily obtainable in peripheral blood would be more useful. It was then examined whether the marker gene obtained in cancer cells could change in peripheral blood mononuclear cells in a similar manner as observed in the cancer cells. Specifically peripheral blood was taken from three normal individuals (volunteers) and peripheral blood mononuclear cells were purified and treated with (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide for three hours, followed by measurement of change in expression of the precursor gene (pre-mRNA) by qPCR. In cases where an increase in the expression of pre-mRNA is used as a marker, the genes in Table 2 below, as a representative example, were found to be usable.

### (1) Isolation of Peripheral Blood Mononuclear Cells (PBMC)

Ficoll-Paque PLUS solution (Amersham, 17-1440-02) was slowly added to the blood taken (with heparin added) from healthy individuals to form a layer underneath the blood (15 mL of Ficoll-Paque PLUS solution was added to 25 mL of blood). After the mixture was centrifuged at 1500 rpm for 30 min, the upper part containing platelets was removed and then a layer containing mononuclear cells was transferred to another tube. The cells were suspended in PBS and centrifuged at 1500 rpm for 5 min, followed by removal of the supernatant. After these steps were repeated twice, the PBMC was suspended in RPMI1640 (containing 10% FBS, penicillin, and streptomycin) and the number of the cells was then counted.

### (2) Preparation of Total RNA

PBMC was suspended in RPMI1640 medium (containing 10% FBS, penicillin, and streptomycin) to 5×10⁶ cells/ml and 1 ml of the suspension was plated per well of a 24-well plate. Immediately, 111 μl of 10 times-concentrated (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide was added to the well (six wells per each concentration tested). The cells were then cultured in an incubator with 5% carbon dioxide gas at 37° C. Three hours later, the supernatant was collected and centrifuged at 1500 rpm for five minutes. TRI reagent (SIGMA) (1 ml) was added to each well of the plate from which the supernatant was removed to harvest the cells, which was added to the centrifuged pellet to dissolve. RNA was purified in accordance with the protocol of TRI reagent. RNA was further purified using RNeasy (QIAGEN) and, in accordance with the protocol, DNaseI was added to the samples during the procedure. Absorbance at 260 nm was measured to quantify an amount of RNA.

(3) Measurement of the Expression Level of a Precursor Gene (pre-mRNA)

RNA was prepared to 30 ng/μl and cDNA was synthesized using TaqMan Reverse Transcription Reagents (Applied Biosystems). For the expression level of pre-mRNA of ID1, amplification was carried out by using TaqMan Universal PCR Master Mix (Applied Biosystems) with TaqMan Gene Expression Assays (Hs00704053_s1, Applied Biosystems) as a probe. For the expression level of pre-mRNA of DNAJB1, BZW1, NUP54, RIOK3, CDKN1B, STK17B, ADRM1, EIF4A1, FOXK2, GNB2L1, HSPA9B, HSPH1, KLHL18 and VIL2, reagents were prepared in accordance with each protocol by using POWER SYBR GREEN PCR MASTER MIX (Applied Biosystems) and primers with the sequence below (Invitrogen), and the expression level was measured with ABI7900 (Applied Biosystems). 18S rRNA was measured by both the TaqMan and SYBR methods. The expression level of 18S r RNA was measured with Hs99999901_s1 (Applied Biosystems) as primers for TaqMan and with 18S primers included in TaqMan Ribosomal RNA control reagents (4308329: Applied Biosystems) as primers for SYBR. Measured values were corrected using the expression level of 18S rRNA as an internal control. The expression level of each gene was calculated with the expression level in the cells treated without (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide as 1. Genes of which expression level in PBMC reached about 250% or more were shown in Table 2.

DNAJB1:

(SEQ ID NO: 18)

| | |
|---|---|
| Sense: | 5′-GGCCTGATGGGTCTTATCTATGG-3′ |

(SEQ ID NO: 19)

| | |
|---|---|
| Antisense: | 5′-TTAGATGGAAGCTGGCTCAAGAG-3′ |
| BZW1: | |

(SEQ ID NO: 20)

| | |
|---|---|
| Sense: | 5′-GAACTTTGCCTCATTCTTTTGCA-3′ |

(SEQ ID NO: 21)

| | |
|---|---|
| Antisense: | 5′-CTGAGCTCCAGTCTCTTGTATTTCTG-3′ |
| NUP54: | |

(SEQ ID NO: 22)

| | |
|---|---|
| Sense: | 5′-CAAGGTAACCACTTCTAAGACCATAATTC-3′ |

(SEQ ID NO: 23)

| | |
|---|---|
| Antisense: | 5′-CCTGCTTGAAGATTACATAACTTTTTGT-3′ |
| RIOK3: | |

(SEQ ID NO: 24)

| | |
|---|---|
| Sense: | 5′-TCAATGGAGATAGCAAAGGTATTATAACC-3′ |

(SEQ ID NO: 25)

| | |
|---|---|
| Antisense: | 5′-AGATTTACTTAGGAGCACATTATGAGTGTT-3′ |
| CDKN1B (p27): | |

(SEQ ID NO: 26)

| | |
|---|---|
| Sense: | 5′-ATGTTTATCAACGGTCCGGCT-3′ |

(SEQ ID NO: 27)

| | |
|---|---|
| Antisense: | 5′-CATCCCCAGTGGCTTTTAAGG-3′ |
| STK17B: | |

(SEQ ID NO: 28)

| | |
|---|---|
| Sense: | 5′-TGCGCGGAGACAGCATAG-3′ |

(SEQ ID NO: 29)

| | |
|---|---|
| Antisense: | 5′-TGGGTTCAAAATGCCAGGTT-3′ |
| ADRM1: | |

(SEQ ID NO: 30)

| | |
|---|---|
| Sense: | 5′-GGCCCTTCAGAAATGCTTGTC-3′ |

(SEQ ID NO: 31)

| | |
|---|---|
| Antisense: | 5′-CCCATTACACAATTCATGTGCTTAG-3′ |
| EIF4A1: | |

(SEQ ID NO: 32)

| | |
|---|---|
| Sense: | 5′-CAGATCATCTAGAAGCAGCTGGTTT-3′ |

(SEQ ID NO: 33)

| | |
|---|---|
| Antisense: | 5′-ACAGAATCTGGTGCCTACTAACAAAA-3′ |
| FOXK2: | |

(SEQ ID NO: 34)

| | |
|---|---|
| Sense: | 5′-GAGCAGAAGGAAGCGTGGTT-3′ |

(SEQ ID NO: 35)

| | |
|---|---|
| Antisense: | 5′-GACACATGAATTTCCACAACAGTAAA-3′ |
| GNB2L1: | |

(SEQ ID NO: 36)

| | |
|---|---|
| Sense: | 5′-ACGTAATGACATTTTGGTCTGAGTAACT-3′ |

(SEQ ID NO: 37)

| | |
|---|---|
| Antisense: | 5′-AAATGCTGCTAAACATCCTGGAA-3′ |
| HSPA9B: | |

(SEQ ID NO: 38)

| | |
|---|---|
| Sense: | 5′-TGAATCCTGAATACTATGCCTCCTT-3′ |

(SEQ ID NO: 39)

| | |
|---|---|
| Antisense: | 5′-TCCCTTCTTCTCAAGACTACTCAGTATG-3′ |
| HSPH1: | |

(SEQ ID NO: 40)

| | |
|---|---|
| Sense: | 5′-ACCTTCTTCTCACAAGACTTTTTAAGC-3′ |

(SEQ ID NO: 41)

| | |
|---|---|
| Antisense: | 5′-CCTTCTCAGCCACCATGGAA-3′ |
| KLHL18: | |

(SEQ ID NO: 42)

| | |
|---|---|
| Sense: | 5′-CCAGGTTTCCCCTGCAGTT-3′ |

(SEQ ID NO: 43)

| | |
|---|---|
| Antisense: | 5′-CCCCCTGACTTAGTCCTCGTT-3′ |
| VIL2: | |

(SEQ ID NO: 44)

| | |
|---|---|
| Sense: | 5′-CCCACTGCCTTGATCTAGTTGAT-3′ |

(SEQ ID NO: 45)

| | |
|---|---|
| Antisense: | 5′-GTCAAGCAATCTATGGCCTACCA-3′ |

In Table 2, for the gene evaluated, abbreviated name (Gene Symbol), accession number (Accession), alias name (Synonym), gene name (Gene Name), chromosome number (Chromosome), type of strand (Strand), start region (Start), stop region (Stop), and the expression level at 0 hours, three hours, 10 hours and 30 hours after the E7107 treatment were respectively shown.

TABLE 2

| Gene | | | | E7107 (nM) | | | |
|---|---|---|---|---|---|---|---|
| Symbol | Accession | Synonym | Gene Name | 0 | 3 | 10 | 30 |
| DNAJB1 | NM_006145 | HSPF1 // Hdj1 // Hsp40 | DnaJ (Hsp40) homolog, subfamily B, member 1 | 1 | 2.25 | 4.46 | 7.42 |
| BZW1 | XM_001126385 // | BZAP45 // KIAA0005 // Nbla10236 | basic leucine zipper and W2 domains 1 | 1 | 1.72 | 2.34 | 3.26 |
| | NM_014670 | | | | | | |
| NUP54 | NM_017426 | MGC13407 | nucleoporin 54 kDa | 1 | 2.31 | 2.46 | 1.93 |
| RIOK3 | NM_003831 // | DKFZp779L1370 // SUDD | RIO kinase 3 (yeast) | 1 | 2.59 | 3.57 | 2.89 |
| | NM_145906 | | | | | | |
| CDKN1B | NM_004064 | CDKN4 // KIP1 // P27KIP1 | cyclin-dependent kinase inhibitor 1B (p27, Kip1) | 1 | 1.27 | 2.35 | 4.9 |
| STK17B | NM_004226 | DRAK2 | serine/threonine kinase 17b | 1 | 1.94 | 3.44 | 3.99 |
| ID1 | NM_002165 // | ID | inhibitor of DNA binding 1, dominant negative | 1 | 3.31 | 6 | 10.57 |
| | NM_181353 | | helix-loop-helix protein | | | | |
| ADRM1 | NM_007002 // | GP110 // MGC29536 // Rpn13 | adhesion regulating molecule 1 | 1 | 1.8 | 2.68 | 2.59 |
| | NM_175573 | | | | | | |
| EIF4A1 | NM_001418 | DDX2A // EIF-4A // EIF4A | eukaryotic translation initiation factor 4A, isoform 1 | 1 | 2.17 | 3.64 | 4.36 |
| FOXK2 | XM_001134363 // | ILF // ILF-1 // ILF1 | forkhead box K2 | 1 | 2.1 | 2.68 | 2.31 |
| | XM_001134364 // | | | | | | |
| | NM_004514 | | | | | | |
| GNB2L1 | NM_006098 | Gnb2-rs1 // H12.3 // HLC-7 // | guanine nucleotide binding protein (G protein), | 1 | 2.47 | 2.74 | 2.23 |
| | | PIG21 // RACK1 | beta polypeptide 2-like 1 | | | | |
| HSPA9 | NM_004134 | CSA // GRP75 // HSPA9B // | heat shock 70 kDa protein 9 (mortalin) | 1 | 1.87 | 3.45 | 2.33 |
| | | MGC4500 // MOT // MOT2 // | | | | | |
| | | MTHSP75 // PBP74 // mot-2 | | | | | |
| HSPH1 | NM_006644 | DKFZp686M05240 // HSP105 // | heat shock 105 kDa/110 kDa protein 1 | 1 | 1.26 | 1.88 | 2.68 |
| | | HSP105A // HSP105B // KIAA0201 | | | | | |
| | | // NY-CO-25 | | | | | |
| KLHL18 | NM_025010 | FLJ13703 // KIAA0795 | kelch-like 18 (Drosophila) | 1 | 2.36 | 2.97 | 2.6 |
| VIL2 | NM_003379 | CVIL // CVL // DKFZp762NH157 // | villin 2 (ezrin) | 1 | 1.27 | 2.87 | 4.89 |
| | | FLJ26216 // MGC1584 | | | | | |

### Example 5

### Analysis in PBC

Since fractionation of hemocytes is required for employing PBMC, use of PBMC in a clinical test is complicated. If change of pre-mRNA can be confirmed in whole blood (PBC), such a test would be clinically more useful. However, since it is difficult to culture PBC, it is not easy to monitor the change of pre-mRNA in vitro. If expression of mRNA in PBC, like in PBMC, is confirmed, the change of pre-mRNA can be monitored. Accordingly, in regard to the genes of Table 2 confirmed for PBMC (DNAJB1, BZW1, NUP54, RIOK3, CDKN1B, STK17B, and ID1), it is examined whether mRNA can be detected for RNA obtained from PBC by the same RNA purification method as in the clinical setting (Tempus PAX gene).

(1) Preparation of RNA with Tempus Blood RNA Tube (Applied Biosystems)

Human peripheral blood was collected in the tube (3 ml/tube) and combined with Stabilizing Reagent. In accordance with the protocol of the Tube, RNA was purified by the RNA Blood-DNA Method in ABI 6100 PrepStation (Applied Biosystems). Absorbance at 260 nm was measured to quantify an amount of RNA.

(2) Preparation of RNA with PAXgene Blood RNA Tube (QIAGEN)

Human peripheral blood was collected in the tube (2.5 ml/tube), combined with Stabilizing Reagent, and left to stand overnight at room temperature. In accordance with the protocol of the Tube, RNA was purified with PAXgene Blood RNA Kit (QIAGEN). Absorbance at 260 nm was measured to quantify an amount of RNA.

(3) Quantification of the Expression Level of mRNA

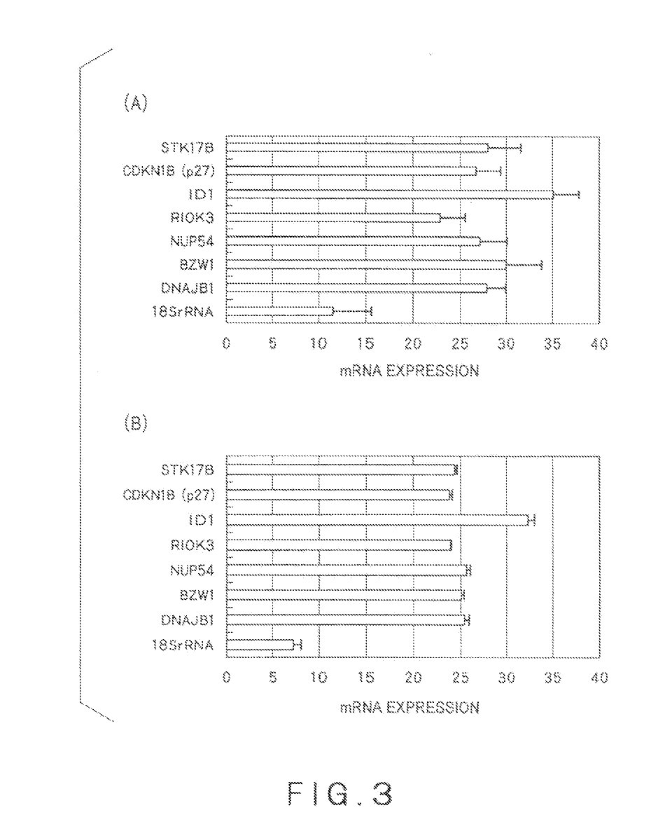

RNA was prepared to 30 ng/μL and cDNA was synthesized by using High Capacity cDNA Reverse Transcription Kits (Applied Biosystems). A probe corresponding respectively to DNAJB1, BZW1, NUP54, RIOK3, CDKN1B, STK17B, ID1 and 18S rRNA was purchased from TaqMan Gene Expression Assays (Applied Biosystems). Reagents were prepared in accordance with the protocol of TaqMan Universal PCR Master Mix (Applied Biosystems) and the expression level of mRNA was measured with ABI7900 (Applied Biosystems). As shown in FIG. 3A (Tempus Blood RNA Tube) and FIG. 3B (PAXgene Blood RNA Tube), it was demonstrated that gene expression could sufficiently be confirmed in RNA obtained by both methods.

### Example 6

### Analysis by Western Blotting

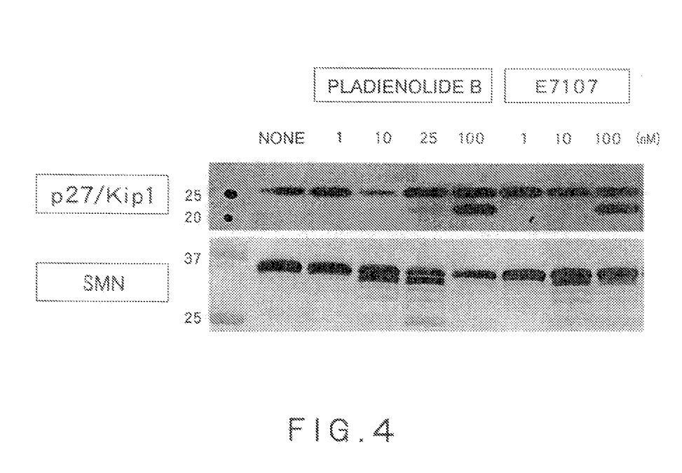

Since pre-mRNA contains a part of the introns, a protein translated from such pre-mRNA is one which does not normally exist. Consequently the protein may serves as a protein marker to monitor the action of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide. Whether the pre-mRNA-dependent abnormal protein practically emerged upon the treatment of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide was examined by using cancer cells (HaLa). As a result, insertion of a intron sequence was found to introduce a termination codon, which led to generation of small-molecule p27 and SMN different from normal CDKN1B (p27) (FIG. 4). Based on these findings, the protein generated from pre-mRNA can serve as the marker as well.

About 3×10⁵ cells of HeLa cells were first seeded in a 24-well culture plate (bottom area: 2 cm²), and 18 hours later, the compound was added thereto at the indicated concentration, followed by additional 24-hour culturing. The cells were washed once with PBS(−), and then were lysed by treating with 0.2 ml of M-PER reagent (PIERCE) for 30 minutes. The collected lysate was filtered with a 0.45 μm filter to remove insoluble matters and then combined with an equal amount of 2×SDS-PAGE sample solution. The mixture was heated to be denatured at 95° C. for five minutes to yield a sample to be analyzed.

The analysis sample (15 μl each) was separated with 5-20% SDS-PAGE and transferred onto a PVDF membrane (Hybond-P: GE-Amersham). The membrane was treated to be blocked with Blockace (Dainippon Pharma Co., Ltd.) by one-hour incubation. The membrane was then incubated with a primary antibody for two hours, washed three times for 10 minutes each, incubated with a secondary antibody for one hour, washed (10 minutes each) three times followed by signal detection.

Detection of p27/Kip1 was carried out as follows. Specifically, a mouse anti-p27/Kip1 monoclonal antibody (BD Bioscience, #610242) was used as the primary antibody at a 1:1000 dilution rate. An HRP-labeled mouse anti-IgG antibody (GE-Amersham) was used as the secondary antibody at a 1:2500 dilution rate. An ECL-Plus reagent (GE-Amersham) was used for the signal detection.

Detection of SMN was carried out as follows. Specifically, a mouse anti-SMN monoclonal antibody (BD Bioscience, #610646) was used as the primary antibody at a 1:1000 dilution rate. An AP-labeled mouse anti-IgG antibody (CHEMICON) was used as the secondary antibody at a 1:2500 dilution rate. NBT/BCIP reagent (PIERCE) was used for signal detection.

As shown in FIG. 4, only a 27 kDa band was detected for p27/Kip1 when the cells were treated without the compounds. On the other hand, appearance of a band with about 22 kDa was confirmed in the cells treated with about 100 nM of Pladienolide B or (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide for 24 hours. In addition, only a 35 kDa band was detected for SMN in the cells treated without the compounds. When the cells were treated with 10 nM and 25 nM of Pladienolide B or 10 nM and 100 nM of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide for 24 hours, multiple bands with a molecular weight of less than 35 kDa were confirmed.

### Example 7

### Analysis in a Nude Mouse

(1) Administration of the Pladienolide Derivative to Nude Mice Subcutaneously Transplanted with WiDr Human Colon Carcinoma Cells.

Nude mice (BALB/cAJcl-nu/nu, 6 weeks old, female) were purchased from CLEA Japan, Inc. After an acclimated period of a week, the mice were subcutaneously transplanted WiDr cells suspended in Hanks' Balanced Salt Solution (GIBCO) (5×10⁶ cells per a mouse). Two weeks after the transplantation, when tumor volume was confirmed to grow to more than 200 mm³, the mice were administrated (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide (30 mg/kg) in a single dose via tail vein injection.

(2) Blood Collection and Isolation of the Tumor

At the time point of 30 minutes, 1 hour, 2 hours, 4 hours, and 8 hours, after the administration of (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide, two mice per each time point were put down by euthanasia with CO₂. Whole blood (with heparin added) was taken from abdominal aorta of each mouse, TRIzol LS Reagent (Invitrogen) was added thereto, and the mixture was stored at −20° C. The tumor was removed, cut in shape of a about 0.5 cm×0.5 cm square, and stored −20° C. in RNAlater (Ambion).

(3) Preparation of RNA

RNA preparation from blood was carried out in accordance with the protocol of TRIzol LS Reagent (Invitrogen). The obtained RNA was further purified with RNeasy (QIAGEN). During the process, a treatment with DNase I was performed in accordance with the protocol. The tumor treated with RNA later (Ambion) was placed in TRI reagent (SIGMA) and grinded by a homogenizer, followed by the operations following the protocol of TRI reagent. The obtained RNA was then purified using RNeasy (QIAGEN). During the process, a treatment with DNase I was performed in accordance with the protocol. Absorbance at 260 nm was measured to quantify an amount of each RNA.

(4) Measurement of the Expression Level of the Precursor Gene (pre-mRNA) in Blood

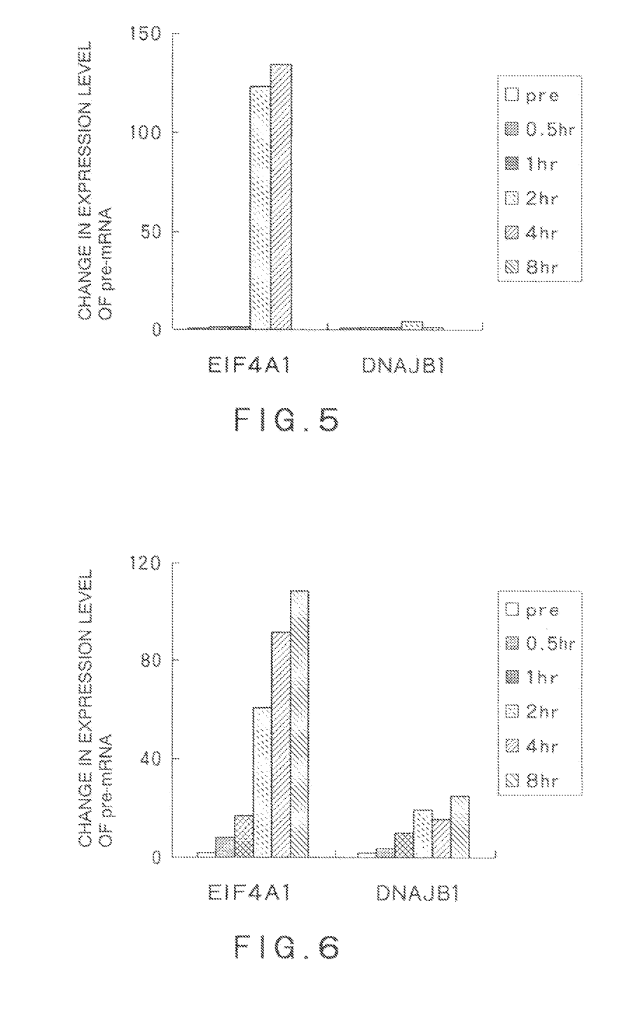

RNA was prepared to 100 ng/μl and cDNA was synthesized by using TaqMan Reverse Transcription Reagents (Applied Biosystems). For the expression level of pre-mRNA of mouse DNAJB1 and mouse EIF4A, reagents were prepared in accordance with each protocol by using POWER SYBR GREEN PCR MASTER MIX (Applied Biosystems) and primers with the sequence below (Invitrogen), and the expression level was measured with ABI7900 (Applied Biosystems). The expression level of 18S rRNA was measured by using 18S primers included in TaqMan Ribosomal RNA control reagents (4308329: Applied Biosystems). Measured values were corrected using the expression level of 18S rRNA as an internal control. The expression level of each gene was calculated with the expression level in the group of mice treated without (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide as 1. Results were shown in FIG. 5.

| | |
|---|---|
| [mouse DNAJB1] | |
| Sense: | |
| 5′-TGCTGTGAGAATAATGGGTTGTG-3′ | (SEQ ID NO: 46) |
| Antisense: | |
| 5′-GGCTGGCTTAGGAGCTTCACT-3′ | (SEQ ID NO: 47) |
| [mouse EIF4A1] | |
| Sense: | |
| 5′-ACCTGCGGTTCCCACTTTATT-3′ | (SEQ ID NO: 48) |
| Antisense: | |
| 5′-ACCACTCCAAATGTCTAAGGTCACT-3′ | (SEQ ID NO: 49) |

(5) Measurement of the expression level of the precursor gene (pre-mRNA) in tumor

RNA was prepared to 100 ng/μl and cDNA was synthesized by using TaqMan Reverse Transcription Reagents (Applied Biosystems). For the expression level of pre-mRNA of human DNAJB1 and human EIF4A, reagents were prepared in accordance with each protocol by using POWER SYBR GREEN PCR MASTER MIX (Applied Biosystems) and primers with the sequence below (Invitrogen), and the expression level was measured with ABI7900 (Applied Biosystems). The expression level of 18S rRNA was measured by using 18S primers included in TaqMan Ribosomal RNA control reagents (4308329: Applied Biosystems). Measured values were corrected using the expression level of 18S rRNA as an internal control. The expression level of each gene was calculated with the expression level in the group of mice treated without (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide as 1. Results were shown in FIG. 6.

| | |
|---|---|
| [Human DNAJB1] | |
| Sense: | |
| 5′-GGCCTGATGGGTCTTATCTATGG-3′ | (SEQ ID NO: 50) |
| Antisense: | |
| 5′-TTAGATGGAAGCTGGCTCAAGAG-3′ | (SEQ ID NO: 51) |
| [Human EIF4A1] | |
| Sense: | |
| 5′-GAACTTTGCCTCATTCTTTTGCA-3′ | (SEQ ID NO: 52) |
| Antisense: | |
| 5′-CTGAGCTCCAGTCTCTTGTATTTCTG-3′ | (SEQ ID NO: 53) |

### Example 8

### Analysis with Microarray (2)

Analysis conditions were reviewed in the data used in Example 3. Genes containing increased introns upon treatment with (8E,12E,14E)-7-((4-cycloheptylpiperazin-1-yl)carbonyl)oxy-3,6,16,21-tetrahydroxy-6,10,12,16,20-pentamethyl-18,19-epoxytricosa-8,12,14-trien-11-olide were identified.

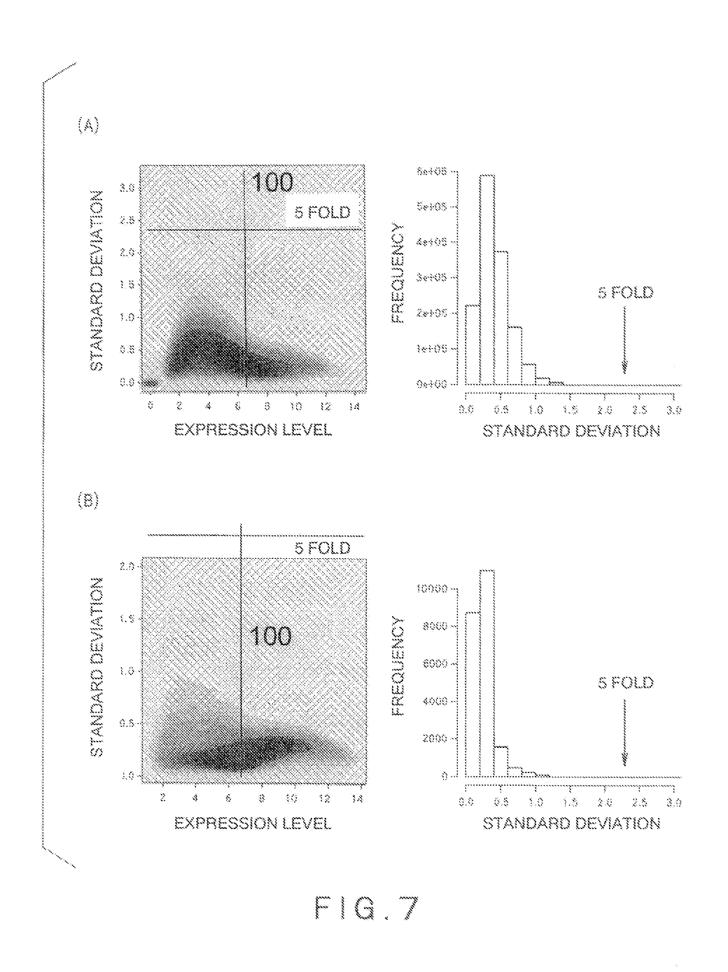

In order to consider the analysis conditions, variations in the data from the samples with identical conditions were studied. FIG. 7 shows a scatter diagram of the expression level of probe sets and genes of the control cells, and standard deviation thereof. It turned out that, by analyzing under conditions where the expression level was more than 100 and change in the expression level was more than five times, a probe set with increased expression can be extracted.

Probe sets showing that the normalized change in the level expression of the probe set in the group with the treatment was more than 5 times; the expression level of the probe set and the gene in the group with the treatment were more than 100; and probe set annotation was “extended, full, free”, were picked out. Welch's t-test was conducted with the probe sets extracted for the treated group and the control group to determine a p and q value. A gene containing the probe sets with the q value of less than 5% was determined as a candidate gene with increased expression level of the intron regions (Table 3).

In Table 3, for each gene evaluated, gene name (Gene Name), abbreviated name (Gene Symbol), accession number (Accession), alias name (Synonym), transcription cluster number in Human Exon 1.0 ST Array (Human Exon 1.0 ST Array Transcript Cluster ID), chromosome number (Chromosome), type of strand (Strand), start region (Start), stop region (Stop), and change in expression level (Fold Change) were respectively shown.

TABLE 3

| | | | | Human Exon | | | | | |
|---|---|---|---|---|---|---|---|---|---|
| | | | | ST 1.0 Array | Human Genome hg18 | | | | |
| Gene Name | Gene Symbol | Accession | Synonym | Transcript Cluster ID | Chromosome | Strand | Start | Stop | Fold Change |
| TSR1, 20S rRNA accumulation, | TSR1 | NM_018128 | FLJ10534 // | 3740998 | chr17 | − | 2172438 | 2187551 | 44.77 |
| translocase of inner mitochondrial | TIMM23 | XM_001133798 // | MGC22767 // | 3289031 | chr10 | − | 51180787 | 51293382 | 28.32 |
| membrane 23 homolog (yeast) | | NM_006327 | PRO1197 // TIM23 // | | | | | | |
| | | | TIMM23B | | | | | | |
| pellino homolog 1 (Drosophila) | PELI1 | NM_020651 | DKFZp686C18116 // | 2556302 | chr2 | − | 64173290 | 64225291 | 27.36 |
| | | | MGC50990 | | | | | | |
| chromosome 15 open reading frame | C15orf42 | NM_152259 | FLJ41618 // | 3607698 | chr15 | + | 87857353 | 87975281 | 26.27 |
| 42 | | | MGC45866 | | | | | | |
| RNA binding motif protein 26 | RBM26 | NM_022118 | C13orf10 // | 3519119 | chr13 | − | 78783968 | 78878613 | 21.52 |
| | | | FLJ20957 // | | | | | | |
| | | | MGC133295 // | | | | | | |
| | | | MGC133296 // | | | | | | |
| | | | PRO1777 // RP11- | | | | | | |
| | | | 255E21.1 // SE70-2 | | | | | | |
| integrin, beta 1 (fibronectin receptor, | ITGB1 | NM_002211 // | CD29 // FNRB // | 3284188 | chr10 | − | 33211051 | 33321367 | 21.20 |
| beta polypeptide, antigen CD29 | | NM_033666 // | GPIIA // MDF2 // | | | | | | |
| includes MDF2, MSK12) | | NM_033667 // | MSK12 // VLAB | | | | | | |
| | | NM_033668 // | | | | | | | |
| | | NM_033669 // | | | | | | | |
| | | NM_133376 | | | | | | | |
| ADP-ribosylation factor guanine | ARFGEF1 | NM_006421 | ARFGEEP1 // BIG1 // | 3139035 | chr8 | − | 68250009 | 68418446 | 21.19 |
| nucleotide-exchange factor | | | D730028O18Rik // | | | | | | |
| 1(brefeldin A-inhibited) | | | DKFZP434L057 // | | | | | | |
| | | | P200 | | | | | | |
| clathrin interactor 1 | CLINT1 | NM_014666 | CLINT // ENTH // | 2883609 | chr5 | − | 157145339 | 157284818 | 20.51 |
| | | | EPN4 // EPNR // | | | | | | |
| | | | EPSINR // KIAA0171 | | | | | | |
| CTD (carboxy-terminal domain, RNA | CTDSPL2 | NM_016396 | FLJ10523 // | 3591909 | chr15 | + | 42506834 | 42607712 | 20.40 |
| polymerase II, polypeptide A) small | | | HSPC058 // | | | | | | |
| phosphatase like 2 | | | HSPC129 | | | | | | |
| cell division cycle associated 4 | CDCA4 | NM_017955 // | FLJ20764 // HEPP // | 3581386 | chr14 | − | 104546975 | 104585458 | 20.39 |
| | | NM_145701 | MGC19517 | | | | | | |
| WD repeat domain 73 | WDR73 | NM_032856 | FLJ14888 // | 3636956 | chr15 | − | 82987018 | 82998719 | 20.03 |
| | | | HSPC264 | | | | | | |
| zinc finger protien 317 // zinc finger | ZNF317 // | NM_020933 // | KIAA1588 // | 3819880 | chr19 | + | 9112073 | 9135082 | 19.64 |
| protein 658 | ZNF658 | XM_001125688 // | DKFZp572C163 // | | | | | | |
| | | NM_033160 | FLJ32813 // | | | | | | |
| | | | MGC35232 | | | | | | |
| integrin, alpha 6 | ITGA6 | NM_000210 // | CD49f // ITGA6B // | 2515627 | chr2 | + | 173000563 | 173079416 | 19.59 |
| | | NM_001079818 | VLA-6 | | | | | | |
| ubiquitin protein ligase E3A (human | UBE3A | NM_000462 // | ANCR // AS // E6- | 3614087 | chr15 | − | 23006655 | 23355108 | 19.35 |
| papilioma virus E6-associated | | NM_130838 // | AP // EPVE6AP // | | | | | | |
| protein, Angelman syndrome) | | NM_130839 | FLJ26981 // HPVE6A | | | | | | |
| muscleblind-like 2 (Drosophila) | MBNL2 | NM_144778 // | DKFZp781H1296 // | 3497586 | chr13 | + | 96672563 | 96844371 | 19.31 |
| | | NM_207304 | MBLL // MBLL39 // | | | | | | |
| | | | MGC120625 // | | | | | | |
| | | | MGC120626 // | | | | | | |
| | | | MGC120628 // | | | | | | |
| | | | PRO2032 | | | | | | |
| chromosome 6 open reading frame | C6orf166 | NM_018064 | FLJ10342 // | 2963784 | chr6 | − | 88441297 | 88476721 | 19.23 |
| 166 | | | dJ486L4.2 | | | | | | |
| homocysteine-inducible, endoplasmic | HERPUD1 | NM_001010989 // | HERP // KIAA0025 // | 3662387 | chr16 | + | 55523269 | 55552122 | 19.03 |
| reticulum stress-inducible, ubiquitin- | | NM_001010990 // | Mif1 // SUP | | | | | | |
| like domain member 1 | | NM_014685 | | | | | | | |
| RAP1 interacting factor homolog | RIF1 | NM_018151 | DKFZp781N1478 // | 2510485 | chr2 | + | 151974674 | 152072768 | 18.85 |
| (yeast) | | | FLJ12870 | | | | | | |
| development and differentiation | DDEF2 | NM_003887 | AMAP2 // FLJ42910 | 2468811 | chr2 | + | 9264345 | 9463243 | 18.43 |
| enhancing factor 2 | | | // KIAA0400 // PAG3 | | | | | | |
| | | | // PAP // Pap-alpha | | | | | | |
| | | | // SHAG1 | | | | | | |
| cell division cycle 20 homolog (S. cerevisiae) | CDC20 | NM_001255 | CDC20A // | 2333136 | chr1 | + | 43597180 | 43601431 | 18.22 |
| | | | MGC102824 // | | | | | | |
| | | | bA276H19.3 // | | | | | | |
| | | | p55CDC | | | | | | |
| BMI1 polycomb ring finger oncogene | BMI1 | NM_005180 | MGC12685 // PCGF4 | 3238491 | chr10 | + | 22650103 | 22660820 | 18.11 |
| | | | // RNF51 | | | | | | |
| golgi-specific brefeldin A resistance | GBF1 | NM_004193 | FLJ21263 // | 3261544 | chr10 | + | 103993260 | 104132642 | 17.47 |
| factor 1 | | | FLJ21500 // | | | | | | |
| | | | KIAA0248 // | | | | | | |
| | | | MGC134877 // | | | | | | |
| | | | MGC134878 | | | | | | |
| strawberry notch homolog 1 | SBNO1 | NM_018183 | FLJ10701 // | 3476130 | chr12 | − | 122342423 | 122435284 | 17.44 |
| (Drosophila) | | | FLJ10833 // | | | | | | |
| | | | FLJ16176 // MOP3 | | | | | | |
| | | | // Sno | | | | | | |
| protein phosphatase 4, regulatory | PPP4R2 | NM_174907 | MGC131930 | 2629693 | chr3 | + | 73019849 | 73201035 | 17.19 |
| subunit 2 | | | | | | | | | |
| brix domain containing 2 | BXDC2 | NM_018321 | BRIX // FLJ11100 | 2806231 | chr5 | + | 34951248 | 34962845 | 17.08 |
| far upstream element (FUSE) binding | FUBP1 | NM_003902 | FBP // FUBP | 2419235 | chr1 | − | 78182330 | 78217881 | 16.59 |
| protein 1 | | | | | | | | | |
| RAE1 RNA export 1 homolog (S. pombe) | RAE1 | NM_001015885 // | FLJ30608 // | 3890555 | chr20 | + | 55359493 | 55386992 | 16.46 |
| | | NM_003610 | MGC117333 // | | | | | | |
| | | | MGC126076 // | | | | | | |
| | | | MGC126077 // MIG14 | | | | | | |
| | | | // MRNP41 // | | | | | | |
| | | | Mnrp41 // dJ481F12.3 | | | | | | |
| | | | // dJ800J21.1 | | | | | | |
| CCHC-type zinc finger, nucleic acid | CNBP | NM_003418 | CNBP1 // DM2 // | 2694644 | chr3 | − | 130369369 | 130385467 | 16.43 |
| binding protein | | | PROMM // RNF163 | | | | | | |
| | | | // ZCCHC22 // ZNF9 | | | | | | |
| acyl-CoA synthetase long-chain | ACSL3 | NM_004457 // | ACS3 // FACL3 // | 2529546 | chr2 | + | 223433916 | 223539136 | 16.27 |
| family member 3 | | NM_203372 | PRO2194 | | | | | | |
| peptidase (mitochondrial processing) | PMPCA | NM_015160 | Alpha-MPP // | 3194338 | chr9 | + | 138424942 | 138438030 | 16.01 |
| alpha | | | INPP5E // KIAA0123 | | | | | | |
| | | | // MGC104197 | | | | | | |
| CGI-09 protein | CGI-09 | NM_015939 | MGC5029 | 3896524 | chr20 | − | 5865881 | 5879370 | 16.01 |
| baculoviral IAP repeat-containing 6 | BIRC6 | NM_016252 | APOLLON // BRUCE | 2476219 | chr2 | + | 32435095 | 32697447 | 15.96 |
| (apollon) | | | // FLJ13726 // | | | | | | |
| | | | FLJ13786 // | | | | | | |
| | | | KIAA1289 | | | | | | |
| BCL2/adenovirus E1B 19 kDa | BNIP2 | NM_004330 | BNIP-2 // NIP2 | 3627076 | chr15 | − | 57742025 | 57768916 | 15.96 |
| interacting protein 2 | | | | | | | | | |
| DEAD (Asp-Glu-Ala-Asp) box | DDX23 | NM_004818 | MGC8416 // PRPF28 | 3453319 | chr12 | − | 47509816 | 47532322 | 15.78 |
| polypeptide 23 | | | // U5-100K // prp28 | | | | | | |
| DEAD (Asp-Glu-Ala-Asp) box | DDX17 | NM_006386 // | DKFZp761H2016 // | 3960629 | chr22 | − | 37202800 | 37232288 | 15.42 |
| polypeptide 17 | | NM_030881 | P72 // RH70 | | | | | | |
| SAPS domain family, member 3 | SAPS3 | NM_018312 | C11orf23 // | 3337618 | chr11 | + | 67984785 | 68173381 | 15.25 |
| | | | DKFZp781E17107 // | | | | | | |
| | | | DKFZp781E2374 // | | | | | | |
| | | | DKFZp781O2362 // | | | | | | |
| | | | FLJ11058 // | | | | | | |
| | | | FLJ43065 // | | | | | | |
| | | | MGC125711 // | | | | | | |
| | | | MGC125712 // | | | | | | |
| | | | PP6R3 // SAP190 // | | | | | | |
| | | | SAPL // SAPLa | | | | | | |
| PAK1 interacting protein 1 // | PAK1IP1 // | NM_017906 // | FLJ20624 // MAK11 | 2894663 | chr6 | + | 10799016 | 10817955 | 15.04 |
| chromosome 20 open reading frame 7 | C20orf7 | NM_001039375 // | // PIP1 // RP11- | | | | | | |
| | | NM_024120 | 421M1.5 // WDR84 // | | | | | | |
| | | | bA421M1.5 // hPIP1 | | | | | | |
| | | | // FLJ22324 // | | | | | | |
| | | | MGC90272 // | | | | | | |
| | | | bA526K24.2 // | | | | | | |
| | | | dJ842G6.1 | | | | | | |
| sprouty-related, EVH1 domain | SPRED2 | NM_181784 | FLJ21897 // | 2556752 | chr2 | − | 65391279 | 65513247 | 15.03 |
| containing 2 | | | FLJ31917 // | | | | | | |
| | | | MGC163164 // | | | | | | |
| | | | Spred-2 | | | | | | |
| kelch-like 18 (Drosophila) | KLHL18 | NM_025010 | FLJ13703 // | 2621275 | chr3 | + | 47298888 | 47367590 | 15.00 |
| | | | KIAA0795 | | | | | | |
| suppressor of var1, 3-like 1 (S. cerevisiae) | SUPV3L1 | NM_003171 | SUV3 | 3250204 | chr10 | + | 70609946 | 70638996 | 14.99 |
| KIAA0174 | KIAA0174 | NM_014761 | MGC117220 | 3667766 | chr16 | + | 70481983 | 70522196 | 14.78 |
| flap structure-specific endonuclease 1 | FEN1 | NM_004111 | FEN-1 // MF1 // | 3333226 | chr11 | + | 61312929 | 61350560 | 14.75 |
| | | | RAD2 | | | | | | |
| atrophin 1 | ATN1 | NM_001007026 // | B37 //D12S755E // | 3403045 | chr12 | + | 6903897 | 6921744 | 14.70 |
| | | NM_001940 | DRPLA // NOD | | | | | | |
| poliovirus receptor | PVR | NM_006505 | CD155 // HVED // | 3835645 | chr19 | + | 49839030 | 49860075 | 14.67 |
| | | | NECL5 // Necl-5 // | | | | | | |
| | | | PVS // TAGE4 | | | | | | |
| KIAA0317 | KIAA0317 | NM_001039479 | — | 3572041 | chr14 | − | 74189599 | 74249655 | 14.49 |
| DEAD (Asp-Glu-Ala-Asp) box | DDX3X | XM_001129278 // | DBX // DDX14 // | 3974838 | chrX | + | 41055591 | 41108668 | 14.39 |
| polypeptide 3, X-linked | | NM_001356 | DDX3 // HLP2 | | | | | | |
| brix domain containing 1 | BXDC1 | NM_032194 | FLJ21087 // RPF2 // | 2921374 | chr6 | + | 111409800 | 111408743 | 14.38 |
| | | | bA397G5.4 | | | | | | |
| actinin, alpha 1 | ACTN1 | NM_001102 | FLJ40884 | 3569814 | chr14 | − | 68409817 | 68515815 | 14.26 |
| Rho guanine nucleotide exchange | ARHGEF12 | NM_015313 | DKFZp698O2372 // | 3352503 | chr11 | + | 119711882 | 119896306 | 14.21 |
| factor (GEF) 12 | | | KIAA0382 // LARG // | | | | | | |
| | | | PRO2792 | | | | | | |
| KIAA0859 | KIAA0859 | NM_001007239 // | 5630401D24Rik // | 2367287 | chr1 | + | 170017287 | 170053841 | 14.17 |
| | | NM_014955 // | CGI-01 // FLJ10310 | | | | | | |
| | | NM_015935 | | | | | | | |
| THO complex 5 | THOC5 | NM_001002877 // | C22orf19 // Fmip // | 3956854 | chr22 | − | 28224935 | 28280423 | 14.15 |
| | | NM_001002878 // | PK1.3 | | | | | | |
| | | NM_001002879 // | | | | | | | |
| | | NM_003678 | | | | | | | |
| polo-like kinase 2 (Drosophila) | PLK2 | NM_006622 | SNK | 2858023 | chr5 | − | 57785572 | 57792624 | 14.01 |
| forkhead box K2 | FOXK2 | XM_001134363 // | ILF // ILF-1 // ILF1 | 3738969 | chr17 | + | 78070883 | 78195807 | 14.01 |
| | | XM_001134364 // | | | | | | | |
| | | NM_004514 | | | | | | | |
| calcium binding protein 39 | CAB39 | NM_016289 | CGI-66 // FLJ22682 | 2531522 | chr2 | + | 231285781 | 231394027 | 13.97 |
| | | | // MO25 | | | | | | |
| ubiquitin specific peptidase 38 | USP38 | NM_032557 | FLJ35970 // | 2745499 | chr4 | + | 144325529 | 144364549 | 13.94 |
| | | | HP43.8KD // | | | | | | |
| | | | KIAA1891 | | | | | | |
| SMAD specific E3 ubiquitin protein | SMURF2 | NM_022739 | DKFZp686F0270 // | 3766960 | chr17 | − | 59968891 | 60088896 | 13.92 |
| ligase 2 | | | MGC138150 | | | | | | |
| RIO kinase 3 (yeast) | RIOK3 | NM_003831 // | DKFZp779L1370 // | 3781654 | chr18 | + | 19161035 | 19320545 | 13.87 |
| | | NM_145906 | SUDD | | | | | | |
| protein phosphatase 1, catalytic | PPP1CC | NM_002710 | PPP1G | 3471374 | chr12 | − | 109642007 | 109665131 | 13.87 |
| subunit, gamma isoform | | | | | | | | | |
| erbb2 interacting protein // caspase | ERBB2IP // | NM_001006600 // | ERBIN // LAP2 // | 2812435 | chr5 | + | 65258079 | 65451372 | 13.87 |
| 6, apoptosis-related cysteine | CASP6 | NM_018695 // | MCH2 | | | | | | |
| peptidase | | NM_001226 // | | | | | | | |
| | | NM_032992 | | | | | | | |
| MKI67 (FHA domain) interacting | MKI67IP | NM_032390 | NIFK // Nopp34 | 2573786 | chr2 | − | 122132489 | 122231052 | 13.76 |
| nucleolar phosphoprotein | | | | | | | | | |
| tyrosine 3- | YWHAB | NM_003404 // | GW128 // HS1 // | 3886639 | chr20 | + | 42927275 | 42970748 | 13.72 |
| monooxygenase/tryptophan 5- | | NM_139323 | KCIP-1 | | | | | | |
| monooxygenase activation protein, | | | | | | | | | |
| Ctr9, Paf1/RNA polymerase II | CTR9 // | NM_014633 // | KIAA0155 // SH2BP1 | 3320301 | chr11 | + | 10684027 | 10774985 | 13.68 |
| complex component, homolog (S. cerevisiae) | TRMU | NM_018006 | // TSBP // p150 // | | | | | | |
| // tRNA 5- | | | p150TSP // | | | | | | |
| methylaminomethyl-2-thiouridylate | | | MGC99627 // MTO2 | | | | | | |
| methyltransferase | | | // MTU1 // TRMT // | | | | | | |
| | | | TRMT1 // TRNT1 | | | | | | |
| karyopherin (importin) beta 1 | KPNB1 | NM_002265 | IMB1 // IPOB // | 3724782 | chr17 | + | 43082272 | 43117868 | 13.68 |
| | | | Impnb // MGC2155 // | | | | | | |
| | | | MGC2156 // | | | | | | |
| | | | MGC2157 // NTF97 | | | | | | |
| A kinase (PRKA) anchor protein 13 | AKAP13 | NM_006738 // | AKAP-Lbc // BRX // | 3606304 | chr15 | + | 83681343 | 84102785 | 13.65 |
| | | NM_007200 // | FLJ11952 // | | | | | | |
| | | NM_144767 | FLJ43341 // HA-3 // | | | | | | |
| | | | Ht31 // LBC // | | | | | | |
| | | | PROTO-LB // | | | | | | |
| | | | PROTO-LBC // c-lbc | | | | | | |
| nucleolar protein 9 | NOL9 | NM_024654 | FLJ23323 // | 2394784 | chr1 | − | 6504004 | 6537329 | 13.58 |
| | | | MGC131821 // | | | | | | |
| | | | MGC138483 | | | | | | |
| ADP-ribosylation factor-like 6 | ARL6IP2 | NM_022374 | ARL3IP2 // ATL2 // | 2548776 | chr2 | − | 38374621 | 38516141 | 13.57 |
| interacting protein 2 | | | FLJ23293 // atlastin2 | | | | | | |
| pumilio homolog 2 (Drosophila) | PUM2 | NM_015317 | FLJ36528 // | 2542816 | chr2 | − | 20291440 | 20450197 | 13.55 |
| | | | KIAA0235 // | | | | | | |
| | | | MGC138251 // | | | | | | |
| | | | MGC138253 // | | | | | | |
| | | | PUMH2 // PUML2 | | | | | | |
| calmodulin 2 (phosphorylase kinase, | CALM2 | NM_001743 | CAMII // PHKD // | 2551924 | chr2 | − | 47232159 | 47286178 | 13.54 |
| delta) | | | PHKD2 | | | | | | |
| nucleolar protein family A, member 1 | NOLA1 | NM_018983 // | GAR1 | 2739242 | chr4 | + | 110956115 | 110965340 | 13.42 |
| (H/ACA small nucleolar RNPs) | | NM_032993 | | | | | | | |
| chromosome 6 open reading frame | C6orf94 // | XM_928657 // | dJ468K18.5 // | 2929036 | chr6 | + | 144194149 | 144227146 | 13.35 |
| 94 // LTV1 homolog (S. cerevisiae) | LTV1 | XM_941265 // | C6orf93 // FLJ14909 | | | | | | |
| | | NM_032860 | // dJ468K18.4 | | | | | | |
| PPAR binding protein | PPARBP | NM_004774 | CRSP1 // CRSP200 | 3755714 | chr17 | − | 34801544 | 34861069 | 13.29 |
| | | | // DRIP205 // | | | | | | |
| | | | DRIP230 // MED1 // | | | | | | |
| | | | MGC71488 // PBP // | | | | | | |
| | | | PPARGBP // RB18A | | | | | | |
| | | | // TRAP220 // TRIP2 | | | | | | |
| 3-hydroxy-3-methylglutaryl- | HMGCR | NM_000859 | — | 2815965 | chr5 | + | 74668800 | 74700136 | 13.12 |
| Coenzyme A reductase | | | | | | | | | |
| 15 kDa selenoprotein | 15-Sep | NM_004261 // | — | 2421271 | chr1 | − | 87100720 | 87153273 | 13.02 |
| | | NM_203341 | | | | | | | |
| phosphatidylinosital binding clathrin | PICALM | NM_001008660 // | CALM // CLTH // | 3385175 | chr11 | − | 85345353 | 85458481 | 13.00 |
| assembly protein | | NM_007166 | LAP | | | | | | |
| proteasome (prosome, macropain) | PSMD3 | NM_002809 | P58 // RPN3 // S3 | 3720636 | chr17 | + | 35390438 | 35407732 | 12.85 |
| 26S subunit, non-ATPase, 3 | | | | | | | | | |
| COP9 constitutive photomorphogenic | COPS2 | NM_004236 | ALIEN // CSN2 // | 3623424 | chr15 | − | 47203877 | 47235198 | 12.73 |
| homolog subunit 2 (Arabidopsis) | | | SGN2 // TRIP15 | | | | | | |
| B-cell CLL/lymphoma 10 | BCL10 | NM_003921 | CARMEN // CIPER | 2420808 | chr1 | − | 85504067 | 85516171 | 12.67 |
| | | | // CLAP // c-E10 // | | | | | | |
| | | | mE10 | | | | | | |
| villin 2 (ezrin) | VIL2 | NM_003379 | CVIL // CVL // | 2981912 | chr6 | − | 159106770 | 159160432 | 12.62 |
| | | | DKFZp762H157 // | | | | | | |
| | | | FLJ26216 // | | | | | | |
| | | | MGC1584 | | | | | | |
| solute carrier family 25, member 32 | SLC25A32 | NM_030780 | FLJ23872 // MFTC | 3147726 | chr8 | − | 104247068 | 104496640 | 12.60 |
| DEAD (Asp-Glu-Ala-Asp) box | DDX21 | NM_004728 | DKFZp686F21172 // | 3250055 | chr10 | + | 70380584 | 70414821 | 12.56 |
| polypeptide 21 | | | GUA // GURDB // | | | | | | |
| | | | RH-II/GU // RH- | | | | | | |
| | | | II/GuA | | | | | | |
| WEE1 homolog (S. pombe) | WEE1 | NM_003390 | DKFZp686I18166 // | 3319937 | chr11 | + | 9550916 | 9579143 | 12.54 |
| | | | FLJ16446 // WEE1hu | | | | | | |
| N-acetyltransferase 10 | NAT10 | NM_024662 | ALP // | 3326252 | chr11 | + | 34083688 | 34125769 | 12.51 |
| | | | DKFZp434C116 // | | | | | | |
| | | | FLJ10774 // | | | | | | |
| | | | FLJ12179 // | | | | | | |
| | | | FLJ23850 // | | | | | | |
| | | | KIAA1709 // hALP | | | | | | |
| transcription elongation factor B | TCEB3 | NM_003198 | SIII // TCEB3A | 2325251 | chr1 | + | 23942459 | 23961135 | 12.38 |
| (SIII), polypeptide 3 (110 kDa, elongin | | | | | | | | | |
| microtubule-associated protein, | MAPRE1 | NM_012325 | EB1 // MGC117374 | 3882069 | chr20 | + | 30871304 | 30901864 | 12.37 |
| RP/EB family, member 1 | | | // MGC129946 | | | | | | |
| Cdk5 and Abl enzyme substrate 1 | CABLES1 | NM_138375 | FLJ35924 // HsT2563 | 3781531 | chr18 | + | 18940699 | 19101596 | 12.37 |
| ubiquitin specific peptidase 47 | USP47 | NM_017944 | DKFZp686C13257// | 3320604 | chr11 | + | 11819556 | 11937446 | 12.32 |
| | | | FLJ20727 // TRFP | | | | | | |
| DEAD (Asp-Glu-Ala-Asp) box | DDX31 | NM_022779 // | FLJ13633 // | 3228191 | chr9 | − | 134456969 | 134535609 | 12.21 |
| polypeptide 31 | | NM_138620 | FLJ14578 // | | | | | | |
| | | | FLJ23349 // helicain | | | | | | |
| | | | A // helicain B // | | | | | | |
| | | | helicain C | | | | | | |
| chromosome 8 open reading frame | C8orf76 // | NM_032847 // | FLJ14825 // | 3151473 | chr8 | − | 124291083 | 124356860 | 12.17 |
| 76 // zinc fingers and homeoboxes 1 | ZHX1 | NM_001017926 // | MGC9784 //— | | | | | | |
| | | NM_007222 | | | | | | | |
| chromoseme 10 open reading frame | C10orf18 | XM_374765 // | DKFZp781E1986 // | 3233322 | chr10 | + | 5766851 | 5846629 | 12.16 |
| 18 | | XM_937970 | FLJ20360 // | | | | | | |
| | | | bA318E3.2 | | | | | | |
| guanine nucleotide binding protein- | GNL2 | NM_013285 | FLJ40906 // | 2407191 | chr1 | − | 37805043 | 37834120 | 12.14 |
| like 2 (nucleolar) | | | HUMAUANTIG // | | | | | | |
| | | | NGP1 // Ngp-1 // | | | | | | |
| | | | dJ423B22.6 | | | | | | |
| eukaryotic translation initiation factor | EIF4G1 | NM_004953 // | DKFZp686A1451 // | 2655688 | chr3 | + | 185514970 | 185535829 | 12.13 |
| 4 gamma, 1 | | NM_182917 // | EIF4F // EIF4G // | | | | | | |
| | | NM_198241 // | p220 | | | | | | |
| | | NM_198242 // | | | | | | | |
| | | NM_198244 | | | | | | | |
| peter pan homolog (Drosophila) // | PPAN // | NM_020230 // | BXDC3 // MGC14226 | 3820342 | chr19 | + | 10077989 | 10082841 | 12.07 |
| PPAN-P2RY11 | PPAN- | NM_001040664 | // MGC45852 // SSF | | | | | | |
| | P2RY11 | | // SSF1 // SSF2 // | | | | | | |
| Rho GTPase activating protein 11A | ARHGAP11A | NM_014783 // | GAP (1-12) // | 3587457 | chr15 | + | 30693622 | 30719422 | 12.06 |
| | | NM_199357 | KIAA0013 // | | | | | | |
| | | | MGC70740 | | | | | | |
| high-mobility group nucleosome | HMGN1 | NM_004965 | FLJ27265 // | 3932270 | chr21 | − | 39636124 | 39643423 | 11.92 |
| binding domain 1 | | | FLJ31471 // HMG14 | | | | | | |
| | | | // MGC104230 // | | | | | | |
| | | | MGC117425 | | | | | | |
| mortality factor 4 // mortality factor | MORF4 // | NM_006792 // | CSR // CSRB // SEN | 3603532 | chr15 | + | 76889894 | 76979806 | 11.90 |
| 4 like 1 | MORF4L1 | NM_006791 // | // SEN1 // Eaf3 // | | | | | | |
| | | NM_206839 | FWP006 // HsT17725 | | | | | | |
| | | | // MGC10631 // | | | | | | |
| | | | MORFRG15 // | | | | | | |
| | | | MRG15 // S863-6 | | | | | | |
| cytotoxic and regulatory T cell | KIAA0690 // | NM_019604 | — | 3302240 | chr10 | − | 99098933 | 99175486 | 11.86 |
| molecule | CRTAM | | | | | | | | |
| TERF1 (TRF1)-interacting nuclear | TINF2 | NM_012461 | TIN2 | 3558012 | chr14 | − | 23776465 | 23781950 | 11.82 |
| factor 2 | | | | | | | | | |
| DCN1, defective in cullin neddylation | DCUN1D5 | NM_032299 | FLJ32431 // | 3388914 | chr11 | − | 102342328 | 102485844 | 11.81 |
| 1, domain containing 5 (S. cerevisiae) | | | MGC2714 | | | | | | |
| isopentenyl-diphosphate delta | IDI1 | NM_004508 | IPP1 // IPPI1 | 3273601 | chr10 | − | 1075869 | 1092634 | 11.81 |
| isomerase 1 | | | | | | | | | |
| exosome component 9 | EXOSC9 | NM_001034194 // | PM/Scl-75 // | 2741768 | chr4 | + | 122941935 | 122957724 | 11.79 |
| | | NM_005033 | PMSCL1 // RRP45 // | | | | | | |
| | | | Rrp45p // p5 // p6 | | | | | | |
| diablo homolog (Drosophila) | DIABLO | NM_019887 // | DIABLO-S // | 3475511 | chr12 | − | 121257461 | 121277963 | 11.73 |
| | | NM_138929 // | FLJ10537 // | | | | | | |
| | | NM_138930 | FLJ25049 // SMAC | | | | | | |
| | | | // SMAC3 | | | | | | |
| eukaryotic translation elongation | EEF2 | NM_001961 | EEF-2 // EF2 | 3846538 | chr19 | − | 3926677 | 3936451 | 11.73 |
| factor 2 | | | | | | | | | |
| calcium activated nucleotidase 1 | CANT1 | NM_138793 | SCAN-1 // SHAPY | 3772775 | chr17 | − | 74499403 | 74517744 | 11.68 |
| moesin | MSN | NM_002444 | — | 3979659 | chrX | + | 64804248 | 64878488 | 11.57 |
| aquarius homolog (mouse) | AQR | NM_014691 | KIAA0560 | 3617757 | chr15 | − | 32935042 | 33160557 | 11.56 |
| EPH receptor A2 | EPHA2 | NM_004431 | ECK | 2397948 | chr1 | − | 16323428 | 16355151 | 11.54 |
| ATPase, Na+/K+ transporting, beta 1 | ATP1B1 | NM_001001787 // | ATP1B // MGC1798 | 2366422 | chr1 | + | 167342204 | 167368946 | 11.44 |
| polypeptide | | NM_001677 | | | | | | | |
| ring finger protein 40 | RNF40 | NM_014771 | BRE1B // | 3656555 | chr16 | + | 30681120 | 30695182 | 11.42 |
| | | | DKFZp686K191 // | | | | | | |
| | | | KIAA0661 // | | | | | | |
| | | | MGC13051 // RBP95 | | | | | | |
| | | | // STARING | | | | | | |
| KIAA0406 | KIAA0406 | NM_014657 | — | 3905073 | chr20 | − | 36044840 | 36095268 | 11.38 |
| TIMP metallopeptidase inhibitor 1 | TIMP1 | NM_003254 | CLGI // EPA // EPO | 3976341 | chrX | + | 47326654 | 47332978 | 11.27 |
| | | | // FLJ90373 // HCI | | | | | | |
| | | | // TIMP | | | | | | |
| DCP1 decapping enzyme homolog A | DCP1A | NM_018403 | HSA275986 // | 2676726 | chr3 | − | 53296412 | 53356692 | 11.27 |
| (S. cerevisiae) | | | Nbla00360 // | | | | | | |
| | | | SMAD4IP1 // SMIF | | | | | | |
| heat shock 70 kDa protein 5 | HSPA5 | NM_005347 | BIP // FLJ29106 // | 3225398 | chr9 | − | 127036953 | 127064590 | 11.27 |
| (glucose-regulated protein, 78 kDa) | | | GRP78 // MIF2 | | | | | | |
| chromosome 4 open reading frame | C4orf23 | NM_152544 | FLJ12891 // | 2717757 | chr4 | + | 8474582 | 8545892 | 11.25 |
| 23 | | | FLJ35725 | | | | | | |
| RAB5A, member RAS oncogene | RAB5A | NM_004162 | RAB5 | 2613386 | chr3 | + | 19963571 | 20002852 | 11.19 |
| importin 7 | IPO7 | NM_006391 | FLJ14581 // | 3319840 | chr11 | + | 9362702 | 9426296 | 11.19 |
| | | | MGC138673 // | | | | | | |
| | | | RANBP7 | | | | | | |
| nucleoporin 214 kDa | NUP214 | NM_005085 | CAIN // CAN // | 3191900 | chr9 | + | 132990780 | 133102339 | 11.18 |
| | | | D9S46E // | | | | | | |
| | | | MGC104525 // N214 | | | | | | |
| PRP8 pre-mRNA processing factor 8 | PRPF8 | NM_006445 | HPRP8 // PRP8 // | 3740479 | chr17 | − | 1500681 | 1534906 | 11.12 |
| homolog (S. cerevisiae) | | | PRPC8 // RP13 | | | | | | |
| Snf2-related CBP activator protein | SRCAP | NM_006662 | KIAA0309 | 3656418 | chr16 | + | 30616551 | 30663998 | 11.09 |
| keratin 18 | KRT18 | NM_000224 // | CYK18 // K18 | 3415576 | chr12 | + | 51596769 | 51632949 | 11.07 |
| | | NM_199187 | | | | | | | |
| adiponectin receptor 2 | ADIPOR2 | NM_024551 | ACDCR2 // | 3400625 | chr12 | + | 1670418 | 1768071 | 11.05 |
| | | | FLJ21432 // | | | | | | |
| | | | MGC4640 // PAQR2 | | | | | | |
| splicing factor, arginine/serine-rich 3 | SFRS3 | NM_003017 | SRp20 | 2905118 | chr6 | + | 36669658 | 36741293 | 11.05 |
| cyclin B1 | CCNB1 | NM_031966 | CCNB | 2813414 | chr5 | + | 68498462 | 68509822 | 11.03 |
| GrpE-like 2, mitochondrial (E. coli) | GRPEL2 | NM_152407 | DKFZp451C205 // | 2835006 | chr5 | + | 148705190 | 148714338 | 11.03 |
| | | | FLJ23713 // | | | | | | |
| | | | FLJ33918 // Mt- | | | | | | |
| | | | GrpE#2 | | | | | | |
| Yes-associated protein 1, 65 kDa | YAP1 | NM_006106 | YAP // YAP2 // | 3346453 | chr11 | + | 101486379 | 101609466 | 11.01 |
| | | | YAP65 | | | | | | |
| nuclear factor (erythroid-derived 2)- | NFE2L3 | NM_004289 | NRF3 | 2993590 | chr7 | + | 26158385 | 26194013 | 11.01 |
| like 3 | | | | | | | | | |
| nucleoporin like 1 | NUPL1 | NM_001008564 // | KIAA0410 // | 3482219 | chr13 | + | 24773258 | 24822202 | 10.97 |
| | | NM_001008565 | PRO2463 | | | | | | |
| | | NM_014089 | | | | | | | |
| RAB14, member RAS oncogene | RAB14 | NM_016322 | FBP // RAB-14 | 3223872 | chr9 | − | 122980236 | 123025093 | 10.96 |
| sperm specific antigen 2 | SSFA2 | NM_006751 | CS-1 // CS1 // | 2518428 | chr2 | + | 182464815 | 182516732 | 10.95 |
| | | | DKFZp313O1039 // | | | | | | |
| | | | DKFZp779G0129 // | | | | | | |
| | | | FLJ45996 // | | | | | | |
| | | | KIAA1927 // KRAP // | | | | | | |
| | | | SPAG13 | | | | | | |
| DnaJ (Hsp40) homolog, subfamily B, | DNAJB1 | NM_006145 | HSPF1 // Hdj1 // | 3852783 | chr19 | − | 14485622 | 14501114 | 10.94 |
| member 1 | | | Hsp40 | | | | | | |
| cyclin D1 | CCND1 | NM_053056 | BCL1 // D11S287E | 3338192 | chr11 | + | 69161881 | 69178408 | 10.91 |
| | | | // PRAD1 // U21B31 | | | | | | |
| chromosome 20 open reading frame | C20orf77 | NM_021215 | CREPT // | 3884450 | chr20 | + | 36095232 | 36189576 | 10.91 |
| 77 | | | DKFZp434P0735 // | | | | | | |
| | | | FLJ44520 // | | | | | | |
| | | | dJ1057B20.2 | | | | | | |
| SWI/SNF related, matrix associated, | SMARCD1 | NM_003076 // | BAF60A // CRACD1 | 3414390 | chr12 | + | 48765279 | 48780742 | 10.90 |
| actin dependent regulator of | | NM_139071 | // Rsc6p | | | | | | |
| chromatin, subfamily d, member 1 | | | | | | | | | |
| kinesin family member 11 | KIF11 | NM_004523 | EG5 // HKSP // | 3258168 | chr10 | + | 94292951 | 94405130 | 10.90 |
| | | | KNSL1 // TRIP5 | | | | | | |
| dickkopf homolog 1 (Xenopus laevis) | DKK1 | NM_012242 | DKK-1 // SK | 3247172 | chr10 | + | 53744067 | 53807545 | 10.89 |
| polo-like kinase 4 (Drosophila) | PLK4 | NM_014264 | SAK // STK18 | 2742985 | chr4 | + | 129021450 | 129039809 | 10.88 |
| OTU domain containing 4 | OTUD4 | NM_017493 // | DKFZp434I0721 // | 2788195 | chr4 | − | 146274170 | 146471942 | 10.88 |
| | | NM_199324 | HIN1 // HSHIN1 // | | | | | | |
| | | | KIAA1046 | | | | | | |
| ring finger and SPRY domain | RSPRY1 | NM_133368 | KIAA1972 | 3662612 | chr16 | + | 55777703 | 55831882 | 10.87 |
| containing 1 | | | | | | | | | |
| leucine rich repeat containing 59 | LRRC59 | NM_018509 | FLJ21675 // | 3762355 | chr17 | − | 45813504 | 45836593 | 10.85 |
| exosome component 2 | EXOSC2 | NM_014285 | RRP4 // Rrp4p // | 3191695 | chr9 | + | 132559000 | 132572797 | 10.83 |
| | | | hRrp4p // p7 | | | | | | |
| polo-like kinase 1 (Drosophila) | PLK1 | NM_005030 | PLK // STPK13 | 3653072 | chr16 | + | 23597664 | 23609180 | 10.82 |
| chromosome 20 open reading frame | C20orf24 | NM_018840 // | PNAS-11 // RIP5 | 3883971 | chr20 | + | 34667571 | 34674368 | 10.81 |
| 24 | | NM_199483 | | | | | | | |
| KIAA1984 // chromosome 9 open | KIAA1984 // | NM_001039374 // | MGC15438 // PARF | 3194635 | chr9 | + | 138810077 | 138855460 | 10.81 |
| reading frame 86 | C9orf86 | NM_024718 | // RP11-216L13.7 // | | | | | | |
| | | | RP11-216L13.9 // | | | | | | |
| | | | bA216L13.7 // | | | | | | |
| | | | FLJ10101 // | | | | | | |
| | | | FLJ13045 // Parf // | | | | | | |
| | | | bA216L13.9 // pp8875 | | | | | | |
| nucleolar protein 1, 120 kDa | NOL1 | NM_001033714 // | MGC117384 // | 3442024 | chr12 | − | 6536290 | 6547817 | 10.79 |
| | | NM_006170 | MGC149287 // | | | | | | |
| | | | MGC149288 // | | | | | | |
| | | | NOP120 // NSUN1 // | | | | | | |
| | | | p120 | | | | | | |
| chromosome 3 open reading frame | C3orf17 | NM_001025072 // | DKFZP434F2021 | 2688882 | chr3 | − | 113926517 | 114253449 | 10.73 |
| 17 | | NM_001025073 // | | | | | | | |
| | | NM_015412 | | | | | | | |
| DOT1-like, histone H3 | DOT1L | NM_032482 | DOT1 // KIAA1814 | 3816264 | chr19 | + | 2114945 | 2183576 | 10.70 |
| methyltransferase (S. cerevisiae) | | | | | | | | | |
| dullard homolog (Xenopus laevis) | DULLARD | NM_015343 | HSA011916 | 3743501 | chr17 | − | 7087644 | 7096514 | 10.70 |
| v-raf-1 murine leukemia viral | RAF1 // | NM_002880 // | CRAF // Raf-1 // c- | 2663244 | chr3 | − | 12600117 | 12680654 | 10.61 |
| oncogene homolog 1 // microtubule- | MAP2 | NM_001039538 // | Raf // | | | | | | |
| associated protein 2 | | NM_002374 // | DKFZp686I2148 // | | | | | | |
| | | NM_031845 // | MAP2A // MAP2B // | | | | | | |
| | | NM_031847 | MAP2C | | | | | | |
| solute carrier family 39 (zinc | SLC39A7 // | NM_001077516 // | D6S115E // | 2903470 | chr6 | + | 33276220 | 33280187 | 10.55 |
| transporter), member 7 // ring finger | RING1 | NM_006979 // | D6S2244E // H2-KE4 | | | | | | |
| protein 1 | | NM_002931 | // HKE4 // KE4 // | | | | | | |
| | | | RING5 // ZIP7 // | | | | | | |
| | | | RNF1 | | | | | | |
| testis derived transcript (3 LIM | TES | NM_015641 // | DKFZP586B2022 // | 3020192 | chr7 | + | 115637811 | 115743800 | 10.53 |
| domains) | | NM_152829 | MGC1146 // TESS // | | | | | | |
| | | | TESS-2 // TESTIN | | | | | | |
| chromosome 12 open reading frame | C12orf11 | NM_018164 | FLJ10630 // | 3448428 | chr12 | − | 26949277 | 26982501 | 10.50 |
| 11 | | | FLJ10637 | | | | | | |
| golgi associated PDZ and coiled-coil | GOPC // | NM_001017408 // | CAL // FIG // | 2971378 | chr6 | − | 117988142 | 118030384 | 10.49 |
| motif containing // v-ros UR2 | ROS1 | NM_020399 // | GOPC1 // PIST // | | | | | | |
| sarcoma virus oncogene homolog 1 | | NM_002944 | dJ94G16.2 // MCF3 | | | | | | |
| (avian) | | | // ROS | | | | | | |
| protein regulator of cytokinesis 1 // | PRC1 // | NM_003981 // | ASE1 // MGC1671 // | 3639031 | chr15 | − | 89310285 | 89338950 | 10.49 |
| interferon-related developmental | IFRD1 | NM_199413 // | MGC3669 // PC4 // | | | | | | |
| regulator 1 | | NM_199414 // | TIS7 | | | | | | |
| | | NM_001007245 // | | | | | | | |
| | | NM_001550 | | | | | | | |
| RNA methyltransferase like 1 | RNMTL1 | NM_018146 | FLJ10581 // HC90 | 3705539 | chr17 | + | 632263 | 642481 | 10.45 |
| forkhead box O1A | FOXO1A | NM_002015 | FKH1 // FKHR // | 3510858 | chr13 | − | 40027817 | 40162098 | 10.41 |
| (rhabdomyosarcoma) | | | FOXO1 | | | | | | |
| mitochondrial ribosomal protein S7 | MRPS7 | NM_015971 | MRP-S // MRP-S7 | 3734760 | chr17 | + | 70769374 | 70774626 | 10.41 |
| | | | // RP-S7 // RPMS7 | | | | | | |
| suppressor of Ty 6 homolog (S. cerevisiae) | SUPT6H | NM_003170 | KIAA0162 // | 3715703 | chr17 | + | 24013260 | 24053809 | 10.38 |
| | | | MGC87943 // SPT6 | | | | | | |
| | | | // SPT6H // emb-5 | | | | | | |
| splicing factor, arginine/serine-rich 1 | SFRS1 | NM_001078166 // | ASF // MGC5228 // | 3764103 | chr17 | − | 53421412 | 53439635 | 10.37 |
| (splicing factor 2, alternate splicing | | NM_006924 | SF2 // SF2p33 // | | | | | | |
| factor) | | | SRp30a | | | | | | |
| heterogeneous nuclear | HNRPM | NM_005968 // | DKFZp547H118 // | 3819543 | chr19 | + | 8415651 | 8459993 | 10.37 |
| ribonucleoprotein M | | NM_031203 | HNRNPM // | | | | | | |
| | | | HNRNPM4 // | | | | | | |
| | | | HNRPM4 // HTGR1 | | | | | | |
| neuroepithelial cell transforming gene 1 | NET1 | NM_001047160 // | ARHGEF8 // NET1A | 3233182 | chr10 | + | 5444535 | 5491001 | 10.35 |
| | | NM_005863 | | | | | | | |
| ribonucleotide reductase M2 | RRM2 | NM_001034 | R2 // RR2M | 2469252 | chr2 | + | 10179982 | 10279478 | 10.35 |
| polypeptide | | | | | | | | | |
| chromosome 16 open reading frame | C16orf80 | NM_013242 | EVORF // GTL3 | 3693511 | chr16 | − | 56683698 | 56739201 | 10.32 |
| 80 | | | | | | | | | |
| exosome component 4 | EXOSC4 | NM_019037 | FLJ20591 // RRP41 | 3120008 | chr8 | + | 145205527 | 145207529 | 10.30 |
| | | | // RRP41A // Rrp41p | | | | | | |
| | | | // SKI6 // Ski6p // | | | | | | |
| | | | hRrp41p // p12A | | | | | | |
| UTP6, small subunit (SSU) | UTP6 | NM_018428 | C17orf40 // HCA66 | 3752437 | chr17 | − | 27211153 | 27252839 | 10.30 |
| processome component, homolog | | | | | | | | | |
| (yeast) | | | | | | | | | |
| polymerase (RNA) I polypeptide C, | POLR1C | NM_004875 // | RPA39 // RPA40 // | 2908052 | chr6 | + | 43592769 | 43605071 | 10.29 |
| 30 kDa | | NM_203290 | RPA5 // RPAC1 | | | | | | |
| centromere protein L | CENPL | NM_033319 | C1orf155 // CENP-L | 2444451 | chr1 | − | 172035313 | 172104622 | 10.28 |
| | | | // FLJ31044 // RP3- | | | | | | |
| | | | 383J4.1 // dJ383J4.3 | | | | | | |
| coronin, actin binding protein, 1C | CORO1C | NM_014325 | HCRNN4 // coronin-3 | 3470549 | chr12 | − | 107563018 | 107697353 | 10.27 |
| ATP-binding cassette, sub-family F | ABCF3 | NM_018358 | EST201864 // | 2655511 | chr3 | + | 185386580 | 185395316 | 10.26 |
| (GCN20), member 3 | | | FLJ11198 | | | | | | |
| telomeric repeat binding factor 2, | TERF2IP | NM_018975 | DRIP5 // RAP1 | 3669092 | chr16 | + | 74238853 | 74353272 | 10.25 |
| interacting protein | | | | | | | | | |
| ADP-ribosylation factor interacting | ARFIP2 // | NM_012402 // | POR1 // GST // | 3360941 | chr11 | − | 6453496 | 6459171 | 10.24 |
| protein 2 (arfaptin 2) // solute | SLCO6A1 | NM_173488 | MGC26949 // | | | | | | |
| carrier organic anion transporter | | | OATP6A1 // OATPY | | | | | | |
| family, member 6A1 | | | | | | | | | |
| RNA binding motif protein 25 | RBM25 | NM_021239 | MGC105088 // | 3543411 | chr14 | + | 72594776 | 72658577 | 10.23 |
| | | | MGC117168 // | | | | | | |
| | | | RNPC7 // S164 | | | | | | |
| zinc finger protein 410 | ZNF410 | NM_021188 | APA-1 // APA1 | 3543884 | chr14 | + | 73423093 | 73468718 | 10.23 |
| 3-hydroxy-3-methylglutaryl- | HMGCS1 | NM_002130 | HMGCS // | 2855501 | chr5 | − | 43324231 | 43349337 | 10.19 |
| Coenzyme A synthase 1 (soluble) | | | MGC90332 | | | | | | |
| transcription elongation regulator 1 | TCERG1 | NM_001040006 // | CA150 // | 2834093 | chr5 | + | 145754916 | 145871713 | 10.18 |
| | | NM_006706 | MGC133200 // | | | | | | |
| WD repeat domain 74 | WDR74 | XM_001125771 // | FLJ10439 // | 3376235 | chr11 | − | 62356627 | 62365857 | 10.13 |
| | | NM_018093 | FLJ21730 | | | | | | |
| sperm associated antigen 5 | SPAG5 | NM_006461 | DEEPEST // MAP126 | 3750785 | chr17 | − | 23928719 | 23950424 | 10.12 |
| | | | // hMAP126 | | | | | | |
| G protein-coupled receptor 126 | GPR126 | NM_001032394 // | DREG // PS1TP2 // | 2928461 | chr6 | + | 142622079 | 142853981 | 10.05 |
| | | NM_001032395 // | VIGR | | | | | | |
| | | NM_020455 // | | | | | | | |
| | | NM_198569 | | | | | | | |
| DnaJ-like protein // guanine | bA16L21.2.1 | NM_001015882 // | — | 3184940 | chr9 | + | 113412172 | 113474576 | 10.05 |
| nucleotide binding protein (G | // GNG10 | NM_001017998 | | | | | | | |
| protein), gamma 10 | | | | | | | | | |
| general transcription factor IIIC, | GTF3C4 | NM_012204 | FLJ21002 // | 3192525 | chr9 | + | 134535243 | 134581784 | 10.00 |
| polypeptide 4, 90 kDa | | | MGC138450 // | | | | | | |
| | | | TFIII90 // TFIIIC90 // | | | | | | |
| | | | TFIIICdelta // | | | | | | |
| | | | TFiiiC2-90 | | | | | | |
| eukaryotic translation initiation factor 5 | EIF5 | NM_001969 // | EIF-5A | 3553607 | chr14 | + | 102743652 | 102881108 | 10.00 |
| | | NM_183004 | | | | | | | |
| programmed cell death 7 | PDCD7 | NM_005707 | ES18 // HES18 // | 3629475 | chr15 | − | 63196772 | 63213308 | 9.97 |
| | | | MGC22015 | | | | | | |
| dual specificity phosphatase 14 | DUSP14 | NM_007026 | MKP-L // MKP6 | 3719515 | chr17 | + | 32922795 | 32947707 | 9.97 |
| RAD18 homolog (S. cerevisiae) | RAD18 | NM_020165 | RNF73 | 2662020 | chr3 | − | 8792108 | 8980146 | 9.94 |
| myeloid cell leukemia sequence 1 | MCL1 | NM_021960 // | EAT // MCL1L // | 2434438 | chr1 | − | 148799312 | 148818878 | 9.93 |
| (BCL2-related) | | NM_182763 | MCL1S // | | | | | | |
| | | | MGC104264 // | | | | | | |
| oxysterol binding protein-like 2 | OSBPL2 | NM_014835 // | FLJ20223 // | 3892565 | chr20 | + | 60245389 | 60304834 | 9.91 |
| | | NM_144498 | KIAA0772 // | | | | | | |
| | | | MGC4307 // | | | | | | |
| | | | MGC8342 // ORP-2 | | | | | | |
| | | | // ORP2 | | | | | | |
| inhibitor of DNA binding 2B, dominant | ID2B //ID2 | NM_001039082 // | —// GIG8 // ID2A // | 2468622 | chr2 | + | 8513132 | 8750492 | 9.91 |
| negative helix-loop-helix protein // | | NM_002166 | ID2H // MGC26389 | | | | | | |
| inhibitor of DNA binding 2, dominant | | | | | | | | | |
| negative helix-loop-helix protein | | | | | | | | | |
| cytoskeleton associated protein 5 | CKAP5 | NM_001008938 // | FLJ35359 // | 3371719 | chr11 | − | 46721250 | 46824660 | 9.90 |
| | | NM_014756 | KIAA0097 // TOG // | | | | | | |
| | | | TOGp // ch-TOG | | | | | | |
| thymosin, beta 10 | TMSB10 | NM_021103 | MIG12 | 2491271 | chr2 | + | 84959319 | 84988131 | 9.85 |
| DEAD (Asp-Glu-Ala-Asp) box | DDX27 | NM_017895 | DKFZp667N057 // | 3888217 | chr20 | + | 47237780 | 47294013 | 9.83 |
| polypeptide 27 | | | FLJ12917 // | | | | | | |
| | | | FLJ20596 // | | | | | | |
| | | | FLJ22238 // | | | | | | |
| | | | HSPC259 // | | | | | | |
| | | | MGC1018 // | | | | | | |
| | | | MGC163147 // | | | | | | |
| | | | PP3241 // RHLP // | | | | | | |
| TAR DNA binding protein | TARDBP | NM_007375 | TDP-43 | 2320048 | chr1 | + | 10995025 | 11013170 | 9.82 |
| pyruvate dehydrogenase complex, | PDHX // | NM_003477 // | DLDBP // E3BP // | 3326540 | chr11 | + | 34870003 | 34998695 | 9.81 |
| component X // mitogen-activated | MAPK10 | NM_002753 // | OPDX // PDX1 // | | | | | | |
| protein kinase 10 | | NM_138980 // | proX // FLJ12099 // | | | | | | |
| | | NM_138981 // | FLJ33785 // JNK3 // | | | | | | |
| | | NM_138982 | JNK3A // MGC50974 | | | | | | |
| | | | // PRKM10 // | | | | | | |
| | | | p493F12 // | | | | | | |
| dihydrouridine synthase 3-like (S. cerevisiae) | DUS3L | NM_020175 | DUS3 // FLJ13896 | 3847420 | chr19 | − | 5735867 | 5759311 | 9.80 |
| splicing factor proline/glutamine-rich | SFPQ | NM_005066 | POMP100 // PSF | 2406064 | chr1 | − | 35351696 | 35506894 | 9.80 |
| (polypyrimidine tract binding protein | | | | | | | | | |
| associated) | | | | | | | | | |
| phosphoinositide-3-kinase, | PIK3R4 | NM_014602 | MGC102700 // | 2695200 | chr3 | − | 131801268 | 132026111 | 9.79 |
| regulatory subunit 4, p150 | | | VPS15 // p150 | | | | | | |
| RNA binding motif, single stranded | RBMS2 | NM_002898 | SCR3 | 3417583 | chr12 | + | 55202025 | 55277435 | 9.78 |
| interacting protein 2 | | | | | | | | | |
| eukaryotic translation initiation factor | EIF2C2 | NM_012154 | AGO2 // MGC3183 | 3156193 | chr8 | − | 141599440 | 141726037 | 9.74 |
| 2C, 2 | | | // Q10 | | | | | | |
| inner centromere protein antigens | INCENP | NM_001040694 // | FLJ31633 // | 3333358 | chr11 | + | 61648046 | 61677757 | 9.73 |
| 135/155 kDa | | NM_020238 | MGC111393 | | | | | | |
| cofilin 1 (non-muscle) | CFL1 | NM_005507 | CFL | 3377886 | chr11 | − | 65347069 | 65386796 | 9.72 |
| heat shock 105 kDa/110 kDa protein 1 | HSPH1 | NM_006644 | DKFZp686M05240 // | 3508330 | chr13 | − | 30558258 | 30651692 | 9.68 |
| | | | HSP105 // HSP105A | | | | | | |
| | | | // HSP105B // | | | | | | |
| | | | KIAA0201 // NY-CO- | | | | | | |
| | | | 25 | | | | | | |
| nuclear import 7 homolog (S. cerevisiae) | NIP7 | NM_016101 | CGI-37 // FLJ10296 | 3666686 | chr16 | + | 67930869 | 67935044 | 9.64 |
| | | | // HSPC031 // KD93 | | | | | | |
| epiregulin | EREG | NM_001432 | ER | 2731513 | chr4 | + | 75440309 | 75473341 | 9.62 |
| mannosidase, alpha, class 1A, | MAN1A2 // | NM_006699 // — | MAN1B // — | 2353881 | chr1 | + | 117689854 | 117931990 | 9.61 |
| member 2 // urothelial cancer | UCA1 | | | | | | | | |
| tumor necrosis factor receptor | TNFRSF1A | NM_001065 | CD120a // FPF // | 3441849 | chr12 | − | 6308185 | 6321522 | 9.60 |
| superfamily, member 1A | | | MGC19588 // TBP1 | | | | | | |
| | | | // TNF-R // TNF-R-I | | | | | | |
| | | | // TNF-R55 // | | | | | | |
| | | | TNFAR // TNFR1 // | | | | | | |
| | | | TNFR55 // TNFR60 | | | | | | |
| | | | // p55 // p55-R // | | | | | | |
| cyclin L1 | CCNL1 | NM_020307 | BM-001 // PRO1073 | 2702307 | chr3 | − | 158305077 | 158361233 | 9.60 |
| | | | // ania-6a | | | | | | |
| RNA binding motif protein 4 | RBM4 | NM_002896 | DKFZp547K0918 // | 3336422 | chr11 | + | 66161951 | 66190547 | 9.60 |
| | | | LARK // MGC75138 | | | | | | |
| | | | // RBM4A // | | | | | | |
| | | | ZCCHC21 // ZCRB3A | | | | | | |
| KIAA1429 | KIAA1429 | NM_015496 // | DKFZP434I116 // | 3145020 | chr8 | − | 95569109 | 95634851 | 9.57 |
| | | NM_183009 | DKFZp781B2117 // | | | | | | |
| | | | MGC138493 // | | | | | | |
| | | | MGC141940 // | | | | | | |
| | | | MSTP054 | | | | | | |
| nucleoporin 153 kDa | NUP153 | NM_005124 | HNUP153 // N153 | 2943808 | chr6 | − | 17723255 | 17855638 | 9.56 |
| zinc finger protein 91 homolog | ZFP91 // | NM_053023 // | FKSG11 // PZF // | 3331730 | chr11 | + | 58101868 | 58149782 | 9.54 |
| (mouse) // ciliary neurotrophic | CNTF | NM_170768 // | ZNF757 // HCNTF | | | | | | |
| factor | | NM_000614 | | | | | | | |
| tetraspanin 5 | TSPAN5 | NM_005723 | NET-4 // TM4SF9 // | 2778856 | chr4 | − | 99589201 | 99798839 | 9.53 |
| | | | TSPAN-5 | | | | | | |
| resistance to inhibitors of | RIC8A | NM_021932 | MGC104517 // | 3315512 | chr11 | + | 197139 | 206202 | 9.52 |
| cholinesterase 8 homolog A (C. elegans) | | | MGC131931 // | | | | | | |
| | | | MGC148073 // | | | | | | |
| | | | MGC148074 // RIC8 | | | | | | |
| | | | // synembryn | | | | | | |
| splicing factor 3b, subunit 1, 155 kDa | SF3B1 | NM_001005526 // | PRP10 // PRPF10 // | 2593670 | chr2 | − | 197962756 | 198039327 | 9.52 |
| | | NM_012433 | SAP155 // SF3b155 | | | | | | |
| DEAD (Asp-Glu-Ala-Asp) box | DDX47 // | NM_016355 // | DKFZp564O176 // | 3405531 | chr12 | + | 12856462 | 12874176 | 9.51 |
| polypeptide 47 // apolipoprotein L | APOLD1 | NM_201224 // | E4-DBP // FLJ30012 | | | | | | |
| domain containing 1 | | NM_030817 | // HQ0256 // | | | | | | |
| | | | MSTP162 // | | | | | | |
| | | | DKFZP434F0318 // | | | | | | |
| tensin 4 | TNS4 | NM_032865 | CTEN // FLJ14950 | 3756262 | chr17 | − | 35881491 | 35911365 | 9.51 |
| | | | // PP14434 | | | | | | |
| heterogeneous nuclear | HNRPD | NM_001003810 // | AUF1 // AUF1A // | 2775463 | chr4 | − | 83492676 | 83515077 | 9.50 |
| ribonucleoprotein D (AU-rich | | NM_002138 // | P37 // hnRNPD0 | | | | | | |
| element RNA binding protein 1, | | NM_031369 // | | | | | | | |
| 37 kDa) | | NM_031370 | | | | | | | |
| v-yes-1 Yamaguchi sarcoma viral | YES1 | NM_005433 | HsT441 // P61-YES | 3795942 | chr18 | − | 711592 | 803236 | 9.48 |
| oncogene homolog 1 | | | // Yes // c-yes | | | | | | |
| nuclear factor (erythroid-derived 2)- | NFE2L2 // | NM_006164 // | NRF2 // — | 2588827 | chr2 | − | 177794809 | 177837753 | 9.48 |
| like 2 // hypothetical protein | DKFZp451M2119 | NM_182585 | | | | | | | |
| DKFZp451M2119 | | | | | | | | | |
| eukaryotic translation initiation factor | EIF4A1 // | NM_001416 // | DDX2A // EIF-4A // | 3708826 | chr17 | + | 7416768 | 7423040 | 9.46 |
| 4A, isoform 1 // | SENP3 | NM_015670 | EIF4A // | | | | | | |
| SUMO1/sentrin/SMT3 specific | | | DKFZP586K0919 // | | | | | | |
| peptidase 3 | | | DKFZp762A152 // | | | | | | |
| | | | SMT3IP1 // SSP3 | | | | | | |
| nucleolar protein 11 | NOL11 | NM_015462 | DKFZP586L0724 | 3732373 | chr17 | + | 63144526 | 63205942 | 9.45 |
| adenylate kinase 2 | AK2 | NM_001625 // | ADK2 | 2405364 | chr1 | − | 33245959 | 33275155 | 9.45 |
| | | NM_013411 | | | | | | | |
| splicing factor, arginine/serine-rich 5 | SFRS5 | NM_001039465 // | HRS // SRP40 | 3542207 | chr14 | + | 69263363 | 69308465 | 9.42 |
| | | NM_006925 | | | | | | | |
| ancient ubiquitous protein 1 // | AUP1 // | NM_181575 // | —// FLJ23757 | 2560254 | chr2 | − | 74607294 | 74610455 | 9.38 |
| DEAQ box polypeptide 1 (RNA- | DQX1 | NM_133637 | | | | | | | |
| dependent ATPase) | | | | | | | | | |
| AF4/FMR2 family, member 4 | AFF4 | NM_014423 | AF5Q31 // MCEF // | 2875555 | chr5 | − | 132238768 | 132327205 | 9.38 |
| | | | MGC75036 | | | | | | |
| guanine nucleotide binding protein (G | GNB2L1 | NM_006098 | Gnb2-rs1 // H12.3 // | 2891052 | chr5 | − | 180596592 | 180612024 | 9.38 |
| protein), beta polypeptide 2-like 1 | | | HLC-7 // PIG21 // | | | | | | |
| | | | RACK1 | | | | | | |
| survival motor neuron domain | SMNDC1 | NM_005871 | SMNR // SPF30 | 3306516 | chr10 | − | 112042540 | 112106248 | 9.38 |
| containing 1 | | | | | | | | | |
| BUB1 budding uninhibited by | BUB1 | NM_004336 | BUB1A // BUB1L // | 2570616 | chr2 | − | 111088456 | 111174950 | 9.36 |
| benzimidazoles 1 homolog (yeast) | | | hBUB1 | | | | | | |
| SET domain containing 5 | SETD5 | XM_926615 // | DKFZp686J18276 // | 2609608 | chr3 | + | 9414309 | 9495916 | 9.36 |
| | | XM_931376 // | FLJ10707 // | | | | | | |
| | | XM_931417 // | KIAA1757 | | | | | | |
| | | XM_931444 // | | | | | | | |
| | | XM_931447 // | | | | | | | |
| | | XM_931455 // | | | | | | | |
| | | XM_931478 // | | | | | | | |
| | | XM_939712 // | | | | | | | |
| | | XM_944260 // | | | | | | | |
| | | XM_944276 // | | | | | | | |
| | | XM_944280 // | | | | | | | |
| | | XM_944295 // | | | | | | | |
| | | XM_944298 // | | | | | | | |
| | | XM_944303 // | | | | | | | |
| | | NM_001080517 | | | | | | | |
| heat shock protein 90 kDa beta | HSP90B1 // | NM_003299 // | ECGP // GP96 // | 3429312 | chr12 | + | 102848291 | 102871551 | 9.34 |
| (Grp94), member 1 // thymine-DNA | TDG // | NM_003211 // — | GRP94 // TRA1 // — | | | | | | |
| glycosylase // heat shock protein | HSP90B2P | | // GRP94P1 // | | | | | | |
| 90 kDa beta (Grp94), member 2 | | | GRP94b // HSP // | | | | | | |
| (pseudogene) | | | HSPCP2 // HsGrp94b | | | | | | |
| | | | // TRA1P1 // TRAP1 | | | | | | |
| actin, gamma 1 | ACTG1 | NM_001614 | ACT // ACTG // | 3773932 | chr17 | − | 77091456 | 77105797 | 9.32 |
| | | | DFNA20 // DFNA26 | | | | | | |
| dual specificity phosphatase 6 | DUSP6 | NM_001946 // | MKP3 // PYST1 | 3464860 | chr12 | − | 88223376 | 88273467 | 9.31 |
| | | NM_022652 | | | | | | | |
| LATS, large tumor suppressor, | LATS2 | NM_014572 | FLJ13161 // KPM | 3504526 | chr13 | − | 20445178 | 20533718 | 9.28 |
| homolog 2 (Drosophila) | | | | | | | | | |
| SERPINE1 mRNA binding protein 1 | SERBP1 | NM_001018067 // | CGI-55 // CHD3IP // | 2417174 | chr1 | − | 67646101 | 67669020 | 9.26 |
| | | NM_001018068 // | DKFZp564M2423 // | | | | | | |
| | | NM_001018069 // | FLJ90489 // HABP4L | | | | | | |
| | | NM_015640 | // PAI-RBP1 // | | | | | | |
| | | | PAIRBP1 | | | | | | |
| DnaJ (Hsp40) homolog, subfamily A, | DNAJA3 | NM_005147 | FLJ45758 // TID1 // | 3646164 | chr16 | + | 4415465 | 4446773 | 9.23 |
| member 3 | | | hTid-1 | | | | | | |
| nuclear cap binding protein subunit 2, | NCBP2 | NM_001042540 // | CBC2 // CBP20 // | 2713074 | chr3 | − | 198146677 | 198154763 | 9.23 |
| 20 kDa | | NM_007362 | NIP1 // PIG55 | | | | | | |
| TRK-fused gene | TFG | NM_001007565 // | FLJ36137 // TF6 // | 2633773 | chr3 | + | 101910681 | 101950800 | 9.22 |
| | | NM_006070 | TRKT3 | | | | | | |
| LIM domain 7 | LMO7 | NM_005358 | FBX20 // FBXO20 // | 3494137 | chr13 | + | 75092571 | 75566974 | 9.22 |
| | | | KIAA0858 // LOMP | | | | | | |
| KIAA0090 | KIAA0090 | NM_015047 | RP1-43E13.1 | 2399620 | chr1 | − | 19413665 | 19450633 | 9.22 |
| RNA binding motif protein 23 // | RBM23 // | NM_001077351 // | CAPERbeta // | 3556888 | chr14 | − | 22439714 | 22458231 | 9.18 |
| chromosome 9 open reading frame | C9orf102 | NM_001077352 // | FLJ10482 // | | | | | | |
| 102 | | NM_018107 // | MGC4458 // PP239 | | | | | | |
| | | NM_001034155 // | // RNPC4 // | | | | | | |
| | | NM_020207 | FLJ37706 // SR278 | | | | | | |
| suppressor of Ty 7 (S. cerevisiae)- | SUPT7L | NM_014860 | ART1 // KIAA0764 // | 2546008 | chr2 | − | 27718957 | 27740160 | 9.15 |
| like | | | MGC90306 // SPT7L | | | | | | |
| | | | // STAF65 // | | | | | | |
| | | | STAF65(gamma) | | | | | | |
| transforming, acidic coiled-coil | TACC3 | NM_006342 | ERIC1 // MGC117382 | 2714955 | chr4 | + | 1692616 | 1716693 | 9.12 |
| containing protein 3 | | | // MGC133242 | | | | | | |
| signal transducing adaptor molecule | STAM | NM_003473 | DKFZp686J2352 // | 3237088 | chr10 | + | 17726140 | 17798809 | 9.11 |
| (SH3 domain and ITAM motif) 1 | | | STAM1 | | | | | | |
| neurotensin receptor 1 (high affinity) | NTSR1 | NM_002531 | NTR | 3892873 | chr20 | + | 60810575 | 60864568 | 9.10 |
| sorting nexin associated golgi protein 1 | SNAG1 | NM_052870 | MGC150827 // | 2809628 | chr5 | + | 53849348 | 53878172 | 9.07 |
| | | | MGC150829 // | | | | | | |
| | | | SH3PX2 // | | | | | | |
| | | | SH3PXD3B // SNX18 | | | | | | |
| DEAD (Asp-Glu-Ala-Asp) box | DDX42 | NM_007372 // | FLJ43179 // RHELP | 3730899 | chr17 | + | 59204975 | 59250510 | 9.05 |
| polypeptide 42 | | NM_203499 | // RNAHP // | | | | | | |
| adhesion regulating molecule 1 | ADRM1 | NM_007002 // | GP110 // MGC29536 | 3892607 | chr20 | + | 60305485 | 60317305 | 9.05 |
| | | NM_175573 | // Rpn13 | | | | | | |
| dyskeratosis congenita 1, dyskerin | DKC1 | NM_001363 | DKC // NAP57 // | 3996667 | chrX | + | 153644243 | 153659150 | 9.04 |
| | | | NOLA4 // XAP101 // | | | | | | |
| | | | dyskerin | | | | | | |
| GNAS complex locus | GNAS | NM_000516 // | AHO // C20orf45 // | 3891163 | chr20 | + | 56848165 | 56919604 | 9.04 |
| | | NM_001077488 // | GNAS1 // GPSA // | | | | | | |
| | | NM_001077489 // | GSA // GSP // | | | | | | |
| | | NM_001077490 // | MGC33735 // PHP1A | | | | | | |
| | | NM_016592 // | // PHP1B // POH // | | | | | | |
| | | NM_080425 // | dJ309F20.1.1 // | | | | | | |
| | | NM_080426 // | dJ806M20.3.3 | | | | | | |
| | | NR_003259 | | | | | | | |
| protein inhibitor of activated STAT, 1 | PIAS1 | NM_016166 | DDXBP1 // GBP // | 3599280 | chr15 | + | 65898917 | 66277975 | 9.03 |
| | | | GU/RH-II // | | | | | | |
| | | | MGC141878 // | | | | | | |
| | | | MGC141879 // ZMIZ3 | | | | | | |
| WD repeat domain 36 | WDR36 | NM_139281 | DKFZp686I1650 // | 2823820 | chr5 | + | 110455769 | 110517812 | 9.03 |
| | | | GLC1G // TA-WDRP | | | | | | |
| | | | // TAWDRP // | | | | | | |
| tripartite motif-containing 44 | TRIM44 | NM_017583 | DIPB // HSA249128 | 3326842 | chr11 | + | 35600246 | 35826001 | 9.03 |
| | | | // MC7 // MGC3490 | | | | | | |
| transforming growth factor beta | TBRG4 | NM_004749 // | CPR2 // FASTKD4 // | 3049025 | chr7 | − | 45103738 | 45128368 | 9.01 |
| regulator 4 | | NM_030900 // | KIAA0948 | | | | | | |
| | | NM_199122 | | | | | | | |
| guanine nucleotide binding protein- | GNL3 | NM_014366 // | C77032 // E2IG3 // | 2624074 | chr3 | + | 52694976 | 52705212 | 9.00 |
| like 3 (nucleolar) | | NM_206825 // | MGC800 // NS | | | | | | |
| | | NM_206826 | | | | | | | |
| BCL2-associated transcription factor 1 | BCLAF1 | NM_001077440 // | BTF // KIAA0164 // | 2975680 | chr6 | − | 136619703 | 136652847 | 8.99 |
| | | NM_001077441 // | bK211L9.1 | | | | | | |
| | | NM_014739 | | | | | | | |
| cyclin L2 // aurora kinase A | CCNL2 // | NM_001039577 // | ANIA-6B // | 4041923 | chr1_random | + | 359569 | 372958 | 8.99 |
| interacting protein 1 | AURKAIP1 | NM_030937 // | DKFZp761A1210 // | | | | | | |
| | | NM_017900 | DKFZp762O195 // | | | | | | |
| | | | HCLA-ISO // HLA- | | | | | | |
| | | | ISO // PCEE // | | | | | | |
| | | | SB138 // AIP // | | | | | | |
| | | | AKIP // FLJ20608 | | | | | | |
| cirrhosis, autosomal recessive 1A | CIRH1A | NM_032830 | CIRHIN // FLJ14728 | 3666566 | chr16 | + | 67722728 | 67761072 | 8.98 |
| (cirhin) | | | // KIAA1988 // NAIC | | | | | | |
| | | | // TEX292 | | | | | | |
| SMAD family member 3 | SMAD3 | NM_005902 | DKFZP586N0721 // | 3598959 | chr15 | + | 65145043 | 65296818 | 8.97 |
| | | | DKFZp686J10186 // | | | | | | |
| | | | HSPC193 // | | | | | | |
| | | | HsT17436 // JV15-2 | | | | | | |
| | | | // MADH3 // | | | | | | |
| | | | MGC60396 // Smad 3 | | | | | | |
| proteasome (prosome, macropain) | PSMB7 | NM_002799 | Z | 3225003 | chr9 | − | 126155580 | 126223284 | 8.97 |
| subunit, beta type, 7 | | | | | | | | | |
| cyclin L2 // aurora kinase A | CCNL2 // | NM_001039577 // | ANIA-6B // | 2391532 | chr1 | − | 1314609 | 1324575 | 8.96 |
| interacting protein 1 | AURKAIP1 | NM_030937 // | DKFZp761A1210 // | | | | | | |
| | | NM_017900 | DKFZp762O195 // | | | | | | |
| | | | HCLA-ISO // HLA- | | | | | | |
| | | | ISO // PCEE // | | | | | | |
| | | | SB138 // AIP // | | | | | | |
| | | | AKIP // FLJ20608 | | | | | | |
| mediator of RNA polymerase II | MED28 | NM_025205 | 1500003D12Rik // | 2720181 | chr4 | + | 17225344 | 17244808 | 8.95 |
| transcription, subunit 28 homolog (S. cerevisiae) | | | DKFZP434N185 // | | | | | | |
| | | | EG1 // magicin | | | | | | |
| Rho GTPase activating protein 12 | ARHGAP12 | NM_018287 | DKFZp779N2050 // | 3283920 | chr10 | − | 32134245 | 32261226 | 8.93 |
| | | | FLJ10971 // | | | | | | |
| | | | FLJ20737 // | | | | | | |
| | | | FLJ21785 // | | | | | | |
| | | | FLJ45709 | | | | | | |
| proteasome (prosome, macropain) | PSMD7 | NM_002811 | MOV34 // P40 // | 3668617 | chr16 | + | 72888182 | 72901528 | 8.92 |
| 26S subunit, non-ATPase, 7 (Mov34 | | | S12 | | | | | | |
| homolog) | | | | | | | | | |
| hypothetical protein MGC10433 | MGC10433 | NM_024321 | — | 3830612 | chr19 | + | 40811767 | 40820425 | 8.91 |
| ubiquitin associated protein 2-like | UBAP2L | NM_014847 | FLJ42300 // | 2360083 | chr1 | + | 152459139 | 152510701 | 8.91 |
| | | | KIAA0144 // NICE-4 | | | | | | |
| solute carrier family 20 (phosphate | SLC20A1 | NM_005415 | DKFZp686J2397 // | 2500919 | chr2 | + | 113117773 | 113137861 | 8.90 |
| transporter), member 1 | | | FLJ41426 // GLVR1 | | | | | | |
| | | | // Glvr-1 // PIT1 // | | | | | | |
| | | | PiT-1 | | | | | | |
| protein phosphatase 1, regulatory | PPP1R10 | NM_002714 | CAT53 // FB19 // | 2948425 | chr6 | − | 30676177 | 30694348 | 8.87 |
| (inhibitor) subunit 10 | | | PNUTS | | | | | | |
| SAP30 binding protein // | SAP30BP // | NM_013260 // | DKFZp586L2022 // | 3735107 | chr17 | + | 71174801 | 71215729 | 8.86 |
| cardiotrophin-like cytokine factor 1 | CLCF1 | NM_013246 | HCNGP // HTRG // | | | | | | |
| | | | HTRP // BSF3 // | | | | | | |
| | | | CISS2 // CLC // | | | | | | |
| | | | NNT1 // NR6 | | | | | | |
| ATP-binding cassette, sub-family E | ABCE1 | NM_001040876 // | ABC38 // OABP // | 2746024 | chr4 | + | 146238626 | 146270126 | 8.86 |
| (OABP), member 1 | | NM_002940 | RLI // RNASEL1 // | | | | | | |
| | | | RNASELI // RNS4I | | | | | | |
| BTAF1 RNA polymerase II, B-TFIID | BTAF1 | NM_003972 | KIAA0940 // | 3257938 | chr10 | + | 93673509 | 93821994 | 8.84 |
| transcription factor-associated, | | | MGC138406 // MOT1 | | | | | | |
| 170 kDa (Mot1 homolog, S. cerevisiae) | | | // TAF(II)170 // | | | | | | |
| | | | TAF172 // TAFII170 | | | | | | |
| syntaxin 5 | STX5 | XM_001128716 // | SED5 // STX5A | 3376193 | chr11 | − | 62330946 | 62356126 | 8.84 |
| | | NM_003164 | | | | | | | |
| cyclin A2 | CCNA2 | NM_001237 | CCN1 // CCNA | 2784113 | chr4 | − | 122956335 | 122964516 | 8.81 |
| integrator complex subunit 7 | INTS7 | NM_015434 | C1orf73 // | 2454532 | chr1 | − | 210180312 | 210275613 | 8.81 |
| | | | DKFZP434B168 // | | | | | | |
| | | | INT7 | | | | | | |
| regulator of chromosome | RCC1 // | NM_001048194 // | CHC1 // RCC1-1 // | 2327482 | chr1 | + | 28705062 | 28802493 | 8.81 |
| condensation 1 // small nucleolar | SNHG3 | NM_001048195 // | RNU17C // RNU17D | | | | | | |
| RNA host gene (non-protein coding) 3 | | NM_001269 // | // U17HG // U17HG-A | | | | | | |
| | | NR_002909 | | | | | | | |
| LEM domain containing 3 | LEMD3 | NM_014319 | MAN1 | 3420079 | chr12 | + | 63806051 | 63928317 | 8.80 |
| upstream binding protein 1 (LBP-1a) | UBP1 | NM_014517 | DKFZp686L1745 // | 2668351 | chr3 | − | 33404835 | 33456874 | 8.80 |
| | | | LBP-1B // LBP-1a | | | | | | |
| | | | // LBP1A // LBP1B | | | | | | |
| zinc finger protein 384 | ZNF384 | NM_001039916 // | CAGH1 // CAGH1A | 3442205 | chr12 | − | 6645924 | 6668961 | 8.79 |
| | | NM_001039917 // | // CIZ // ERDA2 // | | | | | | |
| | | NM_001039918 // | NMP4 // NP // | | | | | | |
| | | NM_001039919 // | TNRC1 | | | | | | |
| | | NM_001039920 // | | | | | | | |
| | | NM_133476 | | | | | | | |
| caveolin 2 | CAV2 | NM_001233 // | CAV // MGC12294 | 3020273 | chr7 | + | 115926553 | 115935830 | 8.78 |
| | | NM_198212 | | | | | | | |
| actin, beta | ACTB | NM_001101 | PS1TP5BP1 | 3036924 | chr7 | − | 5533433 | 5537011 | 8.77 |
| acyl-Coenzyme A binding domain | ACBD3 | NM_022735 | GCP60 // GOCAP1 | 2458701 | chr1 | − | 224398953 | 224463924 | 8.76 |
| containing 3 | | | // GOLPH1 // PAP7 | | | | | | |
| eukaryotic translation initiation factor | EIF3S10 | NM_003750 | EIF3 // EIF3A // | 3309215 | chr10 | − | 120621551 | 120830522 | 8.75 |
| 3, subunit 10 theta, 150/170 kDa | | | KIAA0139 // P167 // | | | | | | |
| | | | eIF3-p170 // eIF3- | | | | | | |
| | | | theta // p180 // p185 | | | | | | |
| CDC42 small effector 2 | CDC42SE2 | NM_001038702 // | FLJ21967 // SPEC2 | 2828146 | chr5 | + | 130616639 | 130781182 | 8.75 |
| | | NM_020240 | | | | | | | |
| zinc finger, AN1-type domain 2B | ZFAND2B | NM_138802 | — | 2528308 | chr2 | + | 219779769 | 219782603 | 8.72 |
| ribosomal protein S20 | RPS20 | NM_001023 | FLJ27451 // | 3136129 | chr8 | − | 57139946 | 57185891 | 8.72 |
| | | | MGC102930 | | | | | | |
| exportin 1 (CRM1 homolog, yeast) // | XPO1 // | NM_003400 // | CRM1 // | 2555490 | chr2 | − | 61558490 | 61619343 | 8.71 |
| thyroid hormone receptor, beta | THRB | NM_000461 | DKFZp686B1823 // | | | | | | |
| (erythroblastic leukemia viral (v-erb- | | | ERBA-BETA // | | | | | | |
| a) oncogene homolog 2, avian) | | | ERBA2 // GRTH // | | | | | | |
| | | | MGC126109 // | | | | | | |
| | | | MGC126110 // | | | | | | |
| | | | NR1A2 // THR1 // | | | | | | |
| | | | THRB1 // THRB2 | | | | | | |
| D4, zinc and double PHD fingers | DPF2 | NM_006268 | MGC10180 // REQ // | 3335089 | chr11 | + | 64857703 | 64877276 | 8.71 |
| family 2 | | | UBID4 // ubi-d4 | | | | | | |
| basic leucine zipper and W2 domains 1 | BZW1 | XM_001126385 // | BZAP45 // KIAA0005 | 2522439 | chr2 | + | 201384456 | 201418386 | 8.71 |
| | | NM_014670 | // Nbla10236 | | | | | | |
| pogo transposable element with ZNF | POGZ | NM_015100 // | KIAA0461 // | 2435044 | chr1 | − | 149641830 | 149753035 | 8.70 |
| domain | | NM_145796 // | MGC71543 // SUHW5 | | | | | | |
| | | NM_207171 | // ZNF635 | | | | | | |
| basic helix-loop-helix domain | BHLHB3 | NM_030762 | DEC2 // SHARP-1 // | 3448088 | chr12 | − | 26141435 | 26179615 | 8.69 |
| containing, class B, 3 | | | SHARP1 | | | | | | |
| family with sequence similarity 83, | FAM83D | NM_030919 | C20orf129 // | 3884892 | chr20 | + | 36988369 | 37015223 | 8.69 |
| member D | | | FLJ38341 // | | | | | | |
| | | | dJ616B8.3 | | | | | | |
| thioredoxin domain containing 14 // | TXNDC14 // | NM_015959 // | CGI-31 // | 3331487 | chr11 | + | 57235839 | 57391291 | 8.68 |
| catenin (cadherin-associated | CTNND1 | XM_001126721 // | DKFZp781O2021 // | | | | | | |
| protein), delta 1 | | XM_001126756 // | MGC111151 // PIG26 | | | | | | |
| | | XM_001126767 // | // TMX2 // CAS // | | | | | | |
| | | XM_001126781 // | CTNND // KIAA0384 | | | | | | |
| | | XM_001126793 // | // P120CAS // | | | | | | |
| | | XM_001126823 // | P120CTN // p120 | | | | | | |
| | | NM_001331 | | | | | | | |
| thymopoietin | TMPO | NM_001032283 // | CMD1T // LAP2 // | 3427767 | chr12 | + | 97395927 | 97502543 | 8.68 |
| | | NM_001032284 // | MGC61508 // | | | | | | |
| | | NM_003276 | PRO0868 // TP | | | | | | |
| jumonji, AT rich interactive domain | JARID1A | NM_001042603 // | RBBP2 // RBP2 | 3439603 | chr12 | − | 269504 | 368944 | 8.67 |
| 1A | | NM_005056 | | | | | | | |
| sorting nexin 19 | SNX19 | NM_014758 | CHET8 // | 3398482 | chr11 | − | 130250320 | 130291615 | 8.67 |
| | | | DKFZp667I205 // | | | | | | |
| | | | KIAA0254 | | | | | | |
| Der1-like domain family, member 1 | DERL1 | NM_024295 | DER-1 // DER1 // | 3151401 | chr8 | − | 124094605 | 124123781 | 8.67 |
| | | | FLJ13784 // | | | | | | |
| | | | FLJ42092 // | | | | | | |
| | | | MGC3067 // | | | | | | |
| eukaryotic translation initiation factor | EIF3S9 | NM_001037283 // | EIF3-ETA // EIF3- | 2987441 | chr7 | + | 2315929 | 2386896 | 8.66 |
| 3, subunit 9 eta, 116 kDa | | NM_003751 | P110 // EIF3-P116 // | | | | | | |
| | | | MGC104664 // | | | | | | |
| | | | MGC131875 // PRT1 | | | | | | |
| | | | // eIF3b | | | | | | |
| WDR45-like | WDR45L | NM_019613 | WIPI-3 // WIPI3 | 3775157 | chr17 | − | 78165748 | 78199803 | 8.65 |
| conserved helix-loop-helix ubiquitous | CHUK | NM_001278 | IKBKA // IKK-alpha | 3303300 | chr10 | − | 101938115 | 101979345 | 8.62 |
| kinase | | | // IKK1 // IKKA // | | | | | | |
| | | | NFKBIKA // TCF16 | | | | | | |
| WD repeat domain 77 // oviductal | WDR77 // | NM_024102 // | HKMT1069 // MEP50 | 2427930 | chr1 | − | 111782351 | 111793512 | 8.60 |
| glycoprotein 1, 120 kDa (mucin 9, | OVGP1 | NM_002557 | // MGC2722 // | | | | | | |
| oviductin) | | | Nbla10071 // RP11- | | | | | | |
| | | | 552M11.3 // CHIT5 // | | | | | | |
| | | | EGP // MUC9 // | | | | | | |
| Paf1, RNA polymerase II associated | PAF1 | NM_019088 | F23149_1 // | 3861978 | chr19 | − | 44568113 | 44592162 | 8.60 |
| factor, homolog (S. cerevisiae) | | | FLJ11123 // PD2 | | | | | | |
| kinesin family member 23 | KIF23 | NM_004856 // | CHO1 // KNSL5 // | 3599811 | chr15 | + | 67493677 | 67527808 | 8.60 |
| | | NM_138555 | MKLP-1 // MKLP1 | | | | | | |
| BMS1-like, ribosome assembly | BMS1L // | NM_014753 // | KIAA0187 // TTC7L1 | 3243708 | chr10 | + | 42597978 | 42682443 | 8.58 |
| protein (yeast) // tetratricopeptide | TTC7B | NM_001010854 | // c14_5685 | | | | | | |
| repeat domain 7B | | | | | | | | | |
| insulin induced gene 2 | INSIG2 | NM_016133 | MGC26273 | 2502424 | chr2 | + | 118558758 | 118593436 | 8.57 |
| ubiquitin specific peptidase 9, X- | USP9X // | NM_001039590 // | DFFRX // FAF // | 3974708 | chrX | + | 40829542 | 40980776 | 8.57 |
| linked // ubiquitin specific peptidase | USP9Y | NM_001039591 // | AZF // AZFA // | | | | | | |
| 9, Y-linked (fat facets-like, | | NM_004654 | DFFRY // SP3 | | | | | | |
| squamous cell carcinoma antigen | SART3 | NM_014706 | KIAA0156 // | 3470253 | chr12 | − | 107440140 | 107479296 | 8.57 |
| recognized by T cells 3 | | | MGC138188 // RP11- | | | | | | |
| | | | 13G14 // TIP110 // | | | | | | |
| | | | p110(nrb) | | | | | | |
| LIM domain only 4 | LMO4 | NM_006769 | — | 2345286 | chr1 | + | 87458333 | 87588817 | 8.56 |
| peptidylprolyl isomerase G | PPIG | NM_004792 | CARS-Cyp // CYP // | 2514441 | chr2 | + | 170149096 | 170206156 | 8.55 |
| (cyclophilin G) | | | MGC133241 // | | | | | | |
| guanine nucleotide binding protein (G | GNAQ | NM_002072 | G-ALPHA-q // GAQ | 3210808 | chr9 | − | 79475043 | 79836455 | 8.53 |
| protein), q polypeptide | | | | | | | | | |
| TSC22 domain family, member 1 | TSC22D1 | NM_006022 // | DKFZp686O19206 // | 3512294 | chr13 | − | 43905532 | 44202063 | 8.53 |
| | | NM_183422 | MGC17597 // RP11- | | | | | | |
| | | | 269C23.2 // TGFB1I4 | | | | | | |
| | | | // TSC22 | | | | | | |
| serine/arginine repetitive matrix 2 | SRRM2 | NM_016333 | 300-KD // | 3645253 | chr16 | + | 2742650 | 2761908 | 8.52 |
| | | | DKFZp686O15166 // | | | | | | |
| | | | FLJ21926 // | | | | | | |
| | | | FLJ22250 // | | | | | | |
| | | | KIAA0324 // | | | | | | |
| | | | MGC40295 // SRL300 | | | | | | |
| | | | // SRm300 | | | | | | |
| methionine adenosyltransferase II, | MAT2A | NM_005911 | MATA2 // MATII // | 2491615 | chr2 | + | 85597809 | 85625908 | 8.51 |
| alpha | | | SAMS2 | | | | | | |
| ubinuclein 1 | UBN1 | NM_001079514 // | VT // VT4 | 3646434 | chr16 | + | 4836677 | 4872358 | 8.51 |
| | | NM_016936 | | | | | | | |
| solute carrier family 38, member 2 | SLC38A2 | NM_018976 | ATA2 // KIAA1382 // | 3452323 | chr12 | − | 45038242 | 45132603 | 8.50 |
| | | | PRO1068 // SAT2 // | | | | | | |
| | | | SNAT2 | | | | | | |
| GrpE-like 1, mitochondrial (E. coli) | GRPEL1 | NM_025196 | FLJ25609 // HMGE | 2759404 | chr4 | − | 7107908 | 7187815 | 8.48 |
| BAT2 domain containing 1 | BAT2D1 | NM_015172 | BAT2-iso // XTP2 | 2367154 | chr1 | + | 169716078 | 169829262 | 8.48 |
| eukaryotic translation initiation factor | EIF3S7 | NM_003753 | MGC126526 // | 3959631 | chr22 | − | 35236857 | 35255429 | 8.48 |
| 3, subunit 7 zeta, 66/67 kDa | | | MGC17258 // eIF3- | | | | | | |
| | | | p66 // eIF3-zeta // | | | | | | |
| | | | eIF3d | | | | | | |
| polymerase (DNA directed) sigma | POLS | NM_006999 | LAK-1 // POLK // | 2800503 | chr5 | + | 6736994 | 6893446 | 8.47 |
| | | | TRF4 // TRF4-1 | | | | | | |
| interleukin enhancer binding factor 2, | ILF2 | NM_004515 | MGC8391 // NF45 // | 2436132 | chr1 | − | 151900372 | 151910103 | 8.46 |
| 45 kDa | | | PRO3063 | | | | | | |
| chromosome X open reading frame | CXorf39 | NM_207318 | — | 3985866 | chrX | + | 103297819 | 103323642 | 8.45 |
| lysosomal-associated membrane | LAMP1 | NM_005561 | CD107a // LAMPA // | 3502570 | chr13 | + | 112999460 | 113025981 | 8.45 |
| protein 1 | | | LGP120 | | | | | | |
| YTH domain containing 1 | YTHDC1 | NM_001031732 // | KIAA1966 // YT521 | 2772017 | chr4 | − | 68858700 | 68934802 | 8.45 |
| | | NM_133370 | // YT521-B | | | | | | |
| RAD21 homolog (S. pombe) | RAD21 | NM_006265 | FLJ25655 // | 3149843 | chr8 | − | 117909861 | 117956284 | 8.45 |
| | | | FLJ40596 // HR21 // | | | | | | |
| | | | HRAD21 // KIAA0078 | | | | | | |
| | | | // MCD1 // NXP1 // | | | | | | |
| | | | SCC1 // hHR21 | | | | | | |
| TATA box binding protein | TBP | NM_003194 | GTF2D // GTF2D1 // | 2937984 | chr6 | + | 170705200 | 170723945 | 8.44 |
| | | | MGC117320 // | | | | | | |
| | | | MGC126054 // | | | | | | |
| | | | MGC126055 // | | | | | | |
| | | | SCA17 // TFIID | | | | | | |
| Rho GTPase activating protein 29 | ARHGAP29 | NM_004815 | PARG1 // RP11- | 2423829 | chr1 | − | 94409906 | 94549874 | 8.43 |
| | | | 255E17.1 | | | | | | |
| PRP4 pre-mRNA processing factor 4 | PRPF4 | NM_004697 | HPRP4 // HPRP4P // | 3185558 | chr9 | + | 115077230 | 115095267 | 8.42 |
| homolog (yeast) | | | PRP4 // Prp4p | | | | | | |
| myosin phosphatase-Rho interacting | M-RIP // | NM_0165134 // | KIAA0864 // RHOIP3 | 3712363 | chr17 | + | 16886542 | 17062286 | 8.42 |
| protein | C17orf84 | NM_201274 | // p116Rip | | | | | | |
| E2F transcription factor 4, | E2F4 | NM_001950 | E2F-4 | 3665288 | chr16 | + | 65783266 | 65790299 | 8.42 |
| p107/p130-binding | | | | | | | | | |
| glutamine-rich 1 | QRICH1 | NM_017730 // | FLJ20259 // | 2673902 | chr3 | − | 49042148 | 49106498 | 8.42 |
| | | NM_198880 | MGC131838 | | | | | | |
| annexin A1 | ANXA1 | NM_000700 | ANX1 // LPC1 | 3174816 | chr9 | + | 74914324 | 74983394 | 8.41 |
| serine/threonine kinase 4 | STK4 | NM_006282 | DKFZp686A2068 // | 3886704 | chr20 | + | 43028534 | 43142041 | 8.38 |
| | | | KRS2 // MST1 // | | | | | | |
| | | | YSK3 | | | | | | |
| chromosome X open reading frame | CXorf15 | NM_018360 | FIAT // FLJ11209 // | 3970166 | chrX | + | 16714433 | 16772561 | 8.36 |
| 15 | | | LSR5 // MGC126621 | | | | | | |
| | | | // MGC126625 | | | | | | |
| transportin 2 (importin 3, karyopherin | TNPO2 | NM_013433 | FLJ12155 // IPO3 // | 3851651 | chr19 | − | 12670912 | 12695776 | 8.35 |
| beta 2b) | | | KPNB2B // TRN2 | | | | | | |
| pleckstrin homology-like domain, | PHLDB1 | NM_015157 | DKFZp686H039 // | 3351564 | chr11 | + | 117982385 | 118033939 | 8.34 |
| family B, member 1 | | | DKFZp686O24210 // | | | | | | |
| | | | FLJ00141 // | | | | | | |
| | | | FLJ90266 // | | | | | | |
| | | | KIAA0638 // LL5A // | | | | | | |
| | | | MGC111531 | | | | | | |
| tumor necrosis factor receptor | TNFRSF12A | NM_016639 | CD266 // FN14 // | 3645555 | chr16 | + | 3010334 | 3012380 | 8.34 |
| superfamily, member 12A | | | TWEAKR | | | | | | |
| SLC7A5 pseudogene | LAT1-3TM | NR_002593 // | DC49 // MLAS // | 3684100 | chr16 | − | 21320984 | 22449842 | 8.33 |
| | // IMAA | NR_002594 | hLAT1-3TM // MMAA | | | | | | |
| CD3e molecule, epsilon associated | CD3EAP | NM_012099 | ASE-1 // CAST // | 3836243 | chr19 | + | 50597660 | 50605831 | 8.33 |
| protein | | | MGC118851 // PAF49 | | | | | | |
| kinesin family member 2C | KIF2C | NM_006845 | KNSL6 // MCAK | 2334098 | chr1 | + | 44978097 | 45005994 | 8.33 |
| CD55 molecule, decay accelerating | CD55 | NM_000574 | CR // DAF // TC | 2377229 | chr1 | + | 205561488 | 205607671 | 8.32 |
| factor for complement (Cromer blood | | | | | | | | | |
| group) | | | | | | | | | |
| aldolase A, fructose-bisphosphate | ALDOA | NM_000034 // | ALDA // MGC10942 | 3655920 | chr16 | + | 29968869 | 29991009 | 8.31 |
| | | NM_184041 // | // MGC17716 // | | | | | | |
| | | NM_184043 | MGC17767 | | | | | | |
| denticleless homolog (Drosophila) | DTL | NM_016448 | CDT2 // DCAF2 // | 2378937 | chr1 | + | 210275565 | 210399046 | 8.30 |
| | | | L2DTL // RAMP | | | | | | |
| adhesion molecule with Ig-like | AMIGO2 | NM_181847 | ALI1 // DEGA | 3452478 | chr12 | − | 45726359 | 45874177 | 8.30 |
| domain 2 | | | | | | | | | |
| ST3 beta-galactoside alpha-2,3- | ST3GAL1 | NM_003033 // | Gal-NAc6S // | 3154398 | chr8 | − | 134535811 | 134653449 | 8.29 |
| sialyltransferase 1 | | NM_173344 | MGC9183 // SIAT4A | | | | | | |
| | | | // SIATFL // | | | | | | |
| | | | ST3GalA // | | | | | | |
| | | | ST3GalA.1 // | | | | | | |
| tripartite motif-containing 8 | TRIM8 | NM_030912 | GERP // RNF27 | 3261820 | chr10 | + | 104384137 | 104419190 | 8.29 |
| fibroblast growth factor (acidic) | FIBP | NM_004214 // | FGFIBP // FIBP-1 | 3377964 | chr11 | − | 65407787 | 65412586 | 8.29 |
| intracellular binding protein | | NM_198897 | | | | | | | |
| PAP associated domain containing 1 | PAPD1 // | NM₁₃ 018109 // | FLJ10486 // RP11- | 3283378 | chr10 | − | 30511316 | 30764402 | 8.26 |
| // collagen, type XI, alpha 1 | COL11A1 | NM_001854 // | 305E6.3 // mtPAP // | | | | | | |
| | | NM_080629 // | CO11A1 // COLL6 // | | | | | | |
| | | NM_080630 | STL2 | | | | | | |
| exosome component 10 | EXOSC10 | NM_001001998 // | PM-Scl // PM/Scl- | 2396480 | chr1 | − | 11049173 | 11082811 | 8.26 |
| | | NM_002685 | 100 // PMSCL // | | | | | | |
| | | | PMSCL2 // RRP6 // | | | | | | |
| | | | Rrp6p // p2 // p3 // | | | | | | |
| | | | p4 | | | | | | |
| B-cell CLL/lymphoma 9-like | BCL9L | NM_182557 | BCL9-2 // DLNB11 | 3393993 | chr11 | − | 118272076 | 118304947 | 8.23 |
| 1-acylglycerol-3-phosphate O- | AGPAT5 | NM_018361 | 1-AGPAT5 // | 3083936 | chr8 | + | 6553307 | 6633056 | 8.22 |
| acyltransferase 5 (lysophosphatidic | | | LPAAT-e // LPAAT- | | | | | | |
| acid acyltransferase, epsilon) | | | epsilon | | | | | | |
| chaperonin containing TCP1, subunit | CCT6A | NM_001009186 // | CCT-zeta // CCT- | 3003193 | chr7 | + | 56086894 | 56099148 | 8.22 |
| 6A (zeta 1) | | NM_001762 | zeta-1 // CCT6 // | | | | | | |
| | | | Cctz // HTR3 // | | | | | | |
| | | | MGC126214 // | | | | | | |
| | | | MGC126215 // | | | | | | |
| | | | MoDP-2 // TCP-1- | | | | | | |
| | | | zeta // TCP20 // | | | | | | |
| | | | TCPZ // TTCP20 | | | | | | |
| jumonji, AT rich interactive domain | JARID1B | NM_006618 | FLJ10538 // | 2451309 | chr1 | − | 200963111 | 201048571 | 8.22 |
| 1B | | | FLJ12459 // | | | | | | |
| | | | FLJ12491 // | | | | | | |
| | | | FLJ16281 // | | | | | | |
| | | | FLJ23670 // PLU-1 | | | | | | |
| | | | // PUT1 // | | | | | | |
| | | | RBBP2H1A | | | | | | |
| solute carrier family 25, member 37 | SLC25A37 | XM_001128238 // | HT015 // MFRN // | 3090006 | chr8 | + | 23439117 | 23485313 | 8.21 |
| | | XM_001128248 // | MSC // MSCP // | | | | | | |
| | | XM_001128255 // | PRO1278 // | | | | | | |
| | | XM_001128269 // | PRO1584 // | | | | | | |
| | | NM_016612 | PRO2217 | | | | | | |
| nucleoporin 50 kDa | NUP50 | NM_007172 // | MGC39961 // | 3948461 | chr22 | + | 43938390 | 43960160 | 8.21 |
| | | NM_153645 | NPAP60 // NPAP60L | | | | | | |
| mediator of RNA polymerase II | MED8 | NM_001001651 // | ARC32 // MGC17544 | 2409344 | chr1 | − | 43622176 | 43628144 | 8.20 |
| transcription, subunit 8 homolog (S. cerevisiae) | | NM_001001653 // | // MGC19641 | | | | | | |
| | | NM_001001654 // | | | | | | | |
| | | NM_052877 // | | | | | | | |
| | | NM_201542 | | | | | | | |
| eukaryotic translation initiation factor | EIF4A2 | NM_001967 | BM-010 // DDX2B // | 2656738 | chr3 | + | 187961636 | 187990742 | 8.20 |
| 4A, isofom 2 | | | EIF4A // EIF4F | | | | | | |
| GA binding protein transcription | GABPB2 | NM_002041 // | BABPB2 // E4TF1 // | 3623683 | chr15 | − | 48356681 | 48434794 | 8.18 |
| factor, beta subunit 2 | | NM_005254 // | E4TF1-47 // E4TF1- | | | | | | |
| | | NM_016654 // | 53 // E4TF1B // | | | | | | |
| | | NM_016655 // | GABPB // GABPB1 | | | | | | |
| | | NM_181427 | // NRF2B1 // | | | | | | |
| RNA binding motif protein 22 | RBM22 | NM_018047 | FLJ10290 // ZC3H16 | 2881521 | chr5 | − | 150050460 | 150060885 | 8.16 |
| meningioma expressed antigen 5 | MGEA5 | NM_012215 | FLJ11229 // | 3304012 | chr10 | − | 103534201 | 103568420 | 8.15 |
| (hyaluronidase) | | | FLJ23355 // | | | | | | |
| | | | KIAA0679 // MEA5 // | | | | | | |
| | | | NCOAT | | | | | | |
| low density lipoprotein receptor | LDLR | NM_000527 | FH // FHC | 3821015 | chr19 | + | 11061114 | 11115050 | 8.15 |
| (familial hypercholesterolemia) | | | | | | | | | |
| cell division cycle 40 homolog (S. cerevisiae) | CDC40 | NM_015891 | EHB3 // FLJ10564 // | 2921086 | chr6 | + | 110606544 | 110682171 | 8.13 |
| | | | MGC102802 // | | | | | | |
| | | | PRP17 // PRPF17 | | | | | | |
| hypothetical protein HSPC111 | HSPC111 | NM_016391 | HSPC185 | 2888284 | chr5 | − | 175743557 | 175749348 | 8.13 |
| solute carrier family 2 (facilitated | SLC2A1 | NM_006516 | GLUT // GLUT1 // | 2409104 | chr1 | − | 43124794 | 43338329 | 8.12 |
| glucose transporter), member 1 | | | MGC141895 // | | | | | | |
| | | | MGC141896 | | | | | | |
| caspase 2, apoptosis-related | CASP2 | NM_032982 // | CASP-2 // ICH-1L | 3029030 | chr7 | + | 142695524 | 142714904 | 8.11 |
| cysteine peptidase (neural precursor | | NM_032983 | // ICH-1L/1S // | | | | | | |
| cell expressed, developmentally | | | ICH1 // NEDD2 | | | | | | |
| down-regulated 2) | | | | | | | | | |
| HECT domain containing 1 | HECTD1 | NM_015382 | FLJ38315 // | 3559570 | chr14 | − | 30629936 | 31002364 | 8.10 |
| | | | KIAA1131 | | | | | | |
| ariadne homolog, ubiquitin- | ARIH1 | NM_005744 | ARI // HARI // | 3600744 | chr15 | + | 70553731 | 70666726 | 8.09 |
| conjugating enzyme E2 binding | | | HHARI // UBCH7BP | | | | | | |
| protein, 1 (Drosophila) | | | | | | | | | |
| RNA binding motif protein, X-linked 2 | RBMX2 | NM_016024 | CGI-79 | 3990795 | chrX | + | 129356824 | 129381607 | 8.09 |
| family with sequence similarity 29, | FAM29A | NM_017645 | MGC102696 // | 3200611 | chr9 | − | 19042752 | 19093153 | 8.09 |
| member A | | | MGC138798 // | | | | | | |
| | | | MGC138799 // RP11- | | | | | | |
| | | | 296P7.3 | | | | | | |
| NGFI-A binding protein 1 (EGR1 | NAB1 | NM_005966 | — | 2520225 | chr2 | + | 191205069 | 191437200 | 8.08 |
| binding protein 1) | | | | | | | | | |
| zinc finger protein 706 | ZNF706 | NM_001042510 // | HSPC038 // PNAS- | 3147020 | chr8 | − | 102226101 | 102376609 | 8.08 |
| | | NM_001042511 // | 106 // PNAS-113 | | | | | | |
| | | NM_016096 | | | | | | | |
| chaperonin containing TCP1, subunit | CCT4 | NM_006430 | Cctd // MGC126164 | 2555630 | chr2 | − | 61939693 | 61970469 | 8.07 |
| 4 (delta) | | | // MGC126165 // | | | | | | |
| | | | SRB | | | | | | |
| zinc finger, CCHC domain containing | ZCCHC8 | NM_017612 | DKFZp434E2220 | 3475679 | chr12 | − | 121522105 | 121551469 | 8.07 |
| methyltransferase like 2B // | METTL2B // | NM 018396 // | FLJ11350 // | 3730211 | chr17 | + | 57854988 | 57881176 | 8.06 |
| methyltransferase like 2A | METTL2A | NM_001005372 // | FLJ12760 // METL // | | | | | | |
| | | NM_181725 | METTL2 // METTL2A | | | | | | |
| | | | // PSENIP1 | | | | | | |
| fibrosin 1 | FBS1 | NM_022452 | FBS // FLJ11618 | 3656362 | chr16 | + | 30577810 | 30590035 | 8.06 |
| splicing factor, arginine/serine-rich 8 | SFRS8 | NM_004592 | SWAP | 3438417 | chr12 | + | 130761587 | 130852472 | 8.05 |
| (suppressor-of-white-apricot | | | | | | | | | |
| homolog, Drosophila) | | | | | | | | | |
| CCR4-NOT transcription complex, | CNOT7 | NM_013354 // | CAF1 // hCAF-1 | 3125775 | chr8 | − | 17128912 | 17148758 | 8.05 |
| subunit 7 | | NM_054026 | | | | | | | |
| BTB (POZ) domain containing 7 | BTBD7 | NM_001002860 // | DKFZp686N0544 // | 3577277 | chr14 | − | 92773656 | 92966460 | 8.05 |
| | | NM_018167 | FUP1 // MGC48310 | | | | | | |
| chromosome 17 open reading frame | C17orf79 | NM_018405 | FLJ21119 // | 3752424 | chr17 | − | 27203014 | 27210424 | 8.04 |
| 79 | | | HSA272196 // TTP1 | | | | | | |
| transcription factor 12 (HTF4, helix- | TCF12 | NM_003205 // | HEB // HTF4 // | 3595096 | chr15 | + | 54963208 | 55402126 | 8.03 |
| loop-helix transcription factors 4) | | NM_207036 // | HsT17266 | | | | | | |
| | | NM_207037 // | | | | | | | |
| | | NM_207038 // | | | | | | | |
| | | NM_207040 | | | | | | | |
| ATP binding domain 1 famliy, member B | ATPBD1B | NM_018066 | FLJ10349 | 2402838 | chr1 | − | 27073870 | 27089311 | 8.03 |
| PRP3 pre-mRNA processing facter 3 | PRPF3 // | NM_004698 // | HPRP3 // HPRP3P // | 2358171 | chr1 | + | 148560583 | 148592321 | 8.03 |
| homolog (S. cerevisiae) // KIAA0460 | KIAA0460 | NM_015203 | PRP3 // Prp3p // | | | | | | |
| | | | RP18 // FLJ32145 // | | | | | | |
| | | | HSPC099 | | | | | | |
| G1 to S phase transition 1 | GSPT1 | NM_002094 | 551G9.2 // ETF3A // | 3680610 | chr16 | − | 11865619 | 11917408 | 8.00 |
| | | | GST1 // eRF3a | | | | | | |
| zinc finger protein 408 | ZNF408 | NM_024741 | FLJ12827 | 3329461 | chr11 | + | 46678944 | 46684037 | 7.99 |
| chromosome 20 open reading frame 4 | C20orf4 | NM_015511 | CGI-23 // | 3883787 | chr20 | + | 34287657 | 34322234 | 7.99 |
| | | | DKFZp564N1363 // | | | | | | |
| | | | bA234K24.2 | | | | | | |
| DnaJ (Hsp40) homolog, subfamily A, | DNAJA2 | NM_005880 | CPR3 // DJA2 // | 3690084 | chr16 | − | 45546783 | 45565155 | 7.98 |
| member 2 | | | DNAJ // DNJ3 // | | | | | | |
| | | | HIRIP4 // PRO3015 | | | | | | |
| | | | // RDJ2 | | | | | | |
| splicing factor, arginine/serine-rich | SFRS10 | NM_004593 | DKFZp686F18120 // | 2709062 | chr3 | − | 187048509 | 187160523 | 7.98 |
| 10 (transformer 2 homolog, | | | Htra2-beta // | | | | | | |
| Drosophila) | | | SRFS10 // TRA2- | | | | | | |
| | | | BETA // TRA2B | | | | | | |
| trinucleotide repeat containing 6A | TNRC6A | NM_020847 // | CAGH26 // | 3653398 | chr16 | + | 24583665 | 24748478 | 7.98 |
| | | NM_014494 | DKFZp666E117 // | | | | | | |
| | | | FLJ22043 // GW1 // | | | | | | |
| | | | GW182 // KIAA1460 | | | | | | |
| | | | // MGC75384 // | | | | | | |
| | | | TNRC6 | | | | | | |
| testis expressed sequence 10 | TEX10 | NM_017746 | FLJ20287 // | 3217807 | chr9 | − | 102104197 | 102155471 | 7.98 |
| | | | bA208F1.2 | | | | | | |
| nucleoporin 107 kDa | NUP107 | NM_020401 | NUP84 | 3421177 | chr12 | + | 67366998 | 67423025 | 7.97 |
| hypoxia-inducible factor 1, alpha | HIF1AN | NM_017902 | DKFZp762F1811 // | 3260666 | chr10 | + | 102279093 | 102303662 | 7.97 |
| subunit inhibitor | | | FIH1 // FLJ20615 // | | | | | | |
| | | | FLJ22027 | | | | | | |
| myosin IE | MYO1E | NM_004998 | HuncM-IC // | 3626826 | chr15 | − | 57215720 | 57452363 | 7.97 |
| | | | MGC104638 // | | | | | | |
| | | | MYO1C | | | | | | |
| v-ref murine sarcoma 3611 viral | ARAF | NM_001654 | A-RAF // ARAF1 // | 3976299 | chrX | + | 47300826 | 47316249 | 7.96 |
| oncogene homolog | | | PKS2 // RAFA1 | | | | | | |
| nucleolar protein family 6 (RNA- | NOL6 | NM_130793 // | FLJ21959 // | 3203582 | chr9 | − | 33442676 | 33464087 | 7.96 |
| associated) | | NM_022917 // | MGC14896 // | | | | | | |
| | | NM_139235 | MGC14921 // | | | | | | |
| | | | MGC20838 // NRAP | | | | | | |
| | | | // UTP22 // | | | | | | |
| | | | bA311H10.1 | | | | | | |
| chromosome 21 open reading frame | C21orf66 | NM_145328 // | BM-020 // FLJ90561 | 3929395 | chr21 | − | 33028084 | 33066030 | 7.96 |
| 66 | | NM_013329 // | // GCFC | | | | | | |
| | | NM_016631 // | | | | | | | |
| | | NM_058191 | | | | | | | |
| casein kinase 1, alpha 1 | CSNK1A1 | NM_001025105 // | CK1 // HLCDGP1 // | 2880932 | chr5 | − | 148800168 | 148922811 | 7.96 |
| | | NM_001892 | PRO2975 | | | | | | |
| hypothetical protein DKFZp762E1312 | DKFZp762E1312 | NM_018410 | hFLEG1 | 2604254 | chr2 | − | 234406876 | 234427931 | 7.95 |
| PRP6 pre-mRNA processing factor 6 | PRPF6 | NM_012469 | ANT-1 // C20orf14 | 3893849 | chr20 | + | 62082901 | 62134891 | 7.95 |
| homolog (S. cerevisiae) | | | // TOM // U5-102K | | | | | | |
| | | | // hPrp6 | | | | | | |
| tumor suppressing subtransferable | TSSC4 | NM_005706 | — | 3317338 | chr11 | + | 2378304 | 2381662 | 7.93 |
| candidate 4 | | | | | | | | | |
| cell division cycle 25 homolog B (S. pombe) | CDC25B | NM_004358 // | — | 3874438 | chr20 | + | 3715655 | 3734756 | 7.93 |
| | | NM_021872 // | | | | | | | |
| | | NM_021873 | | | | | | | |
| megakaryobiastic leukemia | MKL1 | NM_020831 | BSAC // MAL // | 3961496 | chr22 | − | 39136248 | 39362660 | 7.93 |
| (translocation) 1 | | | MRTF-A | | | | | | |
| topoisomerase (DNA) I | TOP1 | NM_003286 | TOPI | 3885464 | chr20 | + | 39090891 | 39186535 | 7.93 |
| WW domain containing adapter with | WAC | NM_016628 // | BM-016 // | 3240340 | chr10 | + | 28828043 | 29005095 | 7.92 |
| coiled-coil | | NM_100264 // | MGC10753 // | | | | | | |
| | | NM_100486 | PRO1741 // Wwp4 // | | | | | | |
| | | | bA48B24 // | | | | | | |
| ubiquitin specific peptidase 36 | USP36 | NM_025090 | DUB1 | 3772581 | chr17 | − | 74295075 | 74348574 | 7.92 |
| t-complex 1 | TCP1 | NM_001008897 // | CCT-alpha // CCT1 | 2982381 | chr6 | − | 160119521 | 160130731 | 7.92 |
| | | NM_030752 | // CCTa // D6S230E | | | | | | |
| | | | // TCP-1-alpha | | | | | | |
| protein phosphatase 6, catalytic | PPP6C | NM_002721 | — | 3225348 | chr9 | − | 126948675 | 126991992 | 7.91 |
| subunit | | | | | | | | | |
| CDC-like kinase 3 | CLK3 | NM_001292 // | FLJ22858 | 3601741 | chr15 | + | 72677906 | 72719105 | 7.91 |
| | | NM_003992 | | | | | | | |
| anillin, actin binding protein | ANLN | NM_018685 | ANILLIN // | 2997376 | chr7 | + | 36395957 | 36462017 | 7.90 |
| | | | DKFZp779A055 // | | | | | | |
| | | | Scraps // scra | | | | | | |
| adenosylmethionine decarboxylase 1 | AMD1 | NM_001033059 // | ADOMETDC // AMD | 2921296 | chr6 | + | 111287588 | 111324441 | 7.90 |
| | | NM_001634 | // DKFZp313L1234 | | | | | | |
| | | | // FLJ26964 | | | | | | |
| farnesyl-diphosphate | FDFT1 | NM_004462 | DGPT // ERG9 // | 3086206 | chr8 | + | 11690497 | 11740987 | 7.90 |
| farnesyltransferase 1 | | | SQS // SS | | | | | | |
| PRP40 pre-mRNA processing factor | PRPF40A | XM_371575 // | FBP-11 // FBP11 // | 2581548 | chr2 | − | 153212414 | 153283546 | 7.89 |
| 40 homolog A (S. cerevisiae) | | XM_938514 | FLAF1 // FLJ20585 | | | | | | |
| | | | // FNBP3 // HIP10 | | | | | | |
| | | | // HYPA // NY-REN-6 | | | | | | |
| death effector domain containing | DEDD | NM_001039711 // | CASP8IP1 // DEDD1 | 2440625 | chr1 | − | 159357395 | 159369167 | 7.89 |
| | | NM_001039712 // | //DEFT // FLDED1 | | | | | | |
| | | NM_032998 | // KE05 | | | | | | |
| integrin, beta 6 | ITGB6 | NM_000888 | — | 2583465 | chr2 | − | 160664438 | 160818586 | 7.88 |
| zinc finger protein 473 | ZNF473 | NM_001006656 // | HZFP100 // ZN473 | 3839142 | chr19 | + | 55220811 | 55248476 | 7.87 |
| | | NM_015428 | | | | | | | |
| HLA-B associated transcript 1 // | BAT1 // | NM_004640 // | D6S81E // DDX39B | 2949038 | chr6 | − | 31605990 | 31622606 | 7.87 |
| ATPase, H+ transporting, lysosomal | ATP6V1G2 | NM_080598 // | // UAP56 // ATP6G | | | | | | |
| 13 kDa, V1 subunit G2 | | NM_130463 // | // ATP6G2 // NG38 | | | | | | |
| | | NM_138282 | // VMA10 | | | | | | |
| casein kinase 2, alpha 1 polypeptide | CSNK2A1 // | NM_001895 // | CK2A1 // CKII // | 3894228 | chr20 | − | 402069 | 472534 | 7.87 |
| // casein kinase 2, alpha 1 | CSNK2A1P | NM_177559 // | CKII alpha // — | | | | | | |
| polypeptide pseudogene | | NM_177560 // | | | | | | | |
| | | NR_002207 | | | | | | | |
| peroxisome proliferator-activated | PPRC1 | NM_015062 | KIAA0595 // | 3261447 | chr10 | + | 103882745 | 103900072 | 7.86 |
| receptor gamma, coactivator-related 1 | | | MGC74642 // PRC // | | | | | | |
| | | | RP11-302K17.6 | | | | | | |
| YME1-like 1 (S. cerevisiae) | YME1L1 | NM_014263 // | FTSH // MEG4 // | 3282213 | chr10 | − | 27439066 | 27484191 | 7.84 |
| | | NM_139312 // | PAMP // YME1L | | | | | | |
| | | NM_139313 | | | | | | | |
| thyroid hormone receptor associated | THRAP1 | NM_005121 | ARC250 // DRIP250 | 3765689 | chr17 | − | 57374750 | 57498031 | 7.84 |
| protein 1 | | | // HSPC221 // | | | | | | |
| | | | KIAA0593 // MED13 | | | | | | |
| | | | // TRAP240 | | | | | | |
| YTH domain family, member 1 | YTHDF1 | NM_017798 | C20orf21 // | 3913712 | chr20 | − | 61297230 | 61330873 | 7.83 |
| B-cell CLL/lymphoma 7B | BCL7B | NM_001707 // | — | 3056108 | chr7 | − | 72588624 | 72610244 | 7.82 |
| | | NM_138707 | | | | | | | |
| HIR histone cell cycle regulation | HIRA | NM_003325 | DGCR1 // TUP1 // | 3952637 | chr22 | − | 17696576 | 17799452 | 7.82 |
| defective homolog A (S. cerevisiae) | | | TUPLE1 | | | | | | |
| Josephin domain containing 3 // | JOSD3 // | NM_024116 // | MGC5306 // ACA25 | 3386814 | chr11 | − | 93102831 | 93114308 | 7.81 |
| small nucleolar RNA, H/ACA box 25 | SNORA25 // | NR_003028 // | // ACA32 // ACA18 | | | | | | |
| // small nucleoler RNA, H/ACA box | SNORA32 // | NR003032 // | | | | | | | |
| 32 // small nucleolar RNA, H/ACA | SNORA18 | NR_002959 | | | | | | | |
| GTP binding protein 4 | GTPBP4 | NM_012341 | CRFG // FLJ10686 | 3231774 | chr10 | + | 942450 | 1055862 | 7.79 |
| | | | // FLJ10690 // | | | | | | |
| | | | // FLJ39774 // NGB | | | | | | |
| eukaryotic translation elongation | EEF1B2 | NM_001037663 // | EEF1B // EEF1B1 // | 2524577 | chr2 | + | 206732303 | 206735884 | 7.78 |
| factor 1 beta 2 | | NM_001959 // | EF1B | | | | | | |
| | | NM_021121 | | | | | | | |
| SMAD family member 5 | SMAD5 | NM_001001419 // | DKFZp781C1895 // | 2830010 | chr5 | + | 135464462 | 135552314 | 7.77 |
| | | NM_001001420 // | DKFZp781O1323 // | | | | | | |
| | | NM_005903 | Dwfc // JV5-1 // | | | | | | |
| | | | MADH5 | | | | | | |
| epidermal growth factor receptor | EGFR | NM_005228 // | ERBB // ERBB1 // | 3002640 | chr7 | + | 55054070 | 55291773 | 7.77 |
| (erythroblastic leukemia viral (v-erb- | | NM_201282 // | mENA | | | | | | |
| b) oncogene homolog, avian) | | NM_201283 // | | | | | | | |
| | | NM_201284 | | | | | | | |
| mitogen-activated protein kinase | MAP3K11 | NM_002419 | MGC17114 // MLK-3 | 3377752 | chr11 | − | 65121806 | 65139693 | 7.77 |
| kinase kinase 11 | | | // MLK3 // PTK1 // | | | | | | |
| | | | SPRK | | | | | | |
| apoptosis inhibitor 5 | API5 | NM_006595 | AAC-11 // AAC11 // | 3327906 | chr11 | + | 43290109 | 43322649 | 7.77 |
| | | | API5L1 | | | | | | |
| coiled-coil-helix-coiled-coil-helix | CHCHD3 | NM_017812 | FLJ20420 | 3073597 | chr7 | − | 132062426 | 132500566 | 7.75 |
| domain containing 3 | | | | | | | | | |
| TBC1 domain family, member 10B | TBC1D10B | NM_015527 | DKFZP434P1750 // | 3687715 | chr16 | − | 30275789 | 30289100 | 7.74 |
| | | | FP2461 | | | | | | |
| nucleolar protein NOP5/NOP58 | NOP5/NOP58 | NM_015934 | HSPC120 | 2523144 | chr2 | + | 202811346 | 202926967 | 7.73 |
| ribosomal protein S6 kinase, 70 kDa, | RPS6KB1 | NM_003161 | PS6K // S6K // S6K1 | 3729294 | chr17 | + | 55325239 | 55429105 | 7.73 |
| polypeptide 1 | | | // STK14A // | | | | | | |
| | | | p70(S6K)-alpha // | | | | | | |
| | | | p70-S6K // p70-alpha | | | | | | |
| GDP dissociation inhibitor 1 | GDI1 | NM_001493 | FLJ41411 // GDIL // | 3996404 | chrX | + | 153318480 | 153324978 | 7.73 |
| | | | MRX41 // MRX48 // | | | | | | |
| | | | OPHN2 // RABGD1A | | | | | | |
| | | | // RABGDIA // XAP- | | | | | | |
| PHD finger protein 12 | PHF12 | NM_001033561 // | KIAA1523 // | 3751184 | chr17 | − | 24256404 | 24302905 | 7.73 |
| | | NM_020889 | MGC131914 // PF1 | | | | | | |
| Smad nuclear interacting protein 1 | SNIP1 | NM_024700 | FLJ12553 // RP3- | 2407163 | chr1 | − | 37774050 | 37792490 | 7.72 |
| | | | 423B22.3 // | | | | | | |
| | | | dJ423B22.2 | | | | | | |
| stress-induced-phosphoprotein 1 | STIP1 | NM_006819 | HOP // IEF-SSP- | 3334224 | chr11 | + | 63709341 | 63728586 | 7.71 |
| (Hsp70/Hsp90-organizing protein) | | | 3521 // P60 // STI1L | | | | | | |
| speckle-type POZ protein | SPOP | NM_001007226 // | TEF2 | 3761905 | chr17 | − | 45031245 | 45110541 | 7.71 |
| | | NM_001007227 // | | | | | | | |
| | | NM_001007228 // | | | | | | | |
| | | NM_001007229 // | | | | | | | |
| | | NM_001007230 // | | | | | | | |
| | | NM_003563 | | | | | | | |
| eukaryotic translation elongation | EEF1A1 | NM_001402 | CCS-3 // CCS3 // | 2960903 | chr6 | − | 74281167 | 74335800 | 7.70 |
| factor 1 alpha 1 | | | EEF-1 // EEF1A // | | | | | | |
| | | | EF-Tu // EF1A // | | | | | | |
| | | | FLJ25721 // GRAF- | | | | | | |
| | | | 1EF // HNGC: 16303 | | | | | | |
| | | | // LENG7 // | | | | | | |
| | | | MGC102687 // | | | | | | |
| | | | MGC131894 // | | | | | | |
| | | | MGC16224 // PTI1 // | | | | | | |
| | | | eEF1A-1 | | | | | | |
| mitogen-activated protein kinase | MAP2K3 | XM_001130488 // | MAPKK3 // MEK3 // | 3714729 | chr17 | + | 21128581 | 21159113 | 7.70 |
| kinase 3 | | NM_145110 // | MKK3 // PRKMK3 | | | | | | |
| | | NM_002756 // | | | | | | | |
| | | NM_145109 | | | | | | | |
| DEAD (Asp-Glu-Ala-Asp) box | DDX18 // | NM_006773 // | FLJ33908 // MrDb // — | 2502300 | chr2 | + | 118243834 | 118323562 | 7.70 |
| polypeptide 18 // wingless-type | WNT8B | NM_003393 | | | | | | | |
| MMTV integration site family. | | | | | | | | | |
| ATPase family, AAA domain | ATAD2 | NM_014109 | DKFZp667N1320 // | 3151534 | chr8 | − | 124393174 | 124477871 | 7.69 |
| containing 2 | | | MGC131938 // | | | | | | |
| | | | MGC142216 // | | | | | | |
| | | | MGC29843 // | | | | | | |
| | | | MGC5254 // | | | | | | |
| chromosome 1 open reading frame | C1orf160 | NM_032125 | DKFZP564D0478 // | 2326954 | chr1 | + | 27521221 | 27535639 | 7.69 |
| 160 | | | MGC111002 // RP11- | | | | | | |
| | | | 4K3_A.4 | | | | | | |
| RNA binding motif protein 5 | RBM5 | NM_005778 | FLJ39876 // G15 // | 2622469 | chr3 | + | 50101372 | 50134000 | 7.69 |
| | | | H37 // LUCA15 // | | | | | | |
| | | | RMB5 | | | | | | |
| thioredoxin domain containing 9 // | TXNDC9 // | NM_005783 // | APACD // LYGH // | 2566740 | chr2 | − | 99301923 | 99319337 | 7.69 |
| lysozyme-like | LYG2 | NM_175735 | MGC119046 // | | | | | | |
| | | | MGC119047 // | | | | | | |
| | | | MGC119049 | | | | | | |
| zinc finger protein 148 | ZNF148 | NM_021964 | BERF-1 // BFCOL1 | 2693081 | chr3 | − | 126427204 | 126577074 | 7.69 |
| | | | // HT-BETA // ZBP- | | | | | | |
| | | | 89 // ZFP148 // | | | | | | |
| | | | pHZ-52 | | | | | | |
| insulin receptor substrate 2 | IRS2 | NM_003749 | — | 3525234 | chr13 | − | 109196024 | 109238228 | 7.68 |
| interleukin enhancer binding factor 3, | ILF3 | NM_004516 // | DRBF // DRBP76 // | 3820663 | chr19 | + | 10625547 | 10664074 | 7.68 |
| 90 kDa | | NM_012218 // | MMP4 // MPHOSPH4 | | | | | | |
| | | NM_153464 | // MPP4 // NF-AT- | | | | | | |
| | | | 90 // NF90 // NFAR | | | | | | |
| | | | // NFAR-1 // TCP80 | | | | | | |
| ubiquitin-conjugating enzyme E2, J1 | UBE2J1 | NM_016021 | CGI-76 // HSPC153 | 2964200 | chr6 | − | 90093065 | 90119338 | 7.66 |
| (UBC6 homolog, yeast) | | | // HSPC205 // | | | | | | |
| | | | HSU93243 // | | | | | | |
| | | | MGC12555 // | | | | | | |
| | | | NCUBE1 // Ubc6p | | | | | | |
| START domain containing 7 | STARD7 | NM_020151 // | GTT1 | 2565143 | chr2 | − | 96214334 | 96238296 | 7.65 |
| | | NM_139267 | | | | | | | |
| DPH1 homolog (S. cerevisiae) // | DPH1 // | NM_001383 // | DPH2L // DPH2L1 // | 3706071 | chr17 | + | 1874907 | 1895099 | 7.64 |
| candidate tumor suppressor in | OVCA2 | NM_080822 | FLJ33211 // OVCA1 | | | | | | |
| ovarian cancer 2 | | | // — | | | | | | |
| non-POU domain containing, | NONO | NM_007363 | NMT55 // NRB54 // | 3980887 | chrX | + | 70420006 | 70437726 | 7.64 |
| octamer-binding | | | P54 // P54NRB | | | | | | |
| scaffold attachment factor B2 | SAFB2 | NM_014649 | KIAA0138 | 3847252 | chr19 | − | 5533105 | 5575015 | 7.64 |
| WD repeat domain 3 | WDR3 | NM_006784 | FLJ12796 | 2354082 | chr1 | + | 118273827 | 118304572 | 7.63 |
| metal response element binding | MTF2 | NM_007358 | M96 // PCL2 // | 2346934 | chr1 | + | 93147511 | 93377202 | 7.63 |
| transcription factor 2 | | | RP5-976O13.1 // | | | | | | |
| | | | dJ976O13.2 | | | | | | |
| general transcription factor IIIC, | GTF3C2 | NM_001035521 // | KIAA0011 // TFIIIC- | 2545681 | chr2 | − | 27402231 | 27433372 | 7.63 |
| polypeptide 2, beta 110 kDa | | NM_001521 | BETA // TFIIIC110 | | | | | | |
| general transcription factor IIH, | GTF2H1 | NM_005316 | BTF2 // TFIIH | 3322717 | chr11 | + | 18300412 | 18345156 | 7.62 |
| polypeptide 1, 62 kDa | | | | | | | | | |
| mitochondrial ribosomal protein L17 | MRPL17 | NM_022061 | MRP-L26 // RPL17L | 3361116 | chr11 | − | 6657673 | 6690484 | 7.62 |
| | | | // RPML26 | | | | | | |
| Yip1 domain family, member 3 // | YIPF3 // | NM_015388 // | C6orf109 // | 2954646 | chr6 | − | 43587500 | 43592701 | 7.61 |
| chromosome 6 open reading frame | C6orf154 | NM_001012974 | DKFZP566C243 // | | | | | | |
| 154 | | | FinGER3 // KLIP1 // | | | | | | |
| | | | dJ337H4.3 // | | | | | | |
| | | | FLJ44836 // | | | | | | |
| | | | MGC131686 // | | | | | | |
| | | | dJ337H4.2 | | | | | | |
| eukaryotic translation initiation factor | EIF4G2 | NM_001042559 // | AAG1 // DAP5 // | 3362719 | chr11 | − | 10487574 | 10788746 | 7.60 |
| 4 gamma, 2 | | NM_001418 | FLJ41344 // NAT1 // | | | | | | |
| | | | p97 | | | | | | |
| MYST histone acetyltransferase 2 | MYST2 | NM_007067 | HBO1 // HBOA | 3725779 | chr17 | + | 45196447 | 45261455 | 7.60 |
| SIN3 homolog A, transcription | SIN3A | NM_015477 | DKFZP434K2235 // | 3633403 | chr15 | − | 73448786 | 73535167 | 7.60 |
| regulator (yeast) | | | FLJ90319 // | | | | | | |
| | | | KIAA0700 | | | | | | |
| centaurin, delta 1 | CENTD1 | NM_015230 // | ARAP2 // FLJ13675 | 2765590 | chr4 | − | 35626248 | 35922356 | 7.60 |
| | | NM_139182 | // FLJ44916 // | | | | | | |
| | | | KIAA0580 // PARX | | | | | | |
| KIT ligand | KITLG | NM_000899 // | DKFZp686F2250 // | 3464747 | chr12 | − | 87349442 | 87498371 | 7.58 |
| | | NM_003994 | KL-1 // Kitl // MGF | | | | | | |
| | | | // SCF // SF | | | | | | |
| F-box and leucine-rich repeat | FBXL11 | NM_012308 | CXXC8 // | 3336696 | chr11 | + | 66643299 | 66782124 | 7.57 |
| protein 11 | | | DKFZP434M1735 // | | | | | | |
| | | | FBL11 // FBL7 // | | | | | | |
| | | | FLJ00115 // | | | | | | |
| | | | FLJ46431 // JHDM1A | | | | | | |
| | | | // KIAA1004 // | | | | | | |
| | | | LILINA | | | | | | |
| POM (POM121 homolog, rat) and | POMZP3 | NM_012230 // | MGC8359 // POM- | 3057755 | chr7 | − | 76037679 | 76196100 | 7.56 |
| ZP3 fusion | | NM_152992 | ZP3 // POM121 | | | | | | |
| VAMP (vesicle-associated membrane | VAPA | NM_003574 // | MGC3745 // VAP-33 | 3778601 | chr18 | + | 9903984 | 9950012 | 7.56 |
| protein)-associated protein A, 33 kDa | | NM_194434 | // VAP-A // VAP33 | | | | | | |
| | | | // hVAP-33 | | | | | | |
| integrin beta 4 binding protein | ITGB4BP | NM_002212 // | CAB // EIF3A // EIF6 | 3903836 | chr20 | − | 33276003 | 33336225 | 7.55 |
| | | NM_181466 // | // b(2)gcn // p27BBP | | | | | | |
| | | NM_181467 // | | | | | | | |
| | | NM_181468 // | | | | | | | |
| | | NM_181469 | | | | | | | |
| chromosome 10 open reading frame | C10orf119 | NM_024834 | FLJ13081 // | 3309755 | chr10 | − | 121568302 | 121642742 | 7.55 |
| 119 | | | FLJ36756 | | | | | | |
| chromosome 11 open reading frame | C11orf57 | NM_018195 | FLJ10726 | 3348891 | chr11 | + | 111450087 | 111461067 | 7.54 |
| 57 | | | | | | | | | |
| heat shock protein 90 kDa alpha | HSP90AA1 | NM_001017963 // | FLJ31884 // HSP86 | 3580179 | chr14 | − | 101616842 | 101676327 | 7.53 |
| (cytosolic), class A member 1 // | // | NM_005348 // | // HSP90A // | | | | | | |
| heat shock protein 90 kDa alpha | HSP90AA2 | NM_001040141 // — | HSP90N // HSPC1 // | | | | | | |
| (cytosolic), class A member 2 // | // | | HSPCA // HSPCAL1 | | | | | | |
| heat shock protein 90 kDa alpha | HSP90AA6P | | // HSPCAL4 // | | | | | | |
| (cytosolic), class A member 6 | | | HSPN // Hsp89 // | | | | | | |
| (pseudogene) | | | Hsp90 // LAP2 // | | | | | | |
| | | | HSP90ALPHA // | | | | | | |
| | | | HSPCAL3 // | | | | | | |
| peroxisomal biogenesis factor 5 | PEX5 | NM_000319 | PTS1R // PXR1 | 3403299 | chr12 | + | 7232607 | 7262437 | 7.53 |
| chromosome 4 open reading frame | C4orf30 | NM_017741 | FLJ20280 // | 2762468 | chr4 | − | 17404173 | 17421482 | 7.53 |
| 30 | | | MGC126765 // | | | | | | |
| | | | MGC126767 | | | | | | |
| transmembrane protein 2 | TMEM2 | NM_013390 | — | 3209384 | chr9 | − | 73452177 | 73573229 | 7.52 |
| forkhead box M1 | FOXM1 | NM_021953 // | FKHL16 // FOXM1B | 3440598 | chr12 | − | 2836207 | 2856564 | 7.52 |
| | | NM_202002 // | // HFH-11 // HFH11 | | | | | | |
| | | NM_202003 | // HNF-3 // INS-1 | | | | | | |
| | | | // MPHOSPH2 // | | | | | | |
| | | | MPP-2 // MPP2 // | | | | | | |
| | | | PIG29 // TRIDENT | | | | | | |
| ceramide kinase | CERK | NM_022766 // | DKFZp434E0211 // | 3964154 | chr22 | − | 45454343 | 45537349 | 7.52 |
| | | NM_182661 | FLJ21430 // | | | | | | |
| | | | FLJ23239 // | | | | | | |
| | | | KIAA1646 // LK4 // | | | | | | |
| | | | MGC131878 // | | | | | | |
| | | | dA59H18.2 // | | | | | | |
| | | | dA59H18.3 // hCERK | | | | | | |
| small nuclear ribonucleoprotein | SNRP70 | NM_001009820 // | RNPU1Z // RPU1 // | 3838185 | chr19 | + | 54280358 | 54304714 | 7.52 |
| 70 kDa polypeptide (RNP antigen) | | NM_003089 | U170K // U1AP // | | | | | | |
| | | | U1RNP | | | | | | |
| anthrax toxin receptor 2 | ANTXR2 | NM_058172 | CMG-2 // CMG2 // | 2774971 | chr4 | − | 80762874 | 81219758 | 7.51 |
| | | | FLJ31074 // ISH // | | | | | | |
| | | | JHF // MGC111533 | | | | | | |
| | | | // MGC45856 | | | | | | |
| antizyme inhibitor 1 | AZIN1 | NM_015878 // | MGC3832 // MGC691 | 3147591 | chr8 | − | 103907725 | 103953967 | 7.51 |
| | | NM_148174 | // OAZI // OAZIN // | | | | | | |
| | | | ODO1L | | | | | | |
| chromosome 12 open reading frame | C12orf44 | NM_021934 | FLJ11773 | 3415273 | chr12 | + | 50749317 | 50757534 | 7.51 |
| 44 | | | | | | | | | |
| cofactor required for Sp1 | CRSP6 | NM_004268 | CRSP77 // DRIP80 | 3344897 | chr11 | + | 93157019 | 93186225 | 7.49 |
| transcriptional activation, subunit 6, | | | // FLJ10812 // | | | | | | |
| 77 kDa | | | MED17 // TRAP80 | | | | | | |
| KIAA1967 | KIAA1967 | NM_021174 // | DBC-1 // DBC1 | 3089597 | chr8 | + | 22518038 | 22534618 | 7.49 |
| | | NM_199205 | | | | | | | |
| serologically defined colon cancer | SDCCAG3 | NM_001039707 // | NY-CO-3 // | 3229943 | chr9 | − | 138416210 | 138425418 | 7.49 |
| antigen 3 // small nuclear RNA | // SNAPC4 | NM_001039708 // | FLJ13451 // | | | | | | |
| activating complex, polypeptide 4, | | NM_006643 // | PTFalpha // | | | | | | |
| 190 kDa | | NM_003086 | SNAP190 | | | | | | |
| potassium channel tetramerisation | KCTD10 | NM_031954 | FLJ41739 // | 3470793 | chr12 | − | 108370845 | 108399528 | 7.48 |
| domain containing 10 | | | MSTP028 // ULRO61 | | | | | | |
| NADH dehydrogenase (ubiquinone) 1 | NDUFA5 | NM_005000 | B13 // CI-13KD-B // | 3070658 | chr7 | − | 122968075 | 122985216 | 7.48 |
| alpha subcomplex, 5, 13 kDa | | | DKFZp781K1356 // | | | | | | |
| | | | FLJ12147 // NUFM | | | | | | |
| | | | // UQOR13 | | | | | | |
| interferon stimulated exonuclease | ISG20L1 | NM_022767 | AEN // FLJ12484 // | 3607232 | chr15 | + | 86948254 | 86976501 | 7.47 |
| gene 20 kDa-like 1 | | | FLJ12562 // pp12744 | | | | | | |
| paxillin | PXN | NM_002859 | FLJ16691 | 3474372 | chr12 | − | 119132644 | 119187926 | 7.47 |
| synaptotagmin binding, cytoplasmic | SYNCRIP | NM_006372 | GRY-RBP // | 2963407 | chr6 | − | 86374062 | 86410282 | 7.47 |
| RNA interacting protein | | | HNRPQ1 // NSAP1 | | | | | | |
| | | | // RP1-3J17.2 // | | | | | | |
| | | | dJ3J17.2 // pp68 | | | | | | |
| proteasome (prosome, macropain) | PSMD6 | NM_014814 | KIAA0107 // S10 // | 2679864 | chr3 | − | 63971281 | 63984700 | 7.47 |
| 26S subunit, non-ATPase, 6 | | | SGA-113M // p44S10 | | | | | | |
| chromosome 7 open reading frame | C7orf30 | NM_138446 | — | 2992863 | chr7 | + | 23305462 | 23317894 | 7.46 |
| heterogeneous nuclear | HNRPK | NM_002140 // | CSBP // FLJ41122 | 3212294 | chr9 | − | 85772377 | 85785355 | 7.45 |
| ribonucleoprotein K | | NM_031262 // | // HNRNPK // TUNP | | | | | | |
| | | NM_031263 | | | | | | | |
| heterogeneous nuclear | HNRPL | NM_001005335 // | FLJ35509 // | 3861617 | chr19 | − | 44018883 | 44034809 | 7.45 |
| ribonucleoprotein L | | NM_001533 | P/OKcl.14 // hnRNP-L | | | | | | |
| SEC14-like 1 (S. cerevisiae) | SEC14L1 | NM_001039573 // | DKFZp686C06176 // | 3735752 | chr17 | + | 72596335 | 72767744 | 7.45 |
| | | NM_003003 | PRELID4A // SEC14L | | | | | | |
| hippocampus abundant transcript- | HIATL1 | XM_001130181 // | FLJ14753 // | 3180263 | chr9 | + | 96176674 | 96325754 | 7.43 |
| like 1 | | NM_032558 | MGC117350 | | | | | | |
| eukaryotic translation initiation factor | EIF5A | NM_001970 | EIF-5A // EIF5A1 // | 3708422 | chr17 | + | 7151037 | 7156478 | 7.42 |
| 5A | | | MGC104255 // | | | | | | |
| | | | MGC99547 // uORF | | | | | | |
| | | | // uORF A | | | | | | |
| discoidin, CUB and LCCL domain | DCBLD2 | NM_080927 | CLCP1 // ESDN | 2686023 | chr3 | − | 99997526 | 100124598 | 7.42 |
| containing 2 | | | | | | | | | |
| voltage-dependent anion channel 2 | VDAC2 | NM_003375 | FLJ23841 | 3252577 | chr10 | + | 76639941 | 76661389 | 7.41 |
| polycomb group ring finger 3 | PCGF3 | NM_006315 | DKFZp686D20235 // | 2714230 | chr4 | + | 689573 | 754428 | 7.41 |
| | | | DONG1 // FLJ36550 | | | | | | |
| | | | // FLJ43813 // | | | | | | |
| | | | MGC129615 // | | | | | | |
| | | | MGC40413 // RNF3 | | | | | | |
| | | | // RNF3A | | | | | | |
| NADH dehydrogenase (ubiquinone) | NDUFS3 | NM_004551 | — | 3329904 | chr11 | + | 47557156 | 47562663 | 7.41 |
| Fe—S protein 3, 30 kDa (NADH- | | | | | | | | | |
| coenzyme Q reductase) | | | | | | | | | |
| signal recognition particle 68 kDa | SRP68 | NM_014230 | — | 3771297 | chr17 | − | 71546467 | 71580323 | 7.41 |
| thioredoxin reductase 1 | TXNRD1 | NM_003330 // | GRIM-12 // MGC9145 | 3429460 | chr12 | + | 103130628 | 103354079 | 7.39 |
| | | NM_182729 // | // TR // TR1 // | | | | | | |
| | | NM_182742 // | TRXR1 // TXNR | | | | | | |
| | | NM_182743 | | | | | | | |
| PDZ domain containing 8 | PDZD8 | NM_173791 | FLJ25412 // | 3308619 | chr10 | − | 119029422 | 119124958 | 7.39 |
| | | | FLJ34427 // PDZK8 | | | | | | |
| | | | // bA129M16.2 | | | | | | |
| IKAROS family zinc finger 5 | IKZF5 | NM_022466 | DKFZp781B0249 // | 3310757 | chr10 | − | 124740316 | 124774190 | 7.38 |
| (Pegasus) | | | FLJ22973 // | | | | | | |
| | | | PEGASUS // | | | | | | |
| TPX2, microtubule-associated, | TPX2 | NM_012112 | C20orf1 // C20orf2 | 3881443 | chr20 | + | 29774755 | 29853260 | 7.37 |
| homolog (Xenopus laevis) | | | // DIL-2 // DIL2 // | | | | | | |
| | | | FLS353 // | | | | | | |
| | | | GD: C20orf1 // | | | | | | |
| | | | HCA519 // HCTP4 // | | | | | | |
| RNA binding motif protein 39 | RBM39 | NM_184237 // | CAPER // | 3904226 | chr20 | − | 33754569 | 33793768 | 7.35 |
| | | NM_184241 // | CAPERalpha // CC1.3 | | | | | | |
| | | NM_184244 // | // DKFZp781C0423 | | | | | | |
| | | NM_004902 // | // FLJ44170 // | | | | | | |
| | | NM_184234 | HCC1 // RNPC2 | | | | | | |
| ubiquitin specific peptidase 53 | USP53 | NM_019050 | DKFZp781E1417 | 2741236 | chr4 | + | 120353195 | 120436746 | 7.35 |
| vesicle-associated membrane protein | VAMP3 | NM_004781 | CEB | 2318637 | chr1 | + | 7753916 | 7764072 | 7.35 |
| 3 (cellubrevin) | | | | | | | | | |
| DEAH (Asp-Glu-Ala-His) box | DHX36 | NM_020865 | DDX36 // KIAA1488 | 2701595 | chr3 | − | 155475346 | 155524965 | 7.33 |
| polypeptide 36 | | | // MLEL1 | | | | | | |
| fibronectin type III domain containing | FNDC3B | NM_022763 | DKFZp686D14170 // | 2652410 | chr3 | + | 173240112 | 173601176 | 7.33 |
| 3B | | | DKFZp762K137 // | | | | | | |
| | | | FAD104 // FLJ23399 | | | | | | |
| | | | // MGC10002 // | | | | | | |
| | | | PRO4979 // | | | | | | |
| | | | YVTM2421 | | | | | | |
| E74-like factor 1 (ets domain | ELF1 | NM_172373 | — | 3511031 | chr13 | − | 40394613 | 40533811 | 7.32 |
| transcription factor) | | | | | | | | | |
| keratin 19 | KRT19 | NM_002276 | CK19 // K19 // K1CS | 3757108 | chr17 | − | 36933431 | 36938160 | 7.32 |
| | | | // MGC15366 | | | | | | |
| Integrin, alpha 2 (CD49B, alpha 2 | ITGA2 | NM_002203 | BR // CD49B // GPIa | 2809245 | chr5 | + | 52320949 | 52432778 | 7.31 |
| subunit of VLA-2 receptor) | | | // VLA-2 // VLAA2 | | | | | | |
| ribosomal protein L30 | RPL30 | NM_000989 | — | 3145953 | chr8 | − | 99123079 | 99127371 | 7.31 |
| IMP4, U3 small nucleolar | IMP4 | NM_033416 | BXDC4 // MGC19606 | 2505501 | chr2 | + | 130816288 | 130828310 | 7.31 |
| ribonucleoprotein, homolog (yeast) | | | | | | | | | |
| origin recognition complex, subunit | ORC2L // | NM_006190 | ORC2 | 2594569 | chr2 | − | 201482783 | 201536750 | 7.31 |
| 2-like (yeast) | MGC39518 | | | | | | | | |
| Kruppel-like factor 10 | KLF10 | NM_001032282 // | EGRA // TIEG // | 3147508 | chr8 | − | 103682306 | 103767558 | 7.30 |
| | | NM_005655 | TIEG1 | | | | | | |
| tight junction protein 1 (zona | TJP1 | NM_003257 // | DKFZp686M05161 // | 3615579 | chr15 | − | 27703228 | 28048334 | 7.30 |
| occludens 1) | | NM_175610 | MGC133289 // ZO-1 | | | | | | |
| tRNA 5-methylaminomethyl-2- | TRMU | NM_018006 | MGC99627 // MTO2 | 3949097 | chr22 | + | 45109982 | 45131896 | 7.29 |
| thiouridylate methyltransferase | | | // MTU1 // TRMT // | | | | | | |
| | | | TRMT1 // TRNT1 | | | | | | |
| ribosomal protein L18 | RPL18 | NM_000979 | — | 3867223 | chr19 | − | 53810441 | 53814585 | 7.29 |
| Nance-Horan syndrome (congenital | NHS | XM_001134060 // | DKFZp781F2016 // | 3970338 | chrX | + | 17303441 | 17664033 | 7.29 |
| cataracts and dental anomalies) | | NM_198270 | DKFZp781L0254 // | | | | | | |
| | | | SCML1 | | | | | | |
| chromosome 16 open reading frame | C16orf72 | NM_014117 | FLJ41272 // | 3647632 | chr16 | + | 9092682 | 9121055 | 7.28 |
| 72 | | | PRO0149 | | | | | | |
| ubiquitin specific peptidase 10 | USP10 | NM_005153 | KIAA0190 // | 3671873 | chr16 | + | 83291080 | 83371023 | 7.27 |
| | | | MGC2621 // UBPO | | | | | | |
| A kinase (PRKA) anchor protein | AKAP12 | NM_005100 // | AKAP250 // | 2931569 | chr6 | + | 151602720 | 151721361 | 7.27 |
| (gravin) 12 | | NM_144497 | DKFZp686M0430 // | | | | | | |
| | | | DKFZp686O0331 | | | | | | |
| ornithine decarboxylase 1 | ODC1 | NM_002539 | — | 2540157 | chr2 | − | 10485474 | 10606340 | 7.27 |
| matrin 3 | MATR3 | NM_018834 // | DKFZp686K0542 // | 2831124 | chr5 | + | 138505686 | 138695245 | 7.26 |
| | | NM_199189 | DKFZp686K23100 // | | | | | | |
| | | | KIAA0723 // | | | | | | |
| | | | MGC9105 | | | | | | |
| chromosome 5 open reading frame | C5orf22 | NM_018356 | DKFZp667N066 // | 2805176 | chr5 | + | 31568161 | 31590902 | 7.26 |
| 22 | | | FLJ11193 // | | | | | | |
| | | | FLJ23805 // | | | | | | |
| | | | MGC33010 | | | | | | |
| nuclear mitotic apparatus protein 1 | NUMA1 | NM_006185 | NUMA | 3380901 | chr11 | − | 71389909 | 71469367 | 7.25 |
| macrophage erythroblast attacher | MAEA | NM_001017405 // | EMP // HLC-10 // | 2714672 | chr4 | + | 1273702 | 1323891 | 7.25 |
| | | NM_005882 | PIG5 | | | | | | |
| nucleolar protein 8 | NOL8 | NM_017948 | C9orf34 // | 3214749 | chr9 | − | 94099471 | 94127688 | 7.25 |
| | | | DKFZp686P12242 // | | | | | | |
| | | | FLJ20736 // Nop132 | | | | | | |
| | | | // bA62C3.3 // | | | | | | |
| | | | bA62C3.4 | | | | | | |
| proliferation-associated 2G4, 38 kDa | PA2G4 // | NM_006191 // | EBP1 // HG4-1 // | 3417309 | chr12 | + | 54784566 | 54793956 | 7.23 |
| // dihydrolipoamide S- | DLST | NM_001933 | p38-2G4 // DLTS | | | | | | |
| succinyltransferase (E2 component | | | | | | | | | |
| of 2-oxo-glutarate complex) | | | | | | | | | |
| mitochondrial ribosomal protein L46 | MRPL46 | NM_022163 | C15orf4 // LIECG2 // | 3638048 | chr15 | − | 86795015 | 86811600 | 7.23 |
| | | | MGC22762 // | | | | | | |
| phosphatidylinositol glycan anchor | PIGG | NM_017733 | FLJ20265 // | 2714025 | chr4 | + | 483010 | 523985 | 7.22 |
| biosynthesis, class G | | | FLJ39925 // GP17 // | | | | | | |
| | | | LAS21 // MGC131903 | | | | | | |
| | | | // PRO4405 // | | | | | | |
| | | | RLGS1930 | | | | | | |
| SON DNA binding protein | SON | NM_032195 // | BASS1 // C21orf50 | 3918696 | chr21 | + | 33836804 | 33872881 | 7.20 |
| | | NM_138927 | // DBP-5 // | | | | | | |
| | | | FLJ21099 // | | | | | | |
| | | | FLJ33914 // | | | | | | |
| | | | KIAA1019 // NREBP | | | | | | |
| | | | // SON3 | | | | | | |
| kelch-like ECH-associated protein 1 | KEAP1 // | NM_012289 // | INrf2 // KIAA0132 // | 3850363 | chr19 | − | 10457571 | 10475233 | 7.20 |
| // ADAM metallopeptidase with | ADAMTS6 | NM_203500 // | KLHL19 // | | | | | | |
| thrombospondin type 1 motif, 6 | | NM_197941 | MGC10630 // | | | | | | |
| | | | MGC1114 // | | | | | | |
| | | | MGC20887 // | | | | | | |
| | | | MGC4407 // | | | | | | |
| | | | MGC9454 // ADAM- | | | | | | |
| bromodomain containing 2 | BRD2 | NM_005104 | D6S113E // | 2903343 | chr6 | + | 33044392 | 33057253 | 7.20 |
| | | | DKFZp686N0336 // | | | | | | |
| | | | FLJ31942 // FSRG1 | | | | | | |
| | | | // KIAA9001 // NAT | | | | | | |
| | | | // RING3 // RNF3 | | | | | | |
| squalene epoxidase | SQLE | NM_003129 | — | 3114832 | chr8 | + | 126063738 | 126105522 | 7.20 |
| lamin B receptor | LBR | NM_002296 // | DHCR14B // LMN2R | 2458289 | chr1 | − | 223655840 | 223683230 | 7.19 |
| | | NM_194442 | // MGC9041 // PHA | | | | | | |
| Ewing sarcoma breakpoint region 1 | EWSR1 | NM_005243 // | EWS | 3941907 | chr22 | + | 27994201 | 28026501 | 7.19 |
| | | NM_013986 | | | | | | | |
| chromosome 19 open reading frame | C19orf48 | NM_199249 // | MGC13170 | 3868659 | chr19 | − | 55992774 | 56012596 | 7.17 |
| 48 | | NM_199250 | | | | | | | |
| upstream transcription factor 1 // | USF1 // | NM_007122 // | FCHL // FCHL1 // | 2440523 | chr1 | − | 159275665 | 159282355 | 7.17 |
| Rho GTPase activating protein 30 | ARHGAP30 | NM_207005 // | HYPLIP1 // MLTF // | | | | | | |
| | | NM_001025598 // | MLTF1 // UEF // | | | | | | |
| | | NM_181720 | FLJ00267 // | | | | | | |
| | | | FLJ44128 // RP11- | | | | | | |
| | | | 544M22.6 | | | | | | |
| WD repeat domain 46 | WDR46 | NM_005452 | BING4 // C6orf11 // | 2950561 | chr6 | − | 33354871 | 33365259 | 7.16 |
| | | | FP221 | | | | | | |
| topoisomerase (DNA) II alpha 170 kDa | TOP2A | NM_001067 | TOP2 // TP2A | 3756193 | chr17 | − | 35717597 | 35847034 | 7.15 |
| ATP synthase, H+ transporting, | ATP5I | NM_007100 | ATP5K // MGC12532 | 2756497 | chr4 | − | 654679 | 658269 | 7.14 |
| mitochondrial F0 complex, subunit E | | | | | | | | | |
| splicing factor 3b, subunit 2, 145 kDa | SF3B2 | NM_006842 | SAP145 // SF3B145 | 3335907 | chr11 | + | 65573499 | 65593343 | 7.14 |
| | | | // SF3b1 // SF3b150 | | | | | | |
| splicing factor, arginine/serine-rich | SFRS11 | NM_004768 | DKFZp686M13204 // | 2341565 | chr1 | + | 70443686 | 70492014 | 7.12 |
| 11 | | | dJ677H15.2 // p54 | | | | | | |
| aminopeptidase puromycin sensitive | NPEPPS | XM_001128588 // | MP100 // PSA | 3724698 | chr17 | + | 42955347 | 43057168 | 7.11 |
| | | NM_006310 | | | | | | | |
| influenza virus NS1A binding protein | IVNS1ABP | NM_006469 // | DKFZp686K06216 // | 2448073 | chr1 | − | 183532162 | 183553081 | 7.10 |
| | | NM_016389 | FLARA3 // FLJ10069 | | | | | | |
| | | | // FLJ10411 // | | | | | | |
| | | | FLJ10962 // | | | | | | |
| | | | FLJ35593 // | | | | | | |
| | | | FLJ36593 // | | | | | | |
| | | | HSPC068 // | | | | | | |
| | | | KIAA0850 // ND1 // | | | | | | |
| | | | NS-1 // NS1-BP // | | | | | | |
| Smg-7 homolog, nonsense mediated | SMG7 | NM_014837 // | C1orf16 // EST1C // | 2371255 | chr1 | + | 181708036 | 181834004 | 7.09 |
| mRNA decay factor (C. elegans) | | NM_173156 // | FLJ23717 // | | | | | | |
| | | NM_201568 // | KIAA0250 // SGA56M | | | | | | |
| | | NM_201569 | // SMG-7 | | | | | | |
| helicase with zinc finger | HELZ | NM_014877 | DRHC // HUMORF5 | 3768015 | chr17 | − | 62485987 | 62672547 | 7.08 |
| | | | // KIAA0054 // | | | | | | |
| | | | MGC163454 | | | | | | |
| cytochrome P450, family 1, subfamily | CYP1B1 | NM_000104 | CP1B // GLC3A | 2548699 | chr2 | − | 38138730 | 38196009 | 7.08 |
| B. polypeptide 1 | | | | | | | | | |
| sprouty homolog 2 (Drosophila) | SPRY2 | NM_005842 | MGC23039 // | 3519309 | chr13 | − | 79683810 | 79813918 | 7.07 |
| golgi phosphoprotein 4 | GOLPH4 | NM_014498 | GIMPC // GPP130 // | 2704267 | chr3 | − | 169208832 | 169296426 | 7.07 |
| | | | P138 | | | | | | |
| Rab geranylgeranyltransferase, beta | RABGGTB | NM_004582 // | GGTB // — | 2342624 | chr1 | + | 76024474 | 76033333 | 7.07 |
| subunit // mutS homolog 4 (E. coli) | // MSH4 | NM_002440 | | | | | | | |
| angiomotin like 2 | AMOTL2 | NM_016201 | LCCP | 2696309 | chr3 | − | 135527277 | 135576097 | 7.06 |
| MAK10 homolog, amino-acid N- | MAK10 | NM_024635 | FLJ21613 // | 3177563 | chr9 | + | 87733620 | 87828205 | 7.06 |
| acetyltransferase subunit, (S. cerevisiae) | | | FLJ22643 // | | | | | | |
| | | | bA379P1.1 | | | | | | |
| NOL1/NOP2/Sun domain family, | NSUN2 | NM_017755 | FLJ20303 // SAKI // | 2847292 | chr5 | − | 6510634 | 6686394 | 7.06 |
| member 2 | | | TRM4 | | | | | | |
| leptin receptor overlapping | LEPROTL1 | NM_015344 | HSPC112 // Vps55 | 3092276 | chr8 | + | 30059542 | 30115386 | 7.06 |
| transcript-like 1 | | | // my047 | | | | | | |
| geminin, DNA replication inhibitor | GMNN | NM_015895 | Gem // RP3- | 2898597 | chr6 | + | 24857262 | 24894306 | 7.06 |
| FAT tumor suppressor homolog 1 | FAT | NM_005245 | CDHF7 // FAT1 // | 2797393 | chr4 | − | 187745970 | 187885048 | 7.05 |
| (Drosophila) | | | ME5 // hFat1 | | | | | | |
| forkhead box J3 | FOXJ3 | NM_014947 | — | 2408855 | chr1 | − | 42414807 | 42574125 | 7.05 |
| nucleoporin 54 kDa | NUP54 | NM_017426 | MGC13407 | 2773997 | chr4 | − | 77254731 | 77288679 | 7.04 |
| kinesin family member 5B | KIF5B | NM_004521 | KINH // KNS // KNS1 | 3283991 | chr10 | − | 32337798 | 32385342 | 7.04 |
| | | | // UKHC | | | | | | |
| interferon gamma receptor 1 | IFNGR1 | NM_000416 | CD119 // FLJ45734 | 2976113 | chr6 | − | 137560317 | 137582279 | 7.04 |
| | | | // IFNGR | | | | | | |
| mortality factor 4 like 2 | MORF4L2 | NM_012286 | KIAA0026 // MORFL2 | 4016572 | chrX | − | 102817114 | 102844278 | 7.03 |
| | | | // MRGX | | | | | | |
| mastermind-like 1 (Drosophila) | MAML1 | XM_001126853 // | KIAA0200 // Mam-1 | 2844410 | chr5 | + | 179092457 | 179151795 | 7.03 |
| | | NM_014757 | // Mam1 | | | | | | |
| zinc finger and BTB domain | ZBTB11 | NM_014415 | FLJ13426 // | 2686727 | chr3 | − | 102780272 | 102878819 | 7.02 |
| containing 11 | | | MGC133303 // ZNF- | | | | | | |
| | | | U69274 | | | | | | |
| mitochondrial ribosomal protein S2 | MRPS2 | NM_016034 | CGI-91 // MRP-S2 | 3193900 | chr9 | + | 137531661 | 137536331 | 7.02 |
| adaptor-related protein complex 2, | AP2A2 | NM_012305 | ADTAB // CLAPA2 | 3316447 | chr11 | + | 912387 | 1002226 | 7.01 |
| alpha 2 subunit | | | // HIP9 // HYPJ | | | | | | |
| serine arginine-rich pre-mRNA | SR-A1 | NM_021228 | SRA1 | 3838757 | chr19 | + | 54837204 | 54853671 | 7.01 |
| splicing factor SR-A1 | | | | | | | | | |
| NMDA receptor regulated 1 | NARG1 | NM_057175 | Ga19 // NATH // | 2744597 | chr4 | + | 140442091 | 140560627 | 7.00 |
| | | | TBDN100 | | | | | | |
| triple functional domain (PTPRF | TRIO | NM_007118 | tgat | 2802398 | chr5 | + | 14196590 | 14585215 | 7.00 |
| interacting) | | | | | | | | | |
| myotubularin related protein 12 | MTMR12 | NM_001040446 | 3-PAP // PIP3AP | 2852274 | chr5 | − | 32262872 | 32348897 | 7.00 |
| cyclin G associated kinase | GAK | XM_001127411 // | FLJ40395 // | 2756673 | chr4 | − | 833110 | 916149 | 7.00 |
| | | NM_005255 | MGC99654 | | | | | | |
| GRB2-associated binding protein 1 | GAB1 | NM_002039 // | — | 2745547 | chr4 | + | 144476834 | 144615158 | 7.00 |
| | | NM_207123 | | | | | | | |
| senataxin | SETX | NM_015046 | ALS4 // AOA2 // | 3228007 | chr9 | − | 134126584 | 134220359 | 7.00 |
| | | | DKFZp781B151 // | | | | | | |
| | | | FLJ12840 // | | | | | | |
| | | | FLJ43459 // | | | | | | |
| | | | KIAA0625 // SCAR1 | | | | | | |
| | | | // bA479K20.2 | | | | | | |
| chromosome 12 open reading frame | C12orf41 | NM_017822 | FLJ12670 // | 3453177 | chr12 | − | 47333281 | 47362268 | 7.00 |
| 41 | | | FLJ20436 | | | | | | |
| WNK lysine deficient protein kinase 1 | WNK1 | NM_018979 | KDP // KIAA0344 // | 3400034 | chr12 | + | 278104 | 891189 | 6.98 |
| | | | PHA2C // PRKWNK1 | | | | | | |
| myotubularin related protein 1 | MTMR1 | NM_003828 | — | 3994846 | chrX | + | 149612503 | 149684226 | 6.97 |
| met proto-oncogene (hepatocyte | MET | NM_000245 | HGFR // RCCP2 | 3020343 | chr7 | + | 116099694 | 116230307 | 6.97 |
| growth factor receptor) | | | | | | | | | |
| ADP-ribosylation factor 4 | ARF4 | XM_001132763 // | — | 2678090 | chr3 | − | 57532172 | 57558635 | 6.96 |
| | | NM_001660 | | | | | | | |
| four and a half LIM domains 2 | FHL2 | NM_001039492 // | AAG11 // DRAL // | 2568687 | chr2 | − | 105335877 | 105501005 | 6.96 |
| | | NM_001450 // | SLIM3 | | | | | | |
| | | NM_201555 // | | | | | | | |
| | | NM_201557 | | | | | | | |
| polypyrimidine tract binding protein 1 | PTBP1 | NM_002819 // | HNRNPI // HNRPI // | 3815165 | chr19 | + | 747768 | 763505 | 6.96 |
| | | NM_031990 // | MGC10830 // | | | | | | |
| | | NM_031991 // | MGC8461 // PTB // | | | | | | |
| | | NM_175847 | PTB-1 // PTB-T // | | | | | | |
| | | | PTB2 // PTB3 // | | | | | | |
| | | | PTB4 // pPTB | | | | | | |
| glutamate-rich WD repeat containing 1 | GRWD1 | NM_031485 | GRWD // KIAA1942 | 3837796 | chr19 | + | 53640874 | 53652090 | 6.95 |
| | | | // RRB1 // WDR28 | | | | | | |
| signal sequence receptor, alpha | SSR1 | NM_003144 | DKFZp781N23103 // | 2940551 | chr6 | − | 7213542 | 7258526 | 6.94 |
| (translocon-associated protein alpha) | | | TRAPA | | | | | | |
| GC-rich promoter binding protein 1- | GPBP1L1 | NM_021639 | RP11-767N6.1 // | 2410330 | chr1 | − | 45865575 | 45926352 | 6.94 |
| like 1 | | | SP192 | | | | | | |
| chromosome 20 open reading frame | C20orf72 | NM_052865 | FLJ14597 // | 3878220 | chr20 | + | 17896912 | 17919759 | 6.94 |
| 72 | | | bA504H3.4 | | | | | | |
| ELAV (embryonic lethal, abnormal | ELAVL1 // | NM_001419 // | ELAV1 // HUR // | 3848689 | chr19 | − | 7929463 | 7999655 | 6.93 |
| vision, Drosophila)-like 1 (Hu antigen | TIMM44 | NM_006351 | Hua // MelG // | | | | | | |
| R) // translocase of inner | | | DKFZp686H05241 // | | | | | | |
| mitochondrial membrane 44 homolog | | | TIM44 | | | | | | |
| (yeast) | | | | | | | | | |
| heat shock protein 90 kDa alpha | HSP90AB1 | NM_007355 | D6S182 // FLJ26984 | 2908474 | chr6 | + | 44319983 | 44330383 | 6.93 |
| (cytosolic), class B member 1 | | | // HSP90-BETA // | | | | | | |
| | | | HSP90B // HSPC2 // | | | | | | |
| | | | HSPCB | | | | | | |
| TSC22 domain family, member 2 | TSC22D2 | NM_014779 | KIAA0669 // TILZ4a | 2647647 | chr3 | + | 151585441 | 151660841 | 6.91 |
| | | | // TILZ4b // TILZ4c | | | | | | |
| Pentatricopeptide repeat domain 3 | PTCD3 // | NM_017952 // | DKFZp666K071 // | 2491935 | chr2 | + | 86186849 | 86222791 | 6.91 |
| // kinesin family member 5A | KIF5A | NM_004984 | FLJ20758 // | | | | | | |
| | | | D12S1889 // MY050 | | | | | | |
| | | | // NKHC // SPG10 | | | | | | |
| kelch-like 20 (Drosophila) | KLHL20 | NM_014458 | KHLHX // KLEIP // | 2367793 | chr1 | + | 171950703 | 172028914 | 6.90 |
| | | | KLHLX // RP3- | | | | | | |
| | | | 383J4.3 | | | | | | |
| sorting nexin 5 | SNX5 | NM_014426 // | FLJ10931 | 3899346 | chr20 | − | 17855335 | 17897603 | 6.89 |
| | | NM_152227 | | | | | | | |
| KIAA0179 | KIAA0179 | NM_015056 | — | 3923218 | chr21 | + | 43903644 | 43940364 | 6.87 |
| protease, serine, 23 | PRSS23 | NM_007173 | MGC5107 // SIG13 | 3343452 | chr11 | + | 86189210 | 86341580 | 6.87 |
| | | | // SPUVE // ZSIG13 | | | | | | |
| transmembrane, prostate androgen | TMEPAI | NM_020182 // | PMEPA1 // STAG1 | 3911217 | chr20 | − | 55656877 | 55781123 | 6.87 |
| induced RNA | | NM_199169 // | | | | | | | |
| | | NM_199170 // | | | | | | | |
| | | NM_199171 | | | | | | | |
| zinc finger protein 330 | ZNF330 | NM_014487 | HSA6591 // NOA36 | 2745220 | chr4 | + | 142336669 | 142381405 | 6.87 |
| chromodomain helicase DNA binding | CHD2 | NM_001042572 // | DKFZp781D1727 | 3609138 | chr15 | + | 91227096 | 91387351 | 6.86 |
| protein 2 | | NM_001271 | | | | | | | |
| development and differentiation | DDEF1 | NM_018482 | AMAP1 // ASAP1 // | 3153428 | chr8 | − | 131133540 | 131525100 | 6.85 |
| enhancing factor 1 | | | KIAA1249 // PAG2 // | | | | | | |
| | | | PAP // ZG14P | | | | | | |
| mitogen-activated protein kinase | MAP3K14 | NM_003954 | FTDCR1B // HS // | 3759704 | chr17 | − | 40696283 | 40750550 | 6.85 |
| kinase kinase 14 | | | HSNIK // NIK | | | | | | |
| zinc finger protein 289, ID1 regulated | ZNF289 | NM_032389 | FLJ14576 // | 3371928 | chr11 | − | 47142451 | 47155114 | 6.84 |
| | | | FLJ26000 // IRZ // | | | | | | |
| | | | Nbla10535 // Zfp289 | | | | | | |
| heterogeneous nuclear | HNRPR | NM_005826 | FLJ25714 // | 2401275 | chr1 | − | 23486545 | 23543926 | 6.84 |
| ribonucleoprotein R | | | HNRNPR // hnRNP-R | | | | | | |
| coiled-coil domain containing 47 | CCDC47 | NM_020198 | GK001 // MSTP041 | 3766334 | chr17 | − | 59176353 | 59204726 | 6.84 |
| CUG triplet repeat, RNA binding | CUGBP1 | NM_001025596 // | BRUNOL2 // CUG- | 3372253 | chr11 | − | 47444068 | 47543613 | 6.83 |
| protein 1 | | NM_006560 // | BP // CUGBP // | | | | | | |
| | | NM_198700 | NAB50 // hNab50 | | | | | | |
| cancer susceptibility candidate 3 | CASC3 | NM_007359 | BTZ // MLN51 | 3720739 | chr17 | + | 35518075 | 35586901 | 6.82 |
| SUMO1/sentrin/SMT3 specific | SENP3 | NM_015670 | DKFZP586K0919 // | 3708798 | chr17 | + | 7405827 | 7416006 | 6.82 |
| peptidase 3 | | | DKFZp762A152 // | | | | | | |
| | | | SMT3IP1 // SSP3 | | | | | | |
| MAX dimerization protein 1 | MXD1 | NM_002357 | MAD // MAD1 // | 2487549 | chr2 | + | 69995330 | 70023575 | 6.82 |
| | | | MGC104659 | | | | | | |
| protein phosphatase 2 (formerly 2A), | PPP2R2A | NM_002717 | B55A // FLJ26613 // | 3090922 | chr8 | + | 26156860 | 26286261 | 6.82 |
| regulatory subunit B, alpha isoform | | | MGC52248 // PR52A | | | | | | |
| | | | // PR55A | | | | | | |
| fusion (involved in t(12; 16) in | FUS | NM_001010850 // | CHOP // FUS-CHOP | 3656904 | chr16 | + | 31093395 | 31111003 | 6.82 |
| malignant liposarcoma) | | NM_004960 | // FUS1 // TLS // | | | | | | |
| | | | TLS/CHOP | | | | | | |
| programmed cell death 6 // aryl- | PDCD6 // | NM_013232 // | ALG-2 // | 2798586 | chr5 | + | 324634 | 491368 | 6.81 |
| hydrocarbon receptor repressor | AHRR | NM_020731 | MGC111017 // | | | | | | |
| | | | MGC119050 // | | | | | | |
| | | | MGC9123 // PEF1B | | | | | | |
| | | | // AHH // AHHR // | | | | | | |
| nucleolar protein 5A (56 kDa with | NOL5A | NM_006392 | NOP56 | 3873874 | chr20 | + | 2580791 | 2587030 | 6.81 |
| KKE/D repeat) | | | | | | | | | |
| WAS protein family, member 2 | WASF2 | NM_006990 | SCAR2 // WAVE2 // | 2403111 | chr1 | − | 27603331 | 27689256 | 6.81 |
| | | | dJ393P12.2 | | | | | | |
| calmodulin regulated spectrin- | CAMSAP1 | NM_015447 | DKFZP434F195 // | 3229529 | chr9 | − | 137840157 | 137938826 | 6.81 |
| associated protein 1 | | | DKFZp434G2311 // | | | | | | |
| | | | FLJ31228 // | | | | | | |
| | | | MGC163452 // | | | | | | |
| | | | PRO2405 // | | | | | | |
| | | | bA100C15.1 | | | | | | |
| chromosome 18 open reading frame | C18orf25 | NM_001008239 // | ARKL1 // MGC12909 | 3787031 | chr18 | + | 42008011 | 42123527 | 6.78 |
| 25 | | NM_145055 | // MGC87799 | | | | | | |
| cullin 3 | CUL3 | NM_003590 | — | 2601544 | chr2 | − | 225025520 | 225158348 | 6.78 |
| methylthioadenosine phosphorylase | MTAP | NM_002451 | MSAP // c86fus | 3164914 | chr9 | + | 21792645 | 22022975 | 6.77 |
| translocase of outer mitochondrial | TOMM70A | NM_014820 | FLJ90470 | 2686371 | chr3 | − | 101565001 | 101614635 | 6.76 |
| membrane 70 homolog A (S. cerevisiae) | | | | | | | | | |
| RNA pseudouridylate synthase | RPUSD4 | NM_032795 | FLJ14494 | 3396883 | chr11 | − | 125577202 | 125586743 | 6.76 |
| domain containing 4 | | | | | | | | | |
| sprouty-related, EVH1 domain | SPRED1 | NM_152594 | FLJ33903 | 3589141 | chr15 | + | 36331548 | 36468943 | 6.76 |
| containing 1 | | | | | | | | | |
| EH-domain containing 4 | EHD4 | NM_139265 | PAST4 | 3620276 | chr15 | − | 39977674 | 40052063 | 6.75 |
| proteasome (prosome, macropain) | PSMD12 | XM_001134070 // | MGC75406 // p55 | 3768103 | chr17 | − | 62764497 | 62804392 | 6.75 |
| 26S subunit, non-ATPase, 12 | | XM_001134072 // | | | | | | | |
| | | XM_001134073 // | | | | | | | |
| | | NM_002816 | | | | | | | |
| nucleoporin 160 kDa | NUP160 | NM_015231 | MGC150678 // | 3371986 | chr11 | − | 47169280 | 47858048 | 6.74 |
| | | | MGC150679 | | | | | | |
| EPS8-like 1 | EPS8L1 | NM_017729 // | DRC3 // EPS8R1 // | 3841949 | chr19 | + | 60273799 | 60291530 | 6.74 |
| | | NM_133180 // | FLJ20258 // | | | | | | |
| | | NM_139204 | MGC23164 // | | | | | | |
| | | | MGC4642 // PP10566 | | | | | | |
| chromatin modifying protein 4B | CHMP4B | NM_176812 | C20orf178 // | 3882681 | chr20 | + | 31862780 | 31905829 | 6.74 |
| | | | CHMP4A // SNF7-2 | | | | | | |
| | | | // Shax1 // | | | | | | |
| thioredoxin interacting protein | TXNIP | NM_006472 | EST01027 // | 2356115 | chr1 | + | 144149846 | 144164251 | 6.72 |
| | | | HHCPA78 // THIF // | | | | | | |
| | | | VDUP1 | | | | | | |
| eukaryotic translation initiation factor | EIF1AX // | XM_001134077 // | EIF1A // EIF4C // | 4002148 | chrX | − | 20052567 | 20069880 | 6.71 |
| 1A, X-linked // eukaryotic | EIF1AP1 | NM_001412 // — | eIF-1A // eIF-4C // — | | | | | | |
| translation initiation factor 1A | | | | | | | | | |
| mitochondrial ribosomal protein S30 | MRPS30 | NM_016640 | DKFZp566B2024 // | 28086122 | chr5 | + | 44844784 | 45054668 | 6.71 |
| | | | MRP-S30 // PAP // | | | | | | |
| | | | PDCD9 | | | | | | |
| Rho GTPase activating protein 21 | ARHGAP21 | NM_020824 | ARHGAP10 // | 3281621 | chr10 | − | 24912552 | 25052583 | 6.71 |
| | | | DKFZp761L0424 // | | | | | | |
| | | | FLJ33323 | | | | | | |
| regulator of G-protein signalling 3 | RGS3 | NM_017790 // | C2PA // FLJ20370 // | 3185643 | chr9 | + | 115246842 | 115399812 | 6.71 |
| | | NM_021106 // | FLJ31516 // | | | | | | |
| | | NM_130795 // | FLJ90496 // PDZ- | | | | | | |
| | | NM_134427 // | RGS3 // RGP3 | | | | | | |
| | | NM_144488 // | | | | | | | |
| | | NM_144489 | | | | | | | |
| NFKB inhibitor interacting Ras-like 2 | NKIRAS2 | NM_001001349 // | DKFZP434N1526 // | 3721579 | chr17 | + | 37422156 | 37431177 | 6.70 |
| | | NM_017595 | KBRAS2 // | | | | | | |
| | | | MGC74742 // | | | | | | |
| polypyrimidine tract binding protein 2 | PTBP2 | NM_021190 | FLJ34897 // MIBP // | 2348060 | chr1 | + | 96959805 | 97144643 | 6.70 |
| | | | PTB // PTBLP // | | | | | | |
| | | | brPTB // nPTB // | | | | | | |
| | | | nPTB5 // nPTB6 // | | | | | | |
| | | | nPTB7 // nPTB8 | | | | | | |
| splicing factor 1 | SF1 | NM_004630 // | D11S636 // ZFM1 // | 3377044 | chr11 | − | 64287827 | 64302817 | 6.69 |
| | | NM_201995 // | ZNF162 | | | | | | |
| | | NM_201997 // | | | | | | | |
| | | NM_201998 | | | | | | | |
| Nipped-B homolog (Drosophila) // | NIPBL // | NM_015384 // | CDLS // | 2806799 | chr5 | + | 36894163 | 37115932 | 6.68 |
| nuclear receptor binding protein 1 | NRBP1 | NM_133433 // | DKFZp434L1319 // | | | | | | |
| | | NM_013392 | FLJ11203 // | | | | | | |
| | | | FLJ12597 // | | | | | | |
| | | | FLJ13354 // | | | | | | |
| | | | FLJ13648 // | | | | | | |
| | | | FLJ44854 // IDN3 // | | | | | | |
| | | | IDN3-B // BCON3 // | | | | | | |
| | | | FLJ27109 // | | | | | | |
| | | | FLJ35541 // MADM | | | | | | |
| | | | // MUDPNP // NRBP | | | | | | |
| ring finger and WD repeat domain 3 | RFWD3 | NM_018124 | FLJ10520 // RNF201 | 3699044 | chr16 | − | 73212799 | 73258283 | 6.68 |
| BCL2-associated athanogene | BAG1 | NM_004323 | — | 3203482 | chr9 | − | 33237838 | 33255392 | 6.68 |
| chromosome 6 open reading frame | C6orf111 // | NM_032870 // | DKFZp564B0769 // | 2966253 | chr6 | − | 99952648 | 99980235 | 6.68 |
| 111 // ubiquitin specific peptidase | USP45 | XM_371838 // | FLJ14752 // | | | | | | |
| 45 | | XM_931094 // | FLJ14853 // | | | | | | |
| | | XM_931115 // | FLJ14992 // | | | | | | |
| | | XM_931124 // | FLJ90147 // | | | | | | |
| | | XM_939661 // | HSPC306 // | | | | | | |
| | | XM_944255 // | MGC104269 // RP11- | | | | | | |
| | | XM_944271 // | 98I9.2 // SRrp 130 // | | | | | | |
| | | XM_944273 // | bA98I9.2 // | | | | | | |
| | | NM_001080481 | MGC14793 | | | | | | |
| dynactin 5 (p25) | DCTN5 | NM_032486 | MGC3248 // p25 | 3653042 | chr16 | + | 23560236 | 23588668 | 6.67 |
| integrator complex subunit 10 | INTS10 | NM_018142 | C8orf35 // FLJ10569 | 3088405 | chr8 | + | 19666766 | 19753848 | 6.67 |
| | | | // INT10 | | | | | | |
| arrestin domain containing 3 | ARRDC3 | NM_020801 | KIAA1376 // TLIMP | 2866704 | chr5 | − | 90700311 | 90782084 | 6.67 |
| CD59 molecule, complement | CD59 | NM_000611 // | 16.3A5 // EJ16 // | 3368707 | chr11 | − | 33676397 | 33714806 | 6.65 |
| regulatory protein | | NM_203329 // | EJ30 // EL32 // | | | | | | |
| | | NM_203330 // | G344 // MGC2354 // | | | | | | |
| | | NM_203331 | MIC11 // MIN1 // | | | | | | |
| | | | MIN2 // MIN3 // | | | | | | |
| | | | MSK21 // | | | | | | |
| | | | PROTECTIN // p18- | | | | | | |
| opioid growth factor receptor | OGFR | NM_007346 | — | 3892941 | chr20 | + | 60906461 | 60915796 | 6.65 |
| chromosome 20 open reading frame | C20orf11 | NM_017896 | TWA1 | 3893072 | chr20 | + | 61039681 | 61050570 | 6.63 |
| 11 | | | | | | | | | |
| ERBB receptor feedback inhibitor 1 | ERRFI1 | NM_018948 | GENE-33 // MIG-6 | 2395177 | chr1 | − | 7987071 | 8194824 | 6.62 |
| | | | // MIG6 // RALT | | | | | | |
| DEAD (Asp-Glu-Ala-Asp) box | DDX5 | NM_004396 | DKFZp686J01190 // | 3766893 | chr17 | − | 59924843 | 59935796 | 6.62 |
| polypeptide 5 | | | G17P1 // HLR1 // | | | | | | |
| | | | HUMP68 // p68 | | | | | | |
| chaperonin containing TCP1, subunit | CCT2 | NM_006431 | 99D8.1 // CCT-beta | 3421630 | chr12 | + | 68265508 | 68281612 | 6.62 |
| 2 (beta) | | | // CCTB // | | | | | | |
| | | | MGC142074 // | | | | | | |
| | | | MGC142076 // | | | | | | |
| | | | PRO1633 // TCP-1- | | | | | | |
| | | | beta | | | | | | |
| TROVE domain family, member 2 | TROVE2 | NM_001042369 // | SSA2 | 2372924 | chr1 | + | 191281274 | 191327514 | 6.62 |
| | | NM_001042370 // | | | | | | | |
| | | NM_004600 | | | | | | | |
| casein kinase 1, delta | CSNK1D | XM_001132734 // | HCKID | 3774823 | chr17 | − | 77789955 | 77824878 | 6.62 |
| | | NM_001893 // | | | | | | | |
| | | NM_139062 | | | | | | | |
| nucleoside phosphorylase | NP | NM_000270 | MGC117396 // | 3527514 | chr14 | + | 20007398 | 20015985 | 6.61 |
| | | | MGC125915 // | | | | | | |
| | | | MGC125916 // PNP | | | | | | |
| | | | // PRO1837 // PUNP | | | | | | |
| ubiquitin C | UBC | XM_001132949 // | HMG20 | 3476741 | chr12 | − | 123962213 | 123965631 | 6.61 |
| | | NM_021009 | | | | | | | |
| general transcription factor IIF, | GTF2F2 | NM_004128 | BTF4 // RAP30 // | 3488094 | chr13 | + | 44592617 | 44756234 | 6.60 |
| polypeptide 2, 30 kDa | | | TF2F2 // TFIIF | | | | | | |
| RAS p21 protein activator (GTPase | RASA1 | NM_002890 // | CM-AVM // CMAVM | 2819044 | chr5 | + | 86599391 | 86724647 | 6.60 |
| activating protein) 1 | | NM_022650 | // DKFZp434N071 // | | | | | | |
| | | | GAP // PKWS // | | | | | | |
| | | | RASA // RASGAP // | | | | | | |
| | | | p120GAP | | | | | | |
| chromosome 8 open reading frame | C8orf53 | NM_032334 | MGC14595 | 3112543 | chr8 | + | 117847923 | 117930859 | 6.60 |
| activator of basal transcription 1 | ABT1 | NM_013375 | hABT1 | 2899519 | chr6 | + | 26661709 | 26708945 | 6.59 |
| DEAD (Asp-Glu-Ala-Asp) box | DDX50 | NM_024045 | GU2 // GUB // | 3250019 | chr10 | + | 70330615 | 70377158 | 6.58 |
| polypeptide 50 | | | MGC3199 // RH- | | | | | | |
| | | | II/GuB | | | | | | |
| component of oligomeric golgi | COG1 | NM_018714 | DKFZp762L1710 // | 3733938 | chr17 | + | 68695573 | 68739952 | 6.58 |
| complex 1 | | | KIAA1381 // LDLB | | | | | | |
| adaptor-related protein complex 1, | AP1G1 | NM_001030007 // | ADTG // CLAPG1 // | 3697799 | chr16 | − | 70320417 | 70400594 | 6.57 |
| gamma 1 subunit | | NM_001128 | MGC18255 | | | | | | |
| UTP15, U3 small nucleolar | UTP15 | NM_032175 | FLJ12787 | 2815455 | chr5 | + | 72897308 | 72915109 | 6.57 |
| ribonucleoprotein, homolog (S. cerevisiae) | | | | | | | | | |
| PR domain containing 4 // protein | PRDM4 // | NM_012406 // | MGC45046 // PFM1 | 3470037 | chr12 | − | 106650784 | 106679235 | 6.56 |
| kinase, cAMP-dependent, regulatory, | PRKAR2B | NM_002736 | // PRKAR2 // RII- | | | | | | |
| type II, beta | | | BETA | | | | | | |
| nuclear transcription factor, X-box | NFXL1 | NM_152995 | FLJ16294 // HOZFP | 2768273 | chr4 | − | 47543715 | 47612453 | 6.55 |
| binding-like 1 | | | | | | | | | |
| YTH domain family, member 3 | YTHDF3 | NM_152758 | FLJ31657 | 3100909 | chr8 | + | 64213088 | 64392289 | 6.55 |
| MAP/microtubule affinity-regulating | MARK2 | NM_001039468 // | EMK1 // MGC99619 | 3334025 | chr11 | + | 63362634 | 63435067 | 6.55 |
| kinase 2 | | NM_001039469 // | // PAR-1 | | | | | | |
| | | NM_004954 // | | | | | | | |
| | | NM_017490 | | | | | | | |
| GC-rich promoter binding protein 1 | GPBP1 | NM_022913 | DKFZp761C169 // | 2810458 | chr5 | + | 56504841 | 56597388 | 6.55 |
| | | | GPBP // MGC126339 | | | | | | |
| CTD (carboxy-terminal domain, RNA | CTDP1 | NM_004715 // | CCFDN // FCP1 | 3795312 | chr18 | + | 75540799 | 75617601 | 6.54 |
| polymerase II, polypeptide A) | | NM_048368 | | | | | | | |
| phosphatase, subunit 1 | | | | | | | | | |
| protein tyrosine phosphatase, non- | PTPN12 | NM_002835 | PTP-PEST // PTPG1 | 3009959 | chr7 | + | 77004361 | 77123074 | 6.54 |
| receptor type 12 | | | | | | | | | |
| ubiquitin-conjugating enzyme E2M | UBE2M // | NM_003969 // | UBC-RS2 // UBC12 | 3872999 | chr19 | − | 63758732 | 63762147 | 6.54 |
| (UBC12 homolog, yeast) // ubiquitin- | UBE2MP1 | NR_002837 | // hUbc12 // — | | | | | | |
| conjugating enzyme E2M pseudogene 1 | | | | | | | | | |
| wingless-type MMTV integration site | WNT11 | NM_004626 | HWNT11 // | 3382523 | chr11 | − | 75542387 | 75599441 | 6.53 |
| family, member 11 | | | MGC141946 // | | | | | | |
| | | | MGC141948 | | | | | | |
| chromosome 14 open reading frame | C14orf32 | NM_144578 | MGC23138 // MISS | 3536663 | chr14 | + | 54588114 | 54606655 | 6.53 |
| 32 | | | // c14_5346 | | | | | | |
| signal sequence receptor, beta | SSR2 | XM_001128341 // | DKFZp686F19123 // | 2437871 | chr1 | − | 154245480 | 154257371 | 6.52 |
| (translocon-associated protein beta) | | NM_003145 | HSD25 // TLAP // | | | | | | |
| | | | TRAPB | | | | | | |
| RAS and EF-hand domain containing | RASEF | NM_152573 | FLJ31614 // RAB45 | 3211938 | chr9 | − | 84772206 | 84937805 | 6.52 |
| cold shock domain protein A | CSDA | NM_003651 | CSDA1 // DBPA // | 3444252 | chr12 | − | 10742960 | 10775038 | 6.51 |
| | | | ZONAB | | | | | | |
| leucine-rich PPR-motif containing | LRPPRC | NM_133259 | CLONE-23970 // | 2550790 | chr2 | − | 43961504 | 44085128 | 6.51 |
| | | | GP130 // LRP130 // | | | | | | |
| | | | LSFC | | | | | | |
| oxysterol binding protein | OSBP | NM_002556 | OSBP1 | 3374698 | chr11 | − | 59098455 | 59140193 | 6.51 |
| MAP/microtubule affinity-regulating | MARK3 | NM_002376 | CTAK1 // KP78 // | 3553690 | chr14 | + | 102921419 | 103370656 | 6.50 |
| kinase 3 | | | PAR1A | | | | | | |
| oxidation resistance 1 | OXR1 | NM_181354 | FLJ10125 // | 3110999 | chr8 | + | 107351659 | 107834092 | 6.50 |
| | | | FLJ38829 // | | | | | | |
| | | | FLJ41673 // | | | | | | |
| | | | FLJ42450 // | | | | | | |
| | | | FLJ45656 // | | | | | | |
| | | | Nbla00307 | | | | | | |
| ATG16 autophagy related 16-like 1 | ATG16L1 | NM_198890 // | APG16L // ATG16L | 2532793 | chr2 | + | 233825030 | 233869050 | 6.50 |
| (S. cerevisiae) | | NM_017974 // | // FLJ00045 // | | | | | | |
| | | NM_030803 | FLJ10035 // | | | | | | |
| | | | FLJ10828 // | | | | | | |
| | | | FLJ22677 // WDR30 | | | | | | |
| ankyrin repeat domain 17 | ANKRD17 | NM_032217 // | FLJ22206 // GTAR | 2773023 | chr4 | − | 74158547 | 74343428 | 6.50 |
| | | NM_198889 | // KIAA0697 // NY- | | | | | | |
| | | | BR-16 | | | | | | |
| small nuclear ribonucleoprotein | SNRPB2 | NM_003092 // | MGC24807 // | 3877776 | chr20 | + | 16645421 | 16670916 | 6.49 |
| polypeptide B″ | | NM_198220 | MGC45309 | | | | | | |
| protein kinase, cAMP-dependent, | PRKAR1A | NM_002734 // | CAR // CNC // | 3732885 | chr17 | + | 64019700 | 64059052 | 6.49 |
| regulatory, type I, alpha (tissue | | NM_212471 // | CNC1 // | | | | | | |
| specific extinguisher 1) | | NM_212472 | DKFZp779L0468 // | | | | | | |
| | | | MGC17251 // PKR1 | | | | | | |
| chromosome 20 open reading frame | C20orf20 | NM_018270 | Eaf7 // FLJ10914 // | 3892918 | chr20 | + | 60898107 | 60903524 | 6.49 |
| 20 | | | MRG15BP // MRGBP | | | | | | |
| ADP-ribosylation-like factor 6 | ARL6IP4 | NM_001002251 // | MGC814 // SR-25 // | 3435681 | chr12 | + | 122030886 | 122033406 | 6.48 |
| interacting protein 4 | | NM_001002252 // | SRp25 | | | | | | |
| | | NM_016638 // | | | | | | | |
| | | NM_018694 | | | | | | | |
| pre-mRNA cleavage factor 1, 59 kDa | FLJ12529 | NM_024811 | FLJ39024 // | 3375340 | chr11 | − | 60926704 | 60954030 | 6.48 |
| subunit | | | MGC9315 | | | | | | |
| RNA binding motif protein 17 | RBM17 | NM_032905 | MGC14439 // SPF45 | 3233547 | chr10 | + | 6170982 | 6225410 | 6.47 |
| actin binding LIM protein family, | ABLIM3 | NM_014945 | HMFN1661 | 2834863 | chr5 | + | 148497619 | 148620192 | 6.47 |
| member 3 | | | | | | | | | |
| asparagine synthetase domain | ASNSD1 | NM_019048 | FLJ20752 // | 2519860 | chr2 | + | 190198920 | 190244286 | 6.46 |
| containing 1 | | | NBLA00058 // | | | | | | |
| | | | NS3TP1 | | | | | | |
| poly(A) polymerase alpha | PAPOLA | NM_032632 | MGC5378 // PAP | 3550392 | chr14 | + | 96038038 | 96104627 | 6.46 |
| transmembrane protein 87A | TMEM87A | NM_015497 | DKFZP564G2022 | 3620515 | chr15 | − | 40289650 | 40353024 | 6.46 |
| KIAA0907 | KIAA0907 | NM_014949 | RP11-336K24.1 | 2437753 | chr1 | − | 154149414 | 154174836 | 6.45 |
| DEAD (Asp-Glu-Ala-Asp) box | DDX10 | NM_004398 | HRH-J8 | 3347831 | chr11 | + | 108041010 | 108316858 | 6.45 |
| polypeptide 10 | | | | | | | | | |
| protein tyrosine phosphatase, non- | PTPN1 | NM_002827 | PTP1B | 3888721 | chr20 | + | 48560298 | 48634700 | 6.45 |
| receptor type 1 | | | | | | | | | |
| insulin-like growth factor 2 mRNA | IGF2BP2 | NM_001007225 // | IMP-2 // IMP2 // | 2708922 | chr3 | − | 186844235 | 187025506 | 6.45 |
| binding protein 2 | | NM_006548 | VICKZ2 // p62 | | | | | | |
| zinc finger protein 592 | ZNF592 | NM_014630 | KIAA0211 // | 3605832 | chr15 | + | 83092594 | 83150653 | 6.44 |
| | | | MGC138437 // | | | | | | |
| | | | MGC138439 | | | | | | |
| HIV-1 Rev binding protein | HRB | XM_941338 // | MGC116938 // | 2530599 | chr2 | + | 228045122 | 228134167 | 6.43 |
| | | NM_004504 | MGC116940 // RAB | | | | | | |
| | | | // RIP | | | | | | |
| mitogen-activated protein kinase | MAP3K4 | NM_005922 // | FLJ42439 // | 2934801 | chr6 | + | 161332723 | 161458399 | 6.42 |
| kinase kinase 4 | | NM_006724 | KIAA0213 // | | | | | | |
| | | | MAPKKK4 // MEKK4 | | | | | | |
| | | | // MTK1 // PRO0412 | | | | | | |
| ubiquitin specific peptidase 15 | USP15 | NM_006313 | KIAA0529 // | 3419147 | chr12 | + | 60940328 | 61086166 | 6.41 |
| | | | MGC131982 // | | | | | | |
| | | | MGC149838 // | | | | | | |
| | | | MGC74854 // UNPH4 | | | | | | |
| cullin 1 | CUL1 | NM_003592 | MGC149834 // | 3030285 | chr7 | + | 148026213 | 148129128 | 6.41 |
| | | | MGC149835 | | | | | | |
| family with sequence similarity 104, | FAM104A | NM_032837 | FLJ14775 | 3769969 | chr17 | − | 68715100 | 68740203 | 6.41 |
| member A | | | | | | | | | |
| nucleoporin 88 kDa | NUP88 | NM_002532 | MGC8530 | 3742652 | chr17 | − | 5204992 | 5264204 | 6.41 |
| kinesin family member 22 | KIF22 | NM_007317 | KID // KNSL4 // | 3655628 | chr16 | + | 29708911 | 29724778 | 6.40 |
| | | | OBP // OBP-1 // | | | | | | |
| zinc finger protein 313 | ZNF313 | NM_018683 | RNF114 | 3888474 | chr20 | + | 47986321 | 48003818 | 6.39 |
| ATG3 autophagy related 3 homolog | ATG3 | NM_022488 | APG3 // APG3L // | 2688759 | chr3 | − | 113726408 | 113769332 | 6.39 |
| (S. cerevisiae) | | | DKFZp564M1178 // | | | | | | |
| | | | FLJ22125 // | | | | | | |
| | | | MGC15201 // PC3-96 | | | | | | |
| PTPRF interacting protein, binding | PPFIBP1 | NM_003622 // | L2 // hSGT2 // | 3409211 | chr12 | + | 27567649 | 27739981 | 6.39 |
| protein 1 (liprin beta 1) | | NM_177444 | hSgt2p | | | | | | |
| flotillin 1 | FLOT1 | NM_005803 | — | 2948587 | chr6 | − | 30796156 | 30818490 | 6.38 |
| isoleucyl-tRNA synthetase | IARS | NM_002161 // | FLJ20736 // IARS1 | 3214668 | chr9 | − | 94012330 | 94096287 | 6.38 |
| | | NM_013417 | // ILRS // PRO0785 | | | | | | |
| TM2 domain containing 2 | TM2D2 | NM_001024380 // | BLP1 // MGC125813 | 3132333 | chr8 | − | 38965485 | 38973467 | 6.38 |
| | | NM_001024381 // | // MGC125814 | | | | | | |
| | | NM_031940 // | | | | | | | |
| | | NM_078473 | | | | | | | |
| hypothetical protein MGC22014 | MGC22014 | XM_371501 // | KIAA0401 | 2489071 | chr2 | + | 74066761 | 74188804 | 6.37 |
| | | XM_942026 | | | | | | | |
| THO complex 1 | THOC1 | NM_005131 | HPR1 // P84 // | 3795680 | chr18 | − | 201783 | 258461 | 6.36 |
| | | | P84N5 | | | | | | |
| chromosome 14 open reading frame | C14orf129 | NM_016472 | HSPC210 // | 3550328 | chr14 | + | 95899075 | 95923378 | 6.35 |
| 129 | | | MGC4945 | | | | | | |
| vascular endothelial zinc finger 1 | VEZF1 | NM_007146 | DB1 // ZNF161 | 3764066 | chr17 | − | 53359568 | 53420600 | 6.34 |
| heat shock 70 kDa protein 8 // Fc | HSPA8 // | NM_006597 // | HSC54 // HSC70 // | 3395416 | chr11 | − | 122429309 | 122438895 | 6.33 |
| fragment of IgG, low affinity IIIb, | FCGR3B | NM_153201 // | HSC71 // HSP71 // | | | | | | |
| receptor (CD16b) | | NM_000570 | HSP73 // HSPA10 // | | | | | | |
| | | | LAP1 // MGC131511 | | | | | | |
| | | | // MGC29929 // | | | | | | |
| | | | NIP71 // CD16 // | | | | | | |
| | | | CD16b // FCG3 // | | | | | | |
| | | | FCGR3 | | | | | | |
| cullin-associated and neddylation- | CAND1 | NM_018448 | DKFZp434M1414 // | 3420713 | chr12 | + | 65946048 | 65994719 | 6.32 |
| dissociated 1 | | | FLJ10114 // | | | | | | |
| | | | FLJ10929 // | | | | | | |
| | | | FLJ38691 // | | | | | | |
| | | | FLJ90441 // | | | | | | |
| | | | KIAA0829 // TIP120 | | | | | | |
| | | | // TIP120A | | | | | | |
| kelch repeat and BTB (POZ) domain | KBTBD4 | NM_016506 // | BKLHD4 // FLJ10450 | 3372337 | chr11 | − | 47550326 | 47557362 | 6.32 |
| containing 4 | | NM_018095 | // HSPC252 | | | | | | |
| karyopherin alpha 4 (importin alpha | KPNA4 | NM_002268 | IPOA3 // MGC12217 | 2703217 | chr3 | − | 161663997 | 161766070 | 6.31 |
| 3) | | | // MGC26703 // | | | | | | |
| | | | QIP1 // SRP3 | | | | | | |
| chromosome 12 open reading frame | C12orf52 | NM_032848 | FLJ14827 | 3432641 | chr12 | + | 112107758 | 112114543 | 6.31 |
| 52 | | | | | | | | | |
| aurora kinase A // serine/threonine | AURKA // | NM_003600 // | AIK // ARK1 // | 3910785 | chr20 | − | 54377855 | 54402553 | 6.30 |
| kinase 6 pseudogene | STK6P | NM_198433 // | AURA // AURORA2 | | | | | | |
| | | NM_198434 // | // BTAK // | | | | | | |
| | | NM_198435 // | MGC34538 // STK15 | | | | | | |
| | | NM_198436 // | // STK6 // STK7 // — | | | | | | |
| | | NM_198437 // | | | | | | | |
| | | NR_001587 | | | | | | | |
| eukaryotic translation initiation factor | EIF2S1 | NM_004094 | EIF-2 // EIF-2A // | 3541137 | chr14 | + | 66896518 | 66922983 | 6.30 |
| 2, subunit 1 alpha, 35 kDa | | | EIF-2alpha // EIF2 // | | | | | | |
| | | | EIF2A | | | | | | |
| ubiquitin specific peptidase 7 (herpes | USP7 | NM_003470 | HAUSP // TEF1 | 3679564 | chr16 | − | 8893458 | 8964832 | 6.30 |
| virus-associated) | | | | | | | | | |
| SH3 domain containing ring finger 2 | SH3RF2 | NM_152550 | FLJ23654 // | 2833924 | chr5 | + | 145296335 | 145441529 | 6.30 |
| | | | MGC149788 // | | | | | | |
| | | | MGC149789 // | | | | | | |
| | | | MGC90410 // | | | | | | |
| hydroxymethylbilane synthase | HMBS | NM_000190 // | PBG-D // PBGD // | 3351841 | chr11 | + | 118460803 | 118470689 | 6.30 |
| | | NM_001024382 | UPS | | | | | | |
| eukaryotic translation initiation factor | EIF4E | NM_001968 | CBP // EIF4E1 // | 2778980 | chr4 | − | 99888790 | 100070801 | 6.30 |
| 4E | | | EIF4EL1 // EIF4F // | | | | | | |
| | | | MGC111573 | | | | | | |
| splicing factor, arginine/serine-rich | SFRS15 | NM_020706 | DKFZP434E098 // | 3928866 | chr21 | − | 31965222 | 32077764 | 6.30 |
| 15 | | | FLJ23364 // | | | | | | |
| | | | KIAA1172 // SRA4 | | | | | | |
| gem (nuclear organelle) associated | GEMIN5 | NM_015465 | DKFZP586M1824 // | 2882897 | chr5 | − | 154247066 | 154309713 | 6.30 |
| protein 5 | | | MGC142174 | | | | | | |
| TNF receptor-associated factor 6 | TRAF6 | NM_004620 // | MGC: 3310 // RNF85 | 3369890 | chr11 | − | 36465173 | 36488940 | 6.29 |
| | | NM_145803 | | | | | | | |
| neurofibromin 2 (bilateral acoustic | NF2 | NM_000268 // | ACN // BANF // | 3942062 | chr22 | + | 28329446 | 28424577 | 6.29 |
| neuroma) | | NM_016418 // | Merlin // SCH | | | | | | |
| | | NM_181825 // | | | | | | | |
| | | NM_181828 // | | | | | | | |
| | | NM_181829 // | | | | | | | |
| | | NM_181830 // | | | | | | | |
| | | NM_181831 // | | | | | | | |
| | | NM_181832 // | | | | | | | |
| | | NM_181833 | | | | | | | |
| structural maintenance of | SMC3 | NM_005445 | BAM // BMH // | 3263790 | chr10 | + | 112317039 | 112365483 | 6.28 |
| chromosomes 3 | | | CSPG6 // HCAP // | | | | | | |
| | | | SMC3L1 | | | | | | |
| dynamin binding protein | DNMBP | NM_015221 | KIAA1010 // TUBA | 3303165 | chr10 | − | 101624846 | 101759656 | 6.28 |
| cystatin B (stefin B) | CSTB | NM_000100 | CST6 // EPM1 // | 3934245 | chr21 | − | 44016298 | 44020687 | 6.28 |
| | | | PME // STFB | | | | | | |
| mucin 20, cell surface associated | MUC20 | NM_152673 | FLJ14408 // | 2659039 | chr3 | + | 196867150 | 196953662 | 6.27 |
| | | | KIAA1359 | | | | | | |
| tuberous sclerosis 1 | TSC1 | NM_000368 // | KIAA0243 // LAM // | 3228373 | chr9 | − | 134756560 | 134809831 | 6.27 |
| | | NM_001008567 | MGC86987 // TSC | | | | | | |
| abl interactor 2 | ABI2 | XM_001126750 // | ABI-2 // ABI2B // | 2523689 | chr2 | + | 203901161 | 204005128 | 6.27 |
| | | NM_005759 | AIP-1 // AblBP3 // | | | | | | |
| | | | SSH3BP2 // argBPIA | | | | | | |
| | | | // argBPIB | | | | | | |
| golgi reassembly stacking protein 2, | GORASP2 | NM_015530 // | DKFZP434D156 // | 2515050 | chr2 | + | 171457150 | 171551619 | 6.27 |
| 55 kDa // dehydrogenase/reductase | // DHRS9 | NM_005771 // | FLJ13139 // GOLPH6 | | | | | | |
| (SDR family) member 9 | | NM_199204 | // GRASP55 // GRS2 | | | | | | |
| | | | // p59 // 3alpha-HSD | | | | | | |
| | | | // RDH15 // RDHL | | | | | | |
| | | | // RETSDR8 | | | | | | |
| ATPase, Ca++ transporting, cardiac | ATP2A2 | NM_001681 // | ATP2B // DAR // DD | 3431483 | chr12 | + | 109203332 | 109352495 | 6.26 |
| muscle, slow twitch 2 | | NM_170665 | // MGC45367 // | | | | | | |
| | | | SERCA2 | | | | | | |
| hypothetical protein HSPC148 | HSPC148 | NM_016403 | — | 3387171 | chr11 | − | 94322815 | 94346692 | 6.26 |
| EH-domain containing 1 | EHD1 | NM_006795 | FLJ42622 // | 3377226 | chr11 | − | 64376790 | 64404202 | 6.26 |
| | | | FLJ44618 // H-PAST | | | | | | |
| | | | // HPAST1 // PAST | | | | | | |
| | | | // PAST1 | | | | | | |
| X-box binding protein 1 | XBP1 | NM_001079539 // | TREB5 // XBP2 | 3956589 | chr22 | − | 27520140 | 27526564 | 6.25 |
| | | NM_005080 | | | | | | | |
| enhancer of zeste homolog 2 | EZH2 | NM_004456 // | ENX-1 // EZH1 // | 3078348 | chr7 | − | 148135436 | 148212647 | 6.25 |
| (Drosophila) | | NM_152998 | MGC9169 | | | | | | |
| SEC24 related gene family, member | SEC24B | XM_001130118 // | MGC48822 // SEC24 | 2739079 | chr4 | + | 110510304 | 110681811 | 6.24 |
| B (S. cerevisiae) | | NM_001042734 // | | | | | | | |
| | | NM_006323 | | | | | | | |
| RAB, member RAS oncogene family- | RABL5 | NM_022777 | DKFZp761N0823 // | 3064638 | chr7 | − | 100742601 | 100751793 | 6.24 |
| like 5 | | | FLJ13225 // | | | | | | |
| | | | FLJ14117 | | | | | | |
| RCD1 required for cell | RQCD1 // | NM_005444 // | CNOT9 // RCD1 // | 2527856 | chr2 | + | 219141552 | 219170027 | 6.24 |
| differentiation 1 homolog (S. pombe) | PLCD4 | NM_032726 | RCD1+ // MGC12837 | | | | | | |
| // phospholipase C, delta 4 | | | | | | | | | |
| poly(rC) binding protein 2 | PCBP2 | NM_005016 // | HNRPE2 // | 3416036 | chr12 | + | 52132173 | 52161417 | 6.23 |
| | | NM_031989 | MGC110998 // | | | | | | |
| | | | hnRNP-E2 | | | | | | |
| solute carrier family 30 (zinc | SLC30A9 | NM_006345 | C4orf1 // GAC63 // | 2725381 | chr4 | + | 41684531 | 41812215 | 6.23 |
| transporter), member 9 | | | HUEL // ZNT9 | | | | | | |
| zinc finger protein 192 | ZNF192 | NM_006298 | LD5-1 // ZKSCAN8 | 2900299 | chr6 | + | 28217285 | 28232872 | 6.23 |
| jumonji domain containing 2A | JMJD2A | NM_014663 | JHDM3A // JMJD2 | 2333429 | chr1 | + | 43888407 | 43943994 | 6.23 |
| | | | // KIAA0677 | | | | | | |
| HtrA serine peptidase 2 | HTRA2 | NM_013247 // | OMI // PARK13 // | 2489411 | chr2 | + | 74609783 | 74614240 | 6.23 |
| | | NM_145074 | PRSS25 | | | | | | |
| epidermal growth factor receptor | EPS8 | NM_004447 | — | 3445908 | chr12 | − | 15664367 | 15833589 | 6.23 |
| pathway substrate 8 | | | | | | | | | |
| glutamine and serine rich 1 | QSER1 | NM_024774 // | FLJ21924 | 3325768 | chr11 | + | 32871321 | 32971438 | 6.22 |
| | | NM_001076786 | | | | | | | |
| phosphomannomutase 2 | PMM2 | NM_000303 | CDG1 // CDG1a // | 3647504 | chr16 | + | 8798971 | 8850664 | 6.22 |
| | | | CDGS | | | | | | |
| DEAH (Asp-Glu-Ala-His) box | DHX15 | NM_001358 | DBP1 // DDX15 // | 2763805 | chr4 | − | 24128159 | 24250940 | 6.22 |
| polypeptide 15 | | | HRH2 // PRP43 // | | | | | | |
| | | | PRPF43 // PrPp43p | | | | | | |
| transforming growth factor, beta | TGFBR2 | NM_001024847 // | AAT3 // FAA3 // | 2615360 | chr3 | + | 30492214 | 30710745 | 6.21 |
| receptor II (70/80 kDa) | | NM_003242 | HNPCC6 // MFS2 // | | | | | | |
| | | | RIIC // TAAD2 // | | | | | | |
| | | | TGFR-2 // TGFbeta- | | | | | | |
| | | | RII | | | | | | |
| tropomyosin 1 (alpha) | TPM1 | NM_000366 // | HTM-alpha // TMSA | 3597338 | chr15 | + | 61105711 | 61151151 | 6.21 |
| | | NM_001018004 // | // TPM1-alpha // | | | | | | |
| | | NM_001018005 // | TPM1-kappa | | | | | | |
| | | NM_001018006 // | | | | | | | |
| | | NM_001018007 // | | | | | | | |
| | | NM_001018008 // | | | | | | | |
| | | NM_001018020 | | | | | | | |
| HLA-B associated transcript 4 | BAT4 | NM_033177 | ANKRD59 // D6S54E | 2949210 | chr6 | − | 31736995 | 31742029 | 6.21 |
| | | | // G5 // GPANK1 // | | | | | | |
| | | | GPATCH10 | | | | | | |
| COX4 neighbor | COX4NB | NM_006067 | C16orf4 // NOC4 | 3703164 | chr16 | − | 84362936 | 84391266 | 6.21 |
| RNA binding motif protein 7 | RBM7 | NM_016090 | FLJ11153 | 3349918 | chr11 | + | 113776539 | 113790335 | 6.20 |
| suppressor of Ty 4 homolog 1 (S. cerevisiae) | SUPT4H1 | NM_003168 | SPT4H // SUPT4H | 3764384 | chr17 | − | 53777538 | 53784705 | 6.19 |
| PRP4 pre-mRNA processing factor 4 | PRPF4B | NM_003913 | KIAA0536 // PR4H // | 2892738 | chr6 | + | 3966500 | 4010215 | 6.19 |
| homolog B (yeast) | | | PRP4 // PRP4H // | | | | | | |
| | | | PRP4K // | | | | | | |
| | | | dJ1013A10.1 | | | | | | |
| phosphoglycerate kinase 1 | PGK1 | NM_000291 | MGC117307 // | 3982462 | chrX | + | 77207320 | 77269191 | 6.19 |
| | | | MGC142128 // | | | | | | |
| | | | MGC8947 // MIG10 | | | | | | |
| | | | // PGKA | | | | | | |
| ATP-binding cassette, sub-family C | ABCC1 | NM_004996 // | ABC29 // ABCC // | 3649890 | chr16 | + | 15950935 | 16144419 | 6.18 |
| (CFTR/MRP), member 1 | | NM_019862 // | DKFZp686N04233 // | | | | | | |
| | | NM_019898 // | DKFZp781G125 // | | | | | | |
| | | NM_019899 // | GS-X // MRP // | | | | | | |
| | | NM_019900 // | MRP1 | | | | | | |
| | | NM_019901 // | | | | | | | |
| | | NM_019902 | | | | | | | |
| transmembrane protein 123 | TMEM123 | NM_052932 | KCT3 // PORIMIN // | 3388631 | chr11 | − | 101772285 | 101828945 | 6.18 |
| | | | PORMIN | | | | | | |
| SEH1-like (S. cerevisiae) | SEH1L | NM_001013437 // | SEC13L // SEH1A // | 3779756 | chr18 | + | 12886655 | 12981306 | 6.17 |
| | | NM_031216 | SEH1B // Seh1 | | | | | | |
| ferritin, heavy polypeptide-like 7 // | FTHL7 // | NR_002202 // | —// FTH // FTHL6 | 3375648 | chr11 | − | 61488337 | 61492603 | 6.16 |
| ferritin, heavy polypeptide 1 // | FTH1 // | NM_002032 // | // MGC104426 // | | | | | | |
| ferritin, heavy polypeptide-like 3 // | FTHL3 // | NR_002201 // —// | PIG15 // PLIF // | | | | | | |
| ferritin, heavy polypeptide | FTHP1 // | NR_002205 // | FTHL5 | | | | | | |
| pseudogene 1 // ferritin, heavy | FTHL12 // | NR_002204 // | | | | | | | |
| polypeptide-like 12 // ferritin, heavy | FTHL11 // | NR_002203 | | | | | | | |
| polypeptide-like 11 // ferritin, heavy | FTHL8 | | | | | | | | |
| DNA polymerase-transactivated | DNAPTP6 | NM_015535 | DKFZp564A2416 | 2522094 | chr2 | + | 200879035 | 201053805 | 6.15 |
| protein 6 | | | | | | | | | |
| serologically defined colon cancer | SDCCAG10 | NM_005869 | NY-CO-10 | 2812120 | chr5 | + | 64100099 | 64417210 | 6.15 |
| antigen 10 | | | | | | | | | |
| SHC (Src homology 2 domain | SHC1 | NM_003029 // | FLJ26504 // SHC // | 2436985 | chr1 | − | 153201334 | 153213510 | 6.15 |
| containing) transforming protein 1 | | NM_183001 | SHCA // p52SHC // | | | | | | |
| | | | p66 // p66SHC | | | | | | |
| tetratricopeptide repeat domain 17 | TTC17 | NM_018259 | DKFZp686D20222 // | 3327948 | chr11 | + | 43337004 | 43473027 | 6.13 |
| | | | FLJ10890 // | | | | | | |
| | | | FLJ13099 // | | | | | | |
| | | | FLJ23673 | | | | | | |
| polymerase (RNA) III (DNA directed) | POLR3E | NM_018119 | RPC5 // SIN | 3652489 | chr16 | + | 22216140 | 22253905 | 6.13 |
| polypeptide E (80 KD) | | | | | | | | | |
| bromodomain containing 1 | BRD1 | NM_014577 | BRL // BRPF1 // | 3965314 | chr22 | − | 48552694 | 48636916 | 6.12 |
| | | | BRPF2 // | | | | | | |
| | | | DKFZp686F0325 | | | | | | |
| SET translocation (myeloid leukemia- | SET | NM_003011 | 2PP2A // I2PP2A // | 3190659 | chr9 | + | 130485775 | 130498483 | 6.12 |
| associated) | | | IGAAD // PHAPII // | | | | | | |
| | | | TAF-IBETA | | | | | | |
| glutamate-cysteine ligase, catalytic | GCLC | NM_001498 | GCS // GLCL // | 2957700 | chr6 | − | 53438876 | 53589510 | 6.12 |
| subunit | | | GLCLC | | | | | | |
| SCY1-like 2 (S. cerevisiae) | SCYL2 | NM_017988 | CVAK104 // | 3428131 | chr12 | + | 99185070 | 99260564 | 6.12 |
| | | | FLJ10074 // | | | | | | |
| ribosomal protein L8 | RPL8 | NM_000973 // | — | 3159040 | chr8 | − | 145985994 | 145988615 | 6.11 |
| | | NM_033301 | | | | | | | |
| protein tyrosine phosphatase type | PTP4A1 | NM_003463 | DKFZp779M0721 // | 2911903 | chr6 | + | 64289625 | 64351448 | 6.10 |
| IVA, member 1 | | | HH72 // PRL-1 // | | | | | | |
| | | | PRL1 // PTP(CAAX1) | | | | | | |
| | | | // PTPCAAX1 | | | | | | |
| Rac GTPase activating protein 1 | RACGAP1 | NM_013277 | H3CYK-4 // ID-GAP | 3454223 | chr12 | − | 48657002 | 48705537 | 6.09 |
| | | | // MgcRacGAP | | | | | | |
| cold shock domain containing E1, | CSDE1 // | NM_001007553 // | D1S155E // | 2429277 | chr1 | − | 115061068 | 115102225 | 6.09 |
| RNA-binding // neuroblastoma RAS | NRAS | NM_007158 // | DKFZp779B0247 // | | | | | | |
| viral (v-ras) oncogene homolog | | NM_002524 | DKFZp779J1455 // | | | | | | |
| | | | FLJ26882 // RP5- | | | | | | |
| | | | 1000E10.3 // UNR // | | | | | | |
| | | | N-ras // NRAS1 | | | | | | |
| translocation protein 1 | TLOC1 | NM_003262 | Dtrp1 // FLJ32803 // | 2651782 | chr3 | + | 171166446 | 171194653 | 6.09 |
| | | | HTP1 // SEC62 | | | | | | |
| muscleblind-like (Drosophila) | MBNL1 | NM_021038 // | DKFZp686P06174 // | 2648141 | chr3 | + | 153444327 | 153670549 | 6.08 |
| | | NM_207292 // | EXP // EXP35 // | | | | | | |
| | | NM_207293 // | EXP40 // EXP42 // | | | | | | |
| | | NM_207294 // | KIAA0428 // MBNL | | | | | | |
| | | NM_207295 // | | | | | | | |
| | | NM_207296 // | | | | | | | |
| | | NM_207297 | | | | | | | |
| chromosome X open reading frame | CXorf40A // | NM_178124 // | CXorf40 // EOLA1 // — | 3994451 | chrX | + | 148413672 | 148439412 | 6.08 |
| 40A // chromosome X open reading | CXorf40B | NM_001013845 | | | | | | | |
| frame 40B | | | | | | | | | |
| ubiquitin B // chromosome 17 open | UBB // | NM_018955 // | FLJ25987 // | 3712041 | chr17 | + | 16223047 | 16228977 | 6.08 |
| reading frame 45 | C17orf45 | NM_152350 | MGC8385 // | | | | | | |
| | | | FLJ25777 // | | | | | | |
| | | | MGC40157 | | | | | | |
| solute carrier family 7 (cationic | SLC7A1 | NM_003045 | ATRC1 // CAT-1 // | 3507710 | chr13 | − | 28981563 | 29067721 | 6.07 |
| amino acid transporter, y+ system), | | | ERR // HCAT1 // | | | | | | |
| member 1 | | | REC1L | | | | | | |
| smu-1 suppressor of mec-8 and | SMU1 | NM_018225 | BWD // FLJ10805 // | 3203382 | chr9 | − | 33031772 | 33072208 | 6.06 |
| unc-52 homolog (C. elegans) | | | FLJ11970 // | | | | | | |
| | | | MGC117363 // RP11- | | | | | | |
| | | | 54K16.3 // SMU-1 | | | | | | |
| annexin A2 | ANXA2 | NM_001002857 // | ANX2 // ANX2L4 // | 3627248 | chr15 | − | 58247806 | 58482354 | 6.06 |
| | | NM_001002858 // | CAL1H // LIP2 // | | | | | | |
| | | NM_004039 | LPC2 // LPC2D // | | | | | | |
| | | | P36 // PAP-IV | | | | | | |
| COP9 constitutive photomorphogenic | COPS7B | NM_022730 | FLJ12612 // | 2532064 | chr2 | + | 232354256 | 232382342 | 6.06 |
| homolog subunit 7B (Arabidopsis) | | | MGC111077 | | | | | | |
| chromosome 1 open reading frame | C1orf174 | NM_207356 | RP13-531C17.2 | 2393816 | chr1 | − | 3791939 | 3812760 | 6.06 |
| 174 | | | | | | | | | |
| v-ets erythroblastosis virus E26 | ETS2 | NM_005239 | — | 3921068 | chr21 | + | 39059147 | 39118744 | 6.06 |
| oncogene homolog 2 (avian) | | | | | | | | | |
| actinin, alpha 4 | ACTN4 | NM_004924 | DKFZp686K23158 // | 3832643 | chr19 | + | 43830130 | 43913003 | 6.06 |
| | | | FSGS // FSGS1 | | | | | | |
| protein phosphatase 2 (formerly 2A), | PPP2CA | NM_002715 | PP2Ac // PP2CA // | 2876046 | chr5 | − | 133557588 | 133589841 | 6.05 |
| catalytic subunit, alpha isoform | | | RP-C | | | | | | |
| glycogen synthase kinase 3 beta | GSK3B | NM_002093 | — | 2691014 | chr3 | − | 121018519 | 121297142 | 6.05 |
| hypothetical protein FLJ14154 | FLJ14154 | NM_024845 | — | 3645901 | chr16 | + | 3433714 | 3476953 | 6.04 |
| poliovirus receptor-related 2 | PVRL2 | NM_001042724 // | CD112 // HVEB // | 3835814 | chr19 | + | 50041409 | 50084314 | 6.04 |
| (herpesvirus entry mediator B) | | NM_002856 | PRR2 // PVRR2 | | | | | | |
| DnaJ homolog subfamily A member 5 | DNAJA5 | NM_001012339 // | — | 2806256 | chr5 | + | 34965441 | 34994822 | 6.03 |
| | | NM_194283 | | | | | | | |
| zinc finger, RAN-binding domain | ZRANB2 | NM_005455 // | DKFZp686J1831 // | 2418000 | chr1 | − | 71292434 | 71355820 | 6.03 |
| containing 2 | | NM_203350 | DKFZp686N09117 // | | | | | | |
| | | | FLJ41119 // ZIS // | | | | | | |
| | | | ZIS1 // ZIS2 // | | | | | | |
| | | | ZNF265 | | | | | | |
| GDP dissociation inhibitor 2 | GDI2 | NM_001494 | FLJ16452 // | 3275132 | chr10 | − | 5844502 | 5924081 | 6.03 |
| | | | FLJ37352 // | | | | | | |
| | | | RABGDIB | | | | | | |
| transmembrane protein 138 | TMEM138 | NM_016464 | HSPC196 | 3332886 | chr11 | + | 60885939 | 60900898 | 6.02 |
| CDC28 protein kinase regulatory | CKS2 | NM_001827 | CKSHS2 | 3178583 | chr9 | + | 91050736 | 91121542 | 6.01 |
| subunit 2 | | | | | | | | | |
| ADAM metallopeptidase domain 10 | ADAM10 | NM_001110 | CD156c // HsT18717 | 3626555 | chr15 | − | 56651514 | 56829469 | 6.00 |
| | | | // MADM // kuz | | | | | | |
| telomeric repeat binding factor 2 | TERF2 | NM_005652 | TRBF2 // TRF2 | 3696571 | chr16 | − | 67946973 | 68001605 | 6.00 |
| elongation factor, RNA polymerase II, | ELL2 | NM_012081 | — | 2867873 | chr5 | − | 95246561 | 95323733 | 5.99 |
| proteasome maturation protein | POMP | NM_015932 | C13orf12 // HSPC014 | 3483348 | chr13 | + | 28125188 | 28151050 | 5.99 |
| | | | // PNAS-110 // | | | | | | |
| | | | UMP1 | | | | | | |
| mitochondrial ribosomal protein L16 | MRPL16 | NM_017840 | FLJ20484 // L16mt | 3374856 | chr11 | − | 59330154 | 59334921 | 5.99 |
| | | | // PNAS-111 | | | | | | |
| glucosaminyl (N-acetyl) transferase | GCNT3 | NM_004751 | C2/4GnT // C2GnT- | 3596147 | chr15 | + | 57628756 | 57719710 | 5.98 |
| 3, mucin type | | | M // C2GnT2 // | | | | | | |
| KIAA0409 | KIAA0409 | NM_015324 | RRP8 | 3360982 | chr11 | − | 6492639 | 6582206 | 5.98 |
| tankyrase 1 binding protein 1, 182 kDa | TNKS1BP1 | NM_033396 | FLJ45975 // | 3373675 | chr11 | − | 56823694 | 56848992 | 5.98 |
| | | | KIAA1741 // TAB182 | | | | | | |
| polymerase (RNA) I polypeptide E, | POLR1E | NMP_022490 | FLJ13390 // | 3168938 | chr9 | + | 37475945 | 37500379 | 5.97 |
| 53 kDa | | | FLJ13970 // PAF53 | | | | | | |
| | | | // PRAF1 // RP11- | | | | | | |
| | | | 405L18.3 | | | | | | |
| E3 ubiquitin protein ligase, HECT | EDD1 | NM_015902 | DD5 // EDD // | 3147321 | chr8 | − | 103333634 | 103493671 | 5.97 |
| domain containing, 1 | | | FLJ11310 // HYD // | | | | | | |
| | | | KIAA0896 // | | | | | | |
| | | | MGC57263 | | | | | | |
| G protein-coupled receptor, family C, | GPRC5A | NM_003979 | GPCR5A // RAI3 // | 3405587 | chr12 | + | 12929011 | 12986488 | 5.96 |
| group 5, member A | | | RAIG1 | | | | | | |
| fragile X mental retardation 1 | FMR1 | NM_002024 | FMRP // FRAXA // | 3994100 | chrX | + | 146801098 | 146840329 | 5.96 |
| | | | MGC87458 | | | | | | |
| thyroid hormone receptor associated | THRAP2 | NM_015335 | DKFZp781D0112 // | 3473083 | chr12 | − | 114868953 | 115419724 | 5.96 |
| protein 2 | | | FLJ21627 // | | | | | | |
| | | | KIAA1025 // MED13L | | | | | | |
| | | | // PROSIT240 // | | | | | | |
| | | | TRAP240L | | | | | | |
| epithelial membrane protein 1 | EMP1 | NM_001423 | CL-20 // EMP-1 // | 3405748 | chr12 | + | 13230082 | 13264499 | 5.95 |
| | | | TMP | | | | | | |
| SUMO1/sentrin specific peptidase 5 | SENP5 | NM_152699 | FLJ42398 // | 2659631 | chr3 | + | 198079101 | 198143960 | 5.94 |
| | | | MGC27076 | | | | | | |
| FUS interacting protein | FUSIP1 | NM_006625 // | FUSIP2 // NSSR // | 4045780 | chr1_random | − | 1493804 | 1514582 | 5.93 |
| (serine/arginine-rich) 1 | | NM_054016 | SFRS13 // SRp38 // | | | | | | |
| | | | SRrp40 // TASR // | | | | | | |
| | | | TASR1 // TASR2 | | | | | | |
| UTP14, U3 small nucleolar | UTP14A | NM_006649 | KIAA0266 // NY-CO- | 3990566 | chrX | + | 128832975 | 128912087 | 5.93 |
| ribonucleoprotein, homolog A (yeast) | | | 16 // SDCCAG16 // | | | | | | |
| | | | dJ537K23.3 | | | | | | |
| cell division cycle associated 3 | CDCA3 | NM_031299 | GRCC8 // MGC2577 | 3442322 | chr12 | − | 6824231 | 6831482 | 5.93 |
| | | | // TOME-1 | | | | | | |
| zinc finger, AN1-type domain 3 | ZFAND3 | NM_021943 | FLJ13222 // TEX27 | 2905664 | chr6 | + | 37895285 | 38231400 | 5.93 |
| glucocorticoid receptor DNA binding | GRLF1 | NM_004491 | GRF-1 // KIAA1722 | 3837001 | chr19 | + | 52113779 | 52200169 | 5.92 |
| factor 1 | | | // MGC10745 // | | | | | | |
| | | | P190-A // P190A // | | | | | | |
| | | | p190RhoGAP | | | | | | |
| GPI-anchored membrane protein 1 | GPIAP1 | NM_005898 // | GPIP137 // M11S1 // | 3326183 | chr11 | + | 34009711 | 34081946 | 5.92 |
| | | NM_203364 | p137GPI | | | | | | |
| AT hook containing transcription | AHCTF1 | XM_001126456 // | DKFZp434N093 // | 2465324 | chr1 | − | 245069027 | 245162066 | 5.92 |
| factor 1 | | NM_015446 | ELYS // MST108 // | | | | | | |
| | | | MSTP108 // TMBS62 | | | | | | |
| aurora kinase B | AURKB | NM_004217 | AIK2 // AIM-1 // | 3744263 | chr17 | − | 8034636 | 8054649 | 5.92 |
| | | | AIM1 // ARK2 // | | | | | | |
| | | | AurB // IPL1 // | | | | | | |
| | | | STK12 // STK5 | | | | | | |
| ariadne homolog 2 (Drosophila) | ARIH2 | NM_006321 | ARI2 // FLJ10938 // | 2621827 | chr3 | + | 48930742 | 48997971 | 5.91 |
| | | | FLJ33921 // TRIAD1 | | | | | | |
| chromosome 11 open reading frame | C11orf68 | NM_031450 | Bles03 // P5326 | 3378007 | chr11 | − | 65440893 | 65443107 | 5.91 |
| 68 | | | | | | | | | |
| nuclear factor of kappa light | NFKBIA | NM_020529 | IKBA // MAD-3 // | 3561039 | chr14 | − | 34940473 | 34944214 | 5.90 |
| polypeptide gene enhancer in B-cells | | | NFKBI | | | | | | |
| ihibitor, alpha | | | | | | | | | |
| calcyclin binding protein | CACYBP | NM_001007214 // | GIG5 // MGC87971 | 2368198 | chr1 | + | 173235214 | 173247469 | 5.90 |
| | | NM_014412 | // PNAS-107 // | | | | | | |
| | | | RP1-102G20.6 // | | | | | | |
| | | | S100A6BP // SIP | | | | | | |
| zinc finger protein 593 | ZNF593 | NM_015871 | ZT86 | 2326311 | chr1 | + | 26368813 | 26371688 | 5.90 |
| proteasome (prosome, macropain) | PSMD2 | NM_002808 | MGC14274 // P97 // | 2655650 | chr3 | + | 185499199 | 185509504 | 5.89 |
| 26S subunit, non-ATPase, 2 | | | S2 // TRAP2 | | | | | | |
| YY1 transcription factor | YY1 | NM_003403 | DELTA // NF-E1 // | 3551677 | chr14 | + | 99750383 | 99818868 | 5.89 |
| | | | UCRBP // YIN- | | | | | | |
| | | | YANG-1 | | | | | | |
| Smith-Magenis syndrome | SMCR7L | NM_019008 | FLJ20232 // | 3945877 | chr22 | + | 38226061 | 38244079 | 5.88 |
| chromosome region, candidate 7-like | | | HSU79252 // | | | | | | |
| | | | dJ1104E15.3 | | | | | | |
| CDC-like kinase 1 | CLK1 | NM_001024646 // | CLK // CLK/STY // | 2594497 | chr2 | − | 201425997 | 201437659 | 5.88 |
| | | NM_004071 | STY | | | | | | |
| phosphatidylinositol transfer protein, | PITPNB | NM_012399 | PI-TP-beta // | 3956290 | chr22 | − | 26577440 | 26645487 | 5.87 |
| beta | | | PtdInsTP // VIB1B | | | | | | |
| A kinase (PRKA) anchor protein 8- | AKAP8L | NM_014371 | DKFZp434L0650 // | 3853345 | chr19 | − | 15351869 | 15390819 | 5.87 |
| like | | | HAP95 // NAKAP // | | | | | | |
| | | | NAKAP95 | | | | | | |
| chromosome 17 open reading frame | C17orf70 | NM_025161 | FLJ22175 // | 3773980 | chr17 | − | 77117366 | 77130137 | 5.87 |
| 70 | | | FLJ30151 | | | | | | |
| ring finger protein 34 | RNF34 | NM_025126 // | FLJ21786 // RFI // | 3434823 | chr12 | + | 120322070 | 120352805 | 5.87 |
| | | NM_194271 | RIF // RIFF | | | | | | |
| solute carrier family 7 (cationic | SLC7A6 | NM_001076785 // | DKFZp686K15246 // | 3666146 | chr16 | + | 66855932 | 66893223 | 5.86 |
| amino acid transporter, y+ system), | | NM_003983 | KIAA0245 // LAT-2 | | | | | | |
| member 6 | | | // LAT3 // y+LAT-2 | | | | | | |
| solute carrier family 39 (zinc | SLC39A14 | NM_015359 | KIAA0062 // LZT-Hs4 | 3089360 | chr8 | + | 22280765 | 22347587 | 5.86 |
| transporter), member 14 | | | // ZIP14 // cig19 | | | | | | |
| PI-3-kinase-related kinase SMG-1 | SMG1 | NM_015092 | 61E3.4 // ATX // | 3683050 | chr16 | − | 18719381 | 18845318 | 5.86 |
| | | | KIAA0421 // LIP | | | | | | |
| zinc finger protein 259 | ZNF259 | NM_003904 | MGC110983 // ZPR1 | 3392871 | chr11 | − | 116153652 | 116163944 | 5.86 |
| NADH dehydrogenase (ubiquinone) 1 | NDUFB11 | NM_019056 | ESSS // FLJ20494 // | 4007009 | chrX | − | 46886563 | 46902114 | 5.86 |
| beta subcomplex, 11, 17.3 kDa | | | MGC111182 // | | | | | | |
| | | | NP17.3 // Np15 // | | | | | | |
| heterogeneous nuclear | HNRPU | NM_004501 // | HNRNPU // SAF-A | 2464499 | chr1 | − | 243078713 | 243094975 | 5.85 |
| ribonucleoprotein LI (scaffold | | NM_031844 | // U21.1 | | | | | | |
| attachment factor A) | | | | | | | | | |
| BCL2-like 1 | BCL2L1 | NM_001191 // | BCL-XL/S // BCL2L | 3902489 | chr20 | − | 29715925 | 29775453 | 5.85 |
| | | NM_138578 | // BCLX // Bcl-X // | | | | | | |
| | | | DKFZp781P2092 // | | | | | | |
| | | | bcl-xL // bcl-xS | | | | | | |
| stem-loop (histone) binding protein | SLBP | NM_006527 | HBP | 2757319 | chr4 | − | 1664276 | 1684080 | 5.85 |
| retinitis pigmentosa 2 (X-linked | RP2 | NM_006915 | KIAA0215 // TBCCD2 | 3975869 | chrX | + | 46581319 | 46637601 | 5.85 |
| recessive) | | | | | | | | | |
| cullin 4A | CUL4A | NM_001008895 // | — | 3502497 | chr13 | + | 112905835 | 112967356 | 5.85 |
| | | NM_003589 | | | | | | | |
| tripartite motif-containing 28 | TRIM28 | NM_005762 | FLJ29029 // KAP1 // | 3844238 | chr19 | + | 63740597 | 63753931 | 5.84 |
| | | | RNF96 // TF1B // | | | | | | |
| | | | TIF1B | | | | | | |
| DEAD (Asp-Glu-Ala-Asp) box | DDX39 | NM_005804 | BAT1 // BAT1L // | 3852691 | chr19 | − | 14380562 | 14404078 | 5.84 |
| polypeptide 39 | | | DDXL // MGC18203 | | | | | | |
| | | | // MGC8417 // | | | | | | |
| | | | URH49 | | | | | | |
| lectin, galactoside-binding, soluble, 8 | LGALS8 | NM_006499 // | Gal-8 // PCTA-1 // | 2386867 | chr1 | + | 234717161 | 234782890 | 5.84 |
| (galectin 8) | | NM_201543 // | PCTA1 // Po66-CBP | | | | | | |
| | | NM_201544 // | | | | | | | |
| | | NM_201545 | | | | | | | |
| ankyrin repeat domain 10 | ANKRD10 | NM_017664 | DKFZp686B07190 // | 3525679 | chr13 | − | 110322146 | 110365417 | 5.83 |
| | | | FLJ20093 | | | | | | |
| zinc finger and BTB domain | ZBTB9 // | NM_152735 // | MGC23166 // | 2903703 | chr6 | + | 33494813 | 33533290 | 5.82 |
| containing 9 // synaptic Ras GTPase | SYNGAP1 | NM_006772 | DKFZp761G1421 // | | | | | | |
| activating protein 1 homolog (rat) | | | KIAA1938 // RASA1 | | | | | | |
| | | | // RASA5 // | | | | | | |
| transmembrane protein 70 | TMEM70 | NM_001040613 // | FLJ20533 | 3103494 | chr8 | + | 75046321 | 75057572 | 5.82 |
| | | NM_017866 | | | | | | | |
| aftiphilin | AFTPH | NM_001002243 // | FLJ20080 // | 2485433 | chr2 | + | 64604969 | 64714437 | 5.82 |
| | | NM_017657 // | FLJ23793 // | | | | | | |
| | | NM_203437 | MGC33965 // | | | | | | |
| | | | Nbla10388 | | | | | | |
| polymerase (RNA) 1 polypeptide A, | POLR1A | NM_015425 | DKFZP586M0122 // | 2562605 | chr2 | − | 86103197 | 86186509 | 5.81 |
| 194 kDa | | | FLJ21915 // | | | | | | |
| | | | MGC87965 // RPA1 | | | | | | |
| | | | // RPO1-4 | | | | | | |
| general transcription factor IIIC, | GTF3C5 | NM_012087 | FLJ20857 // TFIIIC63 | 3192653 | chr9 | + | 134895917 | 134923708 | 5.81 |
| polypeptide 5, 63 kDa | | | // TFIIICepsilon // | | | | | | |
| | | | TFiiiC2-63 | | | | | | |
| tumor necrosis factor receptor | TNFRSF21 | NM_014452 | BM-018 // DR6 // | 2956052 | chr6 | − | 47307230 | 47385639 | 5.80 |
| superfamily, member 21 | | | MGC31965 | | | | | | |
| sema domain, immunoglobulin domain | SEMA4B | NM_020210 // | KIAA1745 // | 3607927 | chr15 | + | 88529156 | 88573879 | 5.80 |
| (Ig), transmembrane domain (TM) and | | NM_198925 | MGC131831 // | | | | | | |
| short cytoplasmic domain, | | | SEMAC // SemC | | | | | | |
| (semaphorin) 4B | | | | | | | | | |
| mitochondrial ribosomal protein S11 | MRPS11 | NM_022839 // | FLJ22512 // | 3607183 | chr15 | + | 86811688 | 86822612 | 5.80 |
| | | NM_176805 | FLJ23406 // HCC-2 | | | | | | |
| nuclear factor of activated T-cells 5, | NFAT5 | NM_006599 // | KIAA0827 // NF-AT5 | 3666779 | chr16 | + | 68156498 | 68296054 | 5.80 |
| tonicity-responsive | | NM_138713 // | // NFATL1 // NFATZ | | | | | | |
| | | NM_138714 // | // OREBP // | | | | | | |
| | | NM_173214 // | TONEBP | | | | | | |
| | | NM_173215 | | | | | | | |
| nuclear receptor subfamily 2, group | NR2F2 | NM_021005 | ARP1 // COUP-TFII | 3610110 | chr15 | + | 94448164 | 94683620 | 5.79 |
| F, member 2 | | | // COUPTFB // | | | | | | |
| | | | MGC117452 // | | | | | | |
| | | | SVP40 // TFCOUP2 | | | | | | |
| SH3 domain containing ring finger 1 | SH3RF1 | NM_020870 | FLJ21602 // | 2793137 | chr4 | − | 170220651 | 170428725 | 5.78 |
| | | | KIAA1494 // POSH | | | | | | |
| | | | // RNF142 // | | | | | | |
| splicing factor 3b, subunit 3, 130 kDa | SF3B3 | NM_012426 | KIAA0017 // RSE1 // | 3667281 | chr16 | + | 69115212 | 69169072 | 5.78 |
| | | | SAP130 // SF3b130 | | | | | | |
| | | | // STAF130 | | | | | | |
| PTC7 protein phosphatase homolog | PPTC7 | NM_139283 | DKFZp686M07120 // | 3471300 | chr12 | − | 109455446 | 109514152 | 5.77 |
| (S. cerevisiae) | | | MGC133072 // TA- | | | | | | |
| | | | PP2C | | | | | | |
| homeodomain interacting protein | HIPK1 | NM_152696 // | KIAA0630 // | 2352758 | chr1 | + | 114273467 | 114322014 | 5.77 |
| kinase 1 | | NM_181358 // | MGC26642 // | | | | | | |
| | | NM_198268 // | MGC33446 // | | | | | | |
| | | NM_198269 | MGC33548 // Myak | | | | | | |
| | | | // Nbak2 | | | | | | |
| nuclear receptor binding protein 1 | NRBP1 | NM_013392 | BCON3 // FLU27109 | 2474527 | chr2 | + | 27504171 | 27518620 | 5.77 |
| | | | // FLJ35541 // | | | | | | |
| | | | MADM // MUDPNP | | | | | | |
| | | | // NRBP | | | | | | |
| cleavage stimulation factor, 3′ pre- | CSTF1 | NM_001033521 // | CstF-50 // CstFp50 | 3890154 | chr20 | + | 54393411 | 54413257 | 5.77 |
| RNA, subunit 1, 50 kDa | | NM_001033522 // | | | | | | | |
| | | NM_001324 | | | | | | | |
| pre-B-cell colony enhancing factor 1 | PBEF1 // | NM_005746 // — | 1110035O14Rik // | 3066818 | chr7 | − | 105625846 | 105713266 | 5.77 |
| // pre-B cell enhancing factor 1 | RP11- | | DKFZP666B131 // | | | | | | |
| pseudogene | 92J19.4 | | MGC117256 // | | | | | | |
| | | | NAMPT // PBEF // | | | | | | |
| | | | LOC646309 | | | | | | |
| transmembrane protein 11 | TMEM11 | NM_003876 | C17orf35 // PM1 // | 3749767 | chr17 | − | 21041205 | 21058509 | 5.77 |
| | | | PMI | | | | | | |
| transmembrane protein 115 | TMEM115 | NM_007024 | PL6 | 2675304 | chr3 | − | 50367198 | 50372046 | 5.76 |
| catenin (cadherin-associated | CTNNAL1 | NM_003798 | CLLP // alpha-CATU | 3219621 | chr9 | − | 110744011 | 110815625 | 5.76 |
| protein), alpha-like 1 | | | | | | | | | |
| clathrin, heavy chain (Hc) | CLTC | NM_004859 | CHC17 // CLH-17 // | 3729172 | chr17 | + | 55052021 | 55127299 | 5.76 |
| | | | CLTCL2 // Hc // | | | | | | |
| | | | KIAA0034 | | | | | | |
| cyclin H | CCNH | NM_001239 | CAK // p34 // p37 | 2865860 | chr5 | − | 86707385 | 86782317 | 5.74 |
| SECIS binding protein 2 | SECISBP2 | NM_024077 | DKFZp686C09169 // | 3178611 | chr9 | + | 91122988 | 91146214 | 5.74 |
| | | | SBP2 | | | | | | |
| WD repeat domain 26 | WDR26 | NM_025160 | FLJ21016 // MIP2 | 2458082 | chr1 | − | 222639474 | 222691338 | 5.74 |
| stomatin (EPB72)-like 2 // Fanconianemia, | STOML2 // | NM_013442 // | HSPC108 // SLP-2 | 3204534 | chr9 | − | 35089540 | 35093157 | 5.74 |
| complementation group G | FANCG | NM_004629 | // FAG // XRCC9 | | | | | | |
| nucleolar and coiled-body | NOLC1 | NM_004741 | KIAA0035 // | 3261492 | chr10 | + | 103901962 | 103913597 | 5.73 |
| phosphoprotein 1 | | | NOPP130 // | | | | | | |
| | | | NOPP140 // | | | | | | |
| | | | NS5ATP13 // P130 | | | | | | |
| ADP-ribosylhydrolase like 2 | ADPRHL2 | NM_017825 | ARH3 // FLJ20446 // | 2330253 | chr1 | + | 36327093 | 36332113 | 5.73 |
| | | | dJ665N4.2 | | | | | | |
| chromosome 1 open reading frame | C1orf164 | NM_018150 | FLJ10597 | 2333907 | chr1 | + | 44643293 | 44889968 | 5.72 |
| 164 | | | | | | | | | |
| platelet-activating factor | PAFAH1B2 | NM_002572 | — | 3350749 | chr11 | + | 116520229 | 116552810 | 5.72 |
| acetylhydrolase, isoform Ib, beta | | | | | | | | | |
| subunit 30 kDa | | | | | | | | | |
| DPH2 homolog (S. cerevisiae) // | DPH2 // | NM_001039589 // | DPH2L2 // B4Gal-T2 | 2333635 | chr1 | + | 44208244 | 44211909 | 5.72 |
| UDP-Gal:betaGlcNAc beta 1,4- | B4GALT2 // | NM_001384 // | // B4Gal-T3 // | | | | | | |
| galactosyltransferase, polypeptide 2 | ATP6V0B | NM_001005417 // | beta4Gal-T2 // | | | | | | |
| // ATPase, H+ transporting, | | NM_003780 // | ATP6F // HATPL // | | | | | | |
| lysosomal 21 kDa, V0 subunit b | | NM_001039457 // | VMA16 | | | | | | |
| | | NM_004047 | | | | | | | |
| chromosome 4 open reading frame | C4orf14 | NM_032313 | MGC3232 // | 2770427 | chr4 | − | 57496489 | 57539746 | 5.72 |
| 14 | | | hAtNOS1 // | | | | | | |
| ST3 beta-galactoside alpha-2,3- | ST3GAL4 | NM_006278 | CGS23 // FLJ11867 | 3355145 | chr11 | + | 125730175 | 126063667 | 5.71 |
| sialyltransferase 4 | | | // NANTA3 // SAT3 | | | | | | |
| | | | // SIAT4 // SIAT4C | | | | | | |
| | | | // ST3Gal IV // | | | | | | |
| | | | ST3GalIV // STZ | | | | | | |
| myeloid differentiation primary | MYD88 | NM_002468 | — | 2617563 | chr3 | + | 38155029 | 38159513 | 5.71 |
| response gene (88) | | | | | | | | | |
| interferon-related developmental | IFRD2 | NM_006764 | FLJ40446 // IFNRP | 2675088 | chr3 | − | 50300134 | 50305282 | 5.71 |
| regulator 2 | | | // SKMc15 // SM15 | | | | | | |
| TNF receptor-associated factor 4 | TRAF4 | NM_145751 // | CART1 // MLN62 // | 3715839 | chr17 | + | 24095168 | 24102103 | 5.71 |
| | | NM_004295 | RNF83 | | | | | | |
| Kruppel-like factor 5 (intestinal) | KLF5 | NM_001730 | BTEB2 // CKLF // | 3493543 | chr13 | + | 72527115 | 72549671 | 5.70 |
| | | | IKLF | | | | | | |
| melanophilin | MLPH | NM_001042467 // | MGC2771 // | 2534252 | chr2 | + | 238025804 | 238135783 | 5.70 |
| | | NM_024101 | MGC59733 // | | | | | | |
| | | | SLAC2-A | | | | | | |
| pleckstrin homology, Sec7 and | PSCD2 | NM_004228 // | ARNO // CTS18 // | 3837836 | chr19 | + | 53664134 | 53690474 | 5.69 |
| coiled-coil domains 2 (cytohesin-2) | | NM_017457 | CTS18.1 // PSCD2L | | | | | | |
| | | | // SEC7L // Sec7p-L | | | | | | |
| | | | // Sec7p-like | | | | | | |
| 5′-3′ exoribonuclease 2 | XRN2 | NM_012255 | — | 3879467 | chr20 | + | 21219705 | 21322172 | 5.69 |
| large subunit GTPase 1 homolog (S. cerevisiae) | LSG1 | NM_018385 | FLJ11301 | 2711818 | chr3 | − | 195842815 | 195874226 | 5.69 |
| ubiquitin protein ligase E3C | UBE3C | NM_014671 | KIAA0010 // KIAA10 | 3033924 | chr7 | + | 156624381 | 156754823 | 5.68 |
| Rho GTPase-activating protein | RICS | NM_014715 | GC-GAP // GRIT // | 3397877 | chr11 | − | 128343040 | 128567297 | 5.68 |
| | | | KIAA0712 // | | | | | | |
| | | | MGC1892 // | | | | | | |
| | | | p200RhoGAP // | | | | | | |
| | | | p250GAP | | | | | | |
| v-raf murine sarcoma viral oncogene | BRAF | NM_004333 | B-raf I // BRAF1 // | 3076340 | chr7 | − | 139994192 | 140274640 | 5.68 |
| homolog B1 | | | MGC126806 // | | | | | | |
| | | | MGC138284 // | | | | | | |
| heterogeneous nuclear | HNRPH3 | NM_012207 // | 2H9 | 3249738 | chr10 | + | 69760947 | 69772949 | 5.68 |
| ribonucleoprotein H3 (2H9) | | NM_021644 | | | | | | | |
| NudC domain containing 1 | NUDCD1 | NM_032869 | CML66 // FLJ14991 | 3148796 | chr8 | − | 110322324 | 110415934 | 5.68 |
| GATA binding protein 6 | GATA6 | NM_005257 | — | 3781245 | chr18 | + | 18003412 | 18060817 | 5.67 |
| preimplantation protein 3 // heat | PREI3 // | NM_015387 // | 2C4D // CGI-95 // | 2521500 | chr2 | + | 198088565 | 198125849 | 5.67 |
| shock 10 kDa protein 1 (chaperonin | HSPE1 | NM_199482 // | MGC12264 // MOB1 | | | | | | |
| 10) | | NM_002157 | // MOB3 // CPN10 | | | | | | |
| | | | // GROES // HSP10 | | | | | | |
| chromosome 14 open reading frame | C14orf151 | NM_032714 | MGC13251 | 3554282 | chr14 | + | 104227008 | 104244902 | 5.67 |
| 151 | | | | | | | | | |
| ADAM metallopeptidase domain 9 | ADAM9 | NM_001005845 // | KIAA0021 // MCMP | 3095002 | chr8 | + | 38973642 | 39082294 | 5.67 |
| meltrin gamma) | | NM_003816 | // MDC9 // Mltng | | | | | | |
| target of EGR1, member 1 (nuclear) | TOE1 | NM_025077 | FLJ13949 | 2334319 | chr1 | + | 45577929 | 45582381 | 5.67 |
| acidic (leucine-rich) nuclear | ANP32B | NM_006401 | APRIL // PHAPI2 // | 3181417 | chr9 | + | 99784907 | 99823167 | 5.66 |
| phosphoprotein 32 family, member B | | | SSP29 | | | | | | |
| cleavage and polyadenylation specific | CPSF4 | NM_001081559 // | CPSF30 // NAR // | 3014764 | chr7 | + | 98874515 | 98905314 | 5.66 |
| factor 4, 30 kDa | | NM_006693 | NEB1 | | | | | | |
| IK cytokine, down-regulator of HLA II | IK | NM_006083 | OSA2 // IK protein // | 2831932 | chr5 | + | 140006890 | 140022229 | 5.66 |
| | | | MGC59741 // RED | | | | | | |
| phosphoribosyl pyrophosphate | PPAT | NM_002703 | ATASE // GPAT | 2770242 | chr4 | − | 56954288 | 57029053 | 5.65 |
| amidotransferase | | | | | | | | | |
| proteasome (prosome, macropain) | PSMC2 | NM_002803 | MGC3004 // MSS1 // | 3017206 | chr7 | + | 102771969 | 102797076 | 5.65 |
| 26S subunit, ATPase, 2 | | | Nbla10058 // S7 | | | | | | |
| N-acetyltransferase 5 | NAT5 | NM_016100 // | NAT3 // dJ1002M8.1 | 3878934 | chr20 | + | 19935711 | 19962808 | 5.65 |
| | | NM_181527 // | | | | | | | |
| | | NM_181528 | | | | | | | |
| USP6 N-terminal like | USP6NL | XM_374768 // | KIAA0019 // RNTRE | 3277468 | chr10 | − | 11523200 | 11725919 | 5.65 |
| | | XM_927409 // | // TRE2NL | | | | | | |
| | | XM_938665 // | | | | | | | |
| | | XM_943800 // | | | | | | | |
| | | NM_001080491 | | | | | | | |
| EMG1 nucleolar protein hemolog (S. cerevisiae) | EMG1 | NM_006331 | C2F // Grcc2f // | 3403140 | chr12 | + | 6949871 | 6966191 | 5.65 |
| | | | NEP1 | | | | | | |
| glutaminase | GLS | NM_014905 | DKFZp686O15119 // | 2520291 | chr2 | + | 191453802 | 191538513 | 5.65 |
| | | | FLJ10358 // GLS1 // | | | | | | |
| | | | KIAA0838 | | | | | | |
| pleiomorphic adenoma gene-like 2 | PLAGL2 | NM_002657 | FLJ23283 | 3902682 | chr20 | − | 30243975 | 30259284 | 5.63 |
| TM2 domain containing 3 | TM2D3 | NM_025141 // | BLP2 | 3642358 | chr15 | − | 99977972 | 100010609 | 5.62 |
| | | NM_078474 | | | | | | | |
| SEC24 related gene family, member | SEC24C | NM_004922 // | KIAA0079 | 3251848 | chr10 | + | 75174138 | 75210008 | 5.62 |
| C (S. cerevisiae) | | NM_198597 | | | | | | | |
| ubiquitin-conjugating enzyme E2D 3 | UBE2D3 | NM_003340 // | E2(17)KB3 // | 2779992 | chr4 | − | 103934623 | 104009473 | 5.61 |
| (UBC4/5 homolog, yeast) | | NM_181886 // | MGC43926 // | | | | | | |
| | | NM_181887 // | MGC5416 // UBC4/5 | | | | | | |
| | | NM_181888 // | // UBCH5C | | | | | | |
| | | NM_181889 // | | | | | | | |
| | | NM_181890 // | | | | | | | |
| | | NM_181891 // | | | | | | | |
| | | NM_181892 // | | | | | | | |
| | | NM_181893 | | | | | | | |
| Rho guanine nucleotide exchange | ARHGEF7 | NM_003899 // | BETA-PIX // COOL1 | 3501661 | chr13 | + | 110565635 | 110756075 | 5.61 |
| factor (GEF) 7 | | NM_145735 | // DKFZp761K1021 | | | | | | |
| | | | // KIAA0142 // | | | | | | |
| | | | KIAA0142 // | | | | | | |
| | | | Nbla10314 // P50 // | | | | | | |
| | | | P50BP // P85 // | | | | | | |
| | | | P85COOL1 // | | | | | | |
| | | | P85SPR // PAK3 // | | | | | | |
| ring-finger protein 139 | RNF139 | NM_007218 | HRCA1 // MGC31961 | 3114618 | chr8 | + | 125556189 | 125570389 | 5.59 |
| | | | // RCA1 // TRC8 | | | | | | |
| valosin-containing protein // Fanconi | VCP // | NM_007126 | IBMPFD // | 3204404 | chr9 | − | 35026106 | 35062648 | 5.58 |
| anemia, complementation group G | FANCG | NM_004629 | MGC131997 // | | | | | | |
| | | | MGC148092 // | | | | | | |
| | | | MGC8560 // TERA // | | | | | | |
| | | | p97 // FAG // | | | | | | |
| ADP-ribosylation factor 1 | ARF1 | NM_001024226 // | — | 2383726 | chr1 | + | 226313262 | 226353506 | 5.58 |
| | | NM_001024227 // | | | | | | | |
| | | NM_001024228 // | | | | | | | |
| | | NM_001658 | | | | | | | |
| bladder cancer associated protein | BLCAP | NM_006698 | BC10 | 3904928 | chr20 | − | 35554050 | 35589711 | 5.57 |
| heterogeneous nuclear | HNRPH1 | NM_005520 | DKFZp686A15170 // | 2890148 | chr5 | − | 178970155 | 178993890 | 5.57 |
| ribonucleoprotein H1 (H) | | | HNRPH // hnRNPH | | | | | | |
| programmed cell death 2-like | PDCD2L | NM_032346 | MGC13096 | 3829751 | chr19 | + | 39587143 | 39608910 | 5.57 |
| TNF receptor-associated factor 2 | TRAF2 | NM_021138 | MGC: 45012 // TRAP | 3194896 | chr9 | + | 138896205 | 138943484 | 5.56 |
| | | | // TRAP3 | | | | | | |
| ring finger protein 43 | RNF43 | NM_017763 | DKFZp781H02126 // | 3764399 | chr17 | − | 53785272 | 53849935 | 5.56 |
| | | | DKFZp781H0392 // | | | | | | |
| | | | FLJ20315 // | | | | | | |
| | | | MGC125630 // | | | | | | |
| | | | RNF124 // URCC | | | | | | |
| tousled-like kinase 1 | TLK1 | NM_012290 | KIAA0137 // PKU- | 2586603 | chr2 | − | 171538552 | 171796060 | 5.55 |
| | | | BETA | | | | | | |
| suppressor of zeste 12 homolog | SUZ12 | NM_015355 | CHET9 // JJAZ1 // | 3717395 | chr17 | + | 27258855 | 27352177 | 5.55 |
| (Drosophila) | | | KIAA0160 | | | | | | |
| dynactin 4 (p62) | DCTN4 | NM_016221 | — | 2881554 | chr5 | − | 150065753 | 150135890 | 5.54 |
| leucine rich repeat containing 1 // | LRRC1 // | NM_018214 // | FLJ10775 // | 2910680 | chr6 | + | 53695560 | 53897416 | 5.54 |
| chromosome 20 open reading frame | C20orf44 | NM_018244 // | FLJ11834 // LANO | | | | | | |
| 44 | | NM_199487 // | // dJ523E19.1 // | | | | | | |
| | | NM_199513 | BFZB // CBP3 // | | | | | | |
| | | | MGC104353 // | | | | | | |
| | | | MGC141902 | | | | | | |
| GTPase activating protein (SH3 | G3BP2 // | NM_012297 // | —// MGC119326 // | 2773756 | chr4 | − | 76786807 | 76869171 | 5.54 |
| domain) binding protein 2 // NMDA | NARG2 // | NM_203504 // | MGC119329 // | | | | | | |
| receptor regulated 2 // RAR-related | RORA | NM_203505 // | NR1F1 // ROR1 // | | | | | | |
| orphan receptor A | | NM_001018089 // | ROR2 // ROR3 // | | | | | | |
| | | NM_024611 // | RZRA | | | | | | |
| | | NM_002943 // | | | | | | | |
| | | NM_134260 // | | | | | | | |
| | | NM_134261 // | | | | | | | |
| | | NM_134262 | | | | | | | |
| ubiquitin specific peptidase 3 | USP3 | NM_006537 | MGC129878 // | 3597603 | chr15 | + | 61554385 | 61673885 | 5.54 |
| | | | MGC129879 // | | | | | | |
| | | | SIH003 // UBP | | | | | | |
| peptidylprolyl isomerase | PPIL4 // | NM_139126 // | HDCME13P // APC1 | 2978876 | chr6 | − | 149811664 | 149908884 | 5.54 |
| (cyclophilin)-like 4 // solute carrier | SLC25A24 | NM_013386 // | // DKFZp586G0123 | | | | | | |
| family 25 (mitochondrial carrier; | | NM_213651 | // SCAMC-1 | | | | | | |
| phosphate carrier), member 24 | | | | | | | | | |
| structural maintenance of | SMC1A | NM_006306 | DKFZp686L19178 // | 4009238 | chrX | − | 53417797 | 53466400 | 5.53 |
| chromosomes 1A | | | DXS423E // | | | | | | |
| | | | KIAA0178 // | | | | | | |
| | | | MGC138332 // SB1.8 | | | | | | |
| | | | // SMC1 // SMC1L1 | | | | | | |
| | | | // SMC1alpha // | | | | | | |
| NM23-LV // non-metastatic cells 1, | NME1-NME2 | NM_001018136 // | NME2 // AWD // | 3726934 | chr17 | + | 46564693 | 46594905 | 5.52 |
| protein (NM23A) expressed in | // NME1 | NM_000269 // | GAAD // NDPKA // | | | | | | |
| | | NM_198175 | NM23 // NM23-H1 | | | | | | |
| taxilin alpha // doublecortin domain | TXLNA // | NM_175852 // | DKFZp451J0118 // | 2328713 | chr1 | + | 32417884 | 32436467 | 5.52 |
| containing 2B | DCDC2B | XM_926306 // | IL14 // MGC118870 | | | | | | |
| | | XM_940631 | // MGC118871 // | | | | | | |
| | | | RP4-622L5.4 // TXLN | | | | | | |
| | | | // — | | | | | | |
| cysteine-rich, angiogenic inducer, 61 | CYR61 | NM_001554 | CCN1 // GIG1 // | 2344888 | chr1 | + | 85816676 | 85867516 | 5.52 |
| | | | IGFBP10 | | | | | | |
| prothymosin, alpha (gene sequence | PTMA // | NM_002823 // | MGC104802 // TMSA | 2532021 | chr2 | + | 232280604 | 232286493 | 5.52 |
| 28) // family with sequence similarity | FAM22G | XM_001127718 // | // — | | | | | | |
| 22, member G | | NM_001045477 | | | | | | | |
| casein kinase 1, epsilon // KIAA1660 | CSNK1E // | NM_001894 // | HCKIE // MGC10398 | 3960478 | chr22 | − | 37010115 | 37150916 | 5.51 |
| protein | KIAA1660 | NM_152221 // | // — | | | | | | |
| | | XM_929784 // | | | | | | | |
| | | XM_940170 | | | | | | | |
| MCM3 minichromosome maintenance | MCM3 | NM_002388 | HCC5 // MGC1157 // | 2957126 | chr6 | − | 52236739 | 52356643 | 5.50 |
| deficient 3 (S. cerevisiae) | | | P1-MCM3 // P1.h // | | | | | | |
| | | | RLFB | | | | | | |
| solute carrier family 25 | SLC25A3 | NM_213612 // | OK/SW-cl.48 // PHC | 3427820 | chr12 | + | 97511471 | 97519906 | 5.50 |
| (mitochondrial carrier; phosphate | | NM_002635 // | | | | | | | |
| carrier), member 3 | | NM_005888 // | | | | | | | |
| | | NM_213611 | | | | | | | |
| potassium channel tetramerisation | KCTD5 | NM_018992 | FLJ20040 | 3645204 | chr16 | + | 2640820 | 2700477 | 5.50 |
| domain containing 5 | | | | | | | | | |
| STE20-like kinase (yeast) | SLK | NM_014720 | KIAA0204 // | 3262433 | chr10 | + | 105716666 | 105778975 | 5.49 |
| | | | MGC133067 // STK2 | | | | | | |
| | | | // bA16H23.1 // | | | | | | |
| | | | se20-9 | | | | | | |
| glutathione reductase | GSR | NM_000637 | MGC78522 | 3130161 | chr8 | − | 30655000 | 30705038 | 5.49 |
| par-3 partitioning defective 3 | PARD3 | NM_019619 | ASIP // Baz // | 3284596 | chr10 | − | 34413632 | 35144249 | 5.49 |
| homolog (C. elegans) | | | Bazooka // FLJ21015 | | | | | | |
| | | | // PAR3 // | | | | | | |
| | | | PAR3alpha // | | | | | | |
| | | | PARD3A // SE2-5L16 | | | | | | |
| | | | // SE2-5LT1 // SE2- | | | | | | |
| UTP18, small subunit (SSU) | UTP18 | NM_016001 | CGI-48 // WDR50 | 3726992 | chr17 | + | 46692081 | 46730289 | 5.49 |
| processome component, homolog | | | | | | | | | |
| (yeast) | | | | | | | | | |
| serine/arginine repetitive matrix 1 // | SRRM1 // | NM_005839 // | 160-KD // MGC39488 | 2325526 | chr1 | + | 24830814 | 24929968 | 5.49 |
| chromosome 1 open reading frame | C1orf130 | NM_001010980 | // POP101 // | | | | | | |
| 130 | | | SRM160 // FLJ42528 | | | | | | |
| conserved nuclear protein NHN1 | NHN1 | NM_144604 | FLJ22664 // | 3673515 | chr16 | + | 87152329 | 87225870 | 5.49 |
| | | | FLJ34530 // | | | | | | |
| | | | FLJ36075 | | | | | | |
| UBX domain containing 8 | UBXD8 | NM_014613 | ETEA // KIAA0887 | 2842570 | chr5 | + | 175807217 | 175870117 | 5.48 |
| elongation protein 4 homolog (S. cerevisiae) | ELP4 | NM_019040 | C11orf19 // | 3325307 | chr11 | + | 31487883 | 31764940 | 5.48 |
| | | | FLJ20498 // | | | | | | |
| | | | PAX6NEB // PAXNEB | | | | | | |
| | | | // dJ68P15A.1 | | | | | | |
| hypothetical protein FLJ20366 | FLJ20366 | NM_017786 | GOLSYN | 3148871 | chr8 | − | 110655383 | 110964912 | 5.48 |
| SYF2 homolog, RNA splicing factor | SYF2 // | NM_015484 // | CBPIN // | 2402068 | chr1 | − | 25324131 | 25431599 | 5.47 |
| (S. cerevisiae) // MAX interactor 1 | MXI1 // | NM_207170 // | DKFZp564O2082 // | | | | | | |
| // chromosome 1 open reading | C1orf63 | NM_001008541 // | NTC31 // P29 // | | | | | | |
| frame 63 | | NM_005962 // | MAD2 // MGC43220 | | | | | | |
| | | NM_130439 // | // MXD2 // MXI // | | | | | | |
| | | NM_020317 | DJ465N24.2.1 // | | | | | | |
| | | | NPD014 // RP3- | | | | | | |
| | | | 465N24.4 | | | | | | |
| exostoses (multiple) 1 | EXT1 | NM_000127 | EXT // ttv | 3150060 | chr8 | − | 118880792 | 119193382 | 5.46 |
| polymerase (RNA) II (DNA directed) | POLR2D // | NM_004805 // | HSRBP4 // HSRPB4 | 2575134 | chr2 | − | 128317597 | 128334003 | 5.45 |
| polypeptide D // WD repeat domain | WDR33 | NM_001006622 // | // RBP4 // FLJ11294 | | | | | | |
| 33 | | NM_001006623 // | // WDC146 | | | | | | |
| | | NM_018383 | | | | | | | |
| ankyrin repeat domain 13 family, | ANKRD13D | XM_001129739 // | MGC50828 | 3336857 | chr11 | + | 66812660 | 66826524 | 5.45 |
| member D | | NM_207354 | | | | | | | |
| serum amyloid A-like 1 | SAAL1 | NM_138421 | FLJ41463 | 3365249 | chr11 | − | 18048076 | 18084203 | 5.45 |
| C1q domain containing 1 | C1QDC1 | NM_001002259 // | EEG-1 // EEG1 // | 3449368 | chr12 | − | 30753755 | 30799712 | 5.44 |
| | | NM_023925 // | FLJ11391 // | | | | | | |
| | | NM_032156 | FLJ22569 // | | | | | | |
| | | | MGC102894 // | | | | | | |
| | | | MGC134847 // | | | | | | |
| | | | MGC134848 | | | | | | |
| chromosome 11 open reading frame | C11orf30 | NM_020193 | EMSY // FLJ90741 | 3340913 | chr11 | + | 75803897 | 75941697 | 5.44 |
| 30 | | | // GL002 | | | | | | |
| ubiquilin 1 // death inducer- | UBQLN1 // | NM_013438 // | DA41 // DSK2 // | 3212143 | chr9 | − | 85464717 | 85512928 | 5.44 |
| obliterator 1 | DIDO1 | NM_053067 // | FLJ90054 // PLIC-1 | | | | | | |
| | | NM_022105 // | // XDRP1 // BYE1 | | | | | | |
| | | NM_033081 // | // C20orf158 // | | | | | | |
| | | NM_080796 // | DATF1 // DIDO2 // | | | | | | |
| | | NM_080797 | DIDO3// DIO-1 // | | | | | | |
| | | | DIO1 // | | | | | | |
| | | | DKFZp434P1115 // | | | | | | |
| | | | FLJ11265 // | | | | | | |
| | | | KIAA0333 // | | | | | | |
| | | | MGC16140 // | | | | | | |
| similar to ribosomal protein P0 // | RPLP0-like | XR_015741 // | BLOCK 23 // LI0E // | 3474344 | chr12 | − | 119118908 | 119123401 | 5.44 |
| ribosomal protein, large, P0 | // RPLP0 | XR_017813 // | MGC111226 // | | | | | | |
| | | NM_001002 // | MGC88175 // P0 // | | | | | | |
| | | NM_053275 | PRLP0 // RPP0 | | | | | | |
| zinc finger, NFX1-type containing 1 | ZNFX1 // | NM_021035 // | FLJ39275 // | 3908831 | chr20 | − | 47233911 | 47420063 | 5.44 |
| // gasdermin-like | GSDML | NM_00104247 // | MGC131926 // | | | | | | |
| | | NM_018530 | PP4052 // PRO2521 | | | | | | |
| exportin 4 | XPO4 | NM_022459 | FLJ13046 // | 3504434 | chr13 | − | 20251014 | 20375187 | 5.43 |
| | | | KIAA1721 | | | | | | |
| centrosomal protein 72 kDa | CEP72 | NM_018140 | FLJ10565 // | 2798777 | chr5 | + | 608522 | 720187 | 5.43 |
| | | | KIAA1519 // | | | | | | |
| | | | MGC5307 | | | | | | |
| basic transcription factor 3 | BTF3 | NM_001037637 // | BETA-NAC // BTF3a | 2815331 | chr5 | + | 72704665 | 72850839 | 5.43 |
| | | NM_001207 | // BTF3b // NACB | | | | | | |
| c-Maf-inducing protein | CMIP | NM_030629 // | KIAA1694 | 3670772 | chr16 | + | 80030441 | 80323852 | 5.42 |
| | | NM_198390 | | | | | | | |
| cerebellar degeneration-related | CDR2 | NM_001802 | CDR62 // Yo | 3684782 | chr16 | − | 22264766 | 22356527 | 5.42 |
| protein 2, 62 kDa | | | | | | | | | |
| ubiquitin-like 3 | UBL3 | NM_007106 | DKFZP434K151 // | 3507798 | chr13 | − | 29200782 | 29371040 | 5.42 |
| | | | FLJ32018 // HCG-1 | | | | | | |
| | | | // PNSC1 | | | | | | |
| ATP-binding cassette, sub-family F | ABCF1 | NM_001025091 // | ABC27 // ABC50 | 2901687 | chr6 | + | 30647103 | 30667268 | 5.42 |
| (GCN20), member 1 | | NM_001090 | | | | | | | |
| RAB22A, member RAS oncogene | RAB22A | NM_020673 | MGC16770 | 3890870 | chr20 | + | 56318177 | 56375962 | 5.42 |
| family | | | | | | | | | |
| axin 1 | AXIN1 | NM_003502 // | AXIN // MGC52315 | 3675047 | chr16 | − | 277442 | 351633 | 5.41 |
| | | NM_181050 | | | | | | | |
| mitochondrial ribosomal protein L44 | MRPL44 | NM_022915 | FLJ12701 // | 2529782 | chr2 | + | 224530378 | 224540666 | 5.41 |
| | | | FLJ13990 | | | | | | |
| zinc finger protein 146 // zinc finger | ZNF146 // | NM_007145 // | MGC125660 // | 3831260 | chr19 | + | 41397873 | 41421503 | 5.40 |
| protein 268 | ZNF268 | NM_152943 // | MGC125661 // OZF | | | | | | |
| | | NM_003415 | // HZF3 // | | | | | | |
| | | | MGC126498 | | | | | | |
| golgi SNAP receptor complex | GOSR1 | NM_001007024 // | GOS28 // | 3716481 | chr17 | + | 25828452 | 25893594 | 5.40 |
| member 1 | | NM_001007025 // | GOS28/P28 // GS28 | | | | | | |
| | | NM_004871 | // P28 | | | | | | |
| zinc finger protein 512 | ZNF512 | NM_032434 | KIAA1805 // | 2474651 | chr2 | + | 27659393 | 27699586 | 5.40 |
| | | | MGC111046 | | | | | | |
| formin binding protein 4 | FNBP4 | NM_015308 | DKFZp779I1064 // | 3372459 | chr11 | − | 47694235 | 47745657 | 5.39 |
| | | | FBP30 // FLJ41904 | | | | | | |
| | | | KIAA1014 | | | | | | |
| zinc finger protein 707 | ZNF707 | NM_173831 | — | 3119765 | chr8 | + | 144771273 | 144849532 | 5.39 |
| FERM domain containing 5 | FRMD5 | NM_032892 | FLJ41022 // | 3621728 | chr15 | − | 41950261 | 42274751 | 5.39 |
| | | | MGC14161 | | | | | | |
| serum/glucocorticoid regulated | SGK | NM_005627 | SGK1 | 2975014 | chr6 | − | 134532088 | 134680889 | 5.38 |
| hypoxia-inducible factor 1, alpha | HIF1A | NM_001530 // | HIF-1 alpha // HIF1- | 3539070 | chr14 | + | 61225495 | 61285222 | 5.38 |
| subunit (basic helix-loop-helix | | NM_181054 | ALPHA // MOP1 // | | | | | | |
| transcription factor) | | | PASD8 | | | | | | |
| deoxyhypusine synthase | DHPS | NM_001930 // | MIG13 | 3851603 | chr19 | − | 12647534 | 12653680 | 5.37 |
| | | NM_013406 // | | | | | | | |
| | | NM_013407 | | | | | | | |
| thioredoxin-like 1 | TXNL1 | NM_004786 | TRP32 // TXL-1 // | 3809324 | chr18 | − | 52334063 | 52469773 | 5.37 |
| | | | TXNL // Txl | | | | | | |
| RAN, member RAS oncogene family | RAN | NM_006325 | ARA24 // Gsp1 // | 3438027 | chr12 | + | 129922411 | 129928164 | 5.36 |
| | | | TC4 | | | | | | |
| dynein, cytoplasmic 1, light | DYNC1LI2 | NM_006141 | DNCLI2 // LIC2 | 3695199 | chr16 | − | 65312304 | 65343212 | 5.36 |
| intermediate chain 2 | | | | | | | | | |
| enchancer of rudimentary homolog | ERH | XM_001130537 // | DROER // FLJ27340 | 3570049 | chr14 | − | 68916605 | 68935306 | 5.35 |
| (Drosophila) | | NM_004450 | | | | | | | |
| transmembrane 4 L six family | TM4SF1 | NM_014220 | H-L6 // L6 // M3S1 | 2700365 | chr3 | − | 150567718 | 150578270 | 5.34 |
| member 1 | | | // TAAL6 | | | | | | |
| lipase, endothelial | LIPG | NM_006033 | EDL // EL // | 3787855 | chr18 | + | 45341077 | 45481323 | 5.34 |
| exportin 6 | XPO6 | NM_015171 | EXP6 // FLJ22519 // | 3686339 | chr16 | − | 28016804 | 28130734 | 5.34 |
| | | | KIAA0370 // | | | | | | |
| | | | RANBP20 | | | | | | |
| hypothetical protein FLJ11171 | FLJ11171 | NM_018348 | — | 3697563 | chr16 | − | 69872813 | 69881003 | 5.34 |
| UPF1 regulator of nonsense | UPF1 | NM_002911 | FLJ43809 // | 3825383 | chr19 | + | 18803757 | 18840030 | 5.34 |
| transcripts homolog (yeast) | | | FLJ46894 // HUPF1 | | | | | | |
| | | | // KIAA0221 // | | | | | | |
| | | | NORF1 // RENT1 // | | | | | | |
| | | | pNORF1 | | | | | | |
| surfeit 6 | SURF6 | NM_006753 | FLJ30322 | 3228621 | chr9 | − | 135186667 | 135193036 | 5.32 |
| neural precursor cell expressed, | NEDD4L // | NM_015277 // | FLJ33870 // | 3789947 | chr18 | + | 53862627 | 54232131 | 5.30 |
| developmentally down-regulated 4- | SEMA4G | NM_017893 | KIAA0439 // RSP5 // | | | | | | |
| like // sema domain, immunoglobulin | | | hNedd4-2 // | | | | | | |
| domain (Ig), transmembrane domain | | | FLJ20590 // | | | | | | |
| (TM) and short cytoplasmic domain, | | | KIAA1619 // | | | | | | |
| (semaphorin) 4G | | | MGC102867 | | | | | | |
| tetraspanin 14 | TSPAN14 | NM_030927 | DC-TM4F2 // | 3254521 | chr10 | + | 82203922 | 82272901 | 5.30 |
| | | | MGC11352 // | | | | | | |
| | | | TM4SF14 | | | | | | |
| core-binding factor, beta subunit | CBFB | NM_001755 // | PEBP2B | 3665116 | chr16 | + | 65620522 | 65692457 | 5.30 |
| | | NM_022845 | | | | | | | |
| baculoviral IAP repeat-containing 5 | BIRC5 | NM_001012270 // | API4 // EPR-1 | 3736290 | chr17 | + | 73721882 | 73733311 | 5.29 |
| (survivin) | | NM_001012271 // | | | | | | | |
| | | NM_001168 | | | | | | | |
| grainyhead-like 2 (Drosophila) | GRHL2 | NM_024915 | BOM // DFNA28 // | 3109687 | chr8 | + | 102573566 | 102751110 | 5.29 |
| | | | FLJ11172 // | | | | | | |
| | | | FLJ13782 // | | | | | | |
| | | | MGC149294 // | | | | | | |
| | | | MGC149295 // | | | | | | |
| | | | TFCP2L3 | | | | | | |
| acdysoneless homolog (Drosophila) | ECD | NM_007265 | GCR2 // HSGT1 | 3294242 | chr10 | − | 74559939 | 74598411 | 5.29 |
| RNA binding motif protein 13 | RBM13 | NM_032509 | MAK16 // MAK16L | 3093259 | chr8 | + | 33462192 | 33478316 | 528 |
| cell division cycle associated 2 | CDCA2 | NM_152562 | FLJ25804 // | 3090697 | chr8 | + | 25362573 | 25431420 | 5.28 |
| | | | MGC129906 // | | | | | | |
| | | | MGC129907 // Repo- | | | | | | |
| | | | Man | | | | | | |
| myeloid/lymphoid or mixed-lineage | MLLT4 | NM_001040000 // | AF-6 // AF6 // | 2936857 | chr6 | + | 167970473 | 168115537 | 5.28 |
| leukemia (trithorax homolog, | | NM_001040001 // | AFADIN // FLJ34371 | | | | | | |
| Drosophila); translocated to, 4 | | NM_005936 | // RP3-431P23.3 | | | | | | |
| nuclear receptor subfamily 1, group | NR1H2 | NM_007121 | LXR-b // LXRB // | 3839276 | chr19 | + | 55524771 | 55578457 | 5.28 |
| H, member 2 | | | NER // NER-1 // | | | | | | |
| | | | RIP15 // UNR | | | | | | |
| tight junction protein 2 (zona | TJP2 | NM_004817 // | MGC26306 // X104 | 3173880 | chr9 | + | 70925894 | 71059931 | 5.27 |
| occludens 2) | | NM_201629 | // ZO-2 // ZO2 | | | | | | |
| eukaryotic translation initiation factor | EIF2A | NM_032025 | CDA02 // EIF-2A // | 2647742 | chr3 | + | 151747168 | 151788822 | 5.27 |
| 2A, 65 kDa | | | MST089 // MSTP004 | | | | | | |
| | | | // MSTP089 | | | | | | |
| histone acetyltransferase 1 | HAT1 | NM_001033085 // | — | 2515369 | chr2 | + | 172487173 | 172556842 | 5.26 |
| | | NM_003642 | | | | | | | |
| ring finger protein 138 | RNF138 | NM_016271 // | HSD-4 // MGC8758 | 3783749 | chr18 | + | 27925835 | 28030948 | 5.26 |
| | | NM_198128 | // NARF // STRIN | | | | | | |
| phosphatase and tensin homolog | PTEN | NM_000314 | BZS // MGC11227 // | 3256689 | chr10 | + | 89598814 | 89722145 | 5.25 |
| (mutated in multiple advanced | | | MHAM // MMAC1 // | | | | | | |
| cancers 1) | | | PTEN1 // TEP1 | | | | | | |
| Ran GTPase activating protein 1 | RANGAP1 | NM_002883 | Fug1 // KIAA1835 // | 3961842 | chr22 | − | 39969442 | 40028027 | 5.24 |
| | | | MGC20266 // SD | | | | | | |
| zinc finger and BTB domain | ZBTB7A | NM_015898 | DKFZp547O146 // | 3846594 | chr19 | − | 3990713 | 4033507 | 5.24 |
| containing 7A | | | FBI-1 // FBI1 // LRF | | | | | | |
| | | | // MGC99631 // | | | | | | |
| | | | ZBTB7 // pokemon | | | | | | |
| methionyl aminopeptidase 2 // | METAP2 // | NM_006838 // | MNPEP // p67 // | 3426917 | chr12 | + | 94391437 | 94696442 | 5.23 |
| hypothetical protein FLJ11292 | FLJ11292 | NM_018382 | p67eIF2 // — | | | | | | |
| small nuclear ribonucleoprotein | SNRPB | NM_003091 // | COD // SNRPB1 // | 3894995 | chr20 | − | 2369715 | 2399499 | 5.23 |
| polypeptides B and B1 | | NM_198216 | SmB/SmB′ // | | | | | | |
| | | | snRNP-B | | | | | | |
| CD151 molecule (Raph blood group) | CD151 // | NM_001039490 // | GP27 // MER2 // | 3316344 | chr11 | + | 822755 | 828824 | 5.23 |
| // FK506 binding protein 9-like | FKBP9L | NM_004357 // | PETA-3 // RAPH // | | | | | | |
| | | NM_139029 // | SFA1 // TSPAN24 // | | | | | | |
| | | NM_139030 // | FKBP9 // MGC20531 | | | | | | |
| | | NM_182827 | | | | | | | |
| wolf-Hirschhorn syndrome candidate 1 | WHSC1 | NM_001042424 // | FLJ23286 // | 2715076 | chr4 | + | 1842909 | 1953717 | 5.23 |
| | | NM_007331 // | KIAA1090 // MMSET | | | | | | |
| | | NM_133330 // | // NSD2 // REIIBP | | | | | | |
| | | NM_133331 // | // TRX5 // WHS | | | | | | |
| | | NM_133334 // | | | | | | | |
| | | NM_133335 // | | | | | | | |
| | | NM_133336 | | | | | | | |
| heterogeneous nuclear | HNRPUL1 | NM_144733 // | E1B-AP5 // E1BAP5 | 3834089 | chr19 | + | 46460006 | 46505508 | 5.22 |
| ribonucleoprotein U-like 1 | | NM_144734 // | // FLJ12944 | | | | | | |
| | | NM_007040 // | | | | | | | |
| | | NM_144732 | | | | | | | |
| ubiquitin-conjugating enzyme E2C // | UBE2C // | NM_007019 // | UBCH10 // dJ447F3.2 | 3887049 | chr20 | + | 43874662 | 43878994 | 5.22 |
| p21 (CDKN1A)-activated kinase 3 | PAK3 | NM_181799 // | // CDKN1A // | | | | | | |
| | | NM_181800 // | MRX30 // MRX47 // | | | | | | |
| | | NM_181801 // | OPHN3 // PAK3beta | | | | | | |
| | | NM_181802 // | // bPaK // hPAK3 | | | | | | |
| | | NM_181803 // | | | | | | | |
| | | NM_002578 | | | | | | | |
| ribosomal L1 domain containing 1 | RSL1D1 | NM_015659 | CSIG // | 3680583 | chr16 | − | 11835208 | 11852988 | 5.22 |
| | | | DKFZP564M182 // | | | | | | |
| | | | L12 // MGC138433 | | | | | | |
| | | | // MGC142259 // | | | | | | |
| Sjogren's syndrome nuclear | SSNA1 | NM_003731 | N14 // NA-14 // | 3195296 | chr9 | + | 139202850 | 139204636 | 5.22 |
| autoantigen 1 | | | NA14 | | | | | | |
| zinc finger protein 227 // zinc finger | ZNF227 // | NM_182490 // | —// MGC161655 // | 3835544 | chr19 | + | 49408524 | 49433254 | 5.21 |
| protein 155 | ZNF155 | NM_003445 // | pHZ-96 | | | | | | |
| | | NM_198089 | | | | | | | |
| splicing factor, arginine/serine-rich 6 | SFRS6 | NM_006275 | B52 // MGC5045 // | 3886050 | chr20 | + | 41519932 | 41526301 | 5.21 |
| | | | SRP55 | | | | | | |
| transmembrane protein 5 | TMEM5 | NM_014254 | HP10481 | 3419585 | chr12 | + | 62440396 | 62489599 | 5.20 |
| peptide deformylase (mitochondrial) | PDF // | NM_0022341 // | —// DOR1 // | 3696524 | chr16 | − | 67914755 | 67931014 | 5.20 |
| // component of oligomeric golgi | COG8 | NM_032382 | FLJ22315 | | | | | | |
| complex 8 | | | | | | | | | |
| membrane-associated ring finger | 6-Mar | NM_005885 | KIAA0597 // | 2801608 | chr5 | + | 10406824 | 10490959 | 5.20 |
| (C3HC4) 6 | | | MARCH-VI // | | | | | | |
| Sp3 transcription factor // Sp3 | SP3 // | NM_001017371 // | DKFZp686O1631 // | 2587520 | chr2 | − | 174445149 | 174610682 | 5.20 |
| transcription factor pseudogene | RP11- | NM_003111 // — | SPR-2 // LOC160824 | | | | | | |
| | 114G1.1 | | | | | | | | |
| ubiquitin specific peptidase like 1 | USPL1 | NM_005800 | C13orf22 // | 3484005 | chr13 | + | 30028543 | 30132897 | 5.20 |
| | | | D13S106E // | | | | | | |
| | | | DKFZp781K2286 // | | | | | | |
| | | | FLJ32952 // RP11- | | | | | | |
| | | | 121O19.1 // | | | | | | |
| | | | bA121O19.1 | | | | | | |
| nucleolin | NCL | NM_005381 | C23 // FLJ45706 | 2603460 | chr2 | − | 232027713 | 232080578 | 5.19 |
| myotubularin related protein 3 | MTMR3 | NM_021090 // | FYVE-DSP1 // | 3942179 | chr22 | + | 28609182 | 28762301 | 5.19 |
| | | NM_153050 // | KIAA0371 // | | | | | | |
| | | NM_153051 | ZFYVE10 | | | | | | |
| Sec61 gamma subunit | SEC61G | NM_001012456 // | SSS1 | 3051395 | chr7 | − | 54753676 | 54847174 | 5.19 |
| | | NM_014302 | | | | | | | |
| signal recognition particle 72 kDa | SRP72 | NM_006947 | — | 2728224 | chr4 | + | 57028214 | 57064696 | 5.19 |
| chromosome 1 open reading frame | C1orf77 | NM_015607 | DKFZP547E1010 // | 2359736 | chr1 | + | 151872948 | 151885444 | 5.18 |
| 77 | | | MGC131924 // | | | | | | |
| | | | MGC86949 // RP1- | | | | | | |
| | | | 178F15.2 // pp7704 | | | | | | |
| zinc finger protein 787 | ZNF787 | NM_001002836 | — | 3871730 | chr19 | − | 61261349 | 61355028 | 5.18 |
| TAF5-like RNA polymerase II, | TAF5L // | NM_001025247 // | PAF65B // | 2459971 | chr1 | − | 227795491 | 227828457 | 5.18 |
| p300/CBP-associated factor | MUC4 | NM_014409 // | HSA276359 | | | | | | |
| (PCAF)-associated factor, 65 kDa // | | XM_001125749 // | | | | | | | |
| mucin 4, cell surface associated | | NM_004532 // | | | | | | | |
| | | NM_018406 // | | | | | | | |
| | | NM_138297 | | | | | | | |
| cyclin T1 | CCNT1 | NM_001240 | CCNT // CYCT1 | 3453218 | chr12 | − | 47372840 | 47397048 | 5.18 |
| A kinase (PRKA) anchor protein 1 | AKAP1 | NM_003488 | AKAP // AKAP121 // | 3728097 | chr17 | + | 52517583 | 52667254 | 5.18 |
| | | | AKAP149 // AKAP84 | | | | | | |
| | | | // D-AKAP1 // | | | | | | |
| | | | MGC1807 // PRKA1 | | | | | | |
| | | | // SAKAP84 | | | | | | |
| proteasome (prosome, macropain) | PSMC5 | NM_002805 | S8 // SUG1 // | 3730941 | chr17 | + | 59258523 | 59263555 | 5.18 |
| 26S subunit, ATPase, 5 | | | TBP10 // TRIP1 // | | | | | | |
| | | | p45 // p45/SUG | | | | | | |
| protein phosphatase 1, regulatory | PPP1R15B | NM_032833 // | FLJ14744 // C2-PI3K | 2452049 | chr1 | − | 202639152 | 202647598 | 5.17 |
| (inhibitor) subunit 15B // | // PIK3C2B | NM_002646 | // DKFZp686G16234 | | | | | | |
| phosphoinositide-3-kinase, class 2, | | | | | | | | | |
| beta polypeptide | | | | | | | | | |
| myristoylated alanine-rich protein | MARCKS | NM_002356 | 80K-L // FLJ14368 | 2922215 | chr6 | + | 114284762 | 114292853 | 5.17 |
| kinase C substrate | | | // FLJ90045 // | | | | | | |
| | | | MACS // MRACKS // | | | | | | |
| | | | PKCSL // PRKCSL | | | | | | |
| M-phase phosphoprotein 10 (U3 | MPHOSPH10 | NM_005791 | MPP10 // MPP10P | 2488078 | chr2 | + | 71210952 | 71237711 | 5.16 |
| small nucleolar ribonucleoprotein) | | | | | | | | | |
| transmembrane protein 170 | TMEM170 | NM_145254 | FLJ37611 | 3699581 | chr16 | − | 74034456 | 74107659 | 5.15 |
| YTH domain family, member 2 | YTHDF2 | NM_016258 | HGRG8 // NY-REN-2 | 2327630 | chr1 | + | 28935740 | 28968859 | 5.15 |
| breast cancer anti-estrogen | BCAR3 | NM_003567 | KIAA0554 // NSP2 // | 2423422 | chr1 | − | 93790445 | 94085633 | 5.15 |
| resistance 3 | | | SH2D3B | | | | | | |
| KH domain containing, RNA binding, | KHDRBS1 | NM_006559 | FLJ34027 // Sam68 | 2328465 | chr1 | + | 32212249 | 32299034 | 5.15 |
| signal transduction associated 1 | | | // p62 | | | | | | |
| reticulon 4 | RTN4 | NM_007008 // | ASY // NI220/250 // | 2553576 | chr2 | − | 55051075 | 55161279 | 5.15 |
| | | NM_020532 // | NOGO // NOGO-A | | | | | | |
| | | NM_153828 // | // NSP // NSP-CL | | | | | | |
| | | NM_207520 // | // Nbla00271 // | | | | | | |
| | | NM_207521 | Nbla10545 // RTN-X | | | | | | |
| | | | // RTN4-A // RTN4- | | | | | | |
| | | | B1 // RTN4-B2 // | | | | | | |
| | | | RTN4-C | | | | | | |
| calcitonin gene-related peptide- | RCP9 | NM_001040647 // | CGRP-RCP // CRCP | 3005332 | chr7 | + | 65131004 | 65262097 | 5.15 |
| receptor component protein | | NM_001040648 // | // MGC111194 // | | | | | | |
| | | NM_014478 | RCP | | | | | | |
| solute carrier family 3 (activators of | SLC3A2 | NM_001012661 // | 4F2 // 4F2HC // | 3333711 | chr11 | + | 62380117 | 62412872 | 5.15 |
| dibasic and neutral amino acid | | NM_001012662 // | 4T2HC // CD98 // | | | | | | |
| transport), member 2 | | NM_001012663 // | CD98HC // MDU1 // | | | | | | |
| | | NM_001012664 // | NACAE | | | | | | |
| | | NM_001013251 // | | | | | | | |
| | | NM_002394 | | | | | | | |
| zinc finger, DHHC-type containing 5 | ZDHHC5 | NM_015457 | DKFZP586K0524 // | 3331433 | chr11 | + | 57192053 | 57225319 | 5.14 |
| | | | KIAA1748 // ZNF375 | | | | | | |
| Smg-5 homolog, nonsense mediated | SMG5 | NM_015327 | EST1B // FLJ34864 | 2438042 | chr1 | − | 154485643 | 154519254 | 5.14 |
| mRNA decay factor (C. elegans) | | | // KIAA1089 // | | | | | | |
| | | | LPTS-RP1 // | | | | | | |
| | | | LPTSRP1 // RP11- | | | | | | |
| | | | 54H19.7 // SMG-5 | | | | | | |
| alkB, alkylation repair homolog 2 (E. coli) | ALKBH2 | NM_001001655 | ABH2 // MGC90512 | 3470689 | chr12 | − | 108010254 | 108015656 | 5.13 |
| | | | // hABH2 | | | | | | |
| BUD31 homolog (S. cerevisiae) | BUD31 | NM_003910 | EDG-2 // EDG2 // | 3014742 | chr7 | + | 98844221 | 98855171 | 5.13 |
| | | | G10 // MGC111202 | | | | | | |
| | | | // YCR063W | | | | | | |
| MIS12, MIND kinetochore complex | MIS12 | NM_024039 | 2510025F08Rik // | 3707759 | chr17 | + | 5330450 | 5334853 | 5.13 |
| component, homolog (yeast) | | | KNTC2AP // | | | | | | |
| | | | MGC2488 // MTW1 | | | | | | |
| | | | // hMis12 | | | | | | |
| tripartite motif-containing 26 | TRIM26 | NM_003449 | AFP // RNF95 // | 2948259 | chr6 | − | 30260217 | 30290029 | 5.13 |
| | | | ZNF173 | | | | | | |
| similar to FRG1 protein (FSHD region | MGC72104 | NM_207350 // | —// FSG1 | 2756029 | chr4 | + | 191051868 | 191138306 | 5.13 |
| gene 1 protein) // FSHD region gene 1 | // FRG1 | NM_004477 | | | | | | | |
| ATPase family, AAA domain | ATAD1 | NM_032810 | AFDC1 // FLJ14600 | 3299255 | chr10 | − | 89500784 | 89591413 | 5.12 |
| containing 1 | | | // FNP001 | | | | | | |
| BTB (POZ) domain containing 3 | BTBD3 | NM_014962 // | KIAA0952 // | 3876645 | chr20 | + | 11722386 | 11855239 | 5.12 |
| | | NM_181443 | MGC130038 // | | | | | | |
| | | | MGC130039 // | | | | | | |
| | | | dJ742J24.1 | | | | | | |
| sterol regulatory element binding | SREBF2 | NM_004599 | SREBP2 | 3947123 | chr22 | + | 40559049 | 40633247 | 5.12 |
| transcription factor 2 | | | | | | | | | |
| splicing factor 3a, subunit 3, 60 kDa | SF3A3 | NM_006802 | PRP9 // PRPF9 // | 2407439 | chr1 | − | 38195241 | 38229180 | 5.11 |
| | | | SAP61 // SF3a60 | | | | | | |
| ATP synthase, H+ transporting, | ATP5B | NM_001686 | ATPMB // ATPSB // | 3458033 | chr12 | − | 55318243 | 55326110 | 5.11 |
| mitochondrial F1 complex, beta | | | MGC5231 | | | | | | |
| polypeptide | | | | | | | | | |
| mitochondrial ribosomal protein L55 | MRPL55 | NM_181441 // | AAVG5835 // | 2459438 | chr1 | − | 226360734 | 226367172 | 5.11 |
| | | NM_181454 // | DKFZp686D1387 // | | | | | | |
| | | NM_181455 // | MGC61802 // | | | | | | |
| | | NM_181456 // | PRO19675 | | | | | | |
| | | NM_181462 // | | | | | | | |
| | | NM_181463 // | | | | | | | |
| | | NM_181464 // | | | | | | | |
| | | NM_181465 | | | | | | | |
| SMAD specific E3 ubiquitin protein | SMURF1 | NM_020429 // | KIAA1625 | 3063083 | chr7 | − | 98449133 | 98579664 | 5.11 |
| ligase 1 | | NM_181349 | | | | | | | |
| myeloid/lymphoid or mixed-lineage | MLL | NM_005933 | ALL-1 // CXXC7 // | 3351385 | chr11 | + | 117811029 | 117902749 | 5.10 |
| leukemia (trithorax homolog, | | | HRX // HTRX1 // | | | | | | |
| Drosophila) | | | MLL/GAS7 // MLL1A | | | | | | |
| | | | // TRX1 | | | | | | |
| family with sequence similarity 89, | FAM89B | NM_152832 | MTVR1 | 3335338 | chr11 | + | 65096490 | 65098230 | 5.10 |
| member B | | | | | | | | | |
| alkB, alkylation repair homolog 3 (E. coli) | ALKBH3 | NM_139178 | ABH3 // DEPC-1 // | 3328214 | chr11 | + | 43858633 | 43898486 | 5.10 |
| | | | DEPC1 // | | | | | | |
| | | | MGC118790 // | | | | | | |
| | | | MGC118792 // | | | | | | |
| SET domain containing 2 | SETD2 | NM_014159 | FLJ16420 // | 2672532 | chr3 | − | 47032936 | 47180418 | 5.09 |
| | | | FLJ22472 // | | | | | | |
| | | | FLJ23184 // | | | | | | |
| | | | FLJ45883 // HIF-1 // | | | | | | |
| | | | HSPC069 // HYPB // | | | | | | |
| | | | KIAA1732 | | | | | | |
| p53 and DNA damage regulated 1 | PDRG1 | NM_030815 | C20orf126 // PDRG | 3902609 | chr20 | − | 29995826 | 30005314 | 5.09 |
| ubiquitin carboxyl-terminal esterase | UCHL3 | NM_006002 | — | 3494102 | chr13 | + | 75021620 | 75078250 | 5.08 |
| L3 (ubiquitin thiolesterase) | | | | | | | | | |
| hepatocyte growth factor-regulated | HGS | NM_004712 | HRS // ZFYVE8 | 3738138 | chr17 | + | 77260785 | 77279549 | 5.08 |
| tyrosine kinase substrate | | | | | | | | | |
| proteasome (prosome, macropain) | PSMC4 | NM_006503 // | MGC13687 // | 3833291 | chr19 | + | 45168772 | 45179181 | 5.08 |
| 26S subunit, ATPase, 4 | | NM_153001 | MGC23214 // | | | | | | |
| | | | MGC8570 // MIP224 | | | | | | |
| | | | // S6 // TBP7 | | | | | | |
| tubulin, beta | TUBB | NM_178014 | M40 // MGC117247 | 2901913 | chr6 | + | 30795977 | 30801165 | 5.08 |
| | | | // MGC16435 // | | | | | | |
| | | | OK/SW-cl.56 // | | | | | | |
| | | | TUBB1 // TUBB5 | | | | | | |
| kinesin family member 20A | KIF20A | NM_005733 | FLJ21151 // MKLP2 | 2830638 | chr5 | + | 137542289 | 137552275 | 5.08 |
| | | | // RAB6KIFL | | | | | | |
| arginyl-tRNA synthetase | RARS | NM_002887 | ArgRS // DALRD1 // | 2839671 | chr5 | + | 167845940 | 167878885 | 5.08 |
| | | | MGC8641 | | | | | | |
| calpain 2, (m/II) large subunit | CAPN2 | NM_001748 | CANPL2 // CANPml | 2382117 | chr1 | + | 221955990 | 222031806 | 5.08 |
| | | | // FLJ39928 // | | | | | | |
| | | | mCANP | | | | | | |
| ribosomal protein S15a | RPS15A | NM_001019 // | FLJ27457 // | 3683018 | chr16 | − | 18700121 | 18709175 | 5.07 |
| | | NM_001030009 | MGC111208 // S15a | | | | | | |
| topoisomerase (DNA) II binding | TOPBP1 | NM_007027 | TOP2BP1 | 2695941 | chr3 | − | 134799729 | 134863527 | 5.07 |
| protein 1 | | | | | | | | | |
| calcium homeostasis endoplasmic | CHERP // | NM_006387 // | DAN16 // SCAF6 // | 3853942 | chr19 | − | 16489710 | 16517113 | 5.06 |
| reticulum protein // cofactor | CRSP7 | NM_004831 | SRA1 // CRSP70 // | | | | | | |
| required for Sp1 transcriptional | | | MED26 | | | | | | |
| activation, subunit 7, 70 kDa | | | | | | | | | |
| echinoderm microtubule associated | EML4 | NM_019063 | C2orf2 // | 2478748 | chr2 | + | 42250017 | 42418636 | 5.06 |
| protein like 4 | | | DKFZp686P18118 // | | | | | | |
| | | | ELP120 // FLJ10942 | | | | | | |
| | | | // FLJ32318 // | | | | | | |
| | | | ROPP120 | | | | | | |
| splicing factor 3b, subunit 4, 49 kDa | SF3B4 | NM_005850 | MGC10828 // SAP49 | 2434159 | chr1 | − | 148161628 | 148167125 | 5.05 |
| | | | // SF3b49 | | | | | | |
| rho/rac guanine nucleotide exchange | ARHGEF18 | NM_015318 | KIAA0521 // | 3818732 | chr19 | + | 7319892 | 7443363 | 5.05 |
| factor (GEF) 18 | | | MGC15913 | | | | | | |
| pescadillo homolog 1, containing | PES1 | NM_014303 | PES | 3957445 | chr22 | − | 29302623 | 29333107 | 5.05 |
| BRCT domain (zebrafish) | | | | | | | | | |
| Ras association (RalGDS/AF-6) | RASSF7 | NM_003475 | C11orf13 // HRAS1 | 3315952 | chr11 | + | 550321 | 554016 | 5.05 |
| domain family 7 | | | // HRC1 // | | | | | | |
| | | | MGC126069 // | | | | | | |
| | | | MGC126070 | | | | | | |
| Wilms tumor 1 associated protein // | WTAP // | NM_004906 // | DKFZp686F20131 // | 2934089 | chr6 | + | 160066607 | 160097332 | 5.04 |
| acetyl-Coenzyme A | ACAT2 | NM_152857 // | KIAA0105 // | | | | | | |
| acetyltransferase 2 (acetoacetyl | | NM_152858 // | MGC3925 // — | | | | | | |
| Coenzyme A thiolase) | | NM_005891 | | | | | | | |
| origin recognition complex, subunit | ORC5L | NM_002553 // | ORC5 // ORC5P // | 3065963 | chr7 | − | 103553146 | 103719295 | 5.04 |
| 5-like (yeast) | | NM_181747 | ORC5T | | | | | | |
| chromodomain protein, Y-like | CDYL | NM_004824 // | CDYL1 // | 2892979 | chr6 | + | 4651344 | 4900768 | 5.03 |
| | | NM_170751 // | DKFZP586C1622 // | | | | | | |
| | | NM_170752 | MGC131936 | | | | | | |
| aprataxin | APTX | NM_017692 // | AOA // AOA1 // | | | | | | |
| | | NM_175069 // | AXA1 // EAOH // | | | | | | |
| | | NM_175071 // | EOAHA // FHA-HIT | 3203311 | chr9 | − | 32962360 | 33015096 | 5.03 |
| | | NM_175072 // | // FLJ20157 // | | | | | | |
| | | NM_175073 | MGC1072 | | | | | | |
| phosphatidylinositol 4-kinase, | PIK4CB // | NM_002651 // | PI4K-BETA // | 2434925 | chr1 | − | 149530589 | 149567792 | 5.03 |
| catalytic, beta polypeptide // | RFX5 | NM_000449 // | PI4KIIIbeta // | | | | | | |
| regulatory factor X, 5 (influences | | NM_001025603 | PI4Kbeta // — | | | | | | |
| HLA class II expression) | | | | | | | | | |
| serine palmitoyltransferase, long | SPTLC2 | NM_004863 | KIAA0526 // LCB2 // | 3573152 | chr14 | − | 77042100 | 77202740 | 5.02 |
| chain base subunit 2 | | | SPT2 | | | | | | |
| cell division cycle associated 5 | CDCA5 | NM_080668 | MGC16386 | 3377423 | chr11 | − | 64592075 | 64608249 | 5.02 |
| myeloid/lymphoid or mixed-lineage | MLL5 | NM_018682 // | FLJ10078 // | 3017547 | chr7 | + | 104436029 | 104542698 | 5.02 |
| leukemia 5 (trithorax homolog, | | NM_182931 | FLJ14026 // | | | | | | |
| Drosophila) | | | HDCMC04P // | | | | | | |
| | | | MGC70452 | | | | | | |
| F-box protein 28 | FBXO28 | NM_015176 | FLJ10766 // Fbx28 | 2382336 | chr1 | + | 222368455 | 222416368 | 5.02 |
| | | | // KIAA0483 | | | | | | |
| eukaryotic translation initiation factor | EIF3S1 | NM_003758 | eIF3-alpha // eIF3- | 3591963 | chr15 | + | 42616580 | 42644124 | 5.02 |
| 3, subunit, 1 alpha, 35 kDa | | | p35 // eIF3j | | | | | | |
| polymerase (RNA) III (DNA directed) | POLR3O | NM_006468 | RPC3 // RPC62 | 2432647 | chr1 | − | 144297192 | 144333220 | 5.01 |
| polypeptide C (62 kD) | | | | | | | | | |
| bromodomain adjacent to zinc finger | BAZ1B | NM_023005 // | WBSCR10 // | 3056044 | chr7 | − | 72492710 | 72574552 | 5.01 |
| domain, 1B | | NM_032408 | WBSCR9 // WSTF | | | | | | |
| isocitrate dehydrogenase 3 (NAD+) | IDH3B | NM_006899 // | FLJ11043 // H-IDHB | 3895075 | chr20 | − | 2586736 | 2592862 | 5.01 |
| beta | | NM_174855 // | // MGC903 | | | | | | |
| | | NM_174856 | | | | | | | |
| basic transcription factor 3-like 4 | BTF3L4 | NM_152265 | MGC23908 // | 2336271 | chr1 | + | 52293292 | 52339895 | 5.01 |
| | | | MGC88389 // RP4- | | | | | | |
| | | | 800M22.5 | | | | | | |
| Src homology 2 domain containing | SHB // | NM_003028 // | RP11-3J10.8 // | 3205659 | chr9 | − | 37901375 | 38059198 | 5.00 |
| adaptor protein B // cytoskeleton | CKAP2 // | NM_018204 // | bA3J10.2 // | | | | | | |
| associated protein 2 // mitochondrial | MCART1 | NM_033412 | FLJ10749 // LB1 // | | | | | | |
| carrier triple repeat 1 | | | CG7943 // | | | | | | |

### Example 9

### Analysis with Microarray (3)

Using the PBMC samples in Example 4(2), genes of which introns exhibited increased expression were identified. Ribosomal RNA was removed from 1 μg of each total RNA of control cells (n=3) and cells with the treatment (n=3) using RiboMinus Human/Mouse Transcriptome Isolation Kit (Invitrogen). Single stranded cDNA synthesis, double stranded cDNA synthesis, cRNA synthesis, second single stranded cDNA synthesis, cDNA fragmentation, and cDNA labeling in order were carried out for total RNA from which ribosomal RNA was removed, using GeneChip Whole Transcript Sense Target Labeling and Control Reagents (Affymetrix) to make a cDNA probe. Subsequently, the cDNA probe was used for hybridization with Human Exon 1.0 ST Array (Affymetrix). The array was washed and stained, and luminescence intensity was measured by a scanner.

The expression level of the probe set and the gene was quantified by using Expression Console Ver. 1.0 (Affymetrix) with Summarization Method and Normalization Method being set to “Median polish as used in RMA” and “None”, respectively. The expression level of the probe was normalized with that of the gene.

(1) Identification of a Gene of which Introns Exhibit Increased Expression in a Group with the Treatment at a Concentration of 3 nM

Probe sets showing that the normalized change in the level expression of the probe set in the group with the treatment at 3 nM was more than 5 times; the expression level of the probe set and the gene in the group with the treatment at 3 nM was more than 100; and probe set annotation was “extended, full, free”, were picked out. Welch's t-test was conducted with the probe sets extracted for the group with the treatment at 3 nM and the control group to determine a p value and a q value. A gene containing the probe sets with the q value of less than 5% was determined as a candidate gene with increased expression level of the intron regions (Table 4).

In Table 4, for the gene evaluated, gene name (Gene Name), abbreviated name (Gene Symbol), accession number (Accession), alias name (Synonym), transcription cluster number in Human Exon 1.0 ST Array (Human Exon 1.0 ST Array Transcript Cluster ID), chromosome number (Chromosome), type of strand (Strand), start region (Start), stop region (Stop), and change in expression level (Fold Change) were respectively shown.

TABLE 4

| | | | | Human Exon | | | | | |
|---|---|---|---|---|---|---|---|---|---|
| | | | | ST 1.0 | Human Genome hg18 | | | | |
| Gene Name | Gene Symbol | Accession | Synonym | Transcript Cluster ID | Chromosome | Strand | Start | Stop | Fold Change |
| TIMP metallopeptidase inhibitor 1 | TIMP1 | NM_003254 | CLGI // EPA // | 3976341 | chrX | + | 47326654 | 47332978 | 14.98 |
| | | | EPO // | | | | | | |
| | | | FLJ90373 // | | | | | | |
| | | | HCI // TIMP | | | | | | |
| ADAM metallopeptidase domain 19 (meltrin beta) | ADAM19 | NM_023038 | FKSG34 // | 2883440 | chr5 | − | 156755122 | 156935351 | 9.71 |
| | | // | MADDAM // | | | | | | |
| | | NM_033274 | MLTNB | | | | | | |
| phosphodiesterase 4A, cAMP-specific | PDE4A | NM_006202 | DPDE2 // PDE4 | 3820501 | chr19 | + | 10388560 | 10451531 | 9.64 |
| (phosphodiesterase E2 dunce homolog. | | | | | | | | | |
| Rap guanine nucleotide exchange factor (GEF) 1 | RAPGEF1 | NM_005312 | C3G // | 3227696 | chr9 | − | 133441988 | 133605262 | 9.27 |
| | | // | DKFZp781P1719 | | | | | | |
| | | NM_198679 | // GRF2 | | | | | | |
| myosin, heavy chain 9, non-muscle | MYH9 | NM_002473 | DFNA17 // | 3959451 | chr22 | − | 35007278 | 35113982 | 7.90 |
| | | | EPSTS // FTNS | | | | | | |
| | | | // MGC104539 | | | | | | |
| | | | // MHA // | | | | | | |
| | | | NMHC-II-A // | | | | | | |
| | | | NMMHCA | | | | | | |
| serpin peptidase inhibitor, clade B (ovalbumin), | SERPINB9 // | NM_004155 | CAP-3 // CAP3 | 2939034 | chr6 | − | 2832402 | 2858242 | 7.75 |
| member 9 // serpin peptidase inhibitor, clade B | SERPINB6 | // | // P19 // CAP | | | | | | |
| (ovalbumin), member 6 | | NM_004568 | // | | | | | | |
| | | | DKFZp686I04222 | | | | | | |
| | | | // | | | | | | |
| | | | MGC111370 // | | | | | | |
| | | | MSTP057 // | | | | | | |
| RNA binding motif protein 23 | RBM23 | NM_001077351 | CAPERbeta // | 3556888 | chr14 | − | 22439714 | 22458231 | 7.63 |
| | | // | FLJ10482 // | | | | | | |
| | | NM_001077352 | MGC4458 // | | | | | | |
| | | // | PP239 // | | | | | | |
| | | NM_018107 | RNPC4 | | | | | | |
| polyhomeotic homolog 3 (Drosophila) | PHC3 | NM_024947 | DKFZp313K1221 | 2704894 | chr3 | − | 171287994 | 171381537 | 7.30 |
| | | | // EDR3 // | | | | | | |
| | | | FLJ12729 // | | | | | | |
| | | | FLJ12967 // | | | | | | |
| | | | HPH3 // | | | | | | |
| | | | MGC88144 | | | | | | |
| splicing factor proline/glutamine-rich | SFPQ | NM_005066 | POMP100 // | 2406064 | chr1 | − | 35351696 | 35506894 | 7.08 |
| (polypyrimidine tract binding protein associated) | | | PSF | | | | | | |
| BTB (POZ) domain containing 11 | BTBD11 | NM_001017523 | FLJ33957 // | 3430462 | chr12 | + | 106237825 | 106577543 | 7.07 |
| | | // | FLJ42845 | | | | | | |
| | | NM_001018072 | | | | | | | |
| | | // | | | | | | | |
| | | NM_152322 | | | | | | | |
| v-src sarcoma (Schmidt-Ruppin A-2) viral | SRC | NM_005417 | ASV // SRC1 | 3884191 | chr20 | + | 35406502 | 35467847 | 6.98 |
| oncogene homolog (avian) | | // | // c-SRC // | | | | | | |
| | | NM_198291 | p60-Src | | | | | | |
| signal-induced proliferation-associated 1 like 1 | SIPA1L1 | NM_015556 | DKFZp686G1344 | 3542847 | chr14 | + | 70857592 | 71468470 | 6.93 |
| | | | // E6TP1 // | | | | | | |
| | | | KIAA0440 | | | | | | |
| MYST histone acetyltransferase 2 | MYST2 | NM_007067 | HBO1 // HBOA | 3725779 | chr17 | + | 45196447 | 45261455 | 6.84 |
| GPI-anchored membrane protein 1 | GPIAP1 | NM_005898 | GPIP137 // | 3326183 | chr11 | + | 34009711 | 34081946 | 6.78 |
| | | // | M11S1 // | | | | | | |
| | | NM_203364 | p137GPI | | | | | | |
| integrin, alpha X (complement component 3 | ITGAX | XM_001127869 | CD11C | 3657041 | chr16 | + | 31274012 | 31301819 | 6.76 |
| receptor 4 subunit) | | // | | | | | | | |
| | | NM_000887 | | | | | | | |
| microtubule-associated protein, RP/EB family, | MAPRE1 | NM_012325 | EB1 // | 3882069 | chr20 | + | 30871304 | 30901864 | 6.65 |
| member 1 | | | MGC117374 // | | | | | | |
| | | | MGC129946 | | | | | | |
| RAB6 interacting protein 1 | RAB6IP1 | NM_015213 | FLJ22354 // | 3362263 | chr11 | − | 9116953 | 9243468 | 6.61 |
| | | | FLJ33829 // | | | | | | |
| | | | FLJ43455 // | | | | | | |
| | | | KIAA1091 | | | | | | |
| testis derived transcript (3 LIM domains) | TES | NM_015641 | DKFZP586B2022 | 3020192 | chr7 | + | 115637811 | 115743800 | 6.22 |
| | | // | // MGC1146 | | | | | | |
| | | NM_152829 | // TESS // | | | | | | |
| | | | TESS-2 // | | | | | | |
| t-complex 1 | TCP1 | NM_001008897 | CCT-alpha // | 2982381 | chr6 | − | 160119521 | 160130731 | 6.19 |
| | | // | CCT1 // CCTa | | | | | | |
| | | NM_030752 | // D6S230E // | | | | | | |
| | | | TCP-1-alpha | | | | | | |
| CHMP family, member 7 | CHMP7 | NM_152272 | MGC29816 | 3089853 | chr8 | + | 23157105 | 23176288 | 6.08 |
| toll-like receptor 4 | TLR4 | NM_138554 | CD284 // TOLL | 3186966 | chr9 | + | 119506334 | 119670150 | 6.00 |
| | | | // hToll | | | | | | |
| fibronectin type III domain containing 3B | FNDC3B | NM_022763 | DKFZp686D14170 | 2652410 | chr3 | + | 173240112 | 173601176 | 5.97 |
| | | | // | | | | | | |
| | | | DKFZp762K137 | | | | | | |
| | | | // FAD104 // | | | | | | |
| | | | FLJ23399 // | | | | | | |
| | | | MGC10002 // | | | | | | |
| | | | PRO4979 // | | | | | | |
| | | | YVTM2421 | | | | | | |
| serine/threonine kinase 38 like | STK38L | NM_015000 | KIAA0965 // | 3409081 | chr12 | + | 27288343 | 27370157 | 5.97 |
| | | | NDR2 | | | | | | |
| eukaryotic translation initiation factor 4 gamma, 2 | EIF4G2 | NM_001042559 | AAG1 // DAP5 | 3362719 | chr11 | − | 10487574 | 10788746 | 5.97 |
| | | // | // FLJ41344 // | | | | | | |
| | | NM_001418 | NAT1 // p97 | | | | | | |
| aminopeptidase puromycin sensitive | NPEPPS | XM_001128588 | MP100 // PSA | 3724698 | chr17 | + | 42955347 | 43057168 | 5.96 |
| | | // | | | | | | | |
| | | NM_006310 | | | | | | | |
| membrane protein, palmitoylated 1, 55 kDa | MPP1 | NM_002436 | AAG12 // | 4027585 | chrX | − | 153658739 | 154075618 | 5.94 |
| | | | DXS552 // | | | | | | |
| | | | DXS552E // | | | | | | |
| | | | EMP55 // | | | | | | |
| | | | MRG1 // PEMP | | | | | | |
| eukaryotic translation initiation factor 4 gamma, 1 | EIF4G1 | NM_004953 | DKFZp686A1451 | 2655688 | chr3 | + | 185514970 | 185535829 | 5.83 |
| | | // | // EIF4F // | | | | | | |
| | | NM_182917 | EIF4G // p220 | | | | | | |
| | | // | | | | | | | |
| | | NM_198241 | | | | | | | |
| | | // | | | | | | | |
| | | NM_198242 | | | | | | | |
| | | // | | | | | | | |
| | | NM_198244 | | | | | | | |
| leucine rich repeat containing 8 family, member C | LRRC8C | NM_032270 | AD158 // | 2345929 | chr1 | + | 89871229 | 90008050 | 5.81 |
| | | | DKFZp586J1119 | | | | | | |
| | | | // FAD158 // | | | | | | |
| | | | MGC138551 | | | | | | |
| EH-domain containing 1 | EHD1 | NM_006795 | FLJ42622 // | 3377226 | chr11 | − | 64376790 | 64404202 | 5.78 |
| | | | FLJ44618 // H- | | | | | | |
| | | | PAST // | | | | | | |
| | | | HPAST1 // | | | | | | |
| | | | PAST // PAST1 | | | | | | |
| 3-hydroxy-3-methylglutaryl-Coenzyme A | HMGCS1 | NM_002130 | HMGCS // | 2855501 | chr5 | − | 43324231 | 43349337 | 5.74 |
| synthase 1 (soluble) | | | MGC90332 | | | | | | |
| insulin induced gene 1 | INSIG1 | NM_005542 | CL-6 // | 3033209 | chr7 | + | 154649208 | 154732868 | 5.72 |
| | | // | MGC1405 | | | | | | |
| | | NM_198336 | | | | | | | |
| | | // | | | | | | | |
| | | NM_198337 | | | | | | | |
| chromodomain helicase DNA binding protein 2 | CHD2 | NM_001042572 | DKFZp781D1727 | 3609138 | chr15 | + | 91227096 | 91387351 | 5.70 |
| | | // | | | | | | | |
| | | NM_001271 | | | | | | | |
| DEAD (Asp-Glu-Ala-Asp) box polypeptide 17 | DDX17 | NM_006386 | DKFZp761H2016 | 3960629 | chr22 | − | 37202800 | 37232288 | 5.70 |
| | | // | // P72 // RH70 | | | | | | |
| | | NM_030881 | | | | | | | |
| hypothetical protein KIAA1434 | RP5-1022P6.2 | NM_019593 | FLJ11085 // | 3896370 | chr20 | − | 5473033 | 5546357 | 5.68 |
| | | | KIAA1434 // | | | | | | |
| | | | MGC26147 | | | | | | |
| nuclear factor of kappa light polypeptide gene | NFKB2 | NM_001077493 | LYT-10 // | 3261643 | chr10 | + | 104144262 | 104152716 | 5.61 |
| enhancer in B-cells 2 (p49/p100) | | // | LYT10 | | | | | | |
| | | NM_001077494 | | | | | | | |
| | | // | | | | | | | |
| | | NM_002502 | | | | | | | |
| ubiquitin-activating enzyme E1-like 2 | UBE1L2 | NM_018227 | FLJ10808 // | 2771718 | chr4 | − | 68130308 | 68249472 | 5.57 |
| | | | FLJ23367 | | | | | | |
| ras homolog gene family, member A | RHOA | NM_001664 | ARH12 // | 2674242 | chr3 | − | 49371587 | 49424514 | 5.54 |
| | | | ARHA // | | | | | | |
| | | | RHO12 // | | | | | | |
| HECT, UBA and WWE domain containing 1 | HUWE1 | NM_031407 | ARF-BP1 // | 4009315 | chrX | − | 53575796 | 53740871 | 5.54 |
| | | | HECTH9 // | | | | | | |
| | | | HSPC272 // | | | | | | |
| | | | lb772 // | | | | | | |
| | | | KIAA0312 // | | | | | | |
| | | | LASU1 // | | | | | | |
| | | | MULE // | | | | | | |
| cyclin-dependent kinase 8 | CDK8 | NM_001260 | K35 // | 3482498 | chr13 | + | 25701360 | 25877335 | 5.53 |
| | | | MGC126074 // | | | | | | |
| | | | MGC126075 | | | | | | |
| E3 ubiquitin protein ligase, HECT domain | EDD1 | NM_015902 | DD5 // EDD // | 3147321 | chr8 | − | 103333634 | 103493671 | 5.49 |
| containing, 1 | | | FLJ11310 // | | | | | | |
| | | | HYD // | | | | | | |
| | | | KIAA0896 // | | | | | | |
| | | | MGC57263 | | | | | | |
| dedicator of cytokinesis 4 | DOCK4 | NM_014705 | FLJ34238 // | 3068097 | chr7 | − | 111152902 | 111633744 | 5.42 |
| | | | KIAA0716 // | | | | | | |
| | | | MGC134911 // | | | | | | |
| | | | MGC134912 | | | | | | |
| sterol regulatory element binding transcription | SREBF2 | NM_004599 | SREBP2 | 3947123 | chr22 | + | 40559049 | 40633247 | 5.40 |
| factor 2 | | | | | | | | | |
| heterogeneous nuclear ribonucleoprotein M | HNRPM | NM_005968 | DKFZp547H118 | 3819543 | chr19 | + | 8415651 | 8459993 | 5.34 |
| | | // | // HNRNPM // | | | | | | |
| | | NM_031203 | HNRNPM4 // | | | | | | |
| | | | HNRPM4 // | | | | | | |
| | | | HTGR1 // | | | | | | |
| | | | NAGR1 | | | | | | |
| SH3 domain binding glutamic acid-rich protein | SH3BGRL3 // | NM_031286 | SH3BP-1 // | 2326448 | chr1 | + | 26478669 | 26480591 | 5.34 |
| like 3 // coiled-coil domain containing 21 | CCDC21 | // | TIP-B1 // | | | | | | |
| | | NM_022778 | DKFZP434L0117 | | | | | | |
| | | | // | | | | | | |
| | | | DKFZp434P232 | | | | | | |
| | | | // FLJ13976 // | | | | | | |
| | | | FLJ22000 | | | | | | |
| exportin 4 | XPO4 | NM_022459 | FLJ13046 // | 3504434 | chr13 | − | 20251014 | 20375187 | 5.33 |
| | | | KIAA1721 | | | | | | |
| squalene epoxidase | SQLE | NM_003129 | — | 3114832 | chr8 | + | 126063738 | 126105522 | 5.19 |
| peptidylprolyl isomerase F (cyclophilin F) | PPIF | NM_005729 | CYP3 // Cyp-D | 3253880 | chr10 | + | 80777230 | 80785093 | 5.19 |
| | | | // FLJ90798 // | | | | | | |
| | | | MGC117207 | | | | | | |
| serum response factor (c-fos serum response | SRF | NM_003131 | MCM1 | 2907730 | chr6 | + | 43246765 | 43257189 | 5.15 |
| element-binding transcription factor) | | | | | | | | | |
| c-mer proto-oncogene tyrosine kinase | MERTK | NM_006343 | MER // | 2500550 | chr2 | + | 112372656 | 112513624 | 5.12 |
| | | | MGC133349 // | | | | | | |
| | | | c-mer | | | | | | |
| echinoderm microtubule associated protein like 4 | EML4 | NM_019063 | C2orf2 // | 2478748 | chr2 | + | 42250017 | 42418636 | 5.02 |
| | | | DKFZp686P18118 | | | | | | |
| | | | // ELP120 // | | | | | | |
| | | | FLJ10942 // | | | | | | |
| | | | FLJ32318 // | | | | | | |
| | | | ROPP120 | | | | | | |

(2) Identification of the Gene of which Introns Exhibit Increased Expression in a Dose-Dependent Fashion in Groups with the Treatment at a Concentration of 3 nM, 10 nM, and 30 nM.

Probe sets showing that the normalized change in the level expression of the probe set in the group with the treatment at 30 nM was more than 5 times; the expression level of the probe set and the gene in the group with the treatment at 30 nM was more than 100; and probe set annotation was “extended, full, free”, were picked out. Regression analysis was performed with the probe sets extracted for the control group, the group with the treatment at 3 nM, the group with the treatment at 10 nM, and the group with the treatment at 30 nM to determine a p and q value for the slope of regression formula. A gene containing the probe sets with the q value of less than 5% was determined as a candidate gene with increased expression level of the intron regions (Table 5).

In Table 5, for the gene evaluated, gene name (Gene Name), abbreviated name (Gene Symbol), accession number (Accession), alias name (Synonym), transcription cluster number in Human Exon 1.0 ST Array (Human Exon 1.0 ST Array Transcription Cluster ID), chromosome number (Chromosome), type of strand (Strand), start region (Start), stop region (Stop), and change in expression level per 1 nM (Fold Change/nM) were respectively shown.

TABLE 5

| | | | | Human | | | | | |
|---|---|---|---|---|---|---|---|---|---|
| | | | | Exon ST 1.0 | | | | | Fold |
| | | | | Transcript | Human Genome hg18 | | | | Change/ |
| Gene Name | Gene Symbol | Accession | Synonym | Cluster ID | Chromosome | Strand | Start | Stop | nM |
| epiregulin | EREG | NM_001432 | ER | 2731513 | chr4 | + | 75440309 | 75473341 | 1.08 |
| pellino homolog 1 (Drosophila) | PELI1 | NM_020651 | DKFZp686C18116 | 2556302 | chr2 | − | 64173290 | 64225291 | 1.08 |
| | | | // MGC50990 | | | | | | |
| integrin, alpha V (vitronectin receptor, alpha | ITGAV | NM_002210 | CD51 // MSK8 // | 2519229 | chr2 | + | 187163045 | 187253869 | 1.08 |
| polypeptide, antigen CD51) | | | VNRA | | | | | | |
| heat shock 70 kDa protein 5 (glucose-regulated | HSPA5 | NM_005347 | BIP // FLJ26106 // | 3225398 | chr9 | − | 127036953 | 127064590 | 1.08 |
| protein, 78 kDa) | | | GRP78 // MIF2 | | | | | | |
| villin 2 (ezrin) | VIL2 | NM_003379 | CVIL // CVL // | 2981912 | chr6 | − | 159106770 | 159160432 | 1.08 |
| | | | DKFZp762H157 // | | | | | | |
| | | | FLJ26216 // | | | | | | |
| | | | MGC1584 | | | | | | |
| UDP-Gal:betaGlcNAc beta 1,4- | B4GALT5 | NM_004776 | B4Gal-T5 // | 3908963 | chr20 | − | 47682889 | 47809709 | 1.08 |
| galactosyltransferase, polypeptide 5 | | | BETA4-GALT-IV // | | | | | | |
| | | | MGC138470 // | | | | | | |
| | | | beta4Gal-T5 // | | | | | | |
| | | | beta4GalT-V // gt- | | | | | | |
| pleckstrin homology, Sec7 and coiled-coil | PSCD1 | NM_004762 // | B2-1 // | 3772525 | chr17 | − | 74181731 | 74289973 | 1.08 |
| domains 1(cytohesin 1) | | NM_017456 | CYTOHESIN-1 // | | | | | | |
| | | | D17S811E // | | | | | | |
| | | | FLJ34050 // | | | | | | |
| | | | FLJ41900 // SEC7 | | | | | | |
| EH-domain containing 4 | EHD4 | NM_139265 | PAST4 | 3620276 | chr15 | − | 39977674 | 40052063 | 1.08 |
| TIMP metallopeptidase inhibitor 1 | TIMP1 | NM_003254 | CLGI // EPA // | 3976341 | chrX | + | 47326654 | 47332978 | 1.08 |
| | | | EPO // FLJ90373 | | | | | | |
| | | | // HCI // TIMP | | | | | | |
| interleukin 1 receptor antagonist | IL1RN | NM_000577 // | ICIL-1RA // IL- | 2501204 | chr2 | + | 113573383 | 113608060 | 1.08 |
| | | NM_173841 // | 1ra3 // IL1F3 // | | | | | | |
| | | NM_173842 // | IL1RA // IRAP // | | | | | | |
| | | NM_173843 | MGC10430 | | | | | | |
| PRP40 pre-mRNA processing factor 40 | PRPF40A | XM_371575 // | FBP-11 // FBP11 | 2581548 | chr2 | − | 153212414 | 153283546 | 1.08 |
| homolog A (S. cerevisiae) | | XM_938514 | // FLAF1 // | | | | | | |
| | | | FLJ20585 // | | | | | | |
| | | | FNBP3 // HIP10 // | | | | | | |
| | | | HYPA // NY-REN- | | | | | | |
| ATP-binding cassette, sub-family A (ABC1), | ABCA1 | NM_005502 | ABC-1 // ABC1 // | 3218528 | chr9 | − | 106583121 | 106748222 | 1.08 |
| member 1 | | | CERP // FLJ14958 | | | | | | |
| | | | // HDLDT1 // TGD | | | | | | |
| RAB5A, member RAS oncogene family | RAB5A | NM_004162 | RAB5 | 2613386 | chr3 | + | 19963571 | 20002852 | 1.07 |
| protein phosphatase 2 (formerly 2A), catalytic | PPP2CA | NM_002715 | PP2Ac // PP2CA | 2876046 | chr5 | − | 133557588 | 133589841 | 1.07 |
| subunit, alpha isoform | | | // RP-C | | | | | | |
| membrane protein, palmitoylated 7 (MAGUK p55 | MPP7 | NM_173496 | FLJ32798 | 3282601 | chr10 | − | 28377816 | 28631969 | 1.07 |
| subfamily member 7) | | | | | | | | | |
| RAB6 interacting protein 1 | RAB6IP1 | NM_015213 | FLJ22354 // | 3362263 | chr11 | − | 9116953 | 9243468 | 1.07 |
| | | | FLJ33829 // | | | | | | |
| | | | FLJ43455 // | | | | | | |
| | | | KIAA1091 | | | | | | |
| GRB2-associated binding protein 2 | GAB2 | NM_012296 // | KIAA0571 | 3383227 | chr11 | − | 77603886 | 77989843 | 1.07 |
| | | NM_080491 | | | | | | | |
| chloride intracellular channel 4 // adducin 3 | CLIC4 // | NM_013943 // | CLIC4L // | 2325593 | chr1 | + | 24943202 | 25071376 | 1.07 |
| (gamma) | ADD3 | NM_001121 // | DKFZP566G223 // | | | | | | |
| | | NM_016824 // | FLJ38640 // H1 // | | | | | | |
| | | NM_019903 | huH1 // p64H1 // | | | | | | |
| | | | ADDL | | | | | | |
| TBC1 domain family, member 23 | TBC1D23 | NM_018309 | DKFZp667G062 // | 2633587 | chr3 | + | 101462368 | 101526762 | 1.07 |
| | | | FLJ11046 // | | | | | | |
| | | | NS4ATP1 | | | | | | |
| solute carrier family 16, member 3 | SLC16A3 | NM_001042422 // | MCT3 // MCT4 // | 3738629 | chr17 | + | 77775274 | 77812305 | 1.07 |
| (monocarboxylic acid transporter 4) | | NM_001042423 // | MGC138472 // | | | | | | |
| | | NM_004207 | MGC138474 | | | | | | |
| sestrin 3 | SESN3 | NM_144665 | MGC29667 // | 3387259 | chr11 | − | 94545767 | 94605309 | 1.07 |
| | | | SEST3 | | | | | | |
| nuclear RNA export factor 1 | NXF1 | NM_001081491 // | DKFZp667O0311 // | 3376155 | chr11 | − | 62316178 | 62329529 | 1.07 |
| | | NM_006362 | MEX67 // TAP | | | | | | |
| myristoylated alanine-rich protein kinase C | MARCKS | NM_002356 | 80K-L // FLJ14368 | 2922215 | chr6 | + | 114284762 | 114292853 | 1.07 |
| substrate | | | // FLJ90045 // | | | | | | |
| | | | MACS // MRACKS | | | | | | |
| | | | // PKCSL // | | | | | | |
| | | | PRKCSL | | | | | | |
| retinitis pigmentosa 2 (X-linked recessive) | RP2 | NM_006915 | KIAA0215 // | 3975869 | chrX | + | 46581319 | 46637601 | 1.07 |
| | | | TBCCD2 | | | | | | |
| DnaJ (Hsp40) homolog, subfamily C, member 3 | DNAJC3 | NM_006260 | HP58 // P58 // | 3497270 | chr13 | + | 95125276 | 95245596 | 1.07 |
| | | | P58IPK // PRKRI | | | | | | |
| interleukin 1, alpha | IL1A | NM_000575 | IL-1A // IL1 // | 2571483 | chr2 | − | 113247977 | 113259446 | 1.07 |
| | | | IL1-ALPHA // | | | | | | |
| TANK-binding kinase 1 | TBK1 | NM_013254 | FLJ11330 // NAK | 3419849 | chr12 | + | 63132014 | 63202097 | 1.07 |
| | | | // T2K | | | | | | |
| adaptor-related protein complex 3, beta 1 | AP3B1 | NM_003664 | ADTB3 // ADTB3A | 2863730 | chr5 | − | 77318390 | 77671471 | 1.07 |
| subunit | | | // HPS // HPS2 // | | | | | | |
| | | | PE | | | | | | |
| ATPase type 13A3 | ATP13A3 | XM_931948 // | AFURS1 // | 2711644 | chr3 | − | 195604703 | 195757213 | 1.07 |
| | | XM_942079 | DKFZp686K16189 | | | | | | |
| | | | // FLJ90613 | | | | | | |
| coiled-coil domain containing 131 | CCDC131 | NM_144982 | DKFZp686A0722 // | 3462094 | chr12 | − | 70269806 | 70344289 | 1.07 |
| | | | KIAA0546 // | | | | | | |
| | | | MGC23401 // | | | | | | |
| | | | MGC90200 // | | | | | | |
| | | | PSRC2 | | | | | | |
| calmodulin 2 (phosphorylase kinase, delta) | CALM2 | NM_001743 | CAMII // PHKD // | 2551924 | chr2 | − | 47232159 | 47286178 | 1.07 |
| | | | PHKD2 | | | | | | |
| ubiquitously transcribed tetratricopeptide | UTY // UTX | NM_007125 // | DKFZp686L12190 | 4035017 | chrY | − | 13772846 | 14101944 | 1.07 |
| repeat gene, Y-linked // ubiquitously | | NM_182659 // | // UTY1 // | | | | | | |
| transcribed tetratricopeptide repeat, X | | NM_182660 // | DKFZp686A03225 | | | | | | |
| chromosome | | NM_021140 | // MGC141941 // | | | | | | |
| | | | bA386N14.2 | | | | | | |
| brain abundant, membrane attached signal | BASP1 | NM_006317 | CAP-23 // CAP23 | 2803329 | chr5 | + | 17118727 | 17381190 | 1.07 |
| protein 1 | | | // MGC8555 // | | | | | | |
| | | | NAP-22 // NAP22 | | | | | | |
| GTP cyclohydrolase 1 (dopa-responsive | GCH1 | NM_000161 // | DYT5 // GCH // | 3565524 | chr14 | − | 54375952 | 54439279 | 1.07 |
| dystonia) | | NM_001024024 // | GTP-CH-1 // | | | | | | |
| | | NM_001024070 // | GTPCH1 | | | | | | |
| | | NM_001024071 | | | | | | | |
| proteasome (prosome, macropain) 26S subunit, | PSMD3 | NM_002809 | P58 // RPN3 // S3 | 3720636 | chr17 | + | 35390438 | 35407732 | 1.07 |
| non-ATPase, 3 | | | | | | | | | |
| acyl-CoA synthetase long-chain family member 1 | ACSL1 | NM_001995 | ACS1 // FACL1 // | 2796553 | chr4 | − | 185913758 | 186002178 | 1.07 |
| | | | FACL2 // LACS // | | | | | | |
| | | | LACS1 // LACS2 | | | | | | |
| numb homolog (Drosophila) | NUMB | NM_001005743 // | S171 | 3571347 | chr14 | − | 72811631 | 73000081 | 1.07 |
| | | NM_001005744 // | | | | | | | |
| | | NM_001005745 // | | | | | | | |
| | | NM_003744 | | | | | | | |
| protein kinase C, epsilon | PRKCE | NM_005400 | MGC125656 // | 2480168 | chr2 | + | 45731934 | 46343070 | 1.07 |
| | | | MGC125657 // | | | | | | |
| | | | PKCE // nPKC- | | | | | | |
| | | | epsilon | | | | | | |
| quaking homolog, KH domain RNA binding | QKI | XM_945803 // | DKFZp586I0923 // | 2935475 | chr6 | + | 163684268 | 163960462 | 1.07 |
| (mouse) | | NM_006775 // | Hqk // QK // QK3 | | | | | | |
| | | NM_206853 // | | | | | | | |
| | | NM_206854 // | | | | | | | |
| | | NM_206855 | | | | | | | |
| eukaryotic translation initiation factor 2C, 2 | EIF2C2 | NM_012154 | AGO2 // MGC3183 | 3156193 | chr8 | − | 141599440 | 141726037 | 1.07 |
| | | | // Q10 | | | | | | |
| CD2 molecule | CD2 | NM_001767 | SRBC // T11 | 2353669 | chr1 | + | 117098550 | 117149098 | 1.07 |
| nuclear factor of kappa light polypeptide gene | NFKB1 | NM_003998 | DKFZp686C01211 | 2737717 | chr4 | + | 103568522 | 103757506 | 1.07 |
| enhancer in B-cells 1 (p105) | | | // EBP-1 // KBF1 | | | | | | |
| | | | // MGC54151 // | | | | | | |
| | | | NF-kappa-B // | | | | | | |
| | | | NFKB-p105 // | | | | | | |
| transcription elongation factor B (SIII), | TCEB3 | NM_003198 | SIII // TCEB3A | 2325251 | chr1 | + | 23942459 | 23961135 | 1.07 |
| polypeptide 3 (110 kDa, elongin A) | | | | | | | | | |
| COP9 constitutive photomorphogenic homolog | COPS2 | NM_004236 | ALIEN // CSN2 // | 3623424 | chr15 | − | 47203877 | 47235198 | 1.07 |
| subunit 2 (Arabidopsis) | | | SGN2 // TRIP15 | | | | | | |
| pyridoxal (pyridoxine, vitamin B6) kinase // | PDXK // | NM_003681 // | C21orf97 // PKH // | 3923257 | chr21 | + | 43963416 | 44006601 | 1.07 |
| chromosome 21 open reading frame 124 | C21orf124 | NM_032920 | PNK // FLJ31940 | | | | | | |
| | | | // MGC15873 // | | | | | | |
| | | | PRED79 | | | | | | |
| nucleoporin 98 kDa | NUP98 | NM_005387 // | ADIR2 // NUP196 | 3359910 | chr11 | − | 3652836 | 3775353 | 1.07 |
| | | NM_016320 // | | | | | | | |
| | | NM_139131 // | | | | | | | |
| | | NM_139132 | | | | | | | |
| muskelin 1, intracellular mediator containing | MKLN1 | NM_013255 | FLJ11162 | 3024275 | chr7 | + | 130445315 | 130837940 | 1.07 |
| kelch motifs | | | | | | | | | |
| ceramide kinase | CERK | NM_022766 // | DKFZp434E0211 // | 3964154 | chr22 | − | 45454343 | 45537349 | 1.07 |
| | | NM_182661 | FLJ21430 // | | | | | | |
| | | | FLJ23239 // | | | | | | |
| | | | KIAA1646 // LK4 | | | | | | |
| | | | // MGC131878 // | | | | | | |
| | | | dA59H18.2 // | | | | | | |
| | | | dA59H18.3 // | | | | | | |
| | | | hCERK | | | | | | |
| non-POU domain containing, octamer-binding | NONO | NM_007363 | NMT55 // NRB54 | 3980887 | chrX | + | 70420006 | 70437726 | 1.07 |
| | | | // P54 // P54NRB | | | | | | |
| neuroepithelial cell transforming gene 1 | NET1 | NM_001047160 // | ARHGEF8 // | 3233182 | chr10 | + | 5444535 | 5491001 | 1.07 |
| | | NM_005863 | NET1A | | | | | | |
| mannosyl (alpha-1,3-)-glycoprotein beta-1,4- | MGAT4A // | NM_012214 // | GNT-IV // GNT- | 2566414 | chr2 | − | 98600132 | 98714000 | 1.07 |
| N-acetylglucosaminyltransferase, isozyme A // | MGC52110 | NM_001008215 | IVA // | | | | | | |
| hypothetical protein MGC52110 | | | 6330578E17Rik | | | | | | |
| protein phosphatase 1, regulatory (inhibitor) | PPP1R10 | NM_002714 | CAT53 // FB19 // | 2948425 | chr6 | − | 30676177 | 30694348 | 1.07 |
| subunit 10 | | | PNUTS | | | | | | |
| inositol polyphosphate-5-phosphatase, 40 kDa | INPP5A | XM_001133189 // | 5PTASE // | 3272205 | chr10 | + | 134201308 | 134446967 | 1.07 |
| | | NM_005539 | DKFZp434A1721 // | | | | | | |
| | | | MGC116947 // | | | | | | |
| | | | MGC116949 | | | | | | |
| plasminogen activator, urokinase receptor | PLAUR | NM_001005376 // | CD87 // UPAR // | 3864551 | chr19 | − | 48841865 | 48866539 | 1.06 |
| | | NM_001005377 // | URKR | | | | | | |
| | | NM_002659 | | | | | | | |
| PHD finger protein 12 | PHF12 | NM_001033561 // | KIAA1523 // | 3751184 | chr17 | − | 24256404 | 24302905 | 1.06 |
| | | NM_020889 | MGC131914 // PF1 | | | | | | |
| synapse associated protein 1, SAP47 homolog | SYAP1 | NM_032796 | DKFZp686K221 // | 3970130 | chrX | + | 16639861 | 16690708 | 1.06 |
| (Drosophila) | | | FLJ14495 // | | | | | | |
| | | | FLJ44185 // | | | | | | |
| | | | PRO3113 | | | | | | |
| chromosome 1 open reading frame 152 // | C1orf152 // | NR_003242 // | COAS3 // CMYA2 | 2431886 | chr1 | − | 142444924 | 143804054 | 1.06 |
| phosphodiesterase 4D interacting protein | PDE4DIP | XM_943179 // | // DKFZp781J054 | | | | | | |
| (myomegalin) | | NM_001002810 // | // MGC75440 // | | | | | | |
| | | NM_001002811 // | MMGL | | | | | | |
| | | NM_001002812 // | | | | | | | |
| | | NM_014644 // | | | | | | | |
| | | NM_022359 | | | | | | | |
| homocysteine-inducible, endoplasmic reticulum | HERPUD1 | NM_001010989 // | HERP // KIAA0025 | 3662387 | chr16 | + | 55523269 | 55552122 | 1.06 |
| stress-inducible, ubiquitin-like domain member 1 | | NM_001010990 // | // Mif1 // SUP | | | | | | |
| | | NM_014685 | | | | | | | |
| TNF receptor-associated factor 1 | TRAF1 | NM_005658 | EBI6 // MGC: 10353 | 3223738 | chr9 | − | 122699696 | 122731521 | 1.06 |
| pumilio homolog 2 (Drosophila) | PUM2 | NM_015317 | FLJ36528 // | 2542816 | chr2 | − | 20291440 | 20450197 | 1.06 |
| | | | KIAA0235 // | | | | | | |
| | | | MGC138251 // | | | | | | |
| | | | MGC138253 // | | | | | | |
| | | | PUMH2 // PUML2 | | | | | | |
| Dmx-like 2 | DMXL2 | NM_015263 | FLJ26672 // | 3624145 | chr15 | − | 49527223 | 49702258 | 1.06 |
| | | | KIAA0856 // RC3 | | | | | | |
| RAB8B, member RAS oncogene family | RAB8B | NM_016530 | FLJ38125 | 3597476 | chr15 | + | 61268620 | 61347018 | 1.06 |
| tight junction protein 2 (zona occludens 2) | TJP2 | NM_004817 // | MGC26306 // X104 | 3173880 | chr9 | + | 70925894 | 71059931 | 1.06 |
| | | NM_201629 | // ZO-2 // ZO2 | | | | | | |
| BCL2/adenovirus E1B 19 kDa interacting | BNIP2 | NM_004330 | BNIP-2 // NIP2 | 3627076 | chr15 | − | 57742025 | 57768916 | 1.06 |
| protein 2 | | | | | | | | | |
| sorting nexin 9 // synaptojanin 2 | SNX9 // | NM_016224 // | MST155 // | 2933331 | chr6 | + | 158164282 | 158286097 | 1.06 |
| | SYNJ2 | NM_003898 | MSTP155 // SDP1 | | | | | | |
| | | | // SH3PX1 // | | | | | | |
| | | | SH3PXD3A // WISP | | | | | | |
| | | | // INPP5H // | | | | | | |
| | | | KIAA0348 // | | | | | | |
| | | | MGC44422 | | | | | | |
| programmed cell death 4 (neoplastic | PDCD4 | NM_014456 // | H731 // MGC33046 | 3263944 | chr10 | + | 112621575 | 112649753 | 1.06 |
| transformation inhibitor) | | NM_145341 | // MGC33047 | | | | | | |
| leupaxin | LPXN | NM_004811 | LDPL | 3374402 | chr11 | − | 58050925 | 58102459 | 1.06 |
| solute carrier family 7 (cationic amino acid | SLC7A5 // | NM_003486 // | 4F2LC // CD98 // | 3703885 | chr16 | − | 86421130 | 86460615 | 1.06 |
| transporter, y+ system), member 5 // SLC7A5 | LAT1-3TM | NR_002593 | D16S469E // E16 | | | | | | |
| pseudogene | | | // LAT1 // MPE16 | | | | | | |
| | | | // hLAT1 // DC49 | | | | | | |
| | | | // MLAS // | | | | | | |
| | | | hLAT1-3TM | | | | | | |
| heat shock protein 90 kDa beta (Grp94), member | HSP90B1 // | NM_003299 // | ECGP // GP96 // | 3429312 | chr12 | + | 102848291 | 102871551 | 1.06 |
| 1 // thymine-DNA glycosylase // heat shock | TDG // | NM_003211 // — | GRP94 // TRA1 // | | | | | | |
| protein 90 kDa beta (Grp94), member 2 | HSP90B2P | | —// GRP94P1 // | | | | | | |
| (pseudogene) | | | GRP94b // HSP // | | | | | | |
| | | | HSPCP2 // | | | | | | |
| | | | HsGrp94b // | | | | | | |
| | | | TRA1P1 // TRAP1 | | | | | | |
| TNFAIP3 interacting protein 1 | TNIP1 | NM_006058 | ABIN-1 // | 2881672 | chr5 | − | 150389707 | 150446947 | 1.06 |
| | | | KIAA0113 // NAF1 | | | | | | |
| | | | // VAN | | | | | | |
| NECAP endocytosis associated 2 | NECAP2 | NM_018090 | FLJ10420 // RP4- | 2322389 | chr1 | + | 16639774 | 16659570 | 1.06 |
| | | | 798A10.1 | | | | | | |
| proteasome (prosome, macropain) subunit, alpha | PSMA5 | NM_002790 | MGC117302 // | 2427074 | chr1 | − | 109742573 | 109770611 | 1.06 |
| type, 5 | | | MGC125802 // | | | | | | |
| | | | MGC125803 // | | | | | | |
| | | | MGC125804 // | | | | | | |
| | | | PSC5 // ZETA | | | | | | |
| guanine nucleotide binding protein-like 2 | GNL2 | NM_013285 | FLJ40906 // | 2407191 | chr1 | − | 37805043 | 37834120 | 1.06 |
| (nucleolar) | | | HUMAUANTIG // | | | | | | |
| | | | NGP1 // Ngp-1 // | | | | | | |
| | | | dJ423B22.6 | | | | | | |
| GRIP and coiled-coil domain containing 2 | GCC2 | NM_014635 // | GCC185 // | 2498977 | chr2 | + | 108431315 | 108492470 | 1.06 |
| | | NM_181453 | KIAA0336 | | | | | | |
| RAD50 homolog (S. cerevisiae) | RAD50 | NM_005732 // | RAD50-2 // | 2828564 | chr5 | + | 131774479 | 132009928 | 1.06 |
| | | NM_133482 | hRad50 | | | | | | |
| fibronectin type III domain containing 3B | FNDC3B | NM_022763 | DKFZp686D14170 | 2652410 | chr3 | + | 173240112 | 173601176 | 1.06 |
| | | | // DKFZp762K137 | | | | | | |
| | | | // FAD104 // | | | | | | |
| | | | FLJ23399 // | | | | | | |
| | | | MGC10002 // | | | | | | |
| | | | PRO4979 // | | | | | | |
| | | | YVTM2421 | | | | | | |
| son of sevenless homolog 2 (Drosophila) | SOS2 | NM_006939 | — | 3563734 | chr14 | − | 49654812 | 49768331 | 1.06 |
| heat shock protein 90 kDa alpha (cytosolic), | HSP90AA1 // | NM_001017963 // | FLJ31884 // HSP86 | 3580179 | chr14 | − | 101616842 | 101676327 | 1.06 |
| class A member 1 // heat shock protein 90 kDa | HSP90AA2 // | NM_005348 // | // HSP90A // | | | | | | |
| alpha (cytosolic), class A member 2 // heat | HSP90AA6P | NM_001040141 // — | HSP90N // HSPC1 | | | | | | |
| shock protein 90 kDa alpha (cytosolic), class A | | | // HSPCA // | | | | | | |
| member 6 (pseudogene) | | | HSPCAL1 // | | | | | | |
| | | | HSPCAL4 // HSPN | | | | | | |
| | | | // Hsp89 // Hsp90 | | | | | | |
| | | | // LAP2 // | | | | | | |
| | | | HSP90ALPHA // | | | | | | |
| | | | HSPCAL3 // | | | | | | |
| | | | HSP90Af | | | | | | |
| phosphodiesterase 4B, cAMP-specific | PDE4B | NM_001037339 // | DKFZp686F2182 // | 2340529 | chr1 | + | 66030487 | 66612844 | 1.06 |
| (phosphodiesterase E4 dunce komolog, | | NM_001037340 // | DPDE4 // | | | | | | |
| Drosophila) | | NM_001037341 // | MGC126529 // | | | | | | |
| | | NM_002600 | PDEIVB | | | | | | |
| interleukic-1 receptor-associated kinase 2 | IRAK2 | NM_001570 | IRAK-2 // | 2610359 | chr3 | + | 10181579 | 10260427 | 1.06 |
| | | | MGC150550 | | | | | | |
| chromosome 14 open reading frame 32 | C14orf32 | NM_144578 | MGC23138 // MISS | 3536663 | chr14 | + | 54588114 | 54606655 | 1.06 |
| | | | // c14_5346 | | | | | | |
| nucleoside phosphorylase | NP | NM_000270 | MGC117396 // | 3527514 | chr14 | + | 20007398 | 20015985 | 1.06 |
| | | | MGC125915 // | | | | | | |
| | | | MGC125916 // PNP | | | | | | |
| | | | // PRO1837 // | | | | | | |
| | | | PUNP | | | | | | |
| proline-serine-threonine phosphatase | PSTPIP2 | NM_024430 | MAYP // | 3806211 | chr18 | − | 41817502 | 41906221 | 1.06 |
| interacting protein 2 | | | MGC34175 | | | | | | |
| growth factor receptor-bound protein 2 | GRB2 | NM_002086 // | ASH // EGFRBP- | 3770743 | chr17 | − | 70805871 | 70913384 | 1.06 |
| | | NM_203506 | GRB2 // Grb3-3 // | | | | | | |
| | | | MST084 // | | | | | | |
| | | | MSTP084 | | | | | | |
| signal-induced proliferation-associated 1 like 1 | SIPA1L1 | NM_015556 | DKFZp686G1344 // | 3542847 | chr14 | + | 70857592 | 71468470 | 1.06 |
| | | | E6TP1 // KIAA0440 | | | | | | |
| exportin 1 (CRM1 homolog, yeast) // thyroid | XPO1 // THRB | NM_003400 // | CRM1 // | 2555490 | chr2 | − | 61558490 | 61619343 | 1.06 |
| hormone receptor, beta (erythroblastic leukemia | | NM_000461 | DKFZp686B1823 // | | | | | | |
| viral (v-erb-a) oncogene homolog 2, avian) | | | ERBA-BETA // | | | | | | |
| | | | ERBA2 // GRTH // | | | | | | |
| | | | MGC126109 // | | | | | | |
| | | | MGC126110 // | | | | | | |
| | | | NR1A2 // THR1 // | | | | | | |
| | | | THRB1 // THRB2 | | | | | | |
| centromere protein C 1 | CENPC1 | NM_001812 | CENP-C // CENPC | 2771654 | chr4 | − | 68020183 | 68093837 | 1.06 |
| | | | // MIF2 // hcp-4 | | | | | | |
| v-abl Abelson murine leukemia viral oncogene | ABL2 | NM_005158 // | ABLL // ARG | 2446047 | chr1 | − | 177335093 | 177528267 | 1.06 |
| homolog 2 (arg, Abelson-related gene) | | NM_007314 | | | | | | | |
| rine finger protein 10 | RNF10 | NM_014868 | KIAA0262 // | 3434413 | chr12 | + | 119456489 | 119500226 | 1.06 |
| | | | MGC126758 // | | | | | | |
| | | | MGC126764 // | | | | | | |
| Notch homolog 2 (Drosophila) // neuroblastoma | NOTCH2 // | XM_001133349 // | AGS2 // hN2 // | 2431112 | chr1 | − | 120205744 | 120426930 | 1.06 |
| breakpoint family, member 14 | NBPF14 | NM_024408 // | DJ328E19.C1.1 // | | | | | | |
| | | NM_015383 | FLJ35032 // NBPF | | | | | | |
| endothelin converting enzyme 1 // amyloid beta | ECE1 // | NM_001397 // | ECE // FE65L // | 2400518 | chr1 | − | 21394307 | 21544720 | 1.06 |
| (A4) precursor protein-binding, family B, | APBB2 | NM_173075 | FE65L1 // | | | | | | |
| member 2 (Fe65-like) | | | MGC35575 | | | | | | |
| phosphoinositide-3-kinase adaptor protein 1 | PIK3AP1 | NM_152309 | BCAP // RP11- | 3301914 | chr10 | − | 98342808 | 98470259 | 1.06 |
| | | | 34E5.3 | | | | | | |
| BTB and CNC homology 1, basic leucine zipper | BACH2 | NM_021813 | — | 2964553 | chr6 | − | 90692975 | 91063182 | 1.06 |
| transcription factor 2 | | | | | | | | | |
| ATPase, H+ transporting, lysosomal 56/58 kDa, | ATP6V1B2 | NM_001693 | ATP6B1B2 // | 3088544 | chr8 | + | 20098984 | 20143098 | 1.06 |
| V1 subunit B2 | | | ATP6B2 // HO57 | | | | | | |
| | | | // VATB // VPP3 | | | | | | |
| | | | // Vma2 | | | | | | |
| TRAF2 and NCK interacting kinase | TNIK | NM_015028 | AD 2 | 2705266 | chr3 | − | 172258898 | 172660964 | 1.06 |
| AT-hook transcription factor | AKNA | NM_030767 | KIAA1968 // RP11- | 3221916 | chr9 | − | 116138238 | 116200584 | 1.06 |
| | | | 82I1.4 | | | | | | |
| Fas (TNF receptor superfamily, member 6) | FAS | NM_000043 // | ALPS1A // APO-1 | 3257098 | chr10 | + | 90721519 | 90765521 | 1.06 |
| | | NM_152871 // | // APT1 // CD95 | | | | | | |
| | | NM_152872 // | // FAS1 // FASTM | | | | | | |
| | | NM_152873 // | // TNFRSF6 | | | | | | |
| | | NM_152874 // | | | | | | | |
| | | NM_152875 // | | | | | | | |
| | | NM_152876 // | | | | | | | |
| | | NM_152877 | | | | | | | |
| ankyrin repeat domain 17 | ANKRD17 | NM_032217 // | FLJ22206 // GTAR | 2773023 | chr4 | − | 74158547 | 74343428 | 1.06 |
| | | NM_198889 | // KIAA0697 // | | | | | | |
| | | | NY-BR-16 | | | | | | |
| clathrin interactor 1 | CLINT1 | NM_014666 | CLINT // ENTH // | 2883609 | chr5 | − | 157145339 | 157284818 | 1.06 |
| | | | EPN4 // EPNR // | | | | | | |
| | | | EPSINR // | | | | | | |
| | | | KIAA0171 | | | | | | |
| PRP8 pre-mRNA processing factor 8 homolog | PRPF8 | NM_006445 | HPRP8 // PRP8 // | 3740479 | chr17 | − | 1500681 | 1534906 | 1.06 |
| (S. cerevisiae) | | | PRPC8 // RP13 | | | | | | |
| AF4/FMR2 family, member 4 | AFF4 | NM_014423 | AF5Q31 // MCEF | 2875555 | chr5 | − | 132238768 | 132327205 | 1.06 |
| | | | // MGC75036 | | | | | | |
| v-rel reticuloendotheliosis viral oncogene | REL | NM_002908 | C-Rel | 2484358 | chr2 | + | 60962266 | 61004124 | 1.06 |
| homolog (avian) | | | | | | | | | |
| excision repair cross-complementing rodent | ERCC1 | NM_001983 // | UV20 | 3865378 | chr19 | − | 50602433 | 50673926 | 1.06 |
| repair deficiency, complementation group 1 | | NM_202001 | | | | | | | |
| (includes overlapping antisense sequence) | | | | | | | | | |
| ubiquitously transcribed tetratricopeptide | UTX // UTY | NM_021140 // | DKFZp686A03225 | 3975467 | chrX | + | 44590716 | 44896185 | 1.06 |
| repeat, X chromosome // ubiquitously | | NM_007125 // | // MGC141941 // | | | | | | |
| transcribed tetratricopeptide repeat gene, Y- | | NM_182659 // | bA386N14.2 // | | | | | | |
| linked | | NM_182660 | DKFZp686L12190 | | | | | | |
| | | | // UTY1 | | | | | | |
| ATPase, Ca++ transporting, plasma membrane 1 | ATP2B1 | NM_001001323 // | PMCA1 | 3464983 | chr12 | − | 88505965 | 88620030 | 1.06 |
| | | NM_001682 | | | | | | | |
| v-ets erythroblastosis virus E26 oncogene | ETS2 | NM_005239 | — | 3921068 | chr21 | + | 39059147 | 39118744 | 1.06 |
| homolog 2 (avian) | | | | | | | | | |
| zinc finger protein 91 homolog (mouse) // | ZFP91 // | NM_053023 // | FKSG11 // PZF // | 3331730 | chr11 | + | 58101868 | 58149782 | 1.06 |
| ciliary neurotrophic factor | CNTF | NM_170768 // | ZNF757 // HCNTF | | | | | | |
| | | NM_000614 | | | | | | | |
| integrin, alpha 5 (fibronectin receptor, alpha | ITGA5 | NM_002205 | CD49e // FNRA // | 3456732 | chr12 | − | 53075332 | 53099491 | 1.06 |
| polypeptide) | | | VLA5A | | | | | | |
| IBR domain containing 3 | IBRDC3 | NM_153341 | FLJ90005 | 2405312 | chr1 | − | 33172120 | 33203343 | 1.06 |
| cofactor required for Sp1 transcriptional | CRSP2 | XM_001126052 // | CRSP150 // CSRP | 4005644 | chrX | − | 40392502 | 40480045 | 1.06 |
| activation, subunit 2, 150 kDa | | NM_004229 | // CXorf4 // | | | | | | |
| | | | DRIP150 // EXLM1 | | | | | | |
| | | | // MED14 // | | | | | | |
| | | | MGC104513 // | | | | | | |
| | | | RGR1 // TRAP170 | | | | | | |
| aryl hydrocarbon receptor | AHR | NM_001621 | — | 2991233 | chr7 | + | 17304771 | 17363729 | 1.06 |
| solute carrier family 43, member 2 | SLC43A2 | NM_152346 | FLJ23848 // LAT4 | 3740367 | chr17 | − | 1423948 | 1478920 | 1.06 |
| | | | // MGC34680 | | | | | | |
| chemokine (C-C motif) ligand 20 | CCL20 | NM_004591 | CKb4 // LARC // | 2530713 | chr2 | + | 228386802 | 228427090 | 1.06 |
| | | | MIP-3a // MIP3A | | | | | | |
| | | | // SCYA20 // | | | | | | |
| serpin peptidase inhibiter, clade B (ovalbumin), | SERPINB8 | NM_001031848 // | CAP2 // P18 | 3791996 | chr18 | + | 59787618 | 59823238 | 1.06 |
| member 8 | | NM_002640 // | | | | | | | |
| | | NM_198833 | | | | | | | |
| chaperonin containing TCP1, subunit 4 (delta) | CCT4 | NM_006430 | Cctd // | 2555630 | chr2 | − | 61939693 | 61970469 | 1.06 |
| | | | MGC126164 // | | | | | | |
| | | | MGC126165 // SRB | | | | | | |
| AP2 associated kinase 1 | AAK1 | NM_014911 | KIAA1048 // | 2558150 | chr2 | − | 69538639 | 69783824 | 1.06 |
| | | | MGC138170 | | | | | | |
| musculin (activated B-cell factor-1) | MSC | NM_005098 | ABF-1 // ABF1 // | 3140213 | chr8 | − | 72843959 | 73059105 | 1.06 |
| | | | MYOR | | | | | | |
| Yip1 domain family, member 5 | YIPF5 | NM_001024947 // | DKFZp313L2216 // | 2879509 | chr5 | − | 143463451 | 143565514 | 1.06 |
| | | NM_030799 | FinGER5 // SB140 | | | | | | |
| | | | // SMAP-5 // | | | | | | |
| | | | SMAP5 // YIP1A | | | | | | |
| START domain containing 7 | STARD7 | NM_020151 // | GTT1 | 2565143 | chr2 | − | 96214334 | 96238296 | 1.06 |
| | | NM_139267 | | | | | | | |
| ADP-ribosylation factor guanine nucleotide- | ARFGEF1 | NM_006421 | ARFGEP1 // BIG1 | 3139035 | chr8 | − | 68250009 | 68418446 | 1.06 |
| exchange factor 1(brefeldin A-inhibited) | | | // D730028O18Rik | | | | | | |
| | | | // DKFZP434L057 | | | | | | |
| | | | // P200 | | | | | | |
| myeloid/lymphoid or mixed-lineage leukemia 5 | MLL5 | NM_018682 // | FLJ10078 // | 3017547 | chr7 | + | 104436029 | 104542698 | 1.06 |
| (trithorax homolog, Drosophila) | | NM_182931 | FLJ14026 // | | | | | | |
| | | | HDCMC04P // | | | | | | |
| | | | MGC70452 | | | | | | |
| DEAD (Asp-Glu-Ala-Asp) box polypeptide 3, X- | DDX3X | XM_001129278 // | DBX // DDX14 // | 3974838 | chrX | + | 41055591 | 41108668 | 1.06 |
| linked | | NM_001356 | DDX3 // HLP2 | | | | | | |
| nibrin | NBN | NM_001024688 // | AT-V1 // AT-V2 | 3143970 | chr8 | − | 91014745 | 91066433 | 1.06 |
| | | NM_002485 | // ATV // | | | | | | |
| | | | FLJ10155 // | | | | | | |
| | | | MGC87362 // NBS | | | | | | |
| AT rich interactive domain 4B (RBP1-like) // | ARID4B // | NM_016374 // | BCAA // BRCAA1 | 2461786 | chr1 | − | 233396677 | 233558149 | 1.06 |
| RNA binding motif protein 34 | RBM34 | NM_031371 // | // DFZp313M2420 | | | | | | |
| | | NM_015014 | // MGC163290 // | | | | | | |
| | | | RBBP1L1 // | | | | | | |
| | | | RBP1L1 // SAP180 | | | | | | |
| | | | // KIAA0117 | | | | | | |
| ankyrin repeat domain 44 | ANKRD44 | NM_153697 | MGC21968 // | 2593464 | chr2 | − | 197512324 | 197901798 | 1.06 |
| | | | MGC70444 | | | | | | |
| RB1-inducible coiled-coil 1 | RB1CC1 | NM_014781 | CC1 // DRAGOU14 | 3135184 | chr8 | − | 53641628 | 93789545 | 1.06 |
| | | | // FIP200 | | | | | | |
| ubiquitin specific peptidase 12 | USP12 | NM_182488 | USP12L1 | 3506738 | chr13 | − | 26518485 | 26644264 | 1.06 |
| apoptosis inhibitor 5 | API5 | NM_006595 | AAC-11 // AAC11 | 3327906 | chr11 | + | 43290109 | 43322649 | 1.06 |
| | | | // API5L1 | | | | | | |
| HIR histone cell cycle regulation defective | HIRA | NM_003325 | DGCR1 // TUP1 // | 3952637 | chr22 | − | 17696576 | 17799452 | 1.06 |
| homolog A (S. cerevisiae) | | | TUPLE1 | | | | | | |
| guanylate binding protein 7 // guanylate binding | GBP7 // GBP2 | NM_207398 // | FLJ38822 // | 2421925 | chr1 | − | 89343675 | 89496682 | 1.06 |
| protein 2, interferon-inducible // guanylate | // GBP4 | NM_004120 // | GBP4L // —// | | | | | | |
| binding protein 4 | | NM_052941 | Mpa2 | | | | | | |
| CASP8 and FADD-like apoptosis regulator | CFLAR | NM_003879 | CASH // | 2522616 | chr2 | + | 201689135 | 201744701 | 1.06 |
| | | | CASP8AP1 // | | | | | | |
| | | | CLARP // Casper | | | | | | |
| | | | // FLAME // | | | | | | |
| | | | FLAME-1 // FLIP | | | | | | |
| | | | // I-FLICE // MRIT | | | | | | |
| | | | // USURPIN // c- | | | | | | |
| | | | FLIP // c-FLIPL // | | | | | | |
| human immunodeficiency virus type I enhancer | HIVEP2 | NM_006734 | HIV-EP2 // MBP-2 | 2977265 | chr6 | − | 143065515 | 143308841 | 1.06 |
| binding protein 2 | | | // MIBP1 | | | | | | |
| serpin peptidase inhibitor, clade B (ovalbumin), | SERPINB9 // | NM_004155 // | CAP-3 // CAP3 // | 2939034 | chr6 | − | 2832402 | 2858242 | 1.06 |
| member 9 // serpin peptidase inhibitor, clade B | SERPINB6 | NM_004568 | PI9 // CAP // | | | | | | |
| (ovalbumin), member 6 | | | DKFZp686I04222 // | | | | | | |
| | | | MGC111370 // | | | | | | |
| | | | MSTP057 // PI6 // | | | | | | |
| | | | PTI | | | | | | |
| myxovirus (influenza virus) resistance 2 (mouse) | MX2 | NM_002463 | MXB | 3922037 | chr21 | + | 41655820 | 41703177 | 1.06 |
| solute carrier family 25, member 37 | SLC25A37 | XM_001128238 // | HT015 // MFRN // | 3090006 | chr8 | + | 23439117 | 23485313 | 1.06 |
| | | XM_001128248 // | MSC // MSCP // | | | | | | |
| | | XM_001128255 // | PRO1278 // | | | | | | |
| | | XM_001128269 // | PRO1584 // | | | | | | |
| | | NM_016612 | PRO2217 | | | | | | |
| WW domain binding protein 11 | WBP11 | NM_016312 | DKFZp779M1063 // | 3445670 | chr12 | − | 14829481 | 14848171 | 1.06 |
| | | | NPWBP // SIPP1 | | | | | | |
| bromodomain adjacent to zinc finger domain, 1A | BAZ1A | NM_013448 // | ACF1 // | 3560711 | chr14 | − | 34291692 | 34414604 | 1.06 |
| | | NM_182648 | DKFZP586E0518 // | | | | | | |
| | | | FLJ14383 // | | | | | | |
| | | | WALp1 // | | | | | | |
| | | | WCRF180 // hACF1 | | | | | | |
| aquaporin 9 | AQP9 | NM_020980 | HsT17287 // SSC1 | 3595594 | chr15 | + | 56217766 | 56280468 | 1.06 |
| solute carrier family 39 (zinc transporter), | SLC39A8 | NM_022154 | BIGM103 // LZT- | 2779823 | chr4 | − | 103393022 | 103486067 | 1.06 |
| member 8 | | | Hs6 | | | | | | |
| FYN oncogene related to SRC, FGR, YES | FYN | NM_002037 // | MGC45350 // SLK | 2969886 | chr6 | − | 112088237 | 112301348 | 1.06 |
| | | NM_153047 // | // SYN | | | | | | |
| | | NM_153048 | | | | | | | |
| elongation factor, RNA polymerase II, 2 | ELL2 | NM_012081 | — | 2867873 | chr5 | − | 95246561 | 95323733 | 1.06 |
| Janus kinase 1 (a protein tyrosine kinase) | JAK1 | NM_002227 | JAK1A // JAK1B | 2416522 | chr1 | − | 65071504 | 65278035 | 1.06 |
| v-rel reticuloendotheliosis viral oncogene | RELB | NM_006509 | I-REL | 3835966 | chr19 | + | 50196537 | 50233287 | 1.06 |
| homolog B, nuclear factor of kappa light | | | | | | | | | |
| polypeptide gene enhancer is B-cells 3 (avian) | | | | | | | | | |
| chemokine (C-C motif) ligand 2 | CCL2 | NM_002982 | GDCF-2 // GDCF- | 7385547 | chr17 | + | 29513430 | 29608325 | 1.06 |
| | | | 2 HC11 // HC11 // | | | | | | |
| | | | HSMCR30 // MCAF | | | | | | |
| | | | // MCP-1 // MCP1 | | | | | | |
| | | | // MGC9434 // | | | | | | |
| | | | SCYA2 // SMC-CF | | | | | | |
| hexokinase 2 | HK2 | NM_000189 | DKFZp686M1669 // | 2489545 | chr2 | + | 74913290 | 74974310 | 1.06 |
| | | | HKII // HXK2 | | | | | | |
| ELK3, ETS-domain protein (SRF accessory | ELK3 | NM_005230 | ERP // NET // | 3427098 | chr12 | + | 95112269 | 95187725 | 1.06 |
| protein 2) | | | SAP2 | | | | | | |
| SNF1-like kinase | SNF1LK | NM_173354 | MSK // SIK | 3934111 | chr21 | − | 43637578 | 43705100 | 1.06 |
| lipopolysaccharide-induced TNF factor | LITAF | NM_004862 | CMT1C // | 3680434 | chr16 | − | 11549088 | 11599620 | 1.06 |
| | | | FLJ38636 // | | | | | | |
| | | | MGC116698 // | | | | | | |
| | | | MGC116700 // | | | | | | |
| | | | MGC116701 // | | | | | | |
| | | | MGC125274 // | | | | | | |
| | | | MGC125275 // | | | | | | |
| | | | MGC125276 // | | | | | | |
| | | | PIG7 // SIMPLE // | | | | | | |
| | | | TP53I7 | | | | | | |
| tRNA splicing endonuclease 54 homolog (S. cerevisiae) | TSEN54 | NM_207346 | FLJ37147 // | 3734865 | chr17 | + | 71022989 | 71032409 | 1.06 |
| | | | SEN54L // sen54 | | | | | | |
| chromodomain helicase DNA binding protein 6 | CHD6 | NM_032221 | CHD5 // KIAA1335 | 3906160 | chr20 | − | 39464169 | 39680547 | 1.06 |
| | | | // RIGB | | | | | | |
| chemokine (C—X—C motif) ligand 1 (melanoma | CXCL1 | NM_001511 | GRO1 // GROa // | 2731381 | chr4 | + | 74953973 | 74983954 | 1.06 |
| growth stimulating activity, alpha) | | | MGSA // MGSA | | | | | | |
| | | | alpha // MGSA-a | | | | | | |
| | | | // NAP-3 // | | | | | | |
| myotubularin related protein 3 | MTMR3 | NM_021090 // | FYVE-DSP1 // | 3942179 | chr22 | + | 28609182 | 28762301 | 1.06 |
| | | NM_153050 // | KIAA0371 // | | | | | | |
| | | NM_153051 | ZFYVE10 | | | | | | |
| adaptor-related protein complex 1, beta 1 | AP1B1 | NM_001127 // | ADTB1 // AP105A | 3956781 | chr22 | − | 28053592 | 28206955 | 1.06 |
| subunit | | NM_145730 | // BAM22 // | | | | | | |
| | | | CLAPB2 | | | | | | |
| actinin, alpha 1 | ACTN1 | NM_001102 | FLJ40884 | 3569814 | chr14 | − | 68409817 | 68515815 | 1.06 |
| mitogen-activated protein kinase kinase kinase | MAP4K4 | NM_004834 // | FLH21957 // | 2496727 | chr2 | + | 101600496 | 101877880 | 1.06 |
| kinase 4 | | NM_145686 // | FLJ10410 // | | | | | | |
| | | NM_145687 | FLJ20373 // | | | | | | |
| | | | FLJ90111 /// HGK | | | | | | |
| | | | // KIAA0687 // NIK | | | | | | |
| activated leukocyte cell adhesion molecule | ALCAM | NM_001627 | CD166 // FLJ38514 | 2634494 | chr3 | + | 106304077 | 106816151 | 1.06 |
| | | | // MEMD // | | | | | | |
| | | | MGC71733 | | | | | | |
| valosin-containing protein // Fanconi anemia, | VCP // | NM_007126 // | IBMPFD // | 3204404 | chr9 | − | 35026106 | 35062648 | 1.06 |
| complementation group G | FANCG | NM_004629 | MGC131997 // | | | | | | |
| | | | MGC148092 // | | | | | | |
| | | | MGC8560 // TERA | | | | | | |
| | | | // p97 // FAG // | | | | | | |
| | | | XRCC9 | | | | | | |
| heat shock 105 kDa/110 kDa protein 1 | HSPH1 | NM_006644 | DKFZp686M05240 | 3508330 | chr13 | − | 30558258 | 30651692 | 1.06 |
| | | | // HSP105 // | | | | | | |
| | | | HSP105A // | | | | | | |
| | | | HSP105B // | | | | | | |
| | | | KIAA0201 // NY- | | | | | | |
| | | | CO-25 | | | | | | |
| WAS protein family, member 2 | WASF2 | NM_006990 | SCAR2 // WAVE2 | 2403111 | chr1 | − | 27603331 | 27689256 | 1.06 |
| | | | // dJ393P12.2 | | | | | | |
| RAP1 interacting factor homolog (yeast) | RIF1 | NM_018151 | DKFZp781N1478 // | 2510485 | chr2 | + | 151974674 | 152072768 | 1.06 |
| | | | FLJ12870 | | | | | | |
| chromosome 20 open reading frame 77 | C20orf77 | NM_021215 | CREPT // | 3884450 | chr20 | + | 36095232 | 36189576 | 1.06 |
| | | | DKFZp434P0735 // | | | | | | |
| | | | FLJ44520 // | | | | | | |
| | | | dJ1057B20.2 | | | | | | |
| Wilms tumor 1 associated protein // acetyl- | WTAP // | NM_004906 // | DKFZp686F20131 | 2934089 | chr6 | + | 160066607 | 160097332 | 1.06 |
| Coenzyme A acetyltransferase 2 (acetoacetyl | ACAT2 | NM_152857 // | // KIAA0105 // | | | | | | |
| Coenzyme A thiolase) | | NM_152858 // | MGC3925 // — | | | | | | |
| | | NM_005891 | | | | | | | |
| proline-rich nuclear receptor coactivator 1 | PNRC1 | NM_006813 | B4-2 // PNAS-145 | 2916716 | chr6 | + | 89847048 | 89852045 | 1.06 |
| | | | // PROL2 // PRR2 | | | | | | |
| | | | // RP11-63L7.5 | | | | | | |
| jumonji domain containing 1C | JMJD1C | NM_004241 // | DKFZp761F0118 // | 3291682 | chr10 | − | 64589487 | 64951821 | 1.06 |
| | | NM_032776 | FLJ14374 // | | | | | | |
| | | | KIAA1380 // RP11- | | | | | | |
| | | | 10C13.2 // TRIP8 | | | | | | |
| ELL associated factor 1 | EAF1 | NM_033083 | — | 2612371 | chr3 | + | 15443374 | 15466824 | 1.06 |
| mitogen-activated protein kinase-activated | MAPKAPK2 | NM_004759 // | — | 2376922 | chr1 | + | 204924896 | 204974249 | 1.06 |
| protein kinase 2 | | NM_032960 | | | | | | | |
| importin 7 | IPO7 | NM_006391 | FLJ14581 // | 3319840 | chr11 | + | 9362702 | 9426296 | 1.06 |
| | | | MGC138673 // | | | | | | |
| | | | RANBP7 | | | | | | |
| eukaryotic translation initiation factor 2, subunit | EIF2S1 | NM_004094 | EIF-2 // EIF-2A // | 3541137 | chr14 | + | 66896518 | 66922983 | 1.06 |
| 1 alpha, 35 kDa | | | EIF-2alpha // EIF2 | | | | | | |
| | | | // EIF2A | | | | | | |
| CD82 molecule | CD82 | NM_001024844 // | 4F9 // C33 // | 3328520 | chr11 | + | 44543723 | 44598484 | 1.06 |
| | | NM_002231 | GR15 // IA4 // | | | | | | |
| | | | KAI1 // R2 // | | | | | | |
| | | | SAR2 // ST6 // | | | | | | |
| Wolf-Hirschhorn syndrome candidate 1-like 1 | WHSC1L1 | NM_017778 // | DKFZp667H044 // | 3131916 | chr8 | − | 38251717 | 38359646 | 1.06 |
| | | NM_023034 | FLJ20353 // | | | | | | |
| | | | MGC126766 // | | | | | | |
| | | | MGC142029 // | | | | | | |
| | | | NSD3 // pp14328 | | | | | | |
| ADP-ribosylation factor-like 8B | ARL8B | NM_018184 | ARL10C // | 2608765 | chr3 | + | 5138874 | 5197592 | 1.06 |
| | | | FLJ10702 // Gie1 | | | | | | |
| Rap guanine nucleotide exchange factor (GEF) 2 | RAPGEF2 | XM_376350 // | CNrasGEF // PDZ- | 2749699 | chr4 | + | 160171745 | 160501468 | 1.06 |
| | | XM_934714 // | GEF1 // PDZGEF1 | | | | | | |
| | | XM_934717 // | // RA-GEF // Rap | | | | | | |
| | | XM_934718 // | GEP // Rap-GEP | | | | | | |
| | | XM_944403 // | | | | | | | |
| | | XM_944410 // | | | | | | | |
| | | XM_944412 | | | | | | | |
| guanine nucleotide binding protein (G protein), | GNG2 | NM_053064 | — | 3535628 | chr14 | + | 51383702 | 51506715 | 1.06 |
| gamma 2 | | | | | | | | | |
| influenza virus NS1A binding protein | IVNS1ABP | NM_006469 // | DKFZp686K06216 | 2448073 | chr1 | − | 183532162 | 183553081 | 1.06 |
| | | NM_016389 | // FLARA3 // | | | | | | |
| | | | FLJ10069 // | | | | | | |
| | | | FLJ10411 // | | | | | | |
| | | | FLJ10962 // | | | | | | |
| | | | FLJ35593 // | | | | | | |
| | | | FLJ36593 // | | | | | | |
| | | | HSPC068 // | | | | | | |
| | | | KIAA0850 // ND1 | | | | | | |
| | | | // NS-1 // NS1- | | | | | | |
| | | | BP // NS1BP | | | | | | |
| ancient ubiquitous protein 1 // DEAQ box | AUP1 // DQX1 | NM_181575 // | —// FLJ23757 | 2560254 | chr2 | − | 74607294 | 74610455 | 1.06 |
| polypetide 1 (RNA-dependent ATPase) | | NM_133637 | | | | | | | |
| TERF1 (TRF1)-interacting nuclear factor 2 | TINF2 | NM_012461 | TIN2 | 3558012 | chr14 | − | 23776465 | 23781950 | 1.06 |
| N-myc downstream regulated gene 1 | NDRG1 | NM_006096 | CAP43 // CMT4D | 3154317 | chr8 | − | 134318628 | 134379306 | 1.06 |
| | | | // DRG1 // GC4 // | | | | | | |
| | | | HMSNL // NDR1 // | | | | | | |
| | | | NMSL // PROXY1 | | | | | | |
| | | | // RIT42 // RTP // | | | | | | |
| | | | TARG1 // TDD5 | | | | | | |
| kinesin 2 | KNS2 | NM_005552 // | KLC // KLC1 // | 3553872 | chr14 | + | 103160147 | 103247456 | 1.06 |
| | | NM_182923 | KNS2A // | | | | | | |
| | | | MGC15245 | | | | | | |
| CD44 molecule (Indian blood group) // | CD44 // | NM_000610 // | CDW44 // CSPG8 | 3326635 | chr11 | + | 35117003 | 35210509 | 1.06 |
| mitogen-activated protein kinase 10 | MAPK10 | NM_001001389 // | // ECMR-III // | | | | | | |
| | | NM_001001390 // | HCELL // IN // | | | | | | |
| | | NM_001001391 // | LHR // MC56 // | | | | | | |
| | | NM_001001392 // | MDU2 // MDU3 // | | | | | | |
| | | NM_002753 // | MGC10468 // MIC4 | | | | | | |
| | | NM_138980 // | // MUTCH-I // | | | | | | |
| | | NM_138981 // | Pgp1 // FLJ12099 | | | | | | |
| | | NM_138982 | // FLJ33785 // | | | | | | |
| | | | JNK3 // JNK3A // | | | | | | |
| | | | MGC50974 // | | | | | | |
| | | | PRKM10 // | | | | | | |
| | | | p493F12 // | | | | | | |
| mitogen-activated protein kinase kinase 1 | MAP2K1 | NM_002755 | MAPKK1 // MEK1 | 3598662 | chr15 | + | 64466253 | 64571690 | 1.06 |
| | | | // MKK1 // | | | | | | |
| | | | PRKMK1 | | | | | | |
| chromosome 16 open reading frame 72 | C16orf72 | NM_014117 | FLJ41272 // | 3647632 | chr16 | + | 9092682 | 9121055 | 1.06 |
| | | | PRO0149 | | | | | | |
| tumor necrosis factor receptor superfamily, | TNFRSF1B | NM_001066 | CD120b // TBPII // | 2320727 | chr1 | + | 12149647 | 12192323 | 1.06 |
| member 1B | | | TNF-R-II // TNF- | | | | | | |
| | | | R75 // TNFBR // | | | | | | |
| | | | TNFR2 // TNFR80 | | | | | | |
| | | | // p75 // p75TNFR | | | | | | |
| protein phosphatase 2, regulatory subunit B′, | PPP2R5C | NM_002719 // | B56G // MGC23064 | 3552729 | chr14 | + | 101296771 | 101464066 | 1.06 |
| gamma isoform | | NM_178586 // | // PR61G | | | | | | |
| | | NM_178587 // | | | | | | | |
| | | NM_178588 | | | | | | | |
| TSPY-like 2 // paraoxonase 3 | TSPYL2 // | XM_001128695 // | CDA1 // CINAP // | 3978169 | chrX | + | 53124038 | 53134445 | 1.06 |
| | PON3 | NM_022117 // | CTCL // DENTT // | | | | | | |
| | | NM_000940 | HRIHFB2216 // | | | | | | |
| | | | SE20-4 // — | | | | | | |
| serine/threonine kinase 17a | STK17A | NM_004760 | DRAK1 | 2999485 | chr7 | + | 43573961 | 43642649 | 1.06 |
| ELAV (embryonic lethal, abnormal vision, | ELAVL1 // | NM_001419 // | ELAV1 // HUR // | 3848689 | chr19 | − | 7929463 | 7999655 | 1.06 |
| Drosophila)-like 1 (Hu antigen R) // translocase | TIMM44 | NM_006351 | Hua // MelG // | | | | | | |
| of inner mitochondrial membrane 44 homolog | | | DKFZp686H05241 | | | | | | |
| (yeast) | | | // TIM44 | | | | | | |
| basic leucine zipper and W2 domains 1 | BZW1 | XM_001126385 // | BZAP45 // | 2522439 | chr2 | + | 201384456 | 201418386 | 1.06 |
| | | NM_014670 | KIAA0005 // | | | | | | |
| | | | Nbia10236 | | | | | | |
| fibronectin type III domain containing 3A // | FNDC3A // | NM_001079673 // | FLJ31509 // | 3489212 | chr13 | + | 48428770 | 48681909 | 1.06 |
| cancer/testis antigen CT45-3 | RP13-36C9.3 | NM_014923 // | FNDC3 // | | | | | | |
| | | NM_001017435 | KIAA0970 // | | | | | | |
| | | | bA203I16.1 // | | | | | | |
| | | | bA203I16.5 // | | | | | | |
| | | | CT45-3 // RP13- | | | | | | |
| MAX dimerization protein 1 | MXD1 | NM_002357 | MAD // MAD1 // | 2487549 | chr2 | + | 69995330 | 70023575 | 1.06 |
| | | | MGC104659 | | | | | | |
| TIA1 cytotoxic granule-associated RNA binding | TIAL1 | NM_001033925 // | MGC33401 // | 3309629 | chr10 | − | 121291481 | 121346521 | 1.06 |
| protein-like 1 | | NM_003252 | TCBP // TIAR | | | | | | |
| nuclear receptor subfamily 4, group A, member 3 | NR4A3 | NM_006981 // | CHN // CSMF // | 3181976 | chr9 | + | 101623410 | 101669157 | 1.06 |
| | | NM_173198 // | MINOR // NOR1 // | | | | | | |
| | | NM_173199 // | TEC | | | | | | |
| | | NM_173200 | | | | | | | |
| SAM domain, SH3 domain and nuclear | SAMSN1 | NM_022136 | HACS1 // NASH1 | 3925473 | chr21 | − | 14779427 | 14877594 | 1.06 |
| localization signals 1 | | | | | | | | | |
| sperm specific antigen 2 | SSFA2 | NM_006751 | CS-1 // CS1 // | 2518428 | chr2 | + | 182464815 | 182516732 | 1.06 |
| | | | DKFZp313O1039 // | | | | | | |
| | | | DKFZp779G0129 // | | | | | | |
| | | | FLJ45996 // | | | | | | |
| | | | KIAA1927 // KRAP | | | | | | |
| | | | // SPAG13 | | | | | | |
| zinc finger protein 317 // zinc finger protein | ZNF317 // | NM_020933 // | KIAA1588 // | 3819880 | chr19 | + | 9112073 | 9135082 | 1.06 |
| 658 | ZNF658 | XM_001125688 // | DKFZp572C163 // | | | | | | |
| | | NM_033160 | FLJ32813 // | | | | | | |
| | | | MGC35232 | | | | | | |
| vacuolar protein sorting 35 homolog (S. cerevisiae) | VPS35 | NM_018206 | DKFZp434E1211 // | 3689922 | chr16 | − | 45220566 | 45280598 | 1.06 |
| | | | DKFZp434P1672 // | | | | | | |
| | | | FLJ10752 // | | | | | | |
| | | | FLJ13588 // | | | | | | |
| | | | FLJ20388 // MEM3 | | | | | | |
| CD8a molecule | CD8A | NM_001768 // | CD8 // Leu2 // | 2562932 | chr2 | − | 86857923 | 86889020 | 1.06 |
| | | NM_171827 | MAL // p32 | | | | | | |
| general transcription factor IIB | GTF2B // | NM_001514 | TF2B // TFIIB | 2421753 | chr1 | − | 89090909 | 89130195 | 1.06 |
| | RP11-82K18.3 | | | | | | | | |
| ring finger and WD repeat domain 2 | RFWD2 | NM_001001740 // | COP1 // FLJ10416 | 2445141 | chr1 | − | 174150358 | 174443232 | 1.06 |
| | | NM_022457 | // RNF200 | | | | | | |
| formin binding protein 1 | FNBP1 | NM_015033 | FBP17 // KIAA0554 | 3227159 | chr9 | − | 131689314 | 131845274 | 1.06 |
| | | | // MGC126804 | | | | | | |
| transmembrane protein 23 | TMEM23 | NM_147156 | MGC17342 // MOB | 3289235 | chr10 | − | 51706090 | 52054733 | 1.06 |
| | | | // MOB1 // SMS1 | | | | | | |
| hypoxia-inducible factor 1, alpha subunit (basic | HIF1A | NM_001530 // | HIF-1alpha // | 3539070 | chr14 | + | 61225495 | 61285222 | 1.06 |
| helix-loop-helix transcription factor) | | NM_181054 | HIF1-ALPHA // | | | | | | |
| | | | MOP1 // PASD8 | | | | | | |
| annexin A5 | ANXA5 | NM_001154 | ANX5 // ENX2 // | 2784027 | chr4 | − | 122695163 | 122838353 | 1.06 |
| | | | PP4 | | | | | | |
| T-cell activation GTPase activating protein | TAGAP | NM_054114 // | FKSG15 // | 2982076 | chr6 | − | 159375488 | 159517834 | 1.06 |
| | | NM_138810 // | FLJ32631 // | | | | | | |
| | | NM_152133 | FLJ39771 // | | | | | | |
| | | | MGC133247 // | | | | | | |
| | | | MGC27381 // | | | | | | |
| | | | TAGAP1 | | | | | | |
| dolichyl-phosphate mannosyltransferase | DPM1 | NM_003859 | CDGIE // MPDS | 3909395 | chr20 | − | 48984632 | 49008494 | 1.06 |
| polypeptide 1, catalytic subunit | | | | | | | | | |
| TIA1 cytotoxic granule-associated RNA binding | TIA1 | NM_022037 // | — | 2558511 | chr2 | − | 70290081 | 70329315 | 1.06 |
| protein | | NM_022173 | | | | | | | |
| CD83 molecule | CD83 | NM_001040280 // | BL11 // HB15 | 2895841 | chr6 | + | 14118209 | 14485106 | 1.06 |
| | | NM_004233 | | | | | | | |
| serine/arginine repetitive matrix 2 | SRRM2 | NM_016333 | 300-KD // | 3645253 | chr16 | + | 2742650 | 2761908 | 1.06 |
| | | | DKFZp686O15166 | | | | | | |
| | | | // FLJ21926 // | | | | | | |
| | | | FLJ22250 // | | | | | | |
| | | | KIAA0324 // | | | | | | |
| | | | MGC40295 // | | | | | | |
| | | | SRL300 // SRm300 | | | | | | |
| mitogen-activated protein kinase kinase 3 | MAP2K3 | XM_001130488 // | MAPKK3 // MEK3 | 3714729 | chr17 | + | 21128581 | 21159113 | 1.06 |
| | | NM_145110 // | // MKK3 // | | | | | | |
| | | NM_002756 // | PRKMK3 | | | | | | |
| | | NM_145109 | | | | | | | |
| mitogen-activated protein kinase kinase 1 | MAP2K1IP1 | NM_021970 | MP1 | 2779408 | chr4 | − | 101018538 | 101034726 | 1.06 |
| interacting protein 1 | | | | | | | | | |
| hypothetical protein FLJ31951 | FLJ31951 | NM_144726 | DKFZp686M11215 | 2884216 | chr5 | − | 158516997 | 158569129 | 1.06 |
| capping protein (actin filament) muscle Z-line, | CAPZA1 | NM_006135 | CAPPA1 // CAPZ | 2352228 | chr1 | + | 112945824 | 113015736 | 1.06 |
| alpha 1 | | | // CAZ1 | | | | | | |
| chemokine (C—X—C motif) ligand 10 | CXCL10 | NM_001565 | C7 // IFI10 // | 2773958 | chr4 | − | 77151393 | 77163693 | 1.06 |
| | | | INP10 // IP-10 // | | | | | | |
| | | | SCYB10 // crg-2 | | | | | | |
| | | | // gIP-10 // mob-1 | | | | | | |
| v-raf murine sarcoma 3611 viral oncogene | ARAF | NM_001654 | A-RAF // ARAF1 | 3976299 | chrX | + | 47300826 | 47316249 | 1.06 |
| homolog | | | // PKS2 // RAFA1 | | | | | | |
| ADAM metallopeptidase domain 17 (tumor | ADAM17 | NM_003183 | CD156b // | 2539821 | chr2 | − | 9546070 | 9630637 | 1.06 |
| necrosis factor, alpha, converting enzyme) | | | MGC71942 // | | | | | | |
| | | | TACE // cSVP | | | | | | |
| protein tyrosine phosphatase type IVA, member 1 | PTP4A1 | NM_003463 | DKFZp779M0721 // | 2911903 | chr6 | + | 64289625 | 64351448 | 1.06 |
| | | | HH72 // PRL-1 // | | | | | | |
| | | | PRL1 // | | | | | | |
| | | | PTP(CAAX1) // | | | | | | |
| | | | PTPCAAX1 | | | | | | |
| poly (ADP-ribose) polymerase family, member 9 | PARP9 | NM_031458 | BAL // BAL1 // | 2692060 | chr3 | − | 123729469 | 123766193 | 1.06 |
| | | | DKFZp666B0810 // | | | | | | |
| | | | DKFZp686M15238 | | | | | | |
| | | | // FLJ26637 // | | | | | | |
| | | | FLJ41418 // | | | | | | |
| | | | MGC: 7868 | | | | | | |
| WD repeat domain 74 | WDR74 | XM_001125771 // | FLJ10439 // | 3376235 | chr11 | − | 62356627 | 62365857 | 1.06 |
| | | NM_018093 | FLJ21730 | | | | | | |
| KIAA0256 gene product // trafficking protein | KIAA0256 // | NM_014701 // | —// MGC52424 | 3623320 | chr15 | − | 47055671 | 47126043 | 1.06 |
| particle complex 5 | TRAPPC5 | NM_001042461 // | | | | | | | |
| | | NM_001042462 // | | | | | | | |
| | | NM_174894 | | | | | | | |
| CD47 molecule | CD47 | NM_001025079 // | IAP // MER6 // | 2687739 | chr3 | − | 109143203 | 109306052 | 1.06 |
| | | NM_001777 // | OA3 | | | | | | |
| | | NM_198793 | | | | | | | |
| inositol polyphosphate-5-phosphatase, 145 kDa | INPP5D | XM_929960 // | MGC104855 // | 2532699 | chr2 | + | 233632835 | 233823600 | 1.06 |
| | | NM_001017915 // | MGC142140 // | | | | | | |
| | | NM_005541 | MGC142142 // | | | | | | |
| | | | SHIP // SHIP1 // | | | | | | |
| | | | SIP-145 // hp51CN | | | | | | |
| slingshot homolog 2 (Drosophila) | SSH2 | NM_033389 | KIAA1725 // | 3751625 | chr17 | − | 24977091 | 25281397 | 1.06 |
| | | | MGC78588 // SSH-2 | | | | | | |
| oxysterol binding protein-like 8 | OSBPL8 | NM_001003712 // | DKFZp686A11164 | 3462949 | chr12 | − | 75269250 | 75477720 | 1.06 |
| | | NM_020841 | // MGC126578 // | | | | | | |
| | | | MGC133203 // | | | | | | |
| | | | ORP8 // OSBP10 | | | | | | |
| RAS p21 protein activator 3 | RASA3 | NM_007368 | GAP1IP4BP // | 3526831 | chr13 | − | 113765177 | 113916197 | 1.06 |
| | | | GAPIII // | | | | | | |
| | | | MGC46517 // | | | | | | |
| stromal antigen 2 | STAG2 | NM_001042749 // | DKFZp686P168 // | 3989721 | chrX | + | 122922013 | 123384079 | 1.06 |
| | | NM_001042750 // | DKFZp781H1753 // | | | | | | |
| | | NM_001042751 // | FLJ25871 // SA-2 | | | | | | |
| | | NM_006603 | // SA2 // | | | | | | |
| | | | bA517O1.1 | | | | | | |
| NIMA (never in mitosis gene a)-related kinase 7 | NEK7 | NM_133494 | — | 2373736 | chr1 | + | 196225008 | 196558827 | 1.06 |
| protein tyrosine phosphatase, receptor type, E | PTPRE | NM_006504 // | DKFZp313F1310 // | 3270270 | chr10 | + | 129595315 | 129774152 | 1.06 |
| | | NM_130435 | HPTPE // PTPE // | | | | | | |
| | | | R-PTP-EPSILON | | | | | | |
| solute carrier organic anion transporter family, | SLCO4A1 | NM_016354 | OATP-E // OATP1 | 3892812 | chr20 | + | 60744242 | 60787582 | 1.06 |
| member 4A1 | | | // OATP4A1 // | | | | | | |
| | | | OATPRP1 // POAT | | | | | | |
| | | | // SLC21A12 | | | | | | |
| v-src sarcoma (Schmidt-Ruppin A-2) viral | SRC | NM_005417 // | ASV // SRC1 // c- | 3884191 | chr20 | + | 35406502 | 35467847 | 1.06 |
| oncogene homolog (avian) | | NM_198291 | SRC // p60-Src | | | | | | |
| phosphoinositide-3-kinase, regulatory subunit 5, | PIK3R5 | NM_014308 | F730038I15Rik // | 3744680 | chr17 | − | 8718740 | 8809747 | 1.06 |
| p101 | | | FOAP-2 // P101- | | | | | | |
| | | | PI3K | | | | | | |
| HECT, UBA and WWE domain containing 1 // | HUWE1 // | NM_031407 // | ARF-BP1 // | 4009315 | chrX | − | 53575796 | 53740871 | 1.06 |
| paraoxonase 1 // paraoxonase 3 | PON1 // PON3 | NM_000446 // | HECTH9 // | | | | | | |
| | | NM_000940 | HSPC272 // lb772 | | | | | | |
| | | | // KIAA0312 // | | | | | | |
| | | | LASU1 // MULE // | | | | | | |
| | | | UREB1 // ESA // | | | | | | |
| | | | PON // — | | | | | | |
| antizyme inhibitor 1 | AZIN1 | NM_015878 // | MGC3832 // | 3147591 | chr8 | − | 103907725 | 103953967 | 1.06 |
| | | NM_148174 // | MGC691 // OAZI | | | | | | |
| | | | // OAZIN // | | | | | | |
| A kinase (PRKA) anchor protein 13 | AKAP13 | NM_006738 // | AKAP-Lbc // BRX | 3606304 | chr15 | + | 83681343 | 84102785 | 1.06 |
| | | NM_007200 // | // FLJ11952 // | | | | | | |
| | | NM_144767 | FLJ43341 // HA-3 | | | | | | |
| | | | // Ht31 // LBC // | | | | | | |
| | | | PROTO-LB // | | | | | | |
| | | | PROTO-LBC // c- | | | | | | |
| | | | lbc | | | | | | |
| eukaryotic translation initiation factor 4 gamma, 2 | EIF4G2 | NM_001042559 // | AAG1 // DAP5 // | 3362719 | chr11 | − | 10487574 | 10788746 | 1.06 |
| | | NM_001418 | FLJ41344 // NAT1 | | | | | | |
| | | | // p97 | | | | | | |
| suppressor of Ty 6 homolog (S. cerevisiae) | SUPT6H | NM_003170 | KIAA0162 // | 3715703 | chr17 | + | 24013260 | 24053809 | 1.06 |
| | | | MGC87943 // SPT6 | | | | | | |
| | | | // SPT6H // emb-5 | | | | | | |
| solute carrier family 36 (proton/amino acid | SLC36A4 | NM_152313 | FLJ38932 // PAT4 | 3386638 | chr11 | − | 92485640 | 92570746 | 1.06 |
| symporter), member 4 | | | | | | | | | |
| DOT1-like, histone H3 methyltransferase (S. cerevisiae) | DOT1L | NM_032482 | DOT1 // KIAA1814 | 3816264 | chr19 | + | 2114945 | 2183576 | 1.06 |
| epithelial stromal interaction 1 (breast) // | EPSTI1 // | NM_001002264 // | BRESI1 // | 3511698 | chr13 | − | 42309603 | 42464520 | 1.06 |
| junctophilin 3 | JPH3 | NM_033255 // | MGC29634 // | | | | | | |
| | | NM_020655 | CAGL237 // | | | | | | |
| | | | FLJ44707 // HDL2 | | | | | | |
| | | | // JP-3 // JP3 // | | | | | | |
| | | | TNRC22 | | | | | | |
| promyelocytic leukemia | PML | XM_001132060 // | MYL // PP8675 // | 3601387 | chr15 | + | 72073603 | 72126162 | 1.06 |
| | | NM_002675 // | RNF71 // TRIM19 | | | | | | |
| | | NM_033238 // | | | | | | | |
| | | NM_033239 // | | | | | | | |
| | | NM_033240 // | | | | | | | |
| | | NM_033244 // | | | | | | | |
| | | NM_033246 // | | | | | | | |
| | | NM_033247 // | | | | | | | |
| | | NM_033249 // | | | | | | | |
| | | NM_033250 | | | | | | | |
| DnaJ (Hsp40) homolog, subfamily A, member 1 | DNAJA1 | NM_001539 | DJ-2 // DjA1 // | 3166718 | chr9 | + | 33015173 | 33029946 | 1.06 |
| | | | HDJ2 // HSDJ // | | | | | | |
| | | | HSJ2 // HSPF4 // | | | | | | |
| | | | hDJ-2 | | | | | | |
| TSC22 domain family, member 3 | TSC22D3 | NM_001015881 // | DIP // | 4017381 | chrX | − | 106803586 | 106916423 | 1.06 |
| | | NM_004089 // | DKFZp313A1123 // | | | | | | |
| | | NM_198057 | DSIPI // GILZ // | | | | | | |
| | | | TSC-22R // hDIP | | | | | | |
| RNA binding motif protein 4 | RBM4 | NM_002896 | DKFZp547K0918 // | 3336422 | chr11 | + | 66161951 | 66190547 | 1.06 |
| | | | LARK // MGC75138 | | | | | | |
| | | | // RBM4A // | | | | | | |
| | | | ZCCHC21 // | | | | | | |
| | | | ZCRB3A | | | | | | |
| Smg-7 homolog, nonsense mediated mRNA | SMG7 | NM_014837 // | C1orf16 // EST1C | 2371255 | chr1 | + | 181708036 | 181834004 | 1.06 |
| decay factor (C. elegans) | | NM_173156 // | // FLJ23717 // | | | | | | |
| | | NM_201568 // | KIAA0250 // | | | | | | |
| | | NM_201569 | SGA56M // SMG-7 | | | | | | |
| baculoviral IAP repeat-containing 2 | BIRC2 | NM_001166 | API1 // HIAP2 // | 3346584 | chr11 | + | 101722704 | 101754720 | 1.06 |
| | | | Hiap-2 // MIHB // | | | | | | |
| | | | RNF48 // cIAP1 | | | | | | |
| DEAH (Asp-Glu-Ala-His) box polypeptide 15 | DHX15 | NM_001358 | DBP1 // DDX15 // | 2763805 | chr4 | − | 24128159 | 24250940 | 1.06 |
| | | | HRH2 // PRP43 // | | | | | | |
| | | | PRPF43 // PrPp43p | | | | | | |
| echinoderm microtubule associated protein like 4 | EML4 | NM_019063 | C2orf2 // | 2478748 | chr2 | + | 42250017 | 42418636 | 1.06 |
| | | | DKFZp686P18118 | | | | | | |
| | | | // ELP120 // | | | | | | |
| | | | FLJ10942 // | | | | | | |
| | | | FLJ32318 // | | | | | | |
| | | | ROPP120 | | | | | | |
| protein phosphatase 6, catalytic subunit | PPP6C | NM_002721 | — | 3225348 | chr9 | − | 126948675 | 126991992 | 1.06 |
| bromodomain and WD repeat domain containing 1 | BRWD1 | NM_001007246 // | C21orf107 // | 3932148 | chr21 | − | 39479279 | 39615332 | 1.06 |
| | | NM_018963 // | FLJ43918 // N143 | | | | | | |
| | | NM_033656 | // WDR9 | | | | | | |
| amyloid beta (A4) precursor-like protein 2 | APLP2 | NM_001642 | APPH // APPL2 // | 3356115 | chr11 | + | 129444931 | 129519895 | 1.06 |
| | | | CDEBP | | | | | | |
| sperm associated antigen 9 | SPAG9 | NM_003971 // | FLJ13450 // | 3762519 | chr17 | − | 46394554 | 46553215 | 1.06 |
| | | NM_172345 | FLJ14006 // | | | | | | |
| | | | FLJ34602 // HLC4 | | | | | | |
| | | | // HSS // JLP // | | | | | | |
| | | | KIAA0516 // | | | | | | |
| | | | MGC117291 // | | | | | | |
| | | | MGC14967 // | | | | | | |
| | | | MGC74461 // PHET | | | | | | |
| | | | // PIG6 | | | | | | |
| Rap guanine nucleotide exchange factor (GEF) | RAPGEF6 // | NM_016340 // | DKFZp667N084 // | 2874794 | chr5 | − | 130760272 | 131160628 | 1.06 |
| 6 // folliculin interacting protein 1 | FNIP1 | NM_001008738 // | DKFZp686I15116 // | | | | | | |
| | | NM_133372 | KIA001LB // PDZ- | | | | | | |
| | | | GEF2 // PDZGEF2 | | | | | | |
| | | | // RA-GEF-2 // | | | | | | |
| | | | DKFZp686E18167 | | | | | | |
| | | | // DKFZp781P0215 | | | | | | |
| | | | // KIAA1961 // | | | | | | |
| | | | MGC667 | | | | | | |
| ATPase, Na+/K+ transporting, beta 3 | ATP1B3 | XM_001133533 // | ATPB-3 // CD298 | 2645764 | chr3 | + | 143077611 | 143128338 | 1.06 |
| polypeptide | | XM_001133534 // | // FLJ29027 | | | | | | |
| | | NM_001679 | | | | | | | |
| leukocyte-associated immunoglobulin-like | LAIR1 | NM_002287 // | CD305 // LAIR-1 | 3870824 | chr19 | − | 59557066 | 59578582 | 1.06 |
| receptor 1 | | NM_021706 | | | | | | | |
| interleukin enhancer binding factor 2, 45 kDa | ILF2 | NM_004515 | MGC8391 // NF45 | 2436132 | chr1 | − | 151900372 | 151910103 | 1.06 |
| | | | // PRO3063 | | | | | | |
| solute carrier family 38, member 1 | SLC38A1 | NM_001077484 // | ATA1 // NAT2 // | 3452231 | chr12 | − | 44863120 | 44952390 | 1.06 |
| | | NM_030674 | SAT1 // SNAT1 | | | | | | |
| tudor domain containing 7 | TDRD7 | NM_014290 | KIAA1529 // | 3181193 | chr9 | + | 99202471 | 99298225 | 1.06 |
| | | | PCTAIRE2BP // | | | | | | |
| | | | RP11-508D10.1 // | | | | | | |
| | | | TRAP | | | | | | |
| thioredoxin domain containing 14 // catenin | TXNDC14 // | NM_015959 // | CGI-31 // | 3331487 | chr11 | + | 57235839 | 57391291 | 1.06 |
| (cadherin-associated protein), delta 1 | CTNND1 | XM_001126721 // | DKFZp781O2021 // | | | | | | |
| | | XM_001126756 // | MGC111151 // | | | | | | |
| | | XM_001126767 // | PIG26 // TMX2 // | | | | | | |
| | | XM_001126781 // | CAS // CTNND // | | | | | | |
| | | XM_001126793 // | KIAA0384 // | | | | | | |
| | | XM_001126823 // | P120CAS // | | | | | | |
| | | NM_001331 | P120CTN // p120 | | | | | | |
| zinc finger protein 207 | ZNF207 | NM_001032293 // | DKFZp761N202 | 3717635 | chr17 | + | 27701159 | 27759429 | 1.06 |
| | | NM_003457 | | | | | | | |
| zinc finger, AN1-type domain 3 | ZFAND3 | NM_021943 | FLJ13222 // TEX27 | 2905664 | chr6 | + | 37895285 | 38231400 | 1.06 |
| catenin (cadherin-associated protein), beta 1, | CTNNB1 | XM_001133660 // | CTNNB // | 2618940 | chr3 | + | 41211356 | 41256934 | 1.06 |
| 88 kDa | | XM_001133664 // | FLJ25606 | | | | | | |
| | | XM_001133673 // | | | | | | | |
| | | XM_001133675 // | | | | | | | |
| | | NM_001904 | | | | | | | |
| NCK-associated protein 1-like | NCKAP1L | NM_005337 | HEM1 | 3416577 | chr12 | + | 53177769 | 53224156 | 1.06 |
| upstream binding protein 1 (LBP-1a) | UBP1 | NM_014517 | DKFZp686L1745 // | 2668351 | chr3 | − | 33404835 | 33456874 | 1.06 |
| | | | LBP-1B // LBP-1a | | | | | | |
| | | | // LBP1A // | | | | | | |
| cytoskeleton-associated protein 4 | CKAP4 | NM_006825 | CLIMP-63 // | 3469687 | chr12 | − | 105155796 | 105222139 | 1.06 |
| | | | ERGIC-63 // | | | | | | |
| | | | MGC99554 // p63 | | | | | | |
| ubiquitin B // chromosome 17 open reading | UBB // | NM_018955 // | FLJ25987 // | 3712041 | chr17 | + | 16223047 | 16228977 | 1.06 |
| frame 45 | C17orf45 | NM_152350 | MGC8385 // | | | | | | |
| | | | FLJ25777 // | | | | | | |
| | | | MGC40157 | | | | | | |
| ATPase, Class V, type 10D | ATP10D | NM_020453 | ATPVD // | 2726072 | chr4 | + | 47179942 | 47290260 | 1.06 |
| cytoplasmic polyadenylation element binding | CPEB4 | NM_030627 | KIAA1673 | 2841699 | chr5 | + | 173177911 | 173320580 | 1.06 |
| protein 4 | | | | | | | | | |
| zinc finger protein 451 | ZNF451 // | NM_001031623 // | COASTER // | 2911303 | chr6 | + | 57062631 | 57143064 | 1.06 |
| | KIAA1702 | NM_015555 | FLJ90693 // | | | | | | |
| | | | KIAA0576 // | | | | | | |
| | | | MGC26701 // | | | | | | |
| | | | dJ417I1.1 | | | | | | |
| superoxide dismutase 2, mitochondrial | SOD2 // | NM_000636 // | IPO-B // MNSOD | 2982319 | chr6 | − | 160020086 | 160103541 | 1.06 |
| | MGC5618 | NM_001024465 // | // Mn-SOD | | | | | | |
| | | NM_001024466 | | | | | | | |
| optic atrophy 1 (autosomal dominant) | OPA1 | NM_015560 // | FLJ12460 // | 2658346 | chr3 | + | 194793673 | 194902403 | 1.06 |
| | | NM_130831 // | KIAA0567 // NPG | | | | | | |
| | | NM_130832 // | // NTG // largeG | | | | | | |
| | | NM_130833 // | | | | | | | |
| | | NM_130834 // | | | | | | | |
| | | NM_130835 // | | | | | | | |
| | | NM_130836 // | | | | | | | |
| | | NM_130837 | | | | | | | |
| Rho-associated, coiled-coil containing protein | ROCK1 | NM_005406 | MGC131603 // | 3800619 | chr18 | − | 16783710 | 17017478 | 1.06 |
| kinase 1 | | | MGC43611 // | | | | | | |
| | | | P160ROCK | | | | | | |
| GNAS complex locus | GNAS | NM_000516 // | AHO // C20orf45 | 3891163 | chr20 | + | 56848165 | 56919604 | 1.06 |
| | | NM_001077488 // | // GNAS1 // GPSA | | | | | | |
| | | NM_001077489 // | // GSA // GSP // | | | | | | |
| | | NM_001077490 // | MGC33735 // | | | | | | |
| | | NM_016592 // | PHP1A // PHP1B | | | | | | |
| | | NM_080425 // | // POH // | | | | | | |
| | | NM_080426 // | dJ309F20.1.1 // | | | | | | |
| | | NR_003259 | dJ806M20.3.3 | | | | | | |
| myxovirus (influenza virus) resistance 1, | MX1 | NM_002462 | IFI-78K // IFI78 // | 3922100 | chr21 | + | 41714183 | 41752955 | 1.06 |
| interferon-inducible protein p78 (mouse) | | | MX // MxA | | | | | | |
| SP110 nuclear body protein | SP110 | NM_004509 // | FLJ22835 // IFI41 | 2603051 | chr2 | − | 230740259 | 230797812 | 1.06 |
| | | NM_004510 // | // IFI75 // VODI | | | | | | |
| | | NM_080424 | | | | | | | |
| AP1 gamma subunit binding protein 1 | AP1GBP1 | NM_007247 // | MGC104959 // | 3754677 | chr17 | − | 32949033 | 33043613 | 1.06 |
| | | NM_080550 | SYNG | | | | | | |
| TAR DNA binding protein | TARDBP | NM_007375 | TDP-43 | 2320048 | chr1 | + | 10995025 | 11013170 | 1.06 |
| methionine adenosyltransferase II, alpha | MAT2A | NM_005911 | MATA2 // MATII // | 2491615 | chr2 | + | 85597809 | 85625908 | 1.06 |
| | | | SAMS2 | | | | | | |
| RNA binding motif protein 17 | RBM17 | NM_032905 | MGC14439 // | 3233547 | chr10 | + | 6170982 | 6225410 | 1.06 |
| | | | SPF45 | | | | | | |
| asparaginyl-tRNA synthetase // sideroflexin 3 | NARS // | NM_004539 // | ASNRS // | 3809671 | chr18 | − | 53418770 | 53448841 | 1.06 |
| | SFXN3 | NM_030971 | BA108L7.2 // SFX3 | | | | | | |
| PAN3 polyA specific ribonuclease subunit | PAN3 | NM_175854 | — | 3483159 | chr13 | + | 27572646 | 27773671 | 1.06 |
| homolog (S. cerevisiae) | | | | | | | | | |
| SMAD specific E3 ubiquitin protein ligase 2 | SMURF2 | NM_022739 | DKFZp686F0270 // | 3766960 | chr17 | − | 59968891 | 60088896 | 1.06 |
| | | | MGC138150 | | | | | | |
| ankyrin repeat domain 15 | ANKRD15 | NM_015158 // | DKFZp451G231 // | 3159483 | chr9 | + | 460301 | 746375 | 1.06 |
| | | NM_153186 | KANK // KIAA0172 | | | | | | |
| | | | // MGC43128 | | | | | | |
| structural maintenance of chromosomes flexible | SMCHD1 | XM_113962 // | DKFZp686O0631 // | 3776193 | chr18 | + | 2688138 | 2794997 | 1.06 |
| hinge domain containing 1 | | XM_938891 | KIAA0650 | | | | | | |
| transcription factor 12 (HTF4, helix-loop-helix | TCF12 | NM_003205 // | HEB // HTF4 // | 3595096 | chr15 | + | 54963208 | 55402126 | 1.06 |
| transcription factors 4) | | NM_207036 // | HsT17266 | | | | | | |
| | | NM_207037 // | | | | | | | |
| | | NM_207038 // | | | | | | | |
| | | NM_207040 | | | | | | | |
| ATPase, Ca++ transporting, cardiac muscle, | ATP2A2 | NM_001681 // | ATP2B // DAR // | 3431483 | chr12 | + | 109203332 | 109352495 | 1.06 |
| slow twitch 2 | | NM_170665 | DD // MGC45367 | | | | | | |
| | | | // SERCA2 | | | | | | |
| ubiquitin associated protein 1 | UBAP1 | NM_016525 | MGC119669 // | 3167325 | chr9 | + | 34168995 | 34243205 | 1.06 |
| | | | MGC8710 // | | | | | | |
| | | | NAG20 // UAP // | | | | | | |
| heparan sulfate (glucosamine) 3-O- | HS3ST3B1 | NM_006041 | 30ST3B1 // | 3711262 | chr17 | + | 14145125 | 14193444 | 1.06 |
| sulfotransferase 3B1 | | | 3OST3B1 | | | | | | |
| arrestin domain containing 2 | ARRDC2 | NM_001025604 // | CLONE24945 // | 3824713 | chr19 | + | 17972961 | 17985905 | 1.06 |
| | | NM_015683 | PP2703 | | | | | | |
| casein kinase 1, alpha 1 | CSNK1A1 | NM_001025105 // | CK1 // HLCDGP1 | 2880932 | chr5 | − | 148800168 | 148922811 | 1.06 |
| | | NM_001892 | // PRO2975 | | | | | | |
| CCR4-NOT transcription complex, subunit 2 | CNOT2 | NM_014515 | CDC36 // | 3421897 | chr12 | + | 68901823 | 69035035 | 1.06 |
| | | | HSPC131 // NOT2 | | | | | | |
| mitogen-activated protein kinase kinase kinase 8 | MAP3K8 | NM_005204 | COT // EST // | 3240987 | chr10 | + | 30762872 | 30791283 | 1.06 |
| | | | ESTF // FLJ10486 | | | | | | |
| | | | // TPL2 // Tpl-2 | | | | | | |
| | | | // c-COT | | | | | | |
| mitogen-activated protein kinase kinase kinase 2 | MAP3K2 | XM_001128799 // | MEKK2 // MEKK2B | 2574798 | chr2 | − | 127772731 | 127863022 | 1.06 |
| | | NM_006609 | | | | | | | |
| protein phosphatase 4, regulatory subunit 2 | PPP4R2 | NM_174907 | MGC131930 | 2629693 | chr3 | + | 73019946 | 73201035 | 1.06 |
| topoisomerase (DNA) 1 | TOP1 | NM_003286 | TOP1 | 3885464 | chr20 | + | 39090891 | 39186535 | 1.06 |
| ubiquitin specific peptidase 7 (herpes virus- | USP7 | NM_003470 | HAUSP // TEF1 | 3679564 | chr16 | − | 8893458 | 8964832 | 1.06 |
| associated) | | | | | | | | | |
| RAB21, member RAS oncogene family | RAB21 | NM_014999 | KIAA0118 | 3422269 | chr12 | + | 70434927 | 70470787 | 1.06 |
| MAX interactor 1 | MXI1 | NM_001008541 // | MAD2 // | 3263624 | chr10 | + | 111944059 | 112037108 | 1.06 |
| | | NM_005962 // | MGC43220 // | | | | | | |
| | | NM_130439 | MXD2 // MXI | | | | | | |
| NGFI-A binding protein 1 (EGR1 binding protein | NAB1 | NM_005966 | — | 2520225 | chr2 | + | 191205069 | 191437200 | 1.06 |
| cytochrome P450, family 1, subfamily B, | CYP1B1 | NM_000104 | CP1B // GLC3A | 2548699 | chr2 | − | 38138730 | 38196009 | 1.06 |
| polypeptide 1 | | | | | | | | | |
| chromosome 1 open reading frame 63 | C1orf63 | NM_020317 | DJ465N24.2.1 // | 2402111 | chr1 | − | 25440667 | 25538017 | 1.06 |
| | | | NPD014 // RP3- | | | | | | |
| | | | 465N24.4 | | | | | | |
| nucleoporin 153 kDa | NUP153 | NM_005124 | HNUP153 // N153 | 2943808 | chr6 | − | 17723255 | 17855638 | 1.06 |
| protein phosphatase 1K (PP2C domain | PPM1K | NM_152542 | DKFZp667B084 // | 2777333 | chr4 | − | 89401891 | 89424935 | 1.06 |
| containing) | | | DKFZp761G058 // | | | | | | |
| | | | PTMP // | | | | | | |
| | | | UG0882E07 | | | | | | |
| phosphoinositide-3-kinase, class 3 | PIK3C3 | NM_002647 | MGC61518 // | 3786039 | chr18 | + | 37534028 | 37985705 | 1.05 |
| CCHC-type zinc finger, nucleic acid binding | CNBP | NM_003418 | CNBP1 // DM2 // | 2694644 | chr3 | − | 130369369 | 130385467 | 1.05 |
| protein | | | PROMM // RNF163 | | | | | | |
| | | | // ZCCHC22 // | | | | | | |
| | | | ZNF9 | | | | | | |
| ubiquitin specific peptidase 9, X-linked // | USP9X // | NM_001039590 // | DFFRX // FAF // | 3974708 | chrX | + | 40829542 | 40980776 | 1.05 |
| ubiquitin specific peptidase 9, Y-linked (fat | USP9Y | NM_001039591 // | AZF // AZFA // | | | | | | |
| facets-like, Drosophila) | | NM_004654 | DFFRY // SP3 | | | | | | |
| baculoviral IAP repeat-containing 6 (apollon) | BIRC6 | NM_016252 | APOLLON // | 2476219 | chr2 | + | 32435095 | 32697447 | 1.05 |
| | | | BRUCE // | | | | | | |
| | | | FLJ13726 // | | | | | | |
| | | | FLJ13786 // | | | | | | |
| chromosome 11 open reading frame 30 | C11orf30 | NM_020193 | EMSY // FLJ90741 | 3340913 | chr11 | + | 75803897 | 75941697 | 1.05 |
| | | | // GL002 | | | | | | |
| pleckstrin homology domain containing, family M | PLEKHM1 | XM_001128220 // | AP162 // B2 // | 3759849 | chr17 | − | 40869050 | 40923923 | 1.05 |
| (with RUN domain) member 1 | | NM_014798 | KIAA0356 | | | | | | |
| autism susceptibility candidate 2 | AUTS2 | NM_015570 | KIAA0442 // | 3006572 | chr7 | + | 68695979 | 69896399 | 1.05 |
| | | | MGC13140 | | | | | | |
| YME1-like 1 (S. cerevisiae) | YME1L1 | NM_014263 // | FTSH // MEG4 // | 3282213 | chr10 | − | 27439066 | 27484191 | 1.05 |
| | | NM_139312 // | PAMP // YME1L | | | | | | |
| | | NM_139313 | | | | | | | |
| toll-like receptor 4 | TLR4 | NM_138554 | CD284 // TOLL // | 3186966 | chr9 | + | 119506334 | 119670150 | 1.05 |
| | | | hToll | | | | | | |
| splicing factor 3b, subunit 1, 155 kDa | SF3B1 | NM_001005526 // | PRP10 // PRPF10 | 2593670 | chr2 | − | 197962756 | 198039327 | 1.05 |
| | | NM_012433 | // SAP155 // | | | | | | |
| | | | SF3b155 | | | | | | |
| E3 ubiquitin protein ligase, HECT domain | EDD1 | NM_015902 | DD5 // EDD // | 3147321 | chr8 | − | 103333634 | 103493671 | 1.05 |
| containing, 1 | | | FLJ11310 // HYD | | | | | | |
| | | | // KIAA0896 // | | | | | | |
| | | | MGC57263 | | | | | | |
| ATP-binding cassette, sub-family C | ABCC1 | NM_004996 // | ABC29 // ABCC // | 3649890 | chr16 | + | 15950935 | 16144419 | 1.05 |
| (CFTR/MRP), member 1 | | NM_019862 // | DKFZp686N04233 | | | | | | |
| | | NM_019898 // | // DKFZp781G125 | | | | | | |
| | | NM_019899 // | // GS-X // MRP // | | | | | | |
| | | NM_019900 // | MRP1 | | | | | | |
| | | NM_019901 // | | | | | | | |
| | | NM_019902 | | | | | | | |
| pleckstrin | PLEK | NM_002664 | FLJ27168 // P47 | 2486811 | chr2 | + | 68415853 | 68526386 | 1.05 |
| farnesyltransferase, CAAX box, alpha | FNTA | NM_001018676 // | FPTA // MGC99680 | 3096428 | chr8 | + | 43030413 | 43060080 | 1.05 |
| | | NM_001018677 // | // PGGT1A | | | | | | |
| | | NM_002027 | | | | | | | |
| centrosomal protein 170 kDa // centrosomal | CEP170 // | XM_001125768 // | FAM68A // KAB // | 2463864 | chr1 | − | 241354364 | 241485236 | 1.05 |
| protein 170 kDa-like | CEP170L | NM_001042404 // | KIAA0470 // | | | | | | |
| | | NM_001042405 // | FAM68B // | | | | | | |
| | | NM_014812 // | KIAA0470L // | | | | | | |
| | | XR_015931 // | MGC26143 | | | | | | |
| | | XR_017916 // | | | | | | | |
| | | NR_003135 | | | | | | | |
| forkhead box K2 | FOXK2 | XM_001134363 // | ILF // ILF-1 // | 3738969 | chr17 | + | 78070883 | 78195807 | 1.05 |
| | | XM_001134364 // | ILF1 | | | | | | |
| | | NM_004514 | | | | | | | |
| B-cell CLL/lymphoma 9-like | BCL9L | NM_182557 | BCL9-2 // DLNB11 | 3393993 | chr11 | − | 118272076 | 118304947 | 1.05 |
| cell division cycle 2-like 5 (cholinesterase- | CDC2L5 | NM_003718 // | CDC2L // CHED // | 2998536 | chr7 | + | 39897668 | 40103253 | 1.05 |
| related cell division controller) | | NM_031267 | FLJ35215 // | | | | | | |
| | | | KIAA1791 | | | | | | |
| proteasome (prosome, macropain) 26S subunit, | PSMD11 | NM_002815 | MGC3844 // S9 // | 3717737 | chr17 | + | 27795087 | 27834449 | 1.05 |
| non-ATPase, 11 | | | p44.5 | | | | | | |
| calreticulin | CALR | NM_004343 | FLJ26680 // RO // | 3822049 | chr19 | + | 12910423 | 12916480 | 1.05 |
| | | | SSA // cC1qR | | | | | | |
| schlafen family member 11 | SLFN11 | NM_152270 | FLJ34922 // | 3753500 | chr17 | − | 30696595 | 30724798 | 1.05 |
| | | | SLFN8/9 | | | | | | |
| nuclear receptor subfamily 3, group C, member | NR3C1 | NM_000176 // | GCCR // GCR // | 2879312 | chr5 | − | 142566614 | 142794517 | 1.05 |
| 1 (glucocorticoid receptor) | | NM_001018074 // | GR // GRL | | | | | | |
| | | NM_001018075 // | | | | | | | |
| | | NM_001018076 // | | | | | | | |
| | | NM_001018077 // | | | | | | | |
| | | NM_101020825 // | | | | | | | |
| | | NM_001024094 | | | | | | | |
| dual specificity phosphatase 16 | DUSP16 | NM_030640 | KIAA1700 // | 3444958 | chr12 | − | 12510545 | 12606611 | 1.05 |
| | | | MGC129701 // | | | | | | |
| | | | MGC129702 // | | | | | | |
| | | | MKP-7 // MKP7 | | | | | | |
| glycoprotein V (platelet) // zinc finger protein | GP5 // | NM_004488 // | CD42d // FLJ12298 | 3063337 | chr7 | − | 98922007 | 98935856 | 1.05 |
| 394 | ZNF394 | NM_032164 | // ZKSCAN14 | | | | | | |
| nuclear protein, ataxia-telangiectasia locus | NPAT | NM_002519 | E14 | 3390067 | chr11 | − | 107533176 | 107599902 | 1.05 |
| splicing factor, arginine/serine-rich 3 | SFRS3 | NM_003017 | SRp2O | 2905118 | chr6 | + | 36669658 | 36741293 | 1.05 |
| guanine nucleotide binding protein (G protein) | GNA12 | NM_007353 | MGC104623 // | 3035892 | chr7 | − | 2724003 | 2910140 | 1.05 |
| alpha 12 | | | MGC99644 // NNX3 | | | | | | |
| | | | // RMP // gep | | | | | | |
| chromosome 13 open reading frame 18 | C13orf18 | NM_025113 | FLJ21562 // | 3512948 | chr13 | − | 45814156 | 45917111 | 1.05 |
| | | | FLJ43762 | | | | | | |
| nuclear factor of kappa light polypeptide gene | NFKB2 | NM_001077493 // | LYT-10 // LYT10 | 3261643 | chr10 | + | 104144262 | 104152716 | 1.05 |
| enhancer in B-cells 2 (p49/p100) | | NM_001077494 // | | | | | | | |
| | | NM_002502 | | | | | | | |
| chromosome 22 open reading frame 28 | C22orf28 | NM_014306 | DJ149A16.6 // | 3958253 | chr22 | − | 31113563 | 31138223 | 1.05 |
| | | | HSPC117 // RP1- | | | | | | |
| | | | 149A16.6 | | | | | | |
| ubiquitin specific peptidase 4 (proto-oncogene) | USP4 | NM_003363 // | MGC149848 // | 2674179 | chr3 | − | 49290730 | 49353046 | 1.05 |
| | | NM_199443 | MGC149849 // UNP | | | | | | |
| | | | // Unph | | | | | | |
| F-box protein 11 | FBXO11 | NM_012167 // | FBX11 // FLJ12673 | 2552153 | chr2 | − | 47869608 | 47988296 | 1.05 |
| | | NM_018693 // | // MGC44383 // | | | | | | |
| | | NM_025133 | PRMT9 // | | | | | | |
| | | | UG063H01 // VIT1 | | | | | | |
| PRP4 pre-mRNA processing factor 4 homolog | PRPF4B | NM_003913 | KIAA0536 // PR4H | 2892738 | chr6 | + | 3966500 | 4010215 | 1.05 |
| B (yeast) | | | // PRP4 // PRP4H | | | | | | |
| | | | // PRP4K // | | | | | | |
| | | | dJ1013A10.1 | | | | | | |
| ATPase, aminophospholipid transporter (APLT), | ATP8A1 // | NM_006095 // | ATPASEII // ATPIA | 2767378 | chr4 | − | 42105160 | 42353857 | 1.05 |
| Class I, type 8A, member 1 // protocadherin | PCLKC | NM_017675 | // ATPP2 // | | | | | | |
| LKC | | | MGC130042 // | | | | | | |
| | | | MGC130043 // | | | | | | |
| | | | MGC26327 // | | | | | | |
| | | | FLJ20124 // | | | | | | |
| | | | FLJ20383 // | | | | | | |
| | | | MGC163154 | | | | | | |
| zinc finger protein 267 | ZNF267 | NM_003414 | HZF2 | 3657367 | chr16 | + | 31730255 | 31836159 | 1.05 |
| BAT2 domain containing 1 | BAT2D1 | NM_015172 | BAT2-iso // XTP2 | 2367164 | chr1 | + | 169716078 | 169829262 | 1.05 |
| DEAD (Asp-Glu-Ala-Asp) box polypeptide 18 | DDX18 // | NM_006773 // | FLJ33908 // MrDb | 2502300 | chr2 | + | 118243834 | 118323562 | 1.05 |
| // wingless-type MMTV integration site family, | WNT8B | NM_003393 | // — | | | | | | |
| member 8B | | | | | | | | | |
| Ran GTPase activating protein 1 | RANGAP1 | NM_002883 | Fug1 // KIAA1835 | 3961842 | chr22 | − | 39969442 | 40028027 | 1.05 |
| | | | // MGC20266 // | | | | | | |
| protein kinase, cAMP-dependent, regulatory, | PRKAR1A | NM_002734 // | CAR // CNC // | 3732885 | chr17 | + | 64019700 | 64059052 | 1.05 |
| type I, alpha (tissue specific extinguisher 1) | | NM_212471 // | CNC1 // | | | | | | |
| | | NM_212472 | DKFZp779L0468 // | | | | | | |
| | | | MGC17251 // PKR1 | | | | | | |
| | | | // PRKAR1 // | | | | | | |
| ubiquitin specific peptidase 38 | USP38 | NM_032557 | FLJ35970 // | 2745499 | chr4 | + | 144325529 | 144364549 | 1.05 |
| | | | HP43.8KD // | | | | | | |
| | | | KIAA1891 | | | | | | |
| damage-regulated autophagy modulator | DRAM | NM_018370 | FLJ11259 | 3428783 | chr12 | + | 100795270 | 100930011 | 1.05 |
| SET domain containing 5 | SETD5 | XM_926615 // | DKFZp686J18276 | 2609608 | chr3 | + | 9414309 | 9495916 | 1.05 |
| | | XM_931376 // | // FLJ10707 // | | | | | | |
| | | XM_931417 // | KIAA1757 | | | | | | |
| | | XM_931444 // | | | | | | | |
| | | XM_931447 // | | | | | | | |
| | | XM_931455 // | | | | | | | |
| | | XM_931478 // | | | | | | | |
| | | XM_939712 // | | | | | | | |
| | | XM_944260 // | | | | | | | |
| | | XM_944276 // | | | | | | | |
| | | XM_944280 // | | | | | | | |
| | | XM_944295 // | | | | | | | |
| | | XM_944298 // | | | | | | | |
| | | XM_944303 // | | | | | | | |
| | | NM_001080517 | | | | | | | |
| myeloid differentiation primary response gene | MYD88 | NM_002468 | — | 2617563 | chr3 | + | 38155029 | 38159513 | 1.05 |
| transmembrane protein 1 | TMEM1 | NM_001001723 // | EHOC-1 // EHOC1 | 3923436 | chr21 | + | 44256650 | 44350842 | 1.05 |
| | | NM_003274 | // GT334 // | | | | | | |
| | | | MGC126777 | | | | | | |
| RNA binding motif protein 23 // chromosome 9 | RBM23 // | NM_001077351 // | CAPERbeta // | 3556888 | chr14 | − | 22439714 | 22458231 | 1.05 |
| open reading frame 102 | C9orf102 | NM_001077352 // | FLJ10482 // | | | | | | |
| | | NM_018107 // | MGC4458 // PP239 | | | | | | |
| | | NM_001034155 // | // RNPC4 // | | | | | | |
| | | NM_020207 | FLJ37706 // SR278 | | | | | | |
| myeloid/lymphoid or mixed-lineage leukemia | MLLT3 | NM_004529 | AF9 // FLJ2035 // | 3200982 | chr9 | − | 20334968 | 20612522 | 1.05 |
| (trithorax homolog, Drosophila); translocated to, | | | YEATS3 | | | | | | |
| eukaryotic translation initiation factor 3, subunit | EIF3S10 | NM_003750 | EIF3 // EIF3A // | 3309215 | chr10 | − | 120621551 | 120830522 | 1.05 |
| 10 theta, 150/170 kDa | | | KIAA0139 // P167 | | | | | | |
| | | | // eIF3-p170 // | | | | | | |
| | | | eIF3-theta // p180 | | | | | | |
| | | | // p185 | | | | | | |
| chromosome 3 open reading frame 63 | C3orf63 | NM_015224 | DKFZp686C2456 // | 2677653 | chr3 | − | 56629203 | 56692517 | 1.05 |
| | | | KIAA1105 // | | | | | | |
| | | | RAP140 // se89-1 | | | | | | |
| adenosine monophosphate deaminase (isoform | AMPD3 // | NM_000480 // | —// MGC2237 // | 3320169 | chr11 | + | 10428461 | 10485703 | 1.05 |
| E) // peroxisome proliferator-activated | PPARA // | NM_001025389 // | MGC2452 // | | | | | | |
| receptor alpha // tRNA 5-methylaminomethyl- | TRMU // | NM_001025390 // | NRIC1 // PPAR // | | | | | | |
| 2-thiouridylate methyltransferase // G-2 and | GTSE1 | NM_001001929 // | hPPAR // | | | | | | |
| S-phase expressed 1 | | NM_001001930 // | MGC99627 // | | | | | | |
| | | NM_032644 // | MTO2 // MTU1 // | | | | | | |
| | | NM_001001928 // | TRMT // TRMT1 // | | | | | | |
| | | NM_005036 // | TRNT1 // B99 | | | | | | |
| | | NM_018006 // | | | | | | | |
| | | NM_016426 | | | | | | | |
| retinoic acid receptor, alpha | RARA | NM_001033603 // | NR1B1 // RAR | 3720921 | chr17 | + | 35718982 | 35767411 | 1.05 |
| | | NM_000964 // | | | | | | | |
| | | NM_001024809 | | | | | | | |
| p21 (CDKNIA)-activated kinase 2 | PAK2 | XM_001126110 // | PAK65 // | 2659577 | chr3 | + | 197951145 | 198043895 | 1.05 |
| | | NM_002577 | PAKgamma | | | | | | |
| splicing factor 3b, subunit 2, 145 kDa | SF3B2 | NM_006842 | SAP145 // | 3335907 | chr11 | + | 65573499 | 65593343 | 1.05 |
| | | | SF3B145 // SF3b1 | | | | | | |
| | | | // SF3b150 | | | | | | |
| eukaryotic translation initiation factor 4A, | EIF4A1 // | NM_001416 // | DDX2A // EIF-4A | 3708826 | chr17 | + | 7416768 | 7423040 | 1.05 |
| isoform 1 // SUMO1/sentrin/SMT3 specific | SENP3 | NM_015670 | // EIF4A // | | | | | | |
| peptidase 3 | | | DKFZP586K0919 // | | | | | | |
| | | | DKFZp762A152 // | | | | | | |
| | | | SMT3IP1 // SSP3 | | | | | | |
| syntaxin 6 | STX6 | NM_005819 | — | 2446567 | chr1 | − | 179095658 | 179258656 | 1.05 |
| carbohydrate (chondroitin 4) sulfotransferase | CHST11 | NM_018413 | C4ST // C4ST-1 | 3429566 | chr12 | + | 103374529 | 103679918 | 1.05 |
| 11 | | | // C4ST1 // | | | | | | |
| | | | HSA269537 | | | | | | |
| protein phosphatase 1, catalytic subunit, beta | PPP1CB | NM_206877 // | MGC3672 // PP-1B | 2475209 | chr2 | + | 28828128 | 28879300 | 1.05 |
| isoform | | NM_002709 // | // PPP1CD | | | | | | |
| | | NM_206876 | | | | | | | |
| mitogen-activated protein kinase 6 | MAPK6 | NM_002748 | DKFZp686F03189 | 3694129 | chr15 | + | 50098740 | 50146174 | 1.05 |
| | | | // ERK3 // | | | | | | |
| | | | HsT17250 // | | | | | | |
| | | | PRKM6 // | | | | | | |
| GTP binding protein 4 | GTPBP4 | NM_012341 | CRFG // FLJ10686 | 3231774 | chr10 | + | 942450 | 1055862 | 1.05 |
| | | | // FLJ10690 // | | | | | | |
| | | | FLJ39774 // NGB | | | | | | |
| forkhead box O3A | FOXO3A | NM_001455 // | AF6q21 // | 2920475 | chr6 | + | 108987684 | 109112650 | 1.05 |
| | | NM_201559 | DKFZp781A0677 // | | | | | | |
| | | | FKHRL1 // | | | | | | |
| | | | FKHRL1P2 // | | | | | | |
| | | | MGC12739 // | | | | | | |
| | | | MGC31925 | | | | | | |
| transmembrane protein 2 | TMEM2 | NM_013390 | — | 3209384 | chr9 | − | 73452177 | 73573229 | 1.05 |
| cyclin G associated kinase | GAK | XM_001127411 // | FLJ40395 // | 2756673 | chr4 | − | 833110 | 916149 | 1.05 |
| | | NM_005255 | MGC99654 | | | | | | |
| tumor necrosis factor (ligand) superfamily, | TNFSF8 | NM_001244 | CD153 // CD30L | 3222144 | chr9 | − | 116695445 | 116732620 | 1.05 |
| member 8 | | | // CD30LG // | | | | | | |
| | | | MGC138144 | | | | | | |
| presenilin 1 (Alzheimer disease 3) | PSEN1 | NM_000021 | AD3 // FAD // PS1 | 3543481 | chr14 | + | 72672571 | 72760151 | 1.05 |
| | | | // S182 | | | | | | |
| D4, zinc and double PHD fingers family 2 | DPF2 | NM_006268 | MGC10180 // REQ | 3335089 | chr11 | + | 64857703 | 64877276 | 1.05 |
| | | | // UBID4 // ubi-d4 | | | | | | |
| splicing factor proline/glutamine-rich | SFPQ | NM_005066 | POMP100 // PSF | 2406064 | chr1 | − | 35351696 | 35506894 | 1.05 |
| (polypyrimidine tract binding protein associated) | | | | | | | | | |
| development and differentiation enhancing | DDEF1 | NM_018482 | AMAP1 // ASAP1 | 3153428 | chr8 | − | 131133540 | 131525100 | 1.05 |
| factor 1 | | | // KIAA1249 // | | | | | | |
| | | | PAG2 // PAP // | | | | | | |
| TNF receptor-associated factor 3 | TRAF3 | NM_003300 // | CAP-1 // CD40bp | 3553337 | chr14 | + | 102313566 | 102447585 | 1.05 |
| | | NM_145725 // | // CRAF1 // LAP1 | | | | | | |
| | | NM_145726 | | | | | | | |
| thyroid hormone receptor associated protein 1 | THRAP1 | NM_005121 | ARC250 // | 3765689 | chr17 | − | 57374750 | 57498031 | 1.05 |
| | | | DRIP250 // | | | | | | |
| | | | HSPC221 // | | | | | | |
| | | | KIAA0593 // | | | | | | |
| general transcription factor IIIC, polypeptide 4, | GTF3C4 | NM_012204 | FLJ21002 // | 3192525 | chr9 | + | 134535243 | 134581784 | 1.05 |
| 90 kDa | | | MGC138450 // | | | | | | |
| | | | TFIII90 // TFIIIC90 | | | | | | |
| | | | // TFIIICdelta // | | | | | | |
| | | | TFiiiC2-90 | | | | | | |
| praja 2, RING-H2 motif containing | PJA2 | NM_014819 | KIAA0438 // | 2870397 | chr5 | − | 108614328 | 108773545 | 1.05 |
| | | | Neurodap1 // | | | | | | |
| | | | RNF131 | | | | | | |
| suppressor of var1, 3-like 1 (S. cerevisiae) | SUPV3L1 | NM_003171 | SUV3 | 3250204 | chr10 | + | 70609946 | 70638996 | 1.05 |
| ADP-ribosylation factor 1 | ARF1 | NM_001024226 // | — | 2383726 | chr1 | + | 226313262 | 226353506 | 1.05 |
| | | NM_001024227 // | | | | | | | |
| | | NM_001024228 // | | | | | | | |
| | | NM_001658 | | | | | | | |
| cullin-associated and neddylation-dissociated 1 | CAND1 | NM_018448 | DKFZp434M1414 // | 3420713 | chr12 | + | 65946048 | 65994719 | 1.05 |
| | | | FLJ10114 // | | | | | | |
| | | | FLJ10929 // | | | | | | |
| | | | FLJ38691 // | | | | | | |
| | | | FLJ90441 // | | | | | | |
| | | | KIAA0829 // | | | | | | |
| | | | TIP120 // TIP120A | | | | | | |
| ankylosis, progressive homolog (mouse) | ANKH | XM_001132013 // | ANK // CCAL2 // | 2849469 | chr5 | − | 14757920 | 14924876 | 1.05 |
| | | NM_054027 | CMDJ // CPPDD | | | | | | |
| | | | // FLJ27166 // | | | | | | |
| | | | HANK // MANK | | | | | | |
| UBX domain containing 2 | UBXD2 | NM_014607 | FLJ23318 // | 2507495 | chr2 | + | 136215526 | 136259081 | 1.05 |
| | | | KIAA0242 // | | | | | | |
| | | | UBXDC1 // erasin | | | | | | |
| interferon gamma receptor 1 | IFNGR1 | NM_000416 | CD119 // FLJ45734 | 2976113 | chr6 | − | 137560317 | 137582279 | 1.05 |
| | | | // IFNGR | | | | | | |
| GDP dissociation inhibitor 1 | GDI1 | NM_001493 | FLJ41411 // GDIL | 3996404 | chrX | + | 153318480 | 153324978 | 1.05 |
| | | | // MRX41 // | | | | | | |
| | | | MRX48 // OPHN2 | | | | | | |
| | | | // RABGD1A // | | | | | | |
| | | | RABGDIA // XAP-4 | | | | | | |
| myotubularin related protein 6 | MTMR6 | NM_004685 | — | 3506153 | chr13 | − | 24700317 | 24774271 | 1.05 |
| KIAA0226 | KIAA0226 | XM_032901 // | — | 2713555 | chr3 | − | 198863016 | 198960684 | 1.05 |
| | | XM_936700 | | | | | | | |
| Nipped-B homolog (Drosophila) // nuclear | NIPBL // | NM_015384 // | CDLS // | 2806799 | chr5 | + | 36894163 | 37115932 | 1.05 |
| receptor binding protein 1 | NRBP1 | NM_133433 // | DKFZp434L1319 // | | | | | | |
| | | NM_013392 | FLJ11203 // | | | | | | |
| | | | FLJ12597 // | | | | | | |
| | | | FLJ13354 // | | | | | | |
| | | | FLJ13648 // | | | | | | |
| | | | FLJ44854 // IDN3 | | | | | | |
| | | | // IDN3-B // | | | | | | |
| | | | BCON3 // | | | | | | |
| | | | FLJ27109 // | | | | | | |
| | | | FLJ35541 // MADM | | | | | | |
| | | | // MUDPNP // | | | | | | |
| | | | NRBP | | | | | | |
| DENN/MADD domain containing 4A | DENND4A | NM_005848 | FLJ33949 // IRLB | 3629811 | chr15 | − | 63737458 | 63871676 | 1.05 |
| | | | // KIAA0476 // | | | | | | |
| | | | MYCPBP | | | | | | |
| MKI67 (FHA domain) interacting nucleolar | MKI67IP | NM_032390 | NIFK // Nopp34 | 2573786 | chr2 | − | 122132489 | 122231052 | 1.05 |
| phosphoprotein | | | | | | | | | |
| myeloid/lymphoid or mixed-lineage leukemia 3 | MLL3 // | NM_021230 // | DKFZp686C08112 | 3080033 | chr7 | − | 151462947 | 151763974 | 1.05 |
| // ADAM metallopeptidase with | ADAMTS2 // | NM_170606 // | // FLJ12625 // | | | | | | |
| thrombospondin type 1 motif, 2 // leucine-rich | LRRK1 // | NM_014244 // | FLJ38309 // HALR | | | | | | |
| repeat kinase 1 // B melanoma antigen family, | BAGE3 | NM_021599 // | // KIAA1506 // | | | | | | |
| member 3 | | NM_024652 // | MGC119851 // | | | | | | |
| | | NM_182481 | MGC119852 // | | | | | | |
| | | | MGC119853 // | | | | | | |
| | | | ADAM-TS2 // | | | | | | |
| | | | ADAMTS-3 // NPI | | | | | | |
| | | | // PCINP // PCPNI | | | | | | |
| | | | // hPCPNI // | | | | | | |
| | | | FLJ23119 // | | | | | | |
| | | | FLJ27465 // | | | | | | |
| | | | KIAA1790 // RIPK6 | | | | | | |
| | | | // Roco1 // — | | | | | | |
| cyclin D2 | CCND2 | NM_001759 | KIAK0002 // | 3401704 | chr12 | + | 4232432 | 4284764 | 1.05 |
| | | | MGC102758 | | | | | | |
| isopentenyl-diphosphate delta isomerase 1 | IDI1 | NM_004508 | IPP1 // IPPI1 | 3273601 | chr10 | − | 1075869 | 1092634 | 1.05 |
| ST8 alpha-N-acetyl-neuraminide alpha-2,8- | ST8SIA4 | NM_005668 // | MGC34450 // | 2868904 | chr5 | − | 100116166 | 100293602 | 1.05 |
| sialyltransferase 4 | | NM_175052 | MGC61459 // PST | | | | | | |
| | | | // PST1 // SIAT8D | | | | | | |
| | | | // ST8SIA-IV | | | | | | |
| Kruppel-like factor 9 | KLF9 | NM_001206 | BTEB // BTEB1 | 3208995 | chr9 | − | 72189344 | 72219360 | 1.05 |
| splicing factor, arginine/serine-rich 2, | SFRS2IP | NM_004719 | CASP11 // SIP1 // | 3452145 | chr12 | − | 44599187 | 44672160 | 1.05 |
| interacting protein | | | SRRP129 | | | | | | |
| ubiquitin specific peptidase 47 | USP47 | NM_017944 | DKFZp686C13257 | 3320604 | chr11 | + | 11819556 | 11937446 | 1.05 |
| | | | // FLJ20727 // | | | | | | |
| low density lipoprotein receptor (familial | LDLR | NM_000527 | FH // FHC | 3821015 | chr19 | + | 11061114 | 11115050 | 1.05 |
| hypercholesterolemia) | | | | | | | | | |
| Rho GDP dissociation inhibitor (GDI) beta | ARHGDIB | NM_001175 | D4 // GDIA2 // | 3445786 | chr12 | − | 14986174 | 15005951 | 1.05 |
| | | | GDID4 // LYGDI // | | | | | | |
| | | | Ly-GDI // | | | | | | |
| polymerase (RNA) III (DNA directed) | POLR3E | NM_018119 | RPC5 // SIN | 3652489 | chr19 | + | 22216140 | 22253905 | 1.05 |
| polypeptide E (80 kD) | | | | | | | | | |
| DEAD (Asp-Glu-Ala-Asp) box polypeptide 3, Y- | DDX3Y | NM_004660 | DBY | 4030162 | chrY | + | 13525423 | 13555808 | 1.05 |
| linked | | | | | | | | | |
| adenosine deaminase, RNA-specific | ADAR | NM_001025107 // | ADAR1 // DRADA | 2436754 | chr1 | − | 152821187 | 152867095 | 1.05 |
| | | NM_001111 // | // DSH // DSRAD | | | | | | |
| | | NM_015840 // | // G1P1 // IFI-4 // | | | | | | |
| | | NM_015841 | IFI4 // K88dsRBP | | | | | | |
| | | | // p136 | | | | | | |
| actin binding LIM protein 1 | ABLIM1 | NM_001003407 // | ABLIM // | 3307939 | chr10 | − | 116180867 | 116571234 | 1.05 |
| | | NM_001003408 // | DKFZp781D0148 // | | | | | | |
| | | NM_002313 // | FLJ14564 // | | | | | | |
| | | NM_006720 | KIAA0059 // | | | | | | |
| | | | LIMAB1 // LIMATIN | | | | | | |
| | | | // MGC1224 | | | | | | |
| IBR domain containing 2 | IBRDC2 | NM_182757 | KIAA0161 // | 2897172 | chr6 | + | 18385558 | 18650038 | 1.05 |
| | | | MGC71786 // | | | | | | |
| | | | bA528A10.3 // | | | | | | |
| | | | p53RFP | | | | | | |
| IK cytokine, down-regulator of HLA II | IK | NM_006083 | CSA2 // IK protein | 2831932 | chr5 | + | 140006890 | 140022229 | 1.05 |
| | | | // MGC59741 // | | | | | | |
| | | | RED | | | | | | |
| RAD21 homolog (S. pombe) | RAD21 | NM_006265 | FLJ25655 // | 3149843 | chr8 | − | 117909861 | 117956284 | 1.05 |
| | | | FLJ40596 // HR21 | | | | | | |
| | | | // HRAD21 // | | | | | | |
| | | | KIAA0078 // MCD1 | | | | | | |
| | | | // NXP1 // SCC1 | | | | | | |
| | | | // hHR21 | | | | | | |
| eukaryotic translation initiation factor 4 gamma, 1 | EIF4G1 | NM_004953 // | DKFZp686A1451 // | 2655688 | chr3 | + | 185514970 | 185535829 | 1.05 |
| | | NM_182917 // | EIF4F // EIF4G // | | | | | | |
| | | NM_198241 // | p220 | | | | | | |
| | | NM_198242 // | | | | | | | |
| | | NM_198244 | | | | | | | |
| lactate dehydrogenase A | LDHA | NM_005566 | LDH-M // LDH1 // | 3322775 | chr11 | + | 18372531 | 18386528 | 1.05 |
| | | | PIG19 | | | | | | |
| ubiquitin protein ligase E3A (human papilloma | UBE3A | NM_000462 // | ANCR // AS // E6- | 3614087 | chr15 | − | 23006655 | 23355108 | 1.05 |
| virus E6-associated protein, Angelman | | NM_130838 // | AP // EPVE6AP // | | | | | | |
| syndrome) | | NM_130839 | FLJ26981 // | | | | | | |
| | | | HPVE6A | | | | | | |
| nuclear receptor subfamily 4, group A, member 1 | NR4A1 | NM_002135 // | GFRP1 // HMR // | 3415229 | chr12 | + | 50717248 | 50739539 | 1.05 |
| | | NM_173157 // | MGC9485 // N10 | | | | | | |
| | | NM_173158 | // NAK-1 // NGFIB | | | | | | |
| | | | // NP10 // NUR77 | | | | | | |
| | | | // TR3 | | | | | | |
| diaphanous homolog 1 (Drosophila) | DIAPH1 | NM_001079812 // | DFNA1 // DRF1 // | 2878662 | chr5 | − | 140874773 | 140978866 | 1.05 |
| | | NM_005219 | FLJ25265 // LFHL1 | | | | | | |
| | | | // hDIA1 | | | | | | |
| zinc finger CCCH-type, antiviral 1 | ZC3HAV1 | NM_020119 // | DKFZp686F2052 // | 3075566 | chr7 | − | 138378808 | 138445046 | 1.05 |
| | | NM_024625 | DKFZp686H1869 // | | | | | | |
| | | | DKFZp686O19171 | | | | | | |
| | | | // FLB6421 // | | | | | | |
| | | | FLJ13288 // | | | | | | |
| | | | MGC48898 // ZAP | | | | | | |
| | | | // ZC3H2 // | | | | | | |
| | | | ZC3HDC2 | | | | | | |
| SEC24 related gene family, member C (S. cerevisiae) | SEC24C | NM_004922 | KIAA0079 | 3251848 | chr10 | + | 75174138 | 75210008 | 1.05 |
| | | NM_198597 | | | | | | | |
| calpain 2, (m/ll) large subunit | CAPN2 | NM_001748 | CANPL2 // | 2382117 | chr1 | + | 221955990 | 222031806 | 1.05 |
| | | | CANPml // | | | | | | |
| | | | FLJ39928 // | | | | | | |
| OTU domain containing 4 | OTUD4 | NM_017493 // | DKFZp434I0721 // | 2788195 | chr4 | − | 146274170 | 146471942 | 1.05 |
| | | NM_199324 | HIN1 // HSHIN1 // | | | | | | |
| | | | KIAA1046 | | | | | | |
| Nedd4 binding protein 1 | N4BP1 | NM_153029 | FLJ31821 // | 3690597 | chr16 | − | 47130140 | 47324384 | 1.05 |
| | | | KIAA0615 | | | | | | |
| 6-phosphofructo-2-kinase/fructose-2,6- | PFKFB3 | NM_004566 | IPFK2 // PFK2 | 3233605 | chr10 | + | 6226893 | 6336597 | 1.05 |
| biphosphatase 3 | | | | | | | | | |
| ATPase, H+ transporting, lysosomal 50/57 kDa, | ATP6V1H | NM_015941 // | CGI-11 // | 3135452 | chr8 | − | 54790669 | 54918651 | 1.05 |
| V1 subunit H | | NM_213619 // | MSTP042 // SFD | | | | | | |
| | | NM_213620 | // SFDalpha // | | | | | | |
| | | | SFDbeta // VMA13 | | | | | | |
| ferritin, heavy polypeptide-like 7 // ferritin, | FTHL7 // | NR_002202 // | —// FTH // FTHL6 | 3375648 | chr11 | − | 61488337 | 61492603 | 1.05 |
| heavy polypeptide 1 // ferritin, heavy | FTH1 // | NM_002032 // | // MGC104426 // | | | | | | |
| polypeptide-like 3 // ferritin, heavy polypeptide | FTHL3 // | NR_002201 // —// | PIG15 // PLIF // | | | | | | |
| pseudogene 1 // ferritin, heavy polypeptide-like | FTHP1 // | NR_002205 // | FTHL5 | | | | | | |
| 12 // ferritin, heavy polypeptide-like 11 // | FTHL12 // | NR_002204 // | | | | | | | |
| ferritin, | FTHL11 // | NR_002203 | | | | | | | |
| heavy polypeptide-like 8 | FTHL8 | | | | | | | | |
| protein inhibitor of activated STAT, 1 | PIAS1 | NM_016166 | DDXBP1 // GBP // | 3599280 | chr15 | + | 65898917 | 66277975 | 1.05 |
| | | | GU/RH-II // | | | | | | |
| | | | MGC141878 // | | | | | | |
| | | | MGC141879 // | | | | | | |
| | | | ZMIZ3 | | | | | | |
| interferon-induced protein with | IFIT3 | NM_001031683 // | CIG-49 // GARG- | 3257204 | chr10 | + | 91059126 | 91090705 | 1.05 |
| tetratricopeptide repeats 3 | | NM_001549 | 49 // IFI60 // IFIT4 | | | | | | |
| | | | // IRG2 // ISG60 | | | | | | |
| | | | // RIG-G | | | | | | |
| cullin 4A | CUL4A | NM_001008895 // | — | 3502497 | chr13 | + | 112905835 | 112967356 | 1.05 |
| | | NM_003589 | | | | | | | |
| SH2 domain protein 2A | SH2D2A | NM_003975 | F2771 // TSAd | 2438575 | chr1 | − | 155038957 | 155053637 | 1.05 |
| EH-domain containing 1 | EHD1 | NM_006795 | FLJ42622 // | 3377226 | chr11 | − | 64376790 | 64404202 | 1.05 |
| | | | FLJ44618 // H- | | | | | | |
| | | | PAST // HPAST1 | | | | | | |
| | | | // PAST // PAST1 | | | | | | |
| SH2B adaptor protein 3 | SH2B3 | NM_005475 | LNK | 3431892 | chr12 | + | 110328135 | 110373802 | 1.05 |
| tropomodulin 3 (ubiquitous) | TMOD3 | NM_014547 | UTMOD | 3594066 | chr15 | + | 49909087 | 50026784 | 1.05 |
| SMAD family member 3 | SMAD3 | NM_005902 | DKFZP586N0721 // | 3598959 | chr15 | + | 65145043 | 65296818 | 1.05 |
| | | | DKFZP686J10186 | | | | | | |
| | | | // HSPC193 // | | | | | | |
| | | | HsT17436 // JV15- | | | | | | |
| | | | 2 // MADH3 // | | | | | | |
| | | | MGC60396 // Smad 3 | | | | | | |
| vacuolar protein sorting 53 homolog (S. cerevisiae) | VPS53 | NM_018289 | FLJ10979 // | 3739679 | chr17 | − | 375959 | 564837 | 1.05 |
| | | | MGC39512 // | | | | | | |
| | | | hVps53L // | | | | | | |
| TRAF and TNF receptor associated protein | TTRAP | NM_016614 | AD022 // EAP2 // | 2945645 | chr6 | − | 24758144 | 24775240 | 1.05 |
| | | | MGC111021 // | | | | | | |
| | | | MGC9099 // | | | | | | |
| | | | dJ30M3.3 | | | | | | |
| MAP/microtubule affinity-regulating kinase 2 | MARK2 | NM_001039468 // | EMK1 // MGC99619 | 3334025 | chr11 | + | 63362634 | 63435067 | 1.05 |
| | | NM_001039469 // | // PAR-1 | | | | | | |
| | | NM_004954 // | | | | | | | |
| | | NM_017490 | | | | | | | |
| helicase with zinc finger | HELZ | NM_014877 | DRHC // HUMORF5 | 3768015 | chr17 | − | 62485987 | 62672547 | 1.05 |
| | | | // KIAA0054 // | | | | | | |
| | | | MGC163454 | | | | | | |
| golgi-specific brefeldin A resistance factor 1 | GBF1 | NM_004193 | FLJ21263 // | 3261544 | chr10 | + | 103993260 | 104132642 | 1.05 |
| | | | FLJ21500 // | | | | | | |
| | | | KIAA0248 // | | | | | | |
| | | | MGC134877 // | | | | | | |
| | | | MGC134878 | | | | | | |
| serine/threonine kinase 38 like | STK38L | NM_015000 | KIAA0965 // NDR2 | 3409081 | chr12 | + | 27288343 | 27370157 | 1.05 |
| BCL2-related protein A1 | BCL2A1 | NM_004049 | ACC-1 // ACC-2 | 3635198 | chr15 | − | 78040295 | 78138851 | 1.05 |
| | | | // BCL2L5 // BFL1 | | | | | | |
| | | | // GRS // HBPA1 | | | | | | |
| membrane-associated ring finger (C3HC4) 7 | MARCH7 | NM_022826 | AXO // AXOT // | 2512330 | chr2 | + | 160277236 | 160333585 | 1.05 |
| | | | DKFZP586F1122 // | | | | | | |
| | | | MARCH-VII // | | | | | | |
| | | | RNF177 | | | | | | |
| periphilin 1 | PPHLN1 | NM_016488 // | HSPC206 // | 3412008 | chr12 | + | 40917957 | 41128677 | 1.05 |
| | | NM_201438 // | HSPC232 // | | | | | | |
| | | NM_201439 // | MGC48786 | | | | | | |
| | | NM_201440 // | | | | | | | |
| | | NM_201515 | | | | | | | |
| tankyrase, TRF1-interacting ankyrin-related | TNKS2 | NM_025235 | PARP-5b // PARP- | 3257850 | chr10 | + | 93548049 | 93633726 | 1.05 |
| ADP-ribose polymerase 2 | | | 5c // PARP5B // | | | | | | |
| | | | PARP5C // TANK2 | | | | | | |
| | | | // TNKL | | | | | | |
| kelch-like 6 (Drosophila) | KLHL6 | NM_130446 | FLJ00029 | 2708066 | chr3 | − | 184688052 | 184789879 | 1.05 |
| Kruppel-like factor 10 | KLF10 | NM_001032282 // | EGRA // TIEG // | 3147508 | chr8 | − | 103682306 | 103767558 | 1.05 |
| | | NM_005655 | TIEG1 | | | | | | |
| neugrin, neurite outgrowth associated | NGRN | NM_001033088 // | DSC92 // NEUGRIN | 3607998 | chr15 | + | 88586640 | 88670084 | 1.05 |
| | | NM_016645 | | | | | | | |
| DEAD (Asp-Glu-Ala-Asp) box polypeptide 5 | DDX5 | NM_004396 | DKFZp686J01190 | 3766893 | chr17 | − | 59924843 | 59935796 | 1.05 |
| | | | // G17P1 // HLR1 | | | | | | |
| | | | // HUMP68 // p68 | | | | | | |
| SMEK homolog 2, suppressor of mek1 | SMEK2 | NM_020463 | FLFL2 // FLJ31474 | 2553911 | chr2 | − | 55627936 | 55699519 | 1.05 |
| (Dictyostelium) | | | // KIAA1387 // | | | | | | |
| | | | PSY2 // smk1 | | | | | | |
| thioredoxin reductase 1 | TXNRD1 | NM_003330 // | GRIM-12 // | 3429460 | chr12 | + | 103130628 | 103354079 | 1.05 |
| | | NM_182729 // | MGC9145 // TR // | | | | | | |
| | | NM_182742 // | TR1 // TRXR1 // | | | | | | |
| | | NM_182743 | TXNR | | | | | | |
| Ras and Rab interactor 3 | RIN3 | NM_024832 | DKFZp762H1613 // | 3548929 | chr14 | + | 92049804 | 92225160 | 1.05 |
| | | | FLJ11700 // | | | | | | |
| | | | FLJ22439 | | | | | | |
| itohy homolog E3 ubiquitin protein ligase | ITCH | NM_031483 | AIF4 // AIP4 // | 3882854 | chr20 | + | 32414702 | 32562859 | 1.05 |
| (mouse) | | | NAPP1 // | | | | | | |
| | | | dJ468O1.1 | | | | | | |
| KIAA0174 | KIAA0174 | NM_014761 | MGC117220 | 3667766 | chr16 | + | 70481983 | 70522196 | 1.05 |
| formin-like 3 | FMNL3 | NM_175736 // | DKFZp762B245 // | 3454006 | chr12 | − | 48316548 | 48387433 | 1.05 |
| | | NM_198900 | FHOD3 // | | | | | | |
| | | | FLJ45265 // | | | | | | |
| | | | MGC45819 // | | | | | | |
| ARP3 actin-related protein 3 homolog (yeast) | ACTR3 | NM_005721 | ARP3 | 2501697 | chr2 | + | 114360575 | 114502125 | 1.05 |
| cyclin T2 // aminocarboxymuconate | CCNT2 // | NM_001241 // | FLJ90560 // | 2507209 | chr2 | + | 135392283 | 135432746 | 1.05 |
| semialdehyde decarboxylase | ACMSD | NM_058241 // | MGC134840 // — | | | | | | |
| | | NM_138326 | | | | | | | |
| topoisomerase (DNA) II beta 180 kDa | TOP2B | NM_001068 | TOPIIB // top2beta | 2566478 | chr3 | − | 25614141 | 25681392 | 1.05 |
| RNA binding motif protein 16 // T-cell | RBM16 // | NM_014892 // | KIAA1116 // | 2932360 | chr6 | + | 155029299 | 155324283 | 1.05 |
| lymphoma invasion and metastasis 2 | TIAM2 | NM_001010927 // | FLJ41865 // STEF | | | | | | |
| | | NM_012454 | | | | | | | |
| solute carrier family 3 (activators of dibasic and | SLC3A2 | NM_001012661 // | 4F2 // 4F2HC // | 3333711 | chr11 | + | 62380117 | 62412872 | 1.05 |
| neutral amino acid transport), member 2 | | NM_001012662 // | 4T2HC // CD98 // | | | | | | |
| | | NM_001012663 // | CD98HC // MDU1 | | | | | | |
| | | NM_001012664 // | // NACAE | | | | | | |
| | | NM_001013251 // | | | | | | | |
| | | NM_002394 | | | | | | | |
| Der1-like domain family, member 1 | DERL1 | NM_024295 | DER-1 // DER1 // | 3151401 | chr8 | − | 124094605 | 124123781 | 1.05 |
| | | | FLJ13784 // | | | | | | |
| | | | FL.J42092 // | | | | | | |
| | | | MGC3067 // | | | | | | |
| | | | PRO2577 | | | | | | |
| phosphodiesterase 7A | PDE7A | NM_002603 // | HCP1 // PDE7 | 3138464 | chr8 | − | 66789124 | 67101393 | 1.05 |
| | | NM_002604 | | | | | | | |
| formin-like 1 | FMNL1 | NM_005892 | C17orf1 // | 3723378 | chr17 | + | 40654831 | 40680464 | 1.05 |
| | | | C17orf1B // | | | | | | |
| | | | FHOD4 // FMNL // | | | | | | |
| | | | KW-13 // | | | | | | |
| | | | MGC133052 // | | | | | | |
| | | | MGC1894 // | | | | | | |
| Rap guanine nucleotide exchange factor (GEF) 1 | RAPGEF1 | NM_005312 // | C3G // | 3227696 | chr9 | − | 133441988 | 133605262 | 1.05 |
| | | NM_198679 | DKFZp781P1719 // | | | | | | |
| | | | GRF2 | | | | | | |
| transmembrane BAX inhibitor motif containing 4 | TMBIM4 | NM_016056 | CGI-119 // S1R // | 3460593 | chr12 | − | 64816984 | 64850064 | 1.05 |
| | | | ZPRO | | | | | | |
| B double prime 1, subunit of RNA polymerase III | BDP1 | NM_018429 | DKFZp686C01233 | 2814527 | chr5 | + | 70699177 | 70899402 | 1.05 |
| transcription initiation factor IIIB | | | // DKFZp686KD831 | | | | | | |
| | | | // HSA238520 // | | | | | | |
| | | | KIAA1241 // | | | | | | |
| | | | KIAA1689 // | | | | | | |
| | | | TAF3B1 // TFC5 | | | | | | |
| | | | // TFIIIB″ // | | | | | | |
| | | | TFIIIB150 // | | | | | | |
| | | | TFIIIB90 // TFNR | | | | | | |
| MYST histone acetyltransferase 2 | MYST2 | NM_007067 | HBO1 // HBOA | 3725779 | chr17 | + | 45196447 | 45261455 | 1.05 |
| KIAA0247 | KIAA0247 | NM_014734 | — | 3542145 | chr14 | + | 69145907 | 69256533 | 1.05 |
| ubiquitin specific peptidase 36 | USP36 | NM_025090 | DUB1 | 3772581 | chr17 | − | 74295075 | 74348574 | 1.05 |
| protein kinase, AMP-activated, gamma 2 non- | PRKAG2 | NM_001040633 // | AAKG // AAKG2 // | 3079803 | chr7 | − | 150865031 | 151205160 | 1.05 |
| catalytic subunit | | NM_016203 // | CMH6 // H91620p | | | | | | |
| | | NM_024429 | // WPWS | | | | | | |
| RAB18, member RAS oncogene family | RAB18 | NM_021252 | RAB18LI1 | 3240095 | chr10 | + | 27791811 | 27930577 | 1.05 |
| WW domain containing adaptor with coiled-coil | WAC | NM_016628 // | BM-016 // | 3240340 | chr10 | + | 28828043 | 29005095 | 1.05 |
| | | NM_100264 // | MGC10753 // | | | | | | |
| | | NM_100486 | PRO1741 //Wwp4 | | | | | | |
| | | | // bA48B24 // | | | | | | |
| | | | bA48B24.1 | | | | | | |
| stromal interaction molecule 2 | STIM2 | NM_020860 | FLJ39527 // | 2722377 | chr4 | + | 26415653 | 26636427 | 1.05 |
| | | | KIAA1482 | | | | | | |
| actin, gamma 1 | ACTG1 | NM_001614 | ACT // ACTG // | 3773932 | chr17 | − | 77091456 | 77105797 | 1.05 |
| | | | DFNA20 // DFNA26 | | | | | | |
| ash1 (absent, small, or homeotic)-like | ASH1L | NM_018489 | ASH1 // ASH1L1 | 2437417 | chr1 | − | 153571685 | 153798937 | 1.05 |
| (Drosophila) | | | // FLJ10504 // | | | | | | |
| | | | KIAA1420 | | | | | | |
| FLJ36874 protein | FLJ36874 | NM_152716 | MGC125671 // | 3374746 | chr11 | − | 59160770 | 59193099 | 1.05 |
| | | | MGC125672 | | | | | | |
| thyroid hormone receptor associated protein 2 | THRAP2 | NM_015335 | DKFZp781D0112 // | 3473083 | chr12 | − | 114868953 | 115419724 | 1.05 |
| | | | FLJ21627 // | | | | | | |
| | | | KIAA1025 // | | | | | | |
| | | | MED13L // | | | | | | |
| | | | PROSIT240 // | | | | | | |
| | | | TRAP240L | | | | | | |
| B-cell CLL/lymphoma 10 | BCL10 | NM_003921 | CARMEN // CIPER | 2420808 | chr1 | − | 85504067 | 85516171 | 1.05 |
| | | | // CLAP // c-E10 | | | | | | |
| | | | // mE10 | | | | | | |
| collagen, type IV, alpha 3 (Goodpasture antigen) | COL4A3BP | NM_005731 // | CERT // CERTL // | 2862950 | chr5 | − | 74700067 | 74843717 | 1.05 |
| binding protein | | NM_031361 | FLJ20597 // GPBP | | | | | | |
| | | | // STARD11 | | | | | | |
| wings apart-like homolog (Drosophila) | WAPAL | NM_015045 | FOE // KIAA0261 | 3298738 | chr10 | − | 88184999 | 88286267 | 1.05 |
| | | | // WAPL | | | | | | |
| SFRS protein kinase 2 | SRPK2 | NM_182691 // | FLJ36101 // | 3066267 | chr7 | − | 104537547 | 104842466 | 1.05 |
| | | NM_182692 | SFRSK2 | | | | | | |
| RNA binding motif protein 22 | RBM22 | NM_018047 | FLJ10290 // | 2881521 | chr5 | − | 150050460 | 150060885 | 1.05 |
| | | | ZC3H16 | | | | | | |
| chromodomain helicase DNA binding protein 1 | CHD1 | NM_001270 | DKFZp686E2337 | 2868523 | chr5 | − | 98218502 | 98293051 | 1.05 |
| G1 to S phase transition 1 | GSPT1 | NM_002094 | 551G9.2 // ETF3A | 3680610 | chr16 | − | 11865619 | 11917408 | 1.05 |
| | | | // GST1 // eRF3a | | | | | | |
| ubiquilin 1 // death inducer-obliterator 1 | UBQLN1 // | NM_013438 // | DA41 // DSK2 // | 3212143 | chr9 | − | 85464717 | 85512928 | 1.05 |
| | DIDO1 | NM_053067 // | FLJ90054 // PLIC- | | | | | | |
| | | NM_022105 // | 1 // XDRP1 // | | | | | | |
| | | NM_033081 // | BYE1 // C20orf158 | | | | | | |
| | | NM_080796 // | // DATF1 // DIDO2 | | | | | | |
| | | NM_080797 | // DIDO3 // DIO-1 | | | | | | |
| | | | // DIO1 // | | | | | | |
| | | | DKFZp434P1115 // | | | | | | |
| | | | FLJ11265 // | | | | | | |
| | | | KIAA0333 // | | | | | | |
| | | | MGC16140 // | | | | | | |
| | | | dJ885L7.8 | | | | | | |
| golgi associated, gamma adaptin ear containing, | GGA2 | NM_015044 | FLJ20966 // | 3685183 | chr16 | − | 23381059 | 23429320 | 1.05 |
| ARF binding protein 2 | | | KIAA1080 // VEAR | | | | | | |
| solute carrier family 2 (facilitated glucose | SLC2A3 | NM_006931 | FLJ90380 // | 3442854 | chr12 | − | 7917549 | 7980138 | 1.05 |
| transporter), member 3 | | | GLUT3 | | | | | | |
| protein tyrosine phosphatase, non-receptor | PTPN1 | NM_002827 | PTP1B | 3888721 | chr20 | + | 48560298 | 48634700 | 1.05 |
| type 1 | | | | | | | | | |
| megakaryoblastic leukemia (translocation) 1 | MKL1 | NM_020831 | BSAC // MAL // | 3961496 | chr22 | − | 39136248 | 39362660 | 1.05 |
| | | | MRTF-A | | | | | | |
| sequestosome 1 | SQSTM1 // | NM_003900 | A170 // OSIL // | 2844479 | chr5 | + | 179166014 | 179198853 | 1.05 |
| | OSIL | | PDB3 // ZIP3 // | | | | | | |
| | | | p60 // p62 // p62B | | | | | | |
| family with sequence similarity 78, member A | FAM78A | NM_033387 | C9orf59 // | 3227574 | chr9 | − | 133123292 | 133147120 | 1.05 |
| | | | FLJ00024 | | | | | | |
| coenzyme Q2 homolog, prenyltransferase | COQ2 | NM_015697 | CL640 // FLJ26072 | 2775965 | chr4 | − | 84404005 | 84424988 | 1.05 |
| small nuclear ribonucleoprotein polypeptide B″ | SNRPB2 | NM_003092 // | MGC24807 // | 3877776 | chr20 | + | 16645421 | 16670916 | 1.05 |
| | | NM_198220 | MGC45309 | | | | | | |
| bromodomain adjacent to zinc finger domain, 2A | BAZ2A | NM_013449 | DKFZp781B109 // | 3457947 | chr12 | − | 55275319 | 55316417 | 1.05 |
| | | | FLJ13768 // | | | | | | |
| | | | FLJ13780 // | | | | | | |
| | | | FLJ45876 // | | | | | | |
| | | | KIAA0314 // TIP5 | | | | | | |
| | | | // WALp3 | | | | | | |
| RNA binding motif protein 25 | RBM25 | NM_021239 | MGC105088 // | 3543411 | chr14 | + | 72594776 | 72658577 | 1.05 |
| | | | MGC117168 // | | | | | | |
| | | | RNPC7 // S164 | | | | | | |
| intogrator complex subunit 6 | INTS6 | NM_001039937 // | DBI-1 // DDX26 // | 3514488 | chr13 | − | 50826009 | 50927014 | 1.05 |
| | | NM_001039938 // | DDX26A // DICE1 | | | | | | |
| | | NM_012141 | // DKFZP434B105 | | | | | | |
| | | | // HDB // INT6 // | | | | | | |
| | | | Notchl2 | | | | | | |
| cyclin M3 | CNNM3 | XM_001127059 // | ACDP3 // | 2494749 | chr2 | + | 96843277 | 96864841 | 1.05 |
| | | NM_017623 // | DKFZp434I1016 // | | | | | | |
| | | NM_199078 | FLJ20018 | | | | | | |
| Mdm2, transformed 3T3 cell double minute 2, | MDM2 | NM_002392 // | HDMX // | 3421300 | chr12 | + | 67488176 | 67525479 | 1.05 |
| p53 binding protein (mouse) | | NM_006878 // | MGC71221 // hdm2 | | | | | | |
| | | NM_006879 // | | | | | | | |
| | | NM_006881 // | | | | | | | |
| | | NM_006882 | | | | | | | |
| colony stimulating factor 1 receptor, formerly | CSF1R | NM_005211 | C-FMS // CD115 | 2881187 | chr5 | − | 149412410 | 149473128 | 1.05 |
| McDonough feline sarcoma viral (v-fms) | | | CSFR // FIM2 | | | | | | |
| oncogene homolog | | | // FMS | | | | | | |
| thioredoxin-like 1 | TXNL1 | NM_004786 | TRP32 // TXL-1 // | 3809324 | chr18 | − | 52334063 | 52469773 | 1.05 |
| | | | TXNL // Txl | | | | | | |
| ELOVL family member 5, elongation of long | ELOVL5 | NM_021814 | HELO1 // RP3- | 2957596 | chr6 | − | 53179349 | 53321902 | 1.05 |
| chain fatty acids (FEN1/Elo2, SUR4/Elo3-like, | | | 483K16.1 // | | | | | | |
| yeast) | | | dJ483K16.1 | | | | | | |
| PFTAIRE protein kinase 1 | PFTK1 | NM_012395 | KIAA0834 // | 3012064 | chr7 | + | 90002707 | 90677837 | 1.05 |
| | | | PFTAIRE1 | | | | | | |
| nuclear mitotic apparatus protein 1 | NUMA1 | NM_006185 | NUMA | 3380901 | chr11 | − | 71389909 | 71469367 | 1.05 |
| sparc/osteonectin, cwcv and kazal-like domains | SPOCK2 | NM_014767 | testican-2 | 3293840 | chr10 | − | 73457514 | 73518527 | 1.05 |
| proteoglycan (testican) 2 | | | | | | | | | |
| clathrin, heavy chain (Hc) | CLTC | NM_004859 | CHC17 // CLH-17 | 3729172 | chr17 | + | 55052021 | 55127299 | 1.05 |
| | | | // CLTCL2 // Hc | | | | | | |
| | | | // KIAA0034 | | | | | | |
| F-box and leucine-rich repeat protein 11 | FBXL11 | NM_012308 | CXXC8 // | 3336696 | chr11 | + | 66643299 | 66782124 | 1.05 |
| | | | DKFZP434M1735 // | | | | | | |
| | | | FBL11 // FBL7 // | | | | | | |
| | | | FLJ00115 // | | | | | | |
| | | | FLJ46431 // | | | | | | |
| | | | JHDM1A // | | | | | | |
| | | | KAA1004 // LILINA | | | | | | |
| signal-induced proliferation-associated gene 1 | SIPA1 | NM_006747 // | MGC102688 // | 3335465 | chr11 | + | 65162171 | 65174965 | 1.05 |
| | | NM_153253 | MGC17037 // SPA1 | | | | | | |
| kelch-like 24 (Drosophila) // YEATS domain | KLHL24 // | NM_017644 // | DRE1 // FLJ25796 | 2655113 | chr3 | + | 184836105 | 184882100 | 1.05 |
| containing 2 | YEATS2 | NM_018023 | // FLJ10201 // | | | | | | |
| | | | FLJ12841 // | | | | | | |
| | | | FLJ13308 // | | | | | | |
| | | | KIAA1197 | | | | | | |
| thymopoietin | TMPO | NM_001032283 // | CMD1T // LAP2 // | 3427767 | chr12 | + | 97395927 | 97502543 | 1.05 |
| | | NM_001032284 // | MGC61508 // | | | | | | |
| | | NM_003276 | PRO0866 // TP | | | | | | |
| STT3, subunit of the oligosaccharyltransferase | STT3B | NM_178862 | FLJ90106 // SIMP | 2615600 | chr3 | + | 31544326 | 31662283 | 1.05 |
| complex, homolog B (S. cerevisiae) | | | // STT3-B | | | | | | |
| ATP-binding cassette, sub-family F (GCN20), | ABCF1 | NM_001025091 // | ABC27 // ABC50 | 2901687 | chr6 | + | 30647103 | 30667268 | 1.05 |
| member 1 | | NM_001090 | | | | | | | |
| CUG triplet repeat, RNA binding protein 2 | CUGBP2 | NM_001025076 // | BRUNOL3 // ETR- | 3234760 | chr10 | + | 10544225 | 11418680 | 1.05 |
| | | NM_001025077 // | 3 // NAPOR | | | | | | |
| | | NM_006561 | | | | | | | |
| major histocompatibility complex, class II, DR | HLA-DRA | NM_019111 | HLA-DRA1 | 2903189 | chr6 | + | 32496972 | 32520923 | 1.05 |
| RAN binding protein 2 | RANBP2 // | NM_006267 | NUP358 // TRP1 | 2499158 | chr2 | + | 108702376 | 108768725 | 1.05 |
| | RGPD4 | | // TRP2 | | | | | | |
| G protein-coupled receptor kinase 5 | GRK5 | NM_005308 | GPRK5 | 3267036 | chr10 | + | 120957193 | 121256088 | 1.05 |
| FRY-like | FRYL | XM_001134434 // | DKFZp686E205 // | 2768468 | chr4 | − | 48194140 | 48272015 | 1.05 |
| | | XM_001134456 // | FLJ16177 // | | | | | | |
| | | NM_015030 | KIAA0826 | | | | | | |
| bromodomain containing 1 | BRD1 | NM_014577 | BRL // BRPF1 // | 3965314 | chr22 | − | 48552694 | 48636916 | 1.05 |
| | | | BRPF2 // | | | | | | |
| | | | DKFZp686F0325 | | | | | | |
| nuclear factor of activated T-cells, cytoplasmic, | NFATC2 | NM_012340 // | KIAA0611 // NFAT1 | 3909553 | chr20 | − | 49436909 | 49603743 | 1.05 |
| calcineurin-dependent 2 | | NM_173091 | // NFATP | | | | | | |
| TATA element modulatory factor 1 | TMF1 | NM_007114 | ARA160 | 2681157 | chr3 | − | 69151671 | 69184154 | 1.05 |
| ral guanine nucleotide dissociation stimulator | RALGDS | NM_001042368 // | FLJ20922 // RGF | 3228463 | chr9 | − | 134962931 | 135014381 | 1.05 |
| | | NM_006266 | // RalGEF | | | | | | |
| GATA binding protein 2 | GATA2 | NM_032638 | MGC2306 // NFE1B | 2694314 | chr3 | − | 129675850 | 129700720 | 1.05 |
| forty-two-three domain containing 1 | FYTTD1 | NM_001011537 // | DKFZp761B1514 | 2659887 | chr3 | + | 198960821 | 198996244 | 1.05 |
| | | NM_032288 | | | | | | | |
| splicing factor, arginine/serine-rich 1 (splicing | SFRS1 | NM_001078166 // | ASF // MGC5228 | 3764103 | chr17 | − | 53421412 | 53439635 | 1.05 |
| factor 2, alternate splicing factor) | | NM_006924 | // SF2 // SF2p33 | | | | | | |
| | | | // SRp30a | | | | | | |
| transcription elongation regulator 1 | TCERG1 | NM_001040006 // | CA150 // | 2834093 | chr5 | + | 145754916 | 145871713 | 1.05 |
| | | NM_006706 | MGC133200 // | | | | | | |
| | | | TAF2S | | | | | | |
| 3-hydroxy-3-methylglutaryl-Coenzyme A | HMGCS1 | NM_002130 | HMGCS // | 2855501 | chr5 | − | 43324231 | 43349337 | 1.05 |
| synthase 1 (soluble) | | | MGC90332 | | | | | | |
| inositol 1,4,5-trisphosphate 3-kinase B | ITPKB | NM_002221 | IP3K // IP3K-B // | 2458921 | chr1 | − | 224865889 | 225014518 | 1.05 |
| | | | PIG37 | | | | | | |
| insulin-like growth factor 1 receptor | IGF1R | NM_000875 | CD221 // IGFIR // | 3610804 | chr15 | + | 97010298 | 97320602 | 1.05 |
| | | | JTK13 // | | | | | | |
| | | | MGC142170 // | | | | | | |
| | | | MGC142172 // | | | | | | |
| | | | MGC18216 | | | | | | |
| ribulose-5-phosphate-3-epimerase // | RPE // CCBP2 | NM_006916 // | MGC2636 // RPE2- | 2525852 | chr2 | + | 210575559 | 210637315 | 1.05 |
| chemokine binding protein 2 | | NM_199229 // | 1 // CCR10 // | | | | | | |
| | | NM_001296 | CCR9 // CMKBR9 | | | | | | |
| | | | // D6 // | | | | | | |
| | | | MGC126678 // | | | | | | |
| | | | MGC138250 // hD6 | | | | | | |
| KIAA1429 | KIAA1429 | NM_015496 // | DKFZP434I116 // | 3145020 | chr8 | − | 95569109 | 95634851 | 1.05 |
| | | NM_183009 | DKFZp781B2117 // | | | | | | |
| | | | MGC138493 // | | | | | | |
| | | | MGC141940 // | | | | | | |
| | | | MSTP054 | | | | | | |
| dual-specificity tyrosine-(Y)-phosphorylation | DYRK1A | NM_001396 // | DYRK // DYRK1 // | 3920566 | chr21 | + | 37661116 | 37857051 | 1.05 |
| regulated kinase 1A | | NM_101395 // | HP86 // MNB // | | | | | | |
| | | NM_130436 // | MNBH | | | | | | |
| | | NM_130437 // | | | | | | | |
| | | NM_130438 | | | | | | | |
| coronin 7 | CORO7 | NM_024535 | 0610011B16Rik // | 3678083 | chr16 | − | 4343041 | 4406954 | 1.05 |
| | | | CRN7 // FLJ22021 | | | | | | |
| | | | // FLJ44188 | | | | | | |
| ribosomal protein S6 kinase, 70 kDa, polypeptide 1 | RPS6KB1 | NM_003161 | PS6K // S6K // | 3729294 | chr17 | + | 55325239 | 55429105 | 1.05 |
| | | | S6K1 // STK14A // | | | | | | |
| | | | p70(S6K)-alpha // | | | | | | |
| | | | p70-S6K // p70- | | | | | | |
| | | | alpha | | | | | | |
| MAK10 homolog, amino-acid N- | MAK10 | NM_024635 | FLJ21613 // | 3177563 | chr9 | + | 87733620 | 87828205 | 1.05 |
| acetyltransferase subunit, (S. cerevisiae) | | | FLJ22643 // | | | | | | |
| | | | bA379P1.1 | | | | | | |
| family with sequence similarity 62 (C2 domain | FAM62B | NM_020728 | CHR2SYT // | 3082248 | chr7 | − | 158205107 | 158355198 | 1.05 |
| containing) member B | | | ESYT2 // KIAA1228 | | | | | | |
| guanylate binding protein 4 // guanylate binding | GBP4 // GBP7 | NM_052941 // | Mpa2 // FLJ38822 | 2421995 | chr1 | − | 89419435 | 89437211 | 1.05 |
| protein 7 | | NM_207398 | // GBP4L | | | | | | |
| 5-methyltetrahydrofolate-homocysteine | MTRR | NM_002454 // | MGC129643 // | 2800906 | chr5 | + | 7922217 | 7954221 | 1.05 |
| methyltransferase reductase | | NM_024010 | MSR | | | | | | |
| tripeptidyl peptidase II | TPP2 | NM_003291 | FLJ40359 | 3499453 | chr13 | + | 101981581 | 102131173 | 1.05 |
| acyl-CoA synthetase short-chain family | ACSS1 | NM_032501 | ACAS2L // | 3901696 | chr20 | − | 24934882 | 24987616 | 1.05 |
| member 1 | | | AceCS2L // | | | | | | |
| | | | FLJ45659 // | | | | | | |
| | | | MGC33843 | | | | | | |
| tousled-like kinase 1 | TLK1 | NM_012290 | KIAA0137 // PKU- | 2586603 | chr2 | − | 171538552 | 171796060 | 1.05 |
| | | | BETA | | | | | | |
| secretory carrier membrane protein 1 | SCAMP1 | NM_052822 // | SCAMP // | 2817053 | chr5 | + | 77691978 | 77812317 | 1.05 |
| | | NM_004866 | SCAMP37 | | | | | | |
| tripartite motif-containing 28 | TRIM28 | NM_005762 | FLJ29029 // KAP1 | 3844238 | chr19 | + | 63740597 | 63753931 | 1.05 |
| | | | // RNF96 // TF1B | | | | | | |
| | | | // TIF1B | | | | | | |
| chromosome 11 open reading frame 58 | C11orf58 | NM_014267 | IMAGE145052 // | 3322048 | chr11 | + | 16716744 | 16734468 | 1.05 |
| | | | MGC117265 // | | | | | | |
| | | | SMAP | | | | | | |
| serine incorporator 1 | SERINC1 | NM_020755 | KIAA1253 // TDE1L | 2972310 | chr6 | − | 122804811 | 122948968 | 1.05 |
| | | | // TDE2 // TMS-2 | | | | | | |
| karyopherin (importin) beta 1 | KPNB1 | NM_002265 | IMB1 // IPOB // | 3724782 | chr17 | + | 43082272 | 43117868 | 1.05 |
| | | | Impnb // MGC2155 | | | | | | |
| | | | // MGC2156 // | | | | | | |
| | | | MGC2157 // NTF97 | | | | | | |
| hypothetical FLJ46363 // ataxin 2-like | FLJ46363 // | NM_207434 // | —// A2D // A2LG | 3654859 | chr16 | + | 28617718 | 28756043 | 1.05 |
| | ATXN2L | XM_001127543 // | // A2LP // A2RP | | | | | | |
| | | NM_007245 // | | | | | | | |
| | | NM_017492 // | | | | | | | |
| | | NM_145714 // | | | | | | | |
| | | NM_148414 // | | | | | | | |
| | | NM_148415 // | | | | | | | |
| | | NM_148416 | | | | | | | |
| quiescin Q6 | QSCN6 // | NM_001004128 // | Q6 // QSOX1 | 2369950 | chr1 | + | 178353067 | 178436483 | 1.05 |
| | FLJ23867 | NM_002826 | | | | | | | |
| splicing factor, arginine/serine-rich 6 | SFRS6 | NM_006275 | B52 // MGC5045 | 3886050 | chr20 | + | 41519932 | 41526301 | 1.05 |
| | | | // SRP55 | | | | | | |
| moesin | MSN | NM_002444 | — | 3979659 | chrX | + | 64804248 | 64878488 | 1.05 |
| intersectin 2 | ITSN2 | NM_006277 // | KIAA1256 // | 2544238 | chr2 | − | 24279241 | 24436906 | 1.05 |
| | | NM_019595 // | SH3D1B // SH3P18 | | | | | | |
| | | NM_147152 | // SWA // SWAP | | | | | | |
| SAFB-like, transcription modulator | SLTM | NM_001013843 // | DKFZp762G052 // | 3626704 | chr15 | − | 56949072 | 57013564 | 1.05 |
| | | NM_017968 // | FLJ10005 // | | | | | | |
| | | NM_024755 | FLJ13213 // Met | | | | | | |
| transcription factor 4 | TCF4 | NM_003199 | E2-2 // ITF2 // | 3808854 | chr18 | − | 51010981 | 51506795 | 1.05 |
| | | | MGC149723 // | | | | | | |
| | | | MGC149724 // | | | | | | |
| | | | SEF2 // SEF2-1 // | | | | | | |
| | | | SEF2-1A // SEF2- | | | | | | |
| | | | 1B | | | | | | |
| DEAH (Asp-Glu-Ala-His) box polypeptide 9 | DHX9 | NM_001357 | DDX9 // LKP // | 2370991 | chr1 | + | 181075106 | 181123738 | 1.05 |
| | | | NDH II // NDHII // | | | | | | |
| | | | RHA | | | | | | |
| chromosome 10 open reading frame 119 | C10orf119 | NM_024834 | FLJ13081 // | 3309755 | chr10 | − | 121568302 | 121642742 | 1.05 |
| | | | FLJ36756 | | | | | | |
| SET domain containing 2 | SETD2 | NM_014159 | FLJ16420 // | 2672532 | chr3 | − | 47032936 | 47180418 | 1.05 |
| | | | FLJ22472 // | | | | | | |
| | | | FLJ23184 // | | | | | | |
| | | | FLJ45883 // HIF-1 | | | | | | |
| | | | // HSPC069 // | | | | | | |
| | | | HYPB // KIAA1732 | | | | | | |
| golgi reassembly stacking protein 2, 55 kDa // | GORASP2 // | NM_015530 // | DKFZP434D156 // | 2515050 | chr2 | + | 171457150 | 171551619 | 1.05 |
| dehydrogenase/reductase (SDR family) member 9 | DHRS9 | NM_005771 // | FLJ13139 // | | | | | | |
| | | NM_199204 | GOLPH6 // | | | | | | |
| | | | GRASP55 // GRS2 | | | | | | |
| | | | // p59 // 3alpha- | | | | | | |
| | | | HSD // RDH15 // | | | | | | |
| | | | RDHL // RETSDR8 | | | | | | |
| target of myb1 (chicken) | TOM1 | NM_005488 | FLJ33404 | 3944084 | chr22 | + | 34025845 | 34073962 | 1.05 |
| ATPase, H+ transporting, lysosomal 42 kDa, V1 | ATP6V1C1 | NM_001007254 // | ATP6C // ATP6D | 3110171 | chr8 | + | 104102441 | 104171800 | 1.05 |
| subunit C1 | | NM_001695 | // FLJ20057 // | | | | | | |
| | | | VATC // Vma5 | | | | | | |
| potassium channel tetramerisation domain | KCTD20 | NM_173562 | C6orf69 // | 2905069 | chr6 | + | 36518349 | 36566874 | 1.05 |
| containing 20 | | | MGC14254 // | | | | | | |
| | | | dJ108K11.3 | | | | | | |
| serine/threonine kinase 10 | STK10 | NM_005990 | LOK // PRO2729 | 2887048 | chr5 | − | 171401689 | 171547855 | 1.05 |
| GA binding protein transcription factor, alpha | GABPA // | NM_002040 // —// | E4TF1-60 // | 3916576 | chr21 | + | 26028762 | 26066641 | 1.05 |
| subunit 60 kDa // GA binding protein | GABPAP | NR_002723 | E4TF1A // NFT2 // | | | | | | |
| transcription factor, alpha subunit pseudogene | | | NRF2 // NRF2A // | | | | | | |
| | | | E4TF1 // E4TF1B | | | | | | |
| | | | // GABPB1 | | | | | | |
| centaurin, beta 2 | CENTB2 | NM_012287 | ACAP2 // CNT-B2 | 2712040 | chr3 | − | 196476779 | 196645492 | 1.05 |
| | | | // KIAA0041 | | | | | | |
| fibrosin 1 | FBS1 | NM_022452 | FBS // FLJ11618 | 3656362 | chr16 | + | 30577810 | 30590035 | 1.05 |
| G protein-coupled receptor kinase interactor 2 | GIT2 | NM_014776 // | CAT-2 // | 3471005 | chr12 | − | 108852000 | 108918536 | 1.05 |
| | | NM_057169 // | DKFZp686G01261 | | | | | | |
| | | NM_057170 // | // KIAA0148 // | | | | | | |
| | | NM_139201 | MGC760 | | | | | | |
| vimentin | VIM | NM_003380 | FLJ36605 | 3236958 | chr10 | + | 17296705 | 17357733 | 1.05 |
| nuclear receptor coactivator 2 | NCOA2 | NM_006540 | GRIP1 // | 3139722 | chr8 | − | 71184397 | 71478626 | 1.05 |
| | | | MGC138808 // | | | | | | |
| | | | NCoA-2 // TIF2 | | | | | | |
| thrombospondin 1 | THBS1 | NM_003246 | THBS // TSP // | 3589458 | chr15 | + | 37614946 | 37678406 | 1.05 |
| | | | TSP1 | | | | | | |
| trinucleotide repeat containing 6B | TNRC6B | NM_001024843 // | KIAA1093 | 3946192 | chr22 | + | 38770332 | 39061757 | 1.05 |
| | | NM_015088 | | | | | | | |
| GTPase activating protein (SH3 domain) binding | G3BP2 // | NM_012297 // | —// MGC119326 // | 2773756 | chr4 | − | 76786807 | 76869171 | 1.05 |
| protein 2 // NMDA receptor regulated 2 // | NARG2 // | NM_203504 // | MGC119329 // | | | | | | |
| RAR-related orphan receptor A | RORA | NM_203505 // | NR1F1 // ROR1 // | | | | | | |
| | | NM_001018089 // | ROR2 // ROR3 // | | | | | | |
| | | NM_024611 // | RZRA | | | | | | |
| | | NM_002943 // | | | | | | | |
| | | NM_134260 // | | | | | | | |
| | | NM_134261 // | | | | | | | |
| | | NM_134262 | | | | | | | |
| calcium/calmodulin-dependent protein kinase | CAMK4 | NM_001744 | CaMK-GR // | 2823880 | chr5 | + | 110587242 | 110854177 | 1.05 |
| IV | | | MGC36771 | | | | | | |
| oxidative-stress responsive 1 | OXSR1 | NM_005109 | KIAA1101 // OSR1 | 2617579 | chr3 | + | 38182030 | 38271979 | 1.05 |
| ATPase, Na+/K+ transporting, alpha 1 | ATP1A1 | NM_000701 // | MGC3285 // | 2353477 | chr1 | + | 116706775 | 116754386 | 1.05 |
| polypeptide | | NM_001001586 | MGC51750 | | | | | | |
| transportin 1 | TNPO1 | NM_002270 // | IPO2 // KPNB2 // | 2815043 | chr5 | + | 72148176 | 72245960 | 1.05 |
| | | NM_153188 | MIP // MIP1 // | | | | | | |
| heterogeneous nuclear ribonucleoprotein U-like 1 | HNRPUL1 | NM_144733 // | E1B-AP5 // | 3834089 | chr19 | + | 46460006 | 46505508 | 1.05 |
| | | NM_144734 // | E1BAP5 // | | | | | | |
| | | NM_007040 // | FLJ12944 | | | | | | |
| | | NM_144732 | | | | | | | |
| CD300e molecule // fibrillin 2 (congenital | CD300E // | NM_181449 // | CD300LE // CLM2 | 3770345 | chr17 | − | 70117621 | 70131474 | 1.05 |
| contractural arachnodactyly) | FBN2 | NM_001999 | // IREM2 // CCA | | | | | | |
| CD55 molecule, decay accelerating factor for | CD55 | NM_000574 | CR // DAF // TC | 2377229 | chr1 | + | 205561488 | 205607671 | 1.05 |
| complement (Cromer blood group) | | | | | | | | | |
| TBC1 domain family, member 15 | TBC1D15 | NM_022771 | DKFZp686M1379 // | 3422326 | chr12 | + | 70519754 | 70604360 | 1.05 |
| | | | DKFZp761D0223 // | | | | | | |
| | | | FLJ12085 | | | | | | |
| v-raf murine sarcoma viral oncogene homolog | BRAF | NM_004333 | B-raf1 // BRAF1 | 3076340 | chr7 | − | 139994192 | 140274640 | 1.05 |
| B1 | | | // MGC126806 // | | | | | | |
| | | | MGC138284 // | | | | | | |
| | | | RAFB1 | | | | | | |
| splicing factor, arginine/serine-rich 5 | SFRS5 | NM_001039465 // | HRS // SRP40 | 3542207 | chr14 | + | 69263363 | 69308465 | 1.05 |
| | | NM_006925 | | | | | | | |
| GRAM domain containing 1A | GRAMD1A | NM_020895 | FLJ22411 // | 3830002 | chr19 | + | 40161899 | 40210121 | 1.05 |
| | | | FLJ90346 // | | | | | | |
| | | | KIAA1533 | | | | | | |
| mediator of RNA polymerase II transcription, | MED28 | NM_025205 | 1500003D12Rik // | 2720181 | chr4 | + | 17225344 | 17244808 | 1.05 |
| subunit 28 homolog (S. cerevisiae) | | | DKFZP434N185 // | | | | | | |
| | | | EG1 // magicin | | | | | | |
| jumonji, AT rich interactive domain 1A | JARID1A | NM_001042603 // | RBBP2 // RBP2 | 3439603 | chr12 | − | 259504 | 368944 | 1.05 |
| | | NM_005056 | | | | | | | |
| activating transcription factor 6 | ATF6 | NM_007348 | − | 2363919 | chr1 | + | 160002678 | 160208836 | 1.05 |
| matrin 3 | MATR3 | NM_018834 // | DKFZp686K0542 // | 2831124 | chr5 | + | 138505686 | 138695245 | 1.05 |
| | | NM_199189 | DKFZp686K23100 | | | | | | |
| | | | // KIAA0723 // | | | | | | |
| | | | MGC9105 | | | | | | |
| granzyme B (granzyme 2, cytotoxic T- | GZMB | NM_004131 | CCPI // CGL-1 // | 3558375 | chr14 | − | 24169951 | 24173308 | 1.05 |
| lymphocyte-associated serine esterase 1) | | | CGL1 // CSP-B // | | | | | | |
| | | | CSPB // CTLA1 // | | | | | | |
| | | | CTSGL1 // HLP // | | | | | | |
| | | | SECT | | | | | | |
| cyclin L1 | CCNL1 | NM_020307 | BM-001 // | 2702307 | chr3 | − | 158305077 | 158361233 | 1.05 |
| | | | PRO1073 // ania- | | | | | | |
| neighbor of BRCA1 gene 1 | NBR1 | XM_001124650 // | 1A1-3B // | 3722417 | chr17 | + | 38563761 | 38719231 | 1.05 |
| | | XM_001124827 // | KIAA0049 // M17S2 | | | | | | |
| | | XM_001127781 // | // MIG19 | | | | | | |
| | | NM_005899 // | | | | | | | |
| | | NM_031858 // | | | | | | | |
| | | NM_031862 | | | | | | | |
| tyrosyl-tRNA synthetase | YARS | NM_003680 | CMTDIC // TYRRS | 2405192 | chr1 | − | 33012453 | 33056331 | 1.05 |
| | | | // YRS // YTS | | | | | | |
| solute carrier family 25 (mitochondrial carrier, | SLC25A3 | NM_213612 // | OK/SW-cl.48 // | 3427820 | chr12 | + | 97511471 | 97519906 | 1.05 |
| phosphate carrier), member 3 | | NM_002635 // | PHC | | | | | | |
| | | NM_005888 // | | | | | | | |
| | | NM_213611 | | | | | | | |
| CCR4-NOT transcription complex, subunit 1 | CNOT1 | NM_016284 // | AD-005 // CDC39 | 3693673 | chr16 | − | 57111094 | 57236550 | 1.05 |
| | | NM_206999 | // DKFZp686E0722 | | | | | | |
| | | | // FLJ90644 // | | | | | | |
| | | | KIAA1007 // NOT1 | | | | | | |
| | | | // NOT1H | | | | | | |
| ankyrin repeat domain 13 family, member D | ANKRD13D | XM_001129739 // | MGC50828 | 3336857 | chr11 | + | 66812660 | 66826524 | 1.05 |
| | | NM_207354 | | | | | | | |
| PHD finger protein 10 | PHF10 | NM_018288 // | FLJ10975 // | 2986084 | chr6 | − | 169845929 | 169866056 | 1.05 |
| | | NM_133325 | MGC111009 // | | | | | | |
| | | | XAP135 | | | | | | |
| hypothetical protein KIAA1434 | RP5-1022P6.2 | NM_019593 | FLJ11085 // | 3896370 | chr20 | − | 5473033 | 5546357 | 1.05 |
| | | | KIAA1434 // | | | | | | |
| | | | MGC26147 | | | | | | |
| ribosomal protein S6 kinase, 90 kDa, polypeptide 3 | RPS6KA3 | NM_004586 | CLS // HU-3 // | 4002173 | chrX | − | 20077954 | 20254253 | 1.05 |
| | | | ISPK-1 // | | | | | | |
| | | | MAPKAPK1B // | | | | | | |
| | | | MRX19 // RSK // | | | | | | |
| | | | RSK2 // S6K- | | | | | | |
| | | | alpha3 // p90- | | | | | | |
| | | | RSK2 // pp90RSK2 | | | | | | |
| UPF2 regulator of nonsense transcripts | UPF2 | NM_015542 // | DKFZP434D222 // | 3277662 | chr10 | − | 11984615 | 12125155 | 1.05 |
| homolog (yeast) | | NM_080599 | HUPF2 // | | | | | | |
| | | | KIAA1408 // | | | | | | |
| | | | MGC138834 // | | | | | | |
| | | | MGC138835 // | | | | | | |
| thioredoxin interacting protein | TXNIP | NM_006472 | EST01027 // | 2356115 | chr1 | + | 144149846 | 144164251 | 1.05 |
| | | | HHCPA78 // THIF | | | | | | |
| | | | // VDUP1 | | | | | | |
| protein phosphatase 1, regulatory (inhibitor) | PPP1R16B | NM_015568 | ANKRD4 // | 3884830 | chr20 | + | 36866338 | 36985066 | 1.05 |
| subunit 16B | | | KIAA0823 // TIMAP | | | | | | |
| peptidylprolyl isomerase F (cyclophilin F) | PPIF | NM_005729 | CYP3 // Cyp-D // | 3253880 | chr10 | + | 80777230 | 80785093 | 1.05 |
| | | | FLJ90798 // | | | | | | |
| | | | MGC117207 | | | | | | |
| 3-hydroxy-3-methylglutaryl-Coenzyme A | HMGCR | NM_000859 | — | 2815965 | chr5 | + | 74668800 | 74700136 | 1.05 |
| reductase | | | | | | | | | |
| chloride intracellular channel 1 | CLIC1 | NM_001288 | G6 // NCC27 | 2949330 | chr6 | − | 31806365 | 31815144 | 1.05 |
| HBS1-like (S. cerevisiae) // aldehyde | HBS1L // | NM_006620 // | DKFZp686L13262 | 2975287 | chr6 | − | 135314409 | 135417715 | 1.05 |
| dehydrogenase 8 family, member A1 | ALDH8A1 | NM_022568 // | // EF-1a // ERFS | | | | | | |
| | | NM_170771 | // HBS1 // | | | | | | |
| | | | HSPC276 // | | | | | | |
| | | | ALDH12 // | | | | | | |
| | | | DJ352A20.2 // | | | | | | |
| | | | DKFZp779D2315 // | | | | | | |
| vav 3 oncogene | VAV3 | NM_001079874 // | FLJ40431 | 2426385 | chr1 | − | 107914594 | 108309371 | 1.05 |
| | | NM_006113 | | | | | | | |
| Rho guanine nucleotide exchange factor (GEF) 7 | ARHGEF7 | NM_003899 // | BETA-PIX // | 3501661 | chr13 | + | 110565635 | 110756075 | 1.05 |
| | | NM_145735 | COOL1 // | | | | | | |
| | | | DKFZp761K1021 // | | | | | | |
| | | | KIAA0142 // | | | | | | |
| | | | KIAA0412 // | | | | | | |
| | | | Nbla10314 // P50 | | | | | | |
| | | | // P50BP// P85 | | | | | | |
| | | | // P85COOL1 // | | | | | | |
| | | | P85SPR // PAK3 | | | | | | |
| | | | // PIXB | | | | | | |
| SWI/SNF related, matrix associated, actin | SMARCA5 | NM_003601 | ISWI // SNF2H // | 2745646 | chr4 | + | 144616165 | 144697227 | 1.05 |
| dependent regulator of chromatin, subfamily a, | | | WCRF135 // hISWI | | | | | | |
| member 5 | | | // hSNF2H | | | | | | |
| proteasome maturation protein | POMP | NM_015932 | C13orf12 // | 3483348 | chr13 | + | 28125188 | 28151050 | 1.05 |
| | | | HSPC014 // | | | | | | |
| | | | PNAS-110 // UMP1 | | | | | | |
| DnaJ (Hsp40) hamolog, subfamily B, member 6 | DNAJB6 | NM_005494 // | DKFZp566D0824 // | 3034027 | chr7 | + | 156799667 | 156902887 | 1.05 |
| | | NM_058246 | FLJ42837 // | | | | | | |
| | | | HHDJ1 // HSJ-2 | | | | | | |
| | | | // HSJ2 // | | | | | | |
| | | | MGC1152 // | | | | | | |
| | | | MGC117297 // | | | | | | |
| zinc finger RNA binding protein | ZFR | NM_016107 | FLJ41312 | 2852333 | chr5 | − | 32390101 | 32537319 | 1.05 |
| tetraspanin 14 | TSPAN14 | NM_030927 | DC-TM4F2 // | 3254521 | chr10 | + | 82203922 | 82272901 | 1.05 |
| | | | MGC11352 // | | | | | | |
| | | | TM4SF14 | | | | | | |
| myelin basic protein | MBP | NM_001025081 // | MGC99675 | 3814063 | chr18 | − | 72853742 | 73023063 | 1.05 |
| | | NM_001025090 // | | | | | | | |
| | | NM_001025092 // | | | | | | | |
| | | NM_001025094 // | | | | | | | |
| | | NM_001025098 // | | | | | | | |
| | | NM_001025100 // | | | | | | | |
| | | NM_001025101 // | | | | | | | |
| | | NM_002385 | | | | | | | |
| testis derived transcript (3 LIM domains) | TES | NM_015641 // | DKFZP586B2022 // | 3020192 | chr7 | + | 115637811 | 115743800 | 1.05 |
| | | NM_152829 | MGC1146 // TESS | | | | | | |
| | | | // TESS-2 // | | | | | | |
| | | | TESTIN | | | | | | |
| microtubule-associated protein 1 light chain 3 | MAP1LC3B | NM_022818 | MAP1A/1BLC3 | 3672830 | chr16 | + | 85982917 | 85996864 | 1.05 |
| beta | | | | | | | | | |
| transcription factor binding to IGHM enhancer 3 | TFE3 | NM_006521 | RCCP2 // TFEA | 4007734 | chrX | − | 48771186 | 48787973 | 1.05 |
| CHMP family, member 7 | CHMP7 | NM_152272 | MGC29816 | 3089853 | chr8 | + | 23157105 | 23176288 | 1.05 |
| potassium channel tetramerisation domain | KCTD10 | NM_031954 | FLJ41739 // | 3470793 | chr12 | − | 108370845 | 108399528 | 1.05 |
| containing 10 | | | MSTP028 // | | | | | | |
| | | | ULRO61 | | | | | | |
| ets variant gene 6 (TEL oncogene) | ETV6 | NM_001987 | TEL // TEL/ABL | 3405032 | chr12 | + | 11645490 | 11939588 | 1.05 |
| programmed cell death 6 interacting protein | PDCD6IP | NM_013374 | AIP1 // Alix // | 2616317 | chr3 | + | 33743952 | 33886195 | 1.05 |
| | | | DRIP4 // HP95 // | | | | | | |
| | | | MGC17003 | | | | | | |
| ras homolog gene family, member F (in filopodia) | RHOF | NM_019034 | ARHF // FLJ20247 | 3475324 | chr12 | − | 120698553 | 120716624 | 1.05 |
| | | | // RIF | | | | | | |
| lymphocyte cytosolic protein 2 (SH2 domain | LCP2 | NM_005565 | SLP-76 // SLP76 | 2886595 | chr5 | − | 169607157 | 169657789 | 1.05 |
| containing leukocyte protein of 76 kDa) | | | | | | | | | |
| vitamin D (1,25-dihydroxyvitamin D3) receptor | VDR | NM_000376 // | NR1I1 | 3452818 | chr12 | − | 46521596 | 46606823 | 1.05 |
| | | NM_001017535 | | | | | | | |
| diablo homolog (Drosophila) | DIABLO | NM_019887 // | DIABLO-S // | 3475511 | chr12 | − | 121257461 | 121277963 | 1.05 |
| | | NM_138929 // | FLJ10537 // | | | | | | |
| | | NM_138930 | // FLJ25049 // SMAC | | | | | | |
| | | | // SMAC3 | | | | | | |
| B-cell translocation gene 1, anti-proliferative | BTG1 | NM_001731 | — | 3465409 | chr12 | − | 90902887 | 91063960 | 1.05 |
| CD53 molecule | CD53 | NM_000560 // | MOX44 // | 2351572 | chr1 | + | 111215352 | 111296983 | 1.05 |
| | | NM_001040033 | TSPAN25 | | | | | | |
| damage-specific DNA binding protein 1, 127 kDa | DDB1 | XM_001128974 // | DDBA // UV-DDB1 | 3375245 | chr11 | − | 60823513 | 60857553 | 1.05 |
| | | XM_001128983 // | // XAP1 // XPCE | | | | | | |
| | | NM_001923 | // XPE // XPE-BF | | | | | | |
| ariadne homolog 2 (Drosophila) | ARIH2 | NM_006321 | ARI2 // FLJ10938 | 2621827 | chr3 | + | 48930742 | 48997971 | 1.05 |
| | | | // FLJ33921 // | | | | | | |
| | | | TRIAD1 | | | | | | |
| protein tyrosine phosphatase, non-receptor | PTPN12 | NM_002835 | PTP-PEST // | 3009959 | chr7 | + | 77004361 | 77123074 | 1.05 |
| type 12 | | | PTPG1 | | | | | | |
| meteorin, glial cell differentiation regulator-like | METRNL // | XM_941466 // | MGC99788 // | 3739431 | chr17 | + | 78630769 | 78653225 | 1.05 |
| // mediator of RNA polymerase II transcription, | MED25 | NM_001004431 // | ACID1 // ARC92 // | | | | | | |
| subunit 25 homolog (S. cerevisiae) | | NM_030973 | DKFZp434K0512 // | | | | | | |
| | | | MGC70671 // P78 | | | | | | |
| | | | // TCBAP0758 | | | | | | |
| trinucleotide repeat containing 6A | TNRC6A | NM_020847 // | CAGH26 // | 3653398 | chr16 | + | 24583665 | 24748478 | 1.05 |
| | | NM_014494 | DKFZp666E117 // | | | | | | |
| | | | FLJ22043 // GW1 | | | | | | |
| | | | // GW182 // | | | | | | |
| | | | KIAA1460 // | | | | | | |
| | | | MGC75384 // | | | | | | |
| | | | TNRC6 | | | | | | |
| ubiquitin specific peptidase 10 | USP10 | NM_005153 | KIAA0190 // | 3671873 | chr16 | + | 83291080 | 83371023 | 1.05 |
| | | | MGC2621 // UBPO | | | | | | |
| DnaJ (Hsp40) homolog, subfamily A, member 2 | DNAJA2 | NM_005880 | CPR3 // DJA2 // | 3690084 | chr16 | − | 45546783 | 45565155 | 1.05 |
| | | | DNAJ // DNJ3 // | | | | | | |
| | | | HIRIP4 // PRO3015 | | | | | | |
| | | | // RDJ2 | | | | | | |
| MYC binding protein 2 | MYCBP2 | NM_015057 | DKFZp686M08244 | 3518496 | chr13 | − | 76500672 | 76799212 | 1.05 |
| | | | // FLJ10106 // | | | | | | |
| | | | FLJ13826 // | | | | | | |
| | | | FLJ21597 // | | | | | | |
| | | | FLJ21646 // | | | | | | |
| | | | KIAA0916 // PAM | | | | | | |
| SEC24 related gene family, member B (S. cerevisiae) | SEC24B | XM_001130118 // | MGC48822 // | 2739079 | chr4 | + | 110510304 | 110681811 | 1.05 |
| | | NM_001042734 // | SEC24 | | | | | | |
| | | NM_006323 | | | | | | | |
| interferon gamma receptor 2 (interferon gamma | IFNGR2 | NM_005534 | AF-1 // IFGR2 // | 3918635 | chr21 | + | 33696906 | 33774607 | 1.05 |
| transducer 1) | | | IFNGT1 | | | | | | |
| VAMP (vesicle-associeted membrane protein)- | VAPA | NM_003574 // | MGC3745 // VAP- | 3778601 | chr18 | + | 9903984 | 9950012 | 1.05 |
| associated protein A, 33 kDa | | NM_194434 | 33 // VAP-A // | | | | | | |
| | | | VAP33 // hVAP-33 | | | | | | |
| SP100 nuclear antigen | SP100 | NM_001080391 // | DKFZp686E07254 | 2531377 | chr2 | + | 230985008 | 231142598 | 1.05 |
| | | NM_003113 | // FLJ00340 // | | | | | | |
| | | | FLJ34579 | | | | | | |
| transforming growth factor beta regulator 1 | TBRG1 | NM_032811 | FLJ14621 // | 3354174 | chr11 | + | 123997906 | 124011654 | 1.05 |
| | | | FLJ25020 // | | | | | | |
| | | | FLJ90113 // | | | | | | |
| | | | MGC129890 // | | | | | | |
| | | | NIAM // TB-5 | | | | | | |
| growth arrest and DNA-damage-inducible, beta | GADD45B | NM_015675 | DKFZP566B133 // | 3816509 | chr19 | + | 2425718 | 2469133 | 1.05 |
| | | | GADD45BETA // | | | | | | |
| | | | MYD118 | | | | | | |
| alanyl (membrane) aminopeptidase | ANPEP | NM_001150 | APN // CD13 // | 3638607 | chr15 | − | 88129142 | 88159617 | 1.05 |
| (aminopeptidase N, aminopeptidase M, | | | LAP1 // PEPN // | | | | | | |
| microsomal aminopeptidase, CD13, p150) | | | gp150 | | | | | | |
| proteasome (prosome, macropain) activator | PSME4 | NM_014614 | FLJ21864 // | 2553282 | chr2 | − | 53944710 | 54160919 | 1.05 |
| subunit 4 | | | KIAA0077 // | | | | | | |
| | | | MGC138374 // | | | | | | |
| | | | MGC142228 // | | | | | | |
| | | | PA200 | | | | | | |
| microtubule-associated protein, RP/EB family, | MAPRE1 | NM_012325 | EB1 // MGC117374 | 3882069 | chr20 | + | 30871304 | 30901864 | 1.05 |
| member 1 | | | // MGC129946 | | | | | | |
| BMI1 polycomb ring finger oncogene | BMI1 | NM_005180 | MGC12685 // | 3238491 | chr10 | + | 22650103 | 22660820 | 1.05 |
| | | | PCGF4 // RNF51 | | | | | | |
| CUG triplet repeat, RNA binding protein 1 | CUGBP1 | NM_001025596 // | BRUNOL2 // CUG- | 3372253 | chr11 | − | 47444068 | 47543613 | 1.05 |
| | | NM_006560 // | BP // CUGBP // | | | | | | |
| | | NM_198700 | NAB50 // hNab50 | | | | | | |
| transforming, acidic coiled-coil containing | TACC3 | NM_006342 | ERIC1 // | 2714955 | chr4 | + | 1692616 | 1716693 | 1.05 |
| protein 3 | | | MGC117382 // | | | | | | |
| | | | MGC133242 | | | | | | |
| zinc finger, CCHC domain containing 6 // | ZCCHC6 // | NM_024617 // | DKFZp666B142 // | 3212976 | chr9 | − | 88092469 | 88159805 | 1.05 |
| hypothetical protein MGC13114 | MGC13114 | NM_001040160 // | DKFZp686C11112 | | | | | | |
| | | NM_001040161 // | // DKFZp686F119 | | | | | | |
| | | NM_001040162 // | // DKFZp686I1269 | | | | | | |
| | | NM_001040163 // | // PAPD6 // JFP2 | | | | | | |
| | | NM_001040164 // | | | | | | | |
| | | NM_001040165 // | | | | | | | |
| | | NM_001040166 // | | | | | | | |
| | | NM_032366 | | | | | | | |
| C-type lectin domain family 4, member E | CLEC4E | NM_014358 | CLECSF9 // | 3443183 | chr12 | − | 8577026 | 8584811 | 1.05 |
| | | | MINCLE | | | | | | |
| interferon, gamma-inducible protein 18 | IFI16 | NM_005531 | IFNGIP1 // | 2362394 | chr1 | + | 157215279 | 157291561 | 1.05 |
| | | | MGC9466 // | | | | | | |
| | | | PYHIN2 | | | | | | |
| LMBR1 domain containing 1 | LMBRD1 | NM_018368 | C6orf209 // | 2960010 | chr6 | − | 70361077 | 70634121 | 1.05 |
| | | | FLJ11240 // RP11- | | | | | | |
| | | | 810I22.1 // | | | | | | |
| | | | bA810I22.1 | | | | | | |
| protein kinase D3 | PRKD3 | NM_005813 | EPK2 // PKC-NU | 2548500 | chr2 | − | 37331165 | 37405413 | 1.05 |
| | | | // PKD3 // PRKCN | | | | | | |
| | | | // nPKC-NU | | | | | | |
| CCR4-NOT transcription complex, subunit 7 | CNOT7 | NM_013354 // | CAF1 // hCAF-1 | 3125775 | chr8 | − | 17128912 | 17148758 | 1.05 |
| | | NM_054026 | | | | | | | |
| heterogeneous nuclear ribonucleoprotein M | HNRPM | NM_005968 // | DKFZp547H118 // | 3819543 | chr19 | + | 8415651 | 8459993 | 1.05 |
| | | NM_031203 | HNRNPM // | | | | | | |
| | | | HNRNPM4 // | | | | | | |
| | | | HNRPM4 // HTGR1 | | | | | | |
| | | | // NAGR1 | | | | | | |
| heterogeneous nuclear ribonucleoprotein H1 (H) | HNRPH1 | NM_005520 | DKFZp686A15170 | 2890148 | chr5 | − | 178970155 | 178993890 | 1.05 |
| | | | // HNRPH // | | | | | | |
| adenosine A2a receptor | ADORA2A | NM_000675 | ADORA2 // RDC8 | 3940099 | chr22 | + | 23143709 | 23200243 | 1.05 |
| | | | // hA2aR | | | | | | |
| PPAR binding protein | PPARBP | NM_004774 | CRSP1 // | 3755714 | chr17 | − | 34801544 | 34861069 | 1.05 |
| | | | CRSP200 // | | | | | | |
| | | | DRIP205 // | | | | | | |
| | | | DRIP230 // MED1 | | | | | | |
| | | | // MGC71488 // | | | | | | |
| | | | PBP // PPARGBP | | | | | | |
| | | | // RB18A // | | | | | | |
| serine/threonine kinase 17b | STK17B | NM_004226 | DRAK2 | 2593159 | chr2 | − | 196706552 | 196744596 | 1.05 |
| adhesion regulating molecule 1 | ADRM1 | NM_007002 // | GP110 // | 3892607 | chr20 | + | 60305485 | 60317305 | 1.05 |
| | | NM_175573 | MGC29536 // | | | | | | |
| splicing factor, arginine/serine-rich 9 | SFRS9 | NM_003769 | SRp30c | 3474502 | chr12 | − | 119372834 | 119407274 | 1.05 |
| E2F transcription factor 4, p107/p130-binding | E2F4 | NM_001950 | E2F-4 | 3665288 | chr16 | + | 65783266 | 65790299 | 1.05 |
| chromodomain helicase DNA binding protein 2 | CHD2 | NM_001042572 // | DKFZp781D1727 | 3609138 | chr15 | + | 91227096 | 91387351 | 1.05 |
| | | NM_001271 | | | | | | | |
| serpin peptidase inhibitor, clade B (ovalbumin), | SERPINB2 | NM_002575 | HsT1201 // PAI // | 3791935 | chr18 | + | 59704265 | 59722138 | 1.05 |
| member 2 | | | PAI-2 // PAI2 // | | | | | | |
| | | | PLANH2 | | | | | | |
| phosphatidylinositol 3,4,5-trisphosphate- | PREX1 | NM_020820 | KIAA1415 | 3908631 | chr20 | − | 46674205 | 46938090 | 1.05 |
| dependent RAC exchanger 1 | | | | | | | | | |
| pre-B-cell colony enhancing factor 1 // pre-B | PBEF1 // | NM_005746 // | 1110035O14Rik // | 3066818 | chr7 | − | 105625846 | 105713266 | 1.05 |
| cell enhancing factor 1 pseudogene | RP11-92J19.4 | | DKFZP666B131 // | | | | | | |
| | | | MGC117256 // | | | | | | |
| | | | NAMPT // PBEF // | | | | | | |
| | | | LOC646309 | | | | | | |
| interferon regulatory factor 1 | IRF1 | NM_002198 | IRF-1 // MAR | 2875348 | chr5 | − | 131846280 | 131893043 | 1.05 |
| nuclear receptor coactivator 4 | NCOA4 | NM_005437 | ARA70 // | 3246372 | chr10 | + | 51202191 | 51260947 | 1.05 |
| | | | DKFZp762E1112 // | | | | | | |
| | | | ELE1 // PTC3 // | | | | | | |
| | | | RFG | | | | | | |
| adrenergic, beta, receptor kinase 1 | ADRBK1 | NM_001619 | BARK1 // BETA- | 3336801 | chr11 | + | 66790523 | 66810933 | 1.05 |
| | | | ARK1 // GRK2 | | | | | | |
| BTAF1 RNA polymerase II, B-TFIID | BTAF1 | NM_003972 | KIAA0940 // | 3257938 | chr10 | + | 93673509 | 93821994 | 1.05 |
| transcription factor-associated, 170 kDa (Mot1 | | | MGC138406 // | | | | | | |
| homolog, S. cerevisiae) | | | MOT1 // TAF(II)170 | | | | | | |
| | | | // TAF172 // | | | | | | |
| | | | TAFII170 | | | | | | |
| golgi autoantigen, golgin subfamily a, 4 | GOLGA4 | NM_002078 | GCP2 // GOLG // | 2617041 | chr3 | + | 37259518 | 37452233 | 1.05 |
| | | | MU-RMS-40.18 // | | | | | | |
| | | | p230 | | | | | | |
| ras homolog gene family, member A | RHOA | NM_001664 | ARH12 // ARHA // | 2674242 | chr3 | − | 49371587 | 49424514 | 1.05 |
| | | | RHO12 // RHOH12 | | | | | | |
| regulatory factor X, 5 (influences HLA class II | RFX5 | NM_000449 // | — | 2434971 | chr1 | − | 149578882 | 149586373 | 1.05 |
| expression) | | NM_001025603 | | | | | | | |
| protein phosphatase 2 (formerly 2A), regulatory | PPP2R2A | NM_002717 | B55A // FLJ26613 | 3090922 | chr8 | + | 26156860 | 26286261 | 1.05 |
| subunit B, alpha isoform | | | // MGC52248 // | | | | | | |
| | | | PR52A // PR55A | | | | | | |
| chromosome 20 open reading frame 11 | C20orf11 | NM_017896 | TWA1 | 3893072 | chr20 | + | 61039681 | 61050570 | 1.05 |
| PCI domain containing 2 | PCID2 | NM_018386 | F10 // FLJ11305 // | 3526378 | chr13 | − | 112877970 | 112912799 | 1.05 |
| | | | MGC16774 | | | | | | |
| lymphocyte antigen 9 // SLAM family member 7 | LY9 // | NM_001033667 // | CD229 // SLAMF3 | 2363248 | chr1 | + | 159032527 | 159064855 | 1.05 |
| | SLAMF7 | NM_002348 // | // hly9 // mLY9 // | | | | | | |
| | | NM_021181 | 19A // CD319 // | | | | | | |
| | | | CRACC // CS1 | | | | | | |
| arrestin domain containing 3 | ARRDC3 | NM_020801 | KIAA1376 // TLIMP | 2866704 | chr5 | − | 90700311 | 90782084 | 1.05 |
| chromosome 14 open reading frame 103 | C14orf103 | NM_018036 | FLJ10242 | 3578278 | chr14 | − | 95817349 | 95900740 | 1.05 |
| myosin IXB | MYO9B | NM_004145 | CELIAC4 // MYR5 | 3823982 | chr19 | + | 17047591 | 17185088 | 1.05 |
| t-complex 1 | TCP1 | NM_001008897 // | CCT-alpha // | 2982381 | chr6 | − | 160119521 | 160130731 | 1.05 |
| | | NM_030752 | CCT1 // CCTa // | | | | | | |
| | | | D6S230E // TCP- | | | | | | |
| | | | 1-alpha | | | | | | |
| translocation protein 1 | TLOC1 | NM_003262 | Dtrp1 // FLJ32803 | 2651782 | chr3 | + | 171166446 | 171194653 | 1.05 |
| | | | // HTP1 // SEC62 | | | | | | |
| far upstream element (FUSE) binding protein 1 | FUBP1 | NM_003902 | FBP // FUBP | 2419235 | chr1 | − | 78182330 | 78217881 | 1.05 |
| mannosidase, alpha, class 1A, member 2 // | MAN1A2 // | NM_006699 // — | MAN1B // — | 2353881 | chr1 | + | 117689854 | 117931990 | 1.05 |
| urothelial cancer associated 1 | UCA1 | | | | | | | | |
| engulfment and cell motility 2 | ELMO2 | NM_133171 // | CED-12 // CED12 | 3907830 | chr20 | − | 44426906 | 44495031 | 1.05 |
| | | NM_182764 | // ELMO-2 // | | | | | | |
| | | | FLJ11656 // | | | | | | |
| | | | KIAA1834 | | | | | | |
| erbb2 interacting protein // caspase 6, | ERBB2IP // | NM_001006600 // | ERBIN // LAP2 // | 2812435 | chr5 | + | 65258079 | 65451372 | 1.05 |
| apoptosis-related cysteine peptidase | CASP6 | NM_018695 // | MCH2 | | | | | | |
| | | NM_001226 | | | | | | | |
| | | NM_032992 | | | | | | | |
| heterogeneous nuclear ribonucleoprotein R | HNRPR | NM_005826 | FLJ25714 // | 2401275 | chr1 | − | 23486545 | 23543926 | 1.05 |
| | | | HNRNPR // | | | | | | |
| | | | hnRNP-R | | | | | | |
| membrane-bound transcription factor | MBTPS1 | NM_003791 // | KIAA0091 // | 3702293 | chr16 | − | 82644885 | 82708018 | 1.05 |
| peptidase, site 1 | | NM_201268 | MGC138711 // | | | | | | |
| | | | MGC138712 // | | | | | | |
| | | | PCSK8 // S1P // | | | | | | |
| | | | SKI-1 | | | | | | |
| RAB35, member RAS oncogene family | RAB35 | NM_006861 | H-ray // RAB1C // | 3474228 | chr12 | − | 119017289 | 119039689 | 1.05 |
| | | | RAY | | | | | | |
| farnesyl-diphosphate farnesyltransferase 1 | FDFT1 | NM_004462 | DGPT // ERG9 // | 3086206 | chr8 | + | 11690497 | 11740987 | 1.05 |
| | | | SQS // SS | | | | | | |
| SATB homeobox 1 | SATB1 | NM_002971 | — | 2665199 | chr3 | − | 18364435 | 18462086 | 1.05 |
| RAS guanyl releasing protein 1 (calcium and | RASGRP1 | NM_005739 | CALDAG-GEFI // | 3618736 | chr15 | − | 36567598 | 36809973 | 1.05 |
| DAG-regulated) | | | CALDAG-GEFII // | | | | | | |
| | | | MGC129998 // | | | | | | |
| | | | MGC129999 // | | | | | | |
| | | | RASGRP // V // | | | | | | |
| | | | hRasGRP1 | | | | | | |
| interferon-related developmental regulator 1 | IFRD1 | NM_001007245 // | PC4 // TIS7 | 3019519 | chr7 | + | 111835465 | 111903788 | 1.05 |
| | | NM_001590 | | | | | | | |
| coatomer protein complex, subunit alpha // | COPA // | NM_004371 // | FLJ26320 // HEP- | 2440143 | chr1 | − | 158525015 | 158580073 | 1.05 |
| peroxisomal biogenesis factor 19 | PEX19 | NM_002857 | COP // D1S2223E | | | | | | |
| | | | // HK33 // PMP1 | | | | | | |
| | | | // PMPI // PXF // | | | | | | |
| | | | PXMP1 | | | | | | |
| SCY1-like 2 (S. cerevisiae) | SCYL2 | NM_017988 | CVAK104 // | 3428131 | chr12 | + | 99185070 | 99260564 | 1.05 |
| | | | FLJ10074 // | | | | | | |
| | | | KIAA1360 | | | | | | |
| Ctr9, Paf1/RNA polymerase II complex | CTR9 // | NM_014633 // | KIAA0155 // | 3320301 | chr11 | + | 10684027 | 10774985 | 1.05 |
| component, homolog (S. cerevisiae) // tRNA 5- | TRMU | NM_018006 | SH2BP1 // TSBP | | | | | | |
| methylaminomethyl-2-thiouridylate | | | // p150 // | | | | | | |
| methyltransferase | | | p150TSP // | | | | | | |
| | | | MGC99627 // | | | | | | |
| | | | MTO2 // MTU1 // | | | | | | |
| | | | TRMT // TRMT1 // | | | | | | |
| serine/threonine kinase 40 // erythrocyte | STK40 // | NM_032017 // | MGC4796 // RP11- | 2406677 | chr1 | − | 36577822 | 36626062 | 1.05 |
| membrane protein band 4.1 (elliptocytosis 1, | EPB41 | NM_004437 // | 268J15.4 // SHIK | | | | | | |
| RH-linked) | | NM_203342 // | // SgK495 // 4.1 R | | | | | | |
| | | NM_203343 | // EL1 // HE | | | | | | |
| heterogeneous nuclear ribonucleoprotein U | HNRPU | NM_004501 // | HNRNPU // SAF-A | 2464499 | chr1 | − | 243078713 | 243094575 | 1.05 |
| (scaffold attachment factor A) | | NM_031844 // | U21.1 | | | | | | |
| REST corepressor 1 | RCOR1 | NM_015156 | COREST // | 3553228 | chr14 | + | 102053947 | 102266629 | 1.05 |
| | | | KIAA0071 // RCOR | | | | | | |
| CREB binding protein (Rubinstein-Taybi | CREBEP | NM_001079846 // | CBP // RSTS // | 3677795 | chr16 | − | 3715075 | 3870713 | 1.05 |
| syndrome) | | NM_004380 | RTS | | | | | | |
| golgi apparatus protein 1 // CDC42 small | GLG1 // | NM_012201 // | CFR-1 // ESL-1 // | 3698919 | chr16 | − | 73015604 | 73209803 | 1.05 |
| affecter 1 // sema domain, transmembrane | CDC42SE1 // | NM_001038707 // | FLJ23319 // | | | | | | |
| domain (TM), and cytoplasmic domain, | SEMA6C | NM_020239 // | FLJ23967 // MG- | | | | | | |
| (semaphorin) 6C | | NM_030913 | 160 // MG160 // | | | | | | |
| | | | SCIP1 // SPEC1 // | | | | | | |
| | | | SEMAY // m-SemaY | | | | | | |
| | | | // m-Sema Y2 | | | | | | |
| PHD finger protein 3 | PHF3 | NM_015153 | KIAA0244 // | 2911944 | chr6 | + | 64403666 | 64547188 | 1.05 |
| | | | MGC142210 // | | | | | | |
| | | | MGC142212 | | | | | | |
| WDR45-like | WDR45L | NM_019613 | WIPI-3 // WIPI3 | 3775157 | chr17 | − | 78165748 | 78199803 | 1.05 |
| HMG-box transcription factor 1 | HBP1 | NM_012257 | FLJ16340 | 3018420 | chr7 | + | 106596679 | 106630194 | 1.05 |
| chromosome 2 open reading frame 25 | C2orf25 | NM_015702 | CL25022 | 2580635 | chr2 | − | 150101637 | 150152607 | 1.05 |
| heat shock 70 kDa protein 8 // Fc fragment of | HSPA8 // | NM_006597 // | HSC54 // HSC70 | 3395416 | chr11 | − | 122429305 | 122438895 | 1.05 |
| IgG, low affinity IIIb, receptor (CD16b) | FCGR3B | NM_153201 // | // HSC71 // | | | | | | |
| | | NM_000570 | HSP71 // HSP73 | | | | | | |
| | | | // HSPA10 // | | | | | | |
| | | | LAP1 // | | | | | | |
| | | | MGC131511 // | | | | | | |
| | | | MGC29929 // | | | | | | |
| | | | NIP71 // CD16 // | | | | | | |
| chromosome 6 open reading frame 62 | C6orf62 | NM_030939 | DKFZP564G182 // | 2945677 | chr6 | − | 24798431 | 24851423 | 1.05 |
| | | | FLJ12619 // | | | | | | |
| | | | Nbla00237 // | | | | | | |
| | | | XTP12 // dJ30M3.2 | | | | | | |
| leptin receptor overlapping transcript-like 1 | LEPROTL1 | NM_015344 | HSPC112 // Vps55 | 3092276 | chr8 | + | 30059542 | 30115386 | 1.05 |
| | | | // my047 | | | | | | |
| ubiquitin-conjugating enzyme E2I (UBC9 | UBE2I | NM_003345 // | C358B7.1 // P18 // | 3643703 | chr16 | + | 1289451 | 1317016 | 1.05 |
| homolog, yeast) | | NM_194259 // | UBC9 | | | | | | |
| | | NM_194260 // | | | | | | | |
| | | NM_194261 | | | | | | | |
| nucleoporin 155 kDa | NUP155 | NM_004298 // | KIAA0791 // N155 | 2853768 | chr5 | − | 37325714 | 37407016 | 1.05 |
| | | NM_153485 | | | | | | | |
| lysosomal trafficking regulator | LYST | NM_000081 // | CHS // CHS1 | 2461999 | chr1 | − | 233878999 | 234113611 | 1.05 |
| | | NM_001005736 | | | | | | | |
| mitogen-activated protein kinase 14 | MAPK14 | NM_001315 // | CSBP1 // CSBP2 | 2904877 | chr6 | + | 36103487 | 36186989 | 1.05 |
| | | NM_139012 // | // CSPB1 // EXIP | | | | | | |
| | | NM_139013 // | // Mxi2 // PRKM14 | | | | | | |
| | | NM_139014 | // PRKM15 // RK | | | | | | |
| | | | // SAPK2A // p38 | | | | | | |
| | | | // p38ALPHA | | | | | | |
| nuclear factor (erythroid-derived 2)-like 2 // | NFE2L2 // | NM_006164 // | NRF2 // — | 2588827 | chr2 | − | 177794809 | 177837753 | 1.05 |
| hypothetical protein DKFZp451M2119 | DKFZp451M2119 | NM_182585 | | | | | | | |
| WD repeat domain 26 | WDR26 | NM_025160 | FLJ21016 // MIP2 | 2458082 | chr1 | − | 222639474 | 222691338 | 1.05 |
| heterogeneous nuclear ribonucleoprotein K | HNRPK | NM_002140 // | CSBP // FLJ41122 | 3212294 | chr9 | − | 85772377 | 85785355 | 1.05 |
| | | NM_031262 // | // HNRNPK // | | | | | | |
| | | NM_031263 | TUNP | | | | | | |
| transducin-like enhancer of split 4 (E(sp1) | TLE4 | NM_007005 | BCE-1 // E(spI) // | 3176209 | chr9 | + | 81269283 | 81552230 | 1.05 |
| homolog, Drosophila) | | | ESG // ESG4 // | | | | | | |
| | | | GRG4 | | | | | | |
| bromodomain containing 2 | BRD2 | NM_005104 | D6S113E // | 2903343 | chr6 | + | 33044392 | 33057253 | 1.05 |
| | | | DKFZp686N0336 // | | | | | | |
| | | | FLJ31942 // | | | | | | |
| | | | FSRG1 // | | | | | | |
| | | | KIAA9001 // NAT | | | | | | |
| | | | // RING3 // RNF3 | | | | | | |
| signal transducer and activator of transcription | STAT3 | NM_003150 // | APRF // FLJ20882 | 3757840 | chr17 | − | 37718873 | 37794201 | 1.05 |
| 3 (acute-phase response factor) | | NM_139276 // | // MGC16063 | | | | | | |
| | | NM_213662 | | | | | | | |
| ariadne homolog, ubiquitin-conjugating enzyme | ARIH1 | NM_005744 | ARI // HARI // | 3600744 | chr15 | + | 70553731 | 70666726 | 1.05 |
| E2 binding protein, 1 (Drosophila) | | | HHARI // | | | | | | |
| zinc finger, NFX1-type containing 1 // | ZNFX1 // | NM_021035 // | FLJ39275 // | 3908831 | chr20 | − | 47233911 | 47420063 | 1.05 |
| gasdermin-like | GSDML | NM_001042471 // | MGC131926 // | | | | | | |
| | | NM_018530 | PP4052 // | | | | | | |
| SUMO1/sentrin specific peptidase 6 | SENP6 | NM_015571 | FLJ11355 // | 2913983 | chr6 | + | 76367955 | 76484717 | 1.05 |
| | | | FLJ11887 // | | | | | | |
| | | | KIAA0389 // | | | | | | |
| | | | KIAA0797 // SSP1 | | | | | | |
| | | | // SUSP1 | | | | | | |
| DEAD (Asp-Glu-Ala-Asp) box polypeptide 17 | DDX17 | NM_006386 // | DKFZp761H2016 // | 3960629 | chr22 | − | 37202800 | 37232288 | 1.05 |
| | | NM_030881 | P72 // RH70 | | | | | | |
| ubiquitin specific peptidase 15 | USP15 | NM_006313 | KIAA0529 // | 3419147 | chr12 | + | 60940328 | 61086166 | 1.05 |
| | | | MGC131982 // | | | | | | |
| | | | MGC149838 // | | | | | | |
| | | | MGC74854 // | | | | | | |
| | | | UNPH4 | | | | | | |
| Ras association (RalGDS/AF-6) domain family 2 | RASSF2 | NM_014737 // | DKFZp781O1747 // | 3896034 | chr20 | − | 4655529 | 4752291 | 1.05 |
| | | NM_170774 | KIAA0168 | | | | | | |
| dual specificity phosphatase 6 | DUSP6 | NM_001946 // | MKP3 // PYST1 | 3464860 | chr12 | − | 88223376 | 88273467 | 1.05 |
| | | NM_022652 | | | | | | | |
| E1A binding protein p300 | EP300 | NM_001429 | p300 | 3946615 | chr22 | + | 39817736 | 39906025 | 1.05 |
| solute carrier family 7 (cationic amino acid | SLC7A6 | NM_001076785 // | DKFZp686K15246 | 3666146 | chr16 | + | 66855932 | 66893223 | 1.05 |
| transporter, y+ system), member 6 | | NM_003983 | // KIAA0245 // | | | | | | |
| | | | LAT-2 // LAT3 // | | | | | | |
| | | | y+LAT-2 | | | | | | |
| RNA binding motif protein 5 | RBM5 | NM_005778 | FLJ39876 // G15 | 2622469 | chr3 | + | 50101372 | 50134000 | 1.05 |
| | | | // H37 // LUCA15 | | | | | | |
| | | | // RMB5 | | | | | | |
| nuclear protein localization 4 homolog (S. cerevisiae) | NPLOC4 | NM_017921 | FLJ20657 // | 3774029 | chr17 | − | 77134363 | 77214526 | 1.05 |
| | | | FLJ23742 // | | | | | | |
| | | | KIAA1499 // NPL4 | | | | | | |
| cyclin L2 // aurora kinase A interacting protein 1 | CCNL2 // | NM_001039577 // | ANIA-6B // | 4041923 | chr1_random | + | 359569 | 372958 | 1.05 |
| | AURKAIP1 | NM_030937 // | DKFZp761A1210 // | | | | | | |
| | | NM_017900 | DKFZp762O195 // | | | | | | |
| | | | HCLA-ISO // HLA- | | | | | | |
| | | | ISO // PCEE // | | | | | | |
| | | | SB138 // AIP // | | | | | | |
| | | | AKIP // FLJ20608 | | | | | | |
| poly(rC) binding protein 2 | PCBP2 | NM_005016 // | HNRPE2 // | 3416036 | chr12 | + | 52132173 | 52161417 | 1.05 |
| | | NM_031989 | MGC110998 // | | | | | | |
| | | | hnRNP-E2 | | | | | | |
| aftiphilin | AFTPH | NM_001002243 // | FLJ20080 // | 2485433 | chr2 | + | 64604969 | 64714437 | 1.05 |
| | | NM_017657 // | FLJ23793 // | | | | | | |
| | | NM_203437 | MGC33965 // | | | | | | |
| | | | Nbla10388 | | | | | | |
| GTP binding protein 1 | GTPBP1 | NM_004286 | GP-1 // GP1 // | 3945396 | chr22 | + | 37431694 | 37459514 | 1.05 |
| | | | HSPC018 // | | | | | | |
| | | | MGC20069 | | | | | | |
| protein kinase C, eta | PRKCH | NM_006255 | MGC26269 // | 3538893 | chr14 | + | 60816958 | 61087440 | 1.05 |
| | | | MGC5363 // PKC- | | | | | | |
| | | | L // PKCL // | | | | | | |
| | | | PRKCL // nPKC- | | | | | | |
| glutaminase | GLS | NM_014905 | DKFZp686O15119 | 2520291 | chr2 | + | 191453802 | 191538513 | 1.05 |
| | | | // FLJ10358 // | | | | | | |
| | | | GLS1 // KIAA0838 | | | | | | |
| tubulin, beta | TUBB | NM_178014 | M40 // MGC117247 | 2901913 | chr6 | + | 30795977 | 30801165 | 1.05 |
| | | | // MGC16435 // | | | | | | |
| | | | OK/SW-cl.56 // | | | | | | |
| | | | TUBB1 // TUBB5 | | | | | | |
| neuroguidin, EIF4E binding protein | NGDN | XM_033371 // | C14orf120 // | 3529156 | chr14 | + | 23008821 | 23048896 | 1.05 |
| | | XM_932898 // | DKFZP564O092 // | | | | | | |
| | | XM_932900 // | LCP5 // NGD // | | | | | | |
| | | XM_932903 // | Ipd-2 | | | | | | |
| | | XM_932906 // | | | | | | | |
| | | XM_941350 // | | | | | | | |
| | | XM_945161 // | | | | | | | |
| | | XM_945163 // | | | | | | | |
| | | XM_945166 // | | | | | | | |
| | | XM_945170 // | | | | | | | |
| | | NM_001042635 // | | | | | | | |
| | | NM_015514 | | | | | | | |
| zinc finger, RAN-binding domain containing 2 | ZRANB2 | NM_005455 // | DKFZp686J1831 // | 2418000 | chr1 | − | 71292434 | 71355820 | 1.05 |
| | | NM_203350 | DKFZp686N09117 | | | | | | |
| | | | // FLJ41119 // ZIS | | | | | | |
| | | | // ZIS1 // ZIS2 // | | | | | | |
| | | | ZNF265 | | | | | | |
| DEAD (Asp-Glu-Ala-Asp) box polypeptide 21 | DDX21 | NM_004728 | DKFZp686F21172 | 3250055 | chr10 | + | 70380584 | 70414821 | 1.05 |
| | | | // GUA // GURDB | | | | | | |
| | | | // RH-II/GU // | | | | | | |
| | | | RH-II/GuA | | | | | | |
| sorting nexin 19 | SNX19 | NM_014758 | CHET8 // | 3398482 | chr11 | − | 130250320 | 130291615 | 1.05 |
| | | | DKFZp667I205 // | | | | | | |
| | | | KIAA0254 | | | | | | |
| phosphatidylinositol binding clathrin assembly | PICALM | NM_001008660 // | CALM // CLTH // | 3385175 | chr11 | − | 85345353 | 85458481 | 1.05 |
| protein | | NM_007166 | LAP | | | | | | |
| PI-3-kinase-related kinase SMG-1 | SMG1 | NM_015092 | 61E3.4 // ATX // | 3683050 | chr16 | − | 18719381 | 18845318 | 1.05 |
| | | | KIAA0421 // LIP | | | | | | |
| MON2 homolog (S. cerevisiae) | MON2 | NM_015026 | KIAA1040 // | 3419239 | chr12 | + | 61146751 | 61280506 | 1.05 |
| | | | MGC35493 | | | | | | |
| signal recognition particle receptor (‘docking | SRPR | NM_003139 | DP // MGC17355 | 3396916 | chr11 | − | 125636504 | 125644013 | 1.05 |
| protein’) | | | // MGC3650 // | | | | | | |
| | | | MGC9571 // SRP- | | | | | | |
| | | | alpha // Sralpha | | | | | | |
| heat shock protein 90 kDa alpha (cytosolic), | HSP90AB1 | NM_007355 | D6S182 // | 2908474 | chr6 | + | 44319983 | 44330383 | 1.05 |
| class B member 1 | | | FLJ26984 // | | | | | | |
| | | | HSP90-BETA // | | | | | | |
| | | | HSP90B // HSPC2 | | | | | | |
| | | | // HSPCB | | | | | | |
| eukaryotic translation initiation factor 5 | EIF5 | NM_001969 // | EIF-5A | 3553607 | chr14 | + | 102743652 | 102881108 | 1.05 |
| | | NM_183004 | | | | | | | |
| patatin-like phospholipase domain containing 10 | PNPLA10P // | XM_927062 // | —// | 3067592 | chr7 | − | 107880325 | 107956012 | 1.05 |
| pseudogene // patatin-like phospholipase | PNPLA8 | XM_938392 // | IPLA2(GAMMA) // | | | | | | |
| domain containing 8 | | NM_015723 | IPLA2-2 // IPLA2G | | | | | | |
| 2′-5′-oligoadenylate synthetase 3, 100 KDa | OAS3 | NM_006187 | MGC133260 // | 3432467 | chr12 | + | 111860550 | 111895663 | 1.05 |
| aminopeptidase puromycin sensitive | NPEPPS | XM_001128588 // | MP100 // PSA | 3724698 | chr17 | + | 42955347 | 43057188 | 1.05 |
| | | NM_006310 | | | | | | | |
| SAP30 binding protein // cardiotrophin-like | SAP30BP // | NM_013260 // | DKFZp586L2022 // | 3735107 | chr17 | + | 71174801 | 71215729 | 1.05 |
| cytokine factor 1 | CLCF1 | NM_013246 | HCNGP // HTRG | | | | | | |
| | | | // HTRP // BSF3 | | | | | | |
| | | | // CISS2 // CLC | | | | | | |
| | | | // NNT1 // NR6 | | | | | | |
| bromodomain adjacent to zinc finger domain, 1B | BAZ1B | NM_023005 // | WBSCR10 // | 3056044 | chr7 | − | 72492710 | 72574552 | 1.05 |
| | | NM_032408 | WBSCR9 // WSTF | | | | | | |
| cullin 3 | CUL3 | NM_003590 | — | 2601544 | chr2 | − | 225025520 | 225158348 | 1.05 |
| DEAD (Asp-Glu-Ala-Asp) box polypeptide 42 | DDX42 | NM_007372 // | FLJ43179 // | 3730899 | chr17 | + | 59204975 | 59250510 | 1.05 |
| | | NM_203499 | RHELP // RNAHP | | | | | | |
| | | | // SF3b125 | | | | | | |
| SWI/SNF related, matrix associated, actin | SMARCA2 | NM_003070 // | BAF190 // BRM // | 3159946 | chr9 | + | 1968220 | 2253060 | 1.05 |
| dependent regulator of chromatin, subfamily a, | | NM_139045 | FLJ36757 // | | | | | | |
| member 2 | | | MGC74511 // SNF2 | | | | | | |
| | | | // SNF2L2 // | | | | | | |
| | | | SNF2LA // SWI2 // | | | | | | |
| | | | Sth1p // hBRM // | | | | | | |
| | | | hSNF2a | | | | | | |
| stress-induced-phosphoprotein 1 | STIP1 | NM_006819 | HOP // IEF-SSP- | 3334224 | chr11 | + | 63709341 | 63728586 | 1.05 |
| (Hsp70/Hsp90-organizing protein) | | | 3521 // P60 // | | | | | | |
| | | | STI1L | | | | | | |
| splicing factor, arginine/serine-rich 7, 35 kDa | SFRS7 | NM_001031684 | 9G8 // AAG3 // | 2548970 | chr2 | − | 38824253 | 38832154 | 1.05 |
| | | | HSSG1 // RBM37 | | | | | | |
| | | | // ZCCHC20 // | | | | | | |
| | | | ZCRB2 | | | | | | |
| ribosomal protein L14 // ribosomal protein | RPL14 // | NM_001034996 // | CAG-ISL-7 // | 2618640 | chr3 | + | 40473807 | 40485068 | 1.05 |
| L14-like | RPL14L | NM_003973 // | CTG-B33 // L14 // | | | | | | |
| | | XR_017789 // | MGC88594 // RL14 | | | | | | |
| | | XR_017790 | // hRL14 // | | | | | | |
| | | | bcm1298 | | | | | | |
| acidic (leucine-rich) nuclear phosphoprotein 32 | ANP32C // | NM_012403 // | PP32R1 // C15orf1 | 3630912 | chr15 | − | 66857944 | 66900452 | 1.05 |
| family, member C // acidic (leucine-rich) | ANP32A | NM_006305 | // I1PP2A // LANP | | | | | | |
| nuclear phosphoprotein 32 family, member A | | | // MAPM // | | | | | | |
| | | | MGC119787 // | | | | | | |
| | | | MGC150373 // | | | | | | |
| | | | PHAP1 // PHAPI | | | | | | |
| | | | // PP32 | | | | | | |
| endoglin (Osler-Rendu-Weber syndrome 1) | ENG | NM_000118 | CD105 // END // | 3226097 | chr9 | − | 129615329 | 129660473 | 1.05 |
| | | | FLJ41744 // HHT1 | | | | | | |
| | | | // ORW // ORW1 | | | | | | |
| lysosomal associated multispanning membrane | LAPTM5 | NM_006762 | MGC125860 // | 2404158 | chr1 | − | 30977913 | 31095913 | 1.05 |
| protein 5 | | | MGC125861 | | | | | | |
| family with sequence similarity 108, member A2 | FAM108A2 // | XM_039721 // | C1orf47 // | 3845581 | chr19 | − | 1827984 | 1836490 | 1.05 |
| // family with sequence similarity 108, member | FAM108A1 | XM_933830 // | C19orf27 // | | | | | | |
| A1 | | NM_001080422 // | MGC5244 | | | | | | |
| | | NM_031213 | | | | | | | |
| interleukin 16 (lymphocyte chemoattractant | IL16 | NM_004513 // | FLJ16806 // | 3604287 | chr15 | + | 79262148 | 79392377 | 1.05 |
| factor) | | NM_172217 | FLJ42735 // | | | | | | |
| | | | FLJ44234 // | | | | | | |
| | | | HsT19289 // IL-16 | | | | | | |
| | | | // LCF // prIL-16 | | | | | | |
| SEC14 and spectrin domains 1 | SESTD1 | NM_178123 | DKFZp434O0515 // | 2589929 | chr2 | − | 179644888 | 180002479 | 1.05 |
| | | | SOLO | | | | | | |
| coatomer protein complex, subunit beta 2 (beta | COPB2 | NM_004766 | beta′-COP | 2697792 | chr3 | − | 140555482 | 140591192 | 1.05 |
| prime) | | | | | | | | | |
| Finkel-Biskis-Reilly murine sarcoma virus | FAU | NM_001997 | FAU1 // RPS30 | 3377463 | chr11 | − | 64644697 | 64646234 | 1.05 |
| (FBR-MuSV) ubiquitously expressed (fox | | | | | | | | | |
| derived); ribosomal protein S30 | | | | | | | | | |
| AHA1, activator of heat shock 90 kDa protein | AHSA1 | NM_012111 | AHA1 // C14orf3 | 3545466 | chr14 | + | 76993989 | 77005560 | 1.05 |
| ATPase homolog 1 (yeast) | | | // p38 | | | | | | |
| SLU7 splicing factor homolog (S. cerevisiae) | SLU7 | NM_006425 | 9G8 // MGC9280 | 2884658 | chr5 | − | 159761226 | 159778959 | 1.05 |
| | | | // hSlu7 | | | | | | |
| multiple C2 domains, transmembrane 1 | MCTP1 | NM_001002796 // | FLJ22344 | 2867443 | chr5 | − | 94066156 | 94646035 | 1.05 |
| | | NM_024717 | | | | | | | |
| ubiquitin-conjugating enzyme E2Z (putative) | UBE2Z | NM_023079 | FLJ13855 // | 3725481 | chr17 | + | 44340808 | 44361870 | 1.05 |
| hypothetical protein MGC40489 | MGC40489 | XR_015622 // | FLJ30780 | 3767053 | chr17 | − | 60176255 | 60263758 | 1.05 |
| XR_016048 | | | | | | | | | |
| ATG9 autophagy related 9 homolog A (S. cerevisiae) | ATG9A // | NM_001077198 // | APG9L1 // | 2599955 | chr2 | − | 219781799 | 219802587 | 1.05 |
| // ATP-binding cassette, sub- | ABCB6 | NM_024085 // | MGD3208 // ABC | | | | | | |
| family B (MDR/TAP), member 6 | | NM_005689 | // ABC14 // | | | | | | |
| | | | EST45597 // | | | | | | |
| | | | FLJ22414 // | | | | | | |
| | | | MTABC3 // PRP // | | | | | | |
| | | | umat | | | | | | |
| isoleucyl-tRNA synthetase | IARS | NM_002161 // | FLJ20736 // IARS1 | 3214668 | Chr9 | − | 94012330 | 94096287 | 1.05 |
| | | NM_013417 | // ILRS // | | | | | | |
| | | | PRO0785 | | | | | | |
| LIM domain binding 1 | LDB1 | NM_003893 | CLIM2 // NLJ | 3304215 | chr10 | − | 103830793 | 103883389 | 1.05 |
| YY1 transcription factor | YY1 | NM_003403 | DELTA // NF-E1 | 3551677 | chr14 | + | 99750383 | 99818868 | 1.05 |
| | | | // UCRBP // YIN- | | | | | | |
| | | | YANG-1 | | | | | | |
| WD repeat domain, phosphoinositide interacting | WIPI2 | NM_001033518 // | Atg21 // CGI-50 // | 2988536 | chr7 | + | 5196360 | 5239975 | 1.05 |
| | | NM_001033519 // | DKFZP434J154 // | | | | | | |
| | | NM_001033520 // | DKFZp686P02188 | | | | | | |
| | | NM_015610 // | // FLJ12979 // | | | | | | |
| | | NM_016003 | FLJ14217 // | | | | | | |
| | | | FLJ42984 // WIPI-2 | | | | | | |
| BCL2-associated transcription factor 1 | BCLAF1 | NM_001077440 // | BTF // KIAA0164 | 2975680 | chr6 | − | 136619703 | 136652847 | 1.05 |
| | | NM_001077441 // | // bK211L9.1 | | | | | | |
| | | NM_014739 | | | | | | | |
| ADAM metallopeptidase domain 19 (meltrin | ADAM19 | NM_023038 // | FKSG34 // | 2883440 | chr5 | − | 156755122 | 156935351 | 1.05 |
| beta) | | NM_033274 | MADDAM // | | | | | | |
| RAE1 RNA export 1 homolog (S. pombe) | RAE1 | NM_001015885 // | FLJ30608 // | 3890555 | chr20 | + | 55359493 | 55386992 | 1.05 |
| | | NM_)003610 | MGC117333 // | | | | | | |
| | | | MGC126076 // | | | | | | |
| | | | MGC126077 // | | | | | | |
| | | | MIG14 // MRNP41 | | | | | | |
| | | | // Mnrp41 // | | | | | | |
| | | | dJ481F12.3 // | | | | | | |
| | | | dJ800J21.1 | | | | | | |
| cyclin L2 // aurora kinase A interacting protein 1 | CCNL2 // | NM_001039577 // | ANIA-6B // | 2391532 | chr1 | − | 1314609 | 1324575 | 1.05 |
| | AURKAIP1 | NM_030937 // | DKFZp761A1210 // | | | | | | |
| | | NM_017900 | DKFZp762O195 // | | | | | | |
| | | | HCLA-ISO // HLA- | | | | | | |
| | | | ISO // PCEE // | | | | | | |
| | | | SB138 // AIP // | | | | | | |
| | | | AKIP // FLJ20608 | | | | | | |
| nucleolar and coiled-body phosphoprotein 1 | NOLC1 | NM_004741 | KIAA0035 // | 3261492 | chr10 | + | 103901962 | 103913597 | 1.05 |
| | | | NOPP130 // | | | | | | |
| | | | NOPP140 // | | | | | | |
| | | | NS5ATP13 // P130 | | | | | | |
| B-cell CLL/lymphoma 11B (zinc finger protein) | BCL11B | NM_022898 // | CTIP-2 // CTIP2 | 3579114 | chr14 | − | 98705377 | 98931503 | 1.05 |
| | | NM_138576 | // RIT1 // hRIT1- | | | | | | |
| proteasome (prosome, macropain) 26S subunit, | PSMD7 | NM_002811 | MOV34 // P40 // | 3668617 | chr16 | + | 72888182 | 72901528 | 1.05 |
| non-ATPase, 7 (Mov34 homolog) | | | S12 | | | | | | |
| chromosome 14 open reading frame 118 | C14orf118 | NM_017926 // | FLJ10033 // | 3544905 | chr14 | + | 75688015 | 75800924 | 1.05 |
| | | NM_017972 | FLJ20689 // | | | | | | |
| | | | MGC61896 | | | | | | |
| translocase of inner mitochondrial membrane 23 | TIMM23 | XM_001133798 // | MGC22767 // | | | | | | |
| homoleg (yeast) | | NM_006327 | PRO1197 // TIM23 | 3289031 | chr10 | − | 51180787 | 51293392 | 1.05 |
| | | | // TIMM23B | | | | | | |
| proteasome (prosome, macropain) 26S subunit, | PSMD2 | NM_002808 | MGC14274 // P97 | 2655650 | chr3 | + | 185499199 | 185509504 | 1.05 |
| non-ATPase, 2 | | | // S2 // TRAP2 | | | | | | |
| LSM14A, SOD6 homolog A (S. cerevislee) | LSM14A | NM_015578 | C19orf13 // | 3829575 | chr19 | + | 39355192 | 39416345 | 1.05 |
| | | | DKFZP434D1335 // | | | | | | |
| | | | FAM61A // RAP55 | | | | | | |
| phosphatidylinositol transfer protein, alpha // | PITPNA // | NM_006224 // | MGC99649 // | 3740304 | chr17 | − | 1367883 | 1412932 | 1.05 |
| mysoin IC | MYO1C | NM_001080779 // | PITPN // VIB1A // | | | | | | |
| | | NM_001080950 // | FLJ23603 // MMI- | | | | | | |
| | | NM_033375 | beta // MMIb // | | | | | | |
| | | | NMI // myr2 | | | | | | |
| centaurin, delta 1 | CENTD1 | NM_015230 // | ARAP2 // | 2765590 | chr4 | − | 35626248 | 35922356 | 1.05 |
| | | NM_139182 | FLJ13675 // | | | | | | |
| | | | FLJ44916 // | | | | | | |
| structural maintenance of chromosomes 1A | SMC1A | NM_006306 | DKFZp686L19178 | 4009238 | chrX | − | 53417797 | 53466400 | 1.05 |
| | | | // DXS423E // | | | | | | |
| | | | KIAA0178 // | | | | | | |
| | | | MGC138332 // | | | | | | |
| | | | SB1.8 // SMC1 // | | | | | | |
| | | | SMC1L1 // | | | | | | |
| | | | SMC1alpha // | | | | | | |
| solute carrier family 43, member 3 | SLC43A3 | NM_014096 // | DKFZp762A227 // | 3373845 | chr11 | − | 56931008 | 56951629 | 1.04 |
| | | NM_017611 // | EEG1 // FOAP-13 | | | | | | |
| | | NM_199329 | // PRO1659 // | | | | | | |
| | | | SEEEG-1 | | | | | | |
| ninjurin 1 | NINJ1 | NM_004148 | NIN1 // NINJURIN | 3215146 | chr9 | − | 94923613 | 94939639 | 1.04 |
| CDC-like kinase 3 | CLK3 | NM_001292 // | FLJ22858 | 3601741 | chr15 | + | 72677906 | 72719105 | 1.04 |
| | | NM_003992 | | | | | | | |
| sortilin-related receptor, L(DLR class) A | SORL1 // | NM_003105 | LR11 // LRP9 // | 3352948 | chr11 | + | 120828130 | 121077997 | 1.04 |
| repeats-containing | C11orf32 | | SORLA // SorLA-1 | 3352948 | chr11 | + | 120828130 | 121077997 | 1.04 |
| | | | // gp250 | | | | | | |
| KIAA0831 | KIAA0831 | NM_014924 | MGC126291 // | 3565739 | chr14 | − | 54902008 | 54948457 | 1.04 |
| | | | MGC126292 | | | | | | |
| CD6 molecule | CD6 | NM_006725 | TP120 | 3332663 | chr11 | + | 60495619 | 60544422 | 1.04 |
| pre-mRNA cleavage factor I, 59 kDa subunit | FLJ12529 | NM_024811 | FLJ39024 // | 3375340 | chr11 | − | 60926704 | 60954030 | 1.04 |
| | | | MGC9315 | | | | | | |
| SEC23 interacting protein | SEC23IP | NM_007190 | MSTP053 // P125 | 3267455 | chr10 | + | 121642213 | 121691984 | 1.04 |
| cell division cycle 40 homolog (S. cerevisiae) | CDC40 | NM_015891 | EHB3 // FLJ10564 | 2921086 | chr5 | + | 110606544 | 110682171 | 1.04 |
| | | | // MGC102802 // | | | | | | |
| | | | PRP17 // PRPF17 | | | | | | |
| calnexin | CANX | NM_001024649 // | CNX // FLJ26570 | 2844203 | chr5 | + | 179058077 | 179091243 | 1.04 |
| | | NM_001746 | // IP90 // P90 | | | | | | |
| chromosome 20 open reading frame 112 | C20orf112 | NM_080616 | DKFZP566G1424 // | 3902764 | chr20 | − | 30498759 | 30636537 | 1.04 |
| | | | dJ1184F4.2 | | | | | | |
| splicing factor 3b, subunit 3, 130 kDa | SF3B3 | NM_012426 | KIAA0017 // RSE1 | 3667281 | chr16 | + | 69115212 | 69169072 | 1.04 |
| | | | // SAP130 // | | | | | | |
| | | | SF3b130 // | | | | | | |
| | | | STAF130 | | | | | | |
| family with sequence similarity 44, member A | FAM44A | NM_148894 | FLJ33215 // | 2761285 | chr4 | − | 13179462 | 13207890 | 1.04 |
| | | | KIAA1327 | | | | | | |
| casein kinase 2, alpha 1 polypeptide // casein | CSNK2A1 // | NM_001895 // | CK2A1 // CKII // | 3894228 | chr20 | − | 402069 | 472534 | 1.04 |
| kinase 2, alpha 1 polypeptide pseudogene | CSNK2A1P | NM_177559 // | CKII alpha // — | | | | | | |
| | | NM_177560 // | | | | | | | |
| | | NR_002207 | | | | | | | |
| eukaryotic translation initiation factor 3, subunit | EIF3S7 | NM_003753 | MGC126526 // | 3959631 | chr22 | − | 35236857 | 35255429 | 1.04 |
| 7 zeta, 66/67 kDa | | | MGC17258 // eIF3- | | | | | | |
| | | | p66 // eIF-zeta // | | | | | | |
| | | | eIF3d | | | | | | |
| GTP binding protein 2 | GTPBP2 | NM_019096 | MGC74725 | 29544771 | chr6 | − | 43681031 | 43704877 | 1.04 |
| fusion (involved in t(12; 16) in malignant | FUS | NM_001010850 // | CHOP // FUS- | 3656904 | chr16 | + | 31093395 | 31111003 | 1.04 |
| liposarcoma) | | NM_004960 | CHOP // FUS1 // | | | | | | |
| | | | TLS // TLS/CHOP | | | | | | |
| KIAA0430 | KIAA0430 | NM_014647 | A-362G6.1 // LKAP | 3681956 | chr16 | − | 15595757 | 15654441 | 1.04 |
| lymphocyte cytosolic protein 1 (L-plastin) | LCP1 | NM_002298 | CP64 // | 3512874 | chr13 | − | 45595067 | 45684007 | 1.04 |
| | | | DKFZp781A23186 | | | | | | |
| | | | // FLJ25423 // | | | | | | |
| | | | FLJ26114 // | | | | | | |
| | | | FLJ39956 // L- | | | | | | |
| | | | PLASTIN // LC64P | | | | | | |
| | | | // PLS2 | | | | | | |
| DENN/MADD domain containing 3 | DENND3 | NM_014957 | KIAA0870 | 3118651 | chr8 | + | 142207943 | 142289494 | 1.04 |
| KIAA0317 | KIAA0317 | NM_001039479 | — | 3572041 | chr14 | − | 74189599 | 74249655 | 1.04 |
| ninein (GSK3B interacting protein) | NIN | NM_016350 // | KIAA1565 | 3564071 | chr14 | − | 50256234 | 50403020 | 1.04 |
| | | NM_020921 // | | | | | | | |
| | | NM_182944 // | | | | | | | |
| | | NM_182945 // | | | | | | | |
| | | NM_182946 | | | | | | | |
| transmembrane protein 161B // integrin, alpha | TMEM161B // | NM_153354 // | FLB3342 // | 2866045 | chr5 | − | 87526693 | 87600565 | 1.04 |
| 2b (platelet glycoprotein IIb of IIb/IIIa complex, | ITGA2B | NM_000419 | MGC33214 // | | | | | | |
| antigen CD41) | | | PRO1313 // CD41 | | | | | | |
| | | | // CD41B // GP2B | | | | | | |
| | | | // GPIIb // GTA // | | | | | | |
| | | | HPA3 | | | | | | |
| Ewing sarcoma breakpoint region 1 | EWSR1 | NM_005243 // | EWS | 3941907 | chr22 | + | 27994201 | 28026501 | 1.04 |
| | | NM_013986 | | | | | | | |
| YTH domain containing 1 | YTHDC1 | NM_001031732 // | KIAA1966 // YT521 | 2772017 | chr4 | − | 68858700 | 68934802 | 1.04 |
| | | NM_133370 | // YT521-B | | | | | | |
| squamous cell carcinoma antigen recognized by | SART3 | NM_014706 | KIAA0156 // | 3470253 | chr12 | − | 107440140 | 107479296 | 1.04 |
| T cells 3 | | | MGC138188 // | | | | | | |
| | | | RP11-13G14 // | | | | | | |
| | | | TIP110 // p110(nrb) | | | | | | |
| cytoplasmic linker associated pretein 1 | CLASP1 | NM_015282 | DKFZp686D1968 // | 2573641 | chr2 | − | 121811825 | 122169321 | 1.04 |
| | | | DKFZp686H2039 // | | | | | | |
| | | | FLJ33821 // | | | | | | |
| | | | FLJ41222 // | | | | | | |
| | | | KIAA0622 // | | | | | | |
| | | | MAST1 // | | | | | | |
| nuclear receptor interacting protein 1 | NRIP1 | NM_003489 | RIP140 | 3925639 | chr21 | − | 15255125 | 15359182 | 1.04 |
| protein phosphatase 1, regulatory (inhibitor) | PPP1R12A | NM_002480 | MBS // | 3463571 | chr12 | − | 78690959 | 78853356 | 1.04 |
| subunit 12A | | | MGC133042 // | | | | | | |
| STE20-like kinase (yeast) | SLK | NM_014720 | KIAA0204 // | 3262433 | chr10 | + | 105716666 | 105778975 | 1.04 |
| | | | MGC133067 // | | | | | | |
| | | | STK2 // bA16H23.1 | | | | | | |
| | | | // se20-9 | | | | | | |
| polymerase (RNA) II (DNA directed) polypeptide | POLR2B | NM_000938 | POL2RB // RPB2 | 2728448 | chr4 | + | 57539163 | 57592513 | 1.04 |
| B, 140 kDa | | | // hRPB140 // | | | | | | |
| | | | hsRPB2 | | | | | | |
| HIV-1 Rev binding protein | HRB | XM_941338 // | MGC116938 // | 2530599 | chr2 | + | 228045122 | 228134167 | 1.04 |
| | | NM_004504 | MGC116940 // RAB | | | | | | |
| | | | // RIP | | | | | | |
| zinc finger CCCH-type containing 12A | ZC3H12A | NM_025079 | FLJ23231 // | 2330687 | chr1 | + | 37712740 | 37722550 | 1.04 |
| | | | MCPIP // RP3- | | | | | | |
| | | | 423B22.1 // | | | | | | |
| transforming growth factor, beta 1 | TGFB1 | NM_000660 | CED // DPD1 // | 3863021 | chr19 | − | 46499343 | 46551636 | 1.04 |
| | | | TGFB | | | | | | |
| ADP-ribosylation factor 4 | ARF4 | XM_001132763 // | — | 2678090 | chr3 | − | 57532172 | 57558635 | 1.04 |
| | | NM_001660 | | | | | | | |
| pleiomorphic adenoma gene-like 2 | PLAGL2 | NM_002657 | FLJ23283 | 3902682 | chr20 | − | 30243975 | 30259284 | 1.04 |
| jumonji domain containing 2C | JMJD2C | NM_015061 | FLJ25949 // | 3161566 | chr9 | + | 6747651 | 7165647 | 1.04 |
| | | | GASC1 // JHDM3C | | | | | | |
| | | | // KIAA0780 // | | | | | | |
| | | | bA146B14.1 | | | | | | |
| transmembrane BAX inhibitor motif containing 1 | TMBIM1 | NM_022152 | PP1201 // RECS1 | 2599371 | chr2 | − | 218847185 | 218865515 | 1.04 |
| GPI-anchored membrane protein 1 | GPIAP1 | NM_005898 // | GPIP137 // M11S1 | 3326183 | chr11 | + | 34009711 | 34081946 | 1.04 |
| | | NM_203364 | // p137GPI | | | | | | |
| DEAD (Asp-Glu-Ala-Asp) box polypeptide 47 | DDX47 // | NM_016355 // | DKFZp564O176 // | 3405531 | chr12 | + | 12856462 | 12874176 | 1.04 |
| // apolipoprotein L domain containing 1 | APOLD1 | NM_201224 // | E4-DBP // | | | | | | |
| | | NM_030817 | FLJ30012 // | | | | | | |
| | | | HQ0256 // | | | | | | |
| | | | MSTP162 // | | | | | | |
| | | | DKFZP434F0318 // | | | | | | |
| | | | FLJ25138 | | | | | | |
| SERPINE1 mRNA binding protein 1 | SERBP1 | NM_001018067 // | CGI-55 // CHD3IP | 2417174 | chr1 | − | 67646101 | 67669020 | 1.04 |
| | | NM_001018068 // | // DKFZp564M2423 | | | | | | |
| | | NM_001018069 // | // FLJ90489 // | | | | | | |
| | | NM_015640 | HABP4L // PAI- | | | | | | |
| | | | RBP1 // PAIRBP1 | | | | | | |
| coiled-coil domain containing 47 | CCDC47 | NM_020198 | GK001 // MSTP041 | 3766334 | chr17 | − | 59176353 | 59204726 | 1.04 |
| lamin B receptor | LBR | NM_002296 // | DHOR14B // | 2458289 | chr1 | − | 223655840 | 223683230 | 1.04 |
| | | NM_194442 | LMN2R // | | | | | | |
| | | | MGC9041 // PHA | | | | | | |
| caspase 3, apoptosis-related cysteine | CASP3 | NM_004346 // | CPP32 // CPP32B | 2796484 | chr4 | − | 185782536 | 185807608 | 1.04 |
| peptidase | | NM_032991 | // SCA-1 | | | | | | |
| Burkitt lymphoma receptor 1, GTP binding | BLR1 | NM_001716 // | CD185 // CXCR5 | 3351675 | chr11 | + | 118259777 | 118272180 | 1.04 |
| protein (chemokine (C—X—C motif) receptor 5) | | NM_032966 | // MDR15 // | | | | | | |
| | | | MGC117347 | | | | | | |
| major histocompatibility complex, class I, E | HLA-E | NM_005516 | DKFZp686P19218 | 2901620 | chr6 | + | 30565243 | 30620086 | 1.04 |
| | | | // EA1.2 // EA2.1 | | | | | | |
| | | | // HLA-6.2 // MHC | | | | | | |
| v-yes-1 Yamaguchi sarcoma viral related | LYN | NM_002350 | FLJ26625 // JTK8 | 3098977 | chr8 | + | 56954505 | 57087291 | 1.04 |
| oncogene homolog | | | | | | | | | |
| MYC-associated zinc finger protein (purine- | MAZ // KIF22 | NM_001042539 // | PUR1 // Pur-1 // | 3655665 | chr16 | + | 29724956 | 29729977 | 1.04 |
| binding transcription factor) // kinesin family | | NM_002383 // | SAF-1 // SAF-2 // | | | | | | |
| member 22 | | NM_007317 | ZF87 // ZNF801 // | | | | | | |
| | | | Zif87 // KID // | | | | | | |
| | | | KNSL4 // OBP // | | | | | | |
| | | | OBP-1 // OBP-2 | | | | | | |
| chromosome 3 open reading frame 58 | C3orf58 | NM_173552 | MGC33365 | 2646327 | chr3 | + | 145173603 | 145669744 | 1.04 |
| hect (homologous to the E6-AP (UBE3A) | HERC1 | NM_003922 | p532 // p619 | 3628650 | chr15 | − | 61687879 | 61913158 | 1.04 |
| carboxyl terminus) domain and RCC1 (CHC1)- | | | | | | | | | |
| like domain (RLD) 1 | | | | | | | | | |
| KIAA0652 | KIAA0652 | NM_014741 | FLJ20698 | 3329404 | chr11 | + | 46595671 | 46652934 | 1.04 |
| PX domain containing serine/threonine kinase | PXK | NM_017771 | FLJ20335 // | 2626167 | chr3 | + | 58293657 | 58386884 | 1.04 |
| | | | MONaKA | | | | | | |
| jumonji, AT rich interactive domain 2 | JARID2 // | NM_004973 | JMJ | 2896177 | chr6 | + | 15261674 | 15630231 | 1.04 |
| | C20orf120 | | | | | | | | |
| GGI-09 protein | CGI-09 | NM_015939 | MGC5029 | 3896524 | chr20 | − | 5865881 | 5879370 | 1.04 |
| syndecan binding protein (syntenin) | SDCBP | NM_001007067 // | MDA-9 // ST1 // | 3099750 | chr8 | + | 59605208 | 59658960 | 1.04 |
| | | NM_001007068 // | SYCL // TACIP18 | | | | | | |
| | | NM_001007069 // | | | | | | | |
| | | NM_001007070 // | | | | | | | |
| | | NM_005625 | | | | | | | |
| 2′,3′-cyclic nucleotide 3′ phosphodiesterase | CNP | NM_033133 | CNP1 | 3721548 | chr17 | + | 37372285 | 37383765 | 1.04 |
| family with sequence similarity 48, member A | FAM48A // | NM_001014286 // | C13 // C13orf19 // | 3509910 | chr13 | − | 36480880 | 36556254 | 1.04 |
| // family with sequence similarity 48, member | FAM48B2 | NM_017569 // | FP757 // P38IP // | | | | | | |
| B2 | | XM_293352 | bA421P11.4 // — | | | | | | |
| ubiquitin-activating enzyme E1-like 2 | UBE1L2 | NM_018227 | FLJ10808 // | 2771718 | chr4 | − | 68130308 | 68249472 | 1.04 |
| | | | FLJ23367 | | | | | | |
| solute carrier family 35, member B1 | SLC35B1 | NM_005827 | UGTREL1 | 3761959 | chr17 | − | 45133304 | 45141355 | 1.04 |
| pogo transposable element with ZNF domain | POGZ | NM_015100 // | KIAA0461 // | 2435044 | chr1 | − | 149641830 | 149753035 | 1.04 |
| | | NM_145796 // | MGC71543 // | | | | | | |
| | | NM_207171 | SUHW5 // ZNF635 | | | | | | |
| forkhead box O1A (rhabdomyosarcoma) | FOXO1A | NM_002015 | FKH1 // FKHR // | 3510858 | chr13 | − | 40027817 | 40162098 | 1.04 |
| | | | FOXO1 | | | | | | |
| RAB14, member RAS oncogene family | RAB14 | NM_016322 | FBP // RAB-14 | 3223872 | chr9 | − | 122980236 | 123025093 | 1.04 |
| zinc finger protein 313 | ZNF313 | NM_018683 | RNF114 | 3888474 | chr20 | + | 47986321 | 48003818 | 1.04 |
| DnaJ (Hsp40) homolog, subfamily C, member 1 | DNAJC1 | NM_022365 | DNAJL1 // ERdj1 | 3280902 | chr10 | − | 22078144 | 22332877 | 1.04 |
| | | | // HTJ1 // | | | | | | |
| | | | MGC131954 | | | | | | |
| integrin, beta 1 (fibronectin receptor, beta | ITGB1 | NM_002211 // | CD29 // FNRB // | 3284188 | chr10 | − | 33211051 | 33321367 | 1.04 |
| polypeptide, antigen CD29 includes MDF2, | | NM_033666 // | GPIIA // MDF2 // | | | | | | |
| MSK12) | | NM_033667 // | MSK12 // VLAB | | | | | | |
| | | NM_033668 // | | | | | | | |
| | | NM_033669 // | | | | | | | |
| | | NM_133376 | | | | | | | |
| MMDA receptor regulated 2 | NARG2 | NM_001018089 // | — | 3627363 | chr15 | − | 58500956 | 58558626 | 1.04 |
| | | NM_024611 | | | | | | | |
| mitochondrial ribosomal protein S7 | MRPS7 | NM_015971 | MRP-S // MRP-S7 | 3734760 | chr17 | + | 70769374 | 70774626 | 1.04 |
| | | | // RP-S7 // | | | | | | |
| TATA box binding protein | TBP | NM_003194 | GTF2D // GTF2D1 | 2937984 | chr6 | + | 170705200 | 170723945 | 1.04 |
| | | | // MGC117320 // | | | | | | |
| | | | MGC126054 // | | | | | | |
| | | | MGC126055 // | | | | | | |
| | | | SCAI7 // TFIID | | | | | | |
| polymerase (RNA) I polypeptide C, 30 kDa | POLR1C | NM_004875 // | RPA39 // RPA40 | 2908052 | chr6 | + | 43592769 | 43605071 | 1.04 |
| | | NM_203290 | // RPA5 // RPAC1 | | | | | | |
| spectrin, alpha, non-erythrocytic 1 (alpha- | SPTAN1 | NM_003127 | (ALPHA)II- | 3190558 | chr9 | + | 130354704 | 130435758 | 1.04 |
| fodrin) | | | SPECTRIN // | | | | | | |
| | | | FLJ44613 | | | | | | |
| protein phosphatase 1, catalytic subunit, gamma | PPP1CC | NM_002710 | PPP1G | 3471374 | chr12 | − | 109642007 | 109665131 | 1.04 |
| isoform | | | | | | | | | |
| CDC-like kinase 1 | CLK1 | NM_001024646 // | CLK // CLK/STY | 2594497 | chr2 | − | 201425997 | 201437659 | 1.04 |
| | | NM_004071 | // STY | | | | | | |
| transient receptor potential cation channel, | TRPC4AP | NM_015638 // | C20orf188 // | 3903708 | chr20 | − | 33053899 | 33144297 | 1.04 |
| subfamily C, member 4 associated protein | | NM_199368 | TRRP4AP // | | | | | | |
| actin, beta | ACTB | NM_001101 | PS1TP5BP1 | 3036924 | chr7 | − | 5533433 | 5537011 | 1.04 |
| polypyrimidine tract binding protein 1 | PTBP1 | NM_002819 | HNRNPI // HNRPI | 3815165 | chr19 | + | 747768 | 763505 | 1.04 |
| | | NM_031990 // | // MGC10830 // | | | | | | |
| | | NM_031991 // | MGC8461 // PTB | | | | | | |
| | | NM_175847 | // PTB-1 // PTB- | | | | | | |
| | | | T // PTB2 // PTB3 | | | | | | |
| | | | // PTB4 // pPTB | | | | | | |
| chromosome 6 open reading frame 166 | C6orf166 | NM_018064 | FLJ10342 // | 2963784 | chr6 | − | 88441297 | 88476721 | 1.04 |
| | | | dJ486L4.2 | | | | | | |
| ring finger protein 40 | RNF40 | NM_014771 | BRE1B // | 3656555 | chr16 | + | 30681120 | 30695182 | 1.04 |
| | | | DKFZp686K191 // | | | | | | |
| | | | KIAA0661 // | | | | | | |
| | | | MGC13051 // | | | | | | |
| | | | RBP95 // STARING | | | | | | |
| coiled-coil domain containing 100 | CCDC100 | NM_153223 | DKFZp686106246 // | 2873168 | chr5 | − | 122642038 | 122832582 | 1.04 |
| | | | FLJ36090 // | | | | | | |
| | | | FLJ38327 | | | | | | |
| ubiquitin specific peptidase B | USP8 | NM_005154 | FLJ34456 // | 3593652 | chr15 | + | 48503634 | 48580565 | 1.04 |
| | | | HumORF8 // | | | | | | |
| | | | KIAA0055 // | | | | | | |
| | | | MGC129718 // | | | | | | |
| | | | UBPY | | | | | | |
| splicing factor 1 | SF1 | NM_004630 // | D11S636 // ZFM1 | 3377044 | chr11 | − | 64287827 | 64302817 | 1.04 |
| | | NM_201995 // | // ZNF162 | | | | | | |
| | | NM_201997 // | | | | | | | |
| | | NM_201998 | | | | | | | |
| family with sequence similarity 89, member B | FAM89B | NM_152832 | MTVR1 | 3335338 | chr11 | + | 65096490 | 65098230 | 1.04 |
| eukaryotic translation initiation factor 4A, | EIF4A2 | NM_001967 | BM-010 // DDX2B | 2656738 | chr3 | + | 187961636 | 187990742 | 1.04 |
| isoform 2 | | | // EIF4A // EIF4F | | | | | | |
| leucine-rich PPR-motif containing | LRPPRC | NM_133259 | CLONE-23970 // | 2550790 | chr2 | − | 43961504 | 44085128 | 1.04 |
| | | | GP130 // LRP130 | | | | | | |
| | | | // LSFC | | | | | | |
| v-rel reticuloendothellosis viral oncogene | RELA | NM_021975 | MGC131774 // | 3377789 | chr11 | − | 65176856 | 65204491 | 1.04 |
| homolog A, nuclear factor of kappa light | | | NFKB3 | | | | | | |
| polypeptide gene enhancer in B-cells 3, p65 | | | | | | | | | |
| (avian) | | | | | | | | | |
| chromodomain helicase DNA binding protein 4 | CHD4 | NM_001273 | DKFZp686E06161 | 3442054 | chr12 | − | 6549521 | 6587067 | 1.04 |
| | | | // Mt-2b // Mi2- | | | | | | |
| transferrin receptor (p90, CD71) | TFRC | NM_003234 | CD71 // TFR // | 2712632 | chr3 | − | 197238405 | 197334433 | 1.04 |
| | | | TFR1 // TRFR | | | | | | |
| GRP1 (general receptor for phosphoinositides | GRASP | NM_181711 | — | 3415193 | chr12 | + | 50686988 | 50695938 | 1.04 |
| 1)-associated scaffold protein | | | | | | | | | |
| small nuclear ribonucleoprotein 70 kDa | SNRP70 | NM_001009820 // | RNPU1Z // RPU1 | 3838185 | chr19 | + | 54280358 | 54304714 | 1.04 |
| polypeptide (RNP antigen) | | NM_003089 | // U170K // U1AP | | | | | | |
| | | | // U1RNP | | | | | | |
| 1-acylglycerol-3-phosphate O-acyltransferase 3 | AGPAT3 | NM_001037553 // | LPAAT-GAMMA1 | 3923354 | chr21 | + | 44105141 | 44245644 | 1.04 |
| | | NM_020132 | // MGC4604 | | | | | | |
| vacuolar protein sorting 4 homolog B (S. cerevisiae) | VPS4B | NM_004869 | MIG1 // SKD1 // | 3811497 | chr18 | − | 59207407 | 59240734 | 1.04 |
| | | | VPS4-2 | | | | | | |
| KIAA1128 | KIAA1128 | NM_018999 | FLJ14262 // | 3255402 | chr10 | + | 86020629 | 86268247 | 1.04 |
| | | | FLJ25809 // | | | | | | |
| | | | Gcap14 // | | | | | | |
| | | | bA486O22.1 | | | | | | |
| c-src tyrosine kinase | CSK | NM_004383 | MGC117393 | 3601840 | chr15 | + | 72841734 | 72882558 | 1.04 |
| pleiotropic regulator 1 (PRL1 homolog, | PLRG1 | NM_002669 | MGC110880 // | 2790570 | chr4 | − | 155675613 | 155691014 | 1.04 |
| Arabidopsis) | | | PRL1 | | | | | | |
| WD repeat domain 1 | WDR1 | NM_005112 // | AIP1 // NORI-1 | 2760371 | chr4 | − | 9685078 | 9772600 | 1.04 |
| | | NM_017491 | | | | | | | |
| FBJ murine osteosarcoma viral oncogene | FOSB | NM_006732 | DKFZp686C0818 // | 3836266 | chr19 | + | 50624036 | 50671180 | 1.04 |
| homolog B | | | G0S3 // GOS3 // | | | | | | |
| | | | GOSB // | | | | | | |
| c-Maf-inducing protein | CMIP | NM_030629 // | KIAA1694 | 3670772 | chr16 | + | 80030441 | 80323852 | 1.04 |
| | | NM_198390 | | | | | | | |
| aldolase A, fructose-bisphosphate | ALDOA | NM_000034 // | ALDA // | 3655920 | chr16 | + | 29968869 | 29991009 | 1.04 |
| | | NM_184041 // | MGC10942 // | | | | | | |
| | | NM_184043 | MGC17716 // | | | | | | |
| bolA homolog 1 (E. coli) | BOLA1 | NM_016074 | CGI-143 // | 2357961 | chr1 | + | 148126062 | 148138968 | 1.04 |
| | | | MGC75015 // | | | | | | |
| | | | RP11-196G18.18 | | | | | | |
| ring finger and SPRY domain containing 1 | RSPRY1 | NM_133368 | KIAA1972 | 3662612 | chr16 | + | 55777703 | 55831882 | 1.04 |
| centaurin, delta 2 | CENTD2 | NM_001040118 // | ARAP1 | 3381241 | chr11 | − | 72073021 | 72141062 | 1.04 |
| | | NM_015242 // | KIAA0782 | | | | | | |
| | | NM_139181 | | | | | | | |
| NADH dehydrogenase (ubiquinone) 1 alpha | NDUFA5 | NM_005000 | B13 // Cl-13KD-B | 3070658 | chr7 | − | 122968075 | 122985216 | 1.04 |
| subcomplex, 5, 13 kDa | | | // DKFZp781K1356 | | | | | | |
| | | | // FLJ12147 // | | | | | | |
| | | | // NUFM //UQOR13 | | | | | | |
| brix domain containing 2 | BXDC2 | NM_018321 | BRIX // FLJ11100 | 2806231 | chr5 | + | 34951248 | 34962845 | 1.04 |
| tumor necrosis factor receptor superfamily, | TNFRSF25 // | NM_001039664 // | APO-3 // DDR3 // | 2394699 | chr1 | − | 6443807 | 6448816 | 1.04 |
| member 25 // pleckstrin homology domain | PLEKHG5 | NM_003790 // | DR3 // LARD // | | | | | | |
| containing, family G (with RhoGef domain) | | NM_148965 // | TNFRSF12 // TR3 | | | | | | |
| member 5 | | NM_148966 // | // TRAMP // WSL- | | | | | | |
| | | NM_148967 // | 1 // WSL-LR // | | | | | | |
| | | NM_148970 // | KIAA0720 // RP4- | | | | | | |
| | | NM_001042663 // | 650H14.3 | | | | | | |
| | | NM_001042664 // | | | | | | | |
| | | NM_001042665 // | | | | | | | |
| | | NM_020631 // | | | | | | | |
| | | NM_198681 | | | | | | | |
| nucleoporin like 1 | NUPL1 | NM_001008564 // | KIAA0410 // | 3482219 | chr13 | + | 24773258 | 24822202 | 1.04 |
| | | NM_001008565 // | PRO2463 | | | | | | |
| | | NM_014089 | | | | | | | |
| nuclear factor of kappa light polypeptide gene | NFKBIE | NM_004556 | IKBE | 2955076 | chr6 | − | 44333884 | 44341478 | 1.04 |
| enhancer in B-cells inhibitor, epsilon | | | | | | | | | |
| tripartite motif-containing 8 | TRIM8 | NM_030912 | GERP // RNF27 | 3261820 | chr10 | + | 104384137 | 104419190 | 1.04 |
| WD repeat and SOCS box-containing 1 | WSB1 | NM_015626 // | SWIP1 // WSB-1 | 3715109 | chr17 | + | 22644482 | 22684326 | 1.04 |
| | | NM_134265 | | | | | | | |
| SAPS domain family, member 3 | SAPS3 | NM_018312 | C11orf23 // | 3337618 | chr11 | + | 67984785 | 68173381 | 1.04 |
| | | | DKFZp781E17107 | | | | | | |
| | | | // DKFZp781E2374 | | | | | | |
| | | | // DKFZp781O2362 | | | | | | |
| | | | // FLJ11058 // | | | | | | |
| | | | FLJ43065 // | | | | | | |
| | | | MGC125711 // | | | | | | |
| | | | MGC125712 // | | | | | | |
| | | | PP6R3 // SAP190 | | | | | | |
| | | | // SAPL // SAPLa | | | | | | |
| hypothetical protein FLJ21438 | FLJ21438 | XM_029084 | DKFZp667E013 // | 3853453 | chr19 | − | 15423181 | 15436377 | 1.04 |
| | | | FLJ00087 | | | | | | |
| CD14 molecule | CD14 | NM_000591 // | — | 2878437 | chr5 | − | 139991507 | 139993422 | 1.04 |
| | | NM_001040021 | | | | | | | |
| PRP3 pre-mRNA processing factor 3 homolog | PRPF3 // | NM_004698 // | HPRP3 // HPRP3P | 2358171 | chr1 | + | 148560583 | 148592321 | 1.04 |
| (S. cerevisiae) // KIAA0460 | KIAA0460 | NM_015203 | // PRP3 // Prp3p | | | | | | |
| | | | // RP18 // | | | | | | |
| | | | FLJ32145 // | | | | | | |
| | | | HSPC099 | | | | | | |
| signal transducer and activator of transcription | STAT5B | NM_012448 | STAT5 | 3757770 | chr17 | − | 37604727 | 37682109 | 1.04 |
| jumonji domain containing 1A | JMJD1A | NM_018433 | DKFZp686A24246 | 2492064 | chr2 | + | 86509150 | 86574548 | 1.04 |
| | | | // | | | | | | |
| | | | DKFZp686P07111 | | | | | | |
| | | | // JHMD2A // | | | | | | |
| | | | JMJD1 // | | | | | | |
| IQ motif containing GTPase activating protein 1 | IQGAP1 | NM_003870 | HUMORFA01 // | 3608113 | chr15 | + | 88732454 | 88846463 | 1.04 |
| | | | KIAA0051 // SAR1 | | | | | | |
| | | | // p195 | | | | | | |
| ubiquitin-conjugating enzyme E2B (RAD6 | UBE2B | NM_003337 | E2-17 kDa // | 2829275 | chr5 | + | 133734769 | 133788148 | 1.04 |
| homolog) | | | HHR6B // HR6B // | | | | | | |
| | | | RAD6B // UBC2 | | | | | | |
| splicing factor, arginine/serine-rich 10 | SFRS10 | NM_004593 | DKFZp686F18120 | 2709062 | chr3 | − | 187048509 | 187160523 | 1.04 |
| (transformer 2 homolog, Drosophila) | | | // Htra2-beta // | | | | | | |
| | | | SRFS10 // TRA2- | | | | | | |
| | | | BETA // TRA2B | | | | | | |
| poly(A) polymerase alpha | PAPOLA | NM_032632 | MGC5378 // PAP | 3550392 | chr14 | + | 96038038 | 96104627 | 1.04 |
| kelch-like 18 (Drosophila) | KLHL18 | NM_025010 | FLJ13703 // | 2621275 | chr3 | + | 47298888 | 47367590 | 1.04 |
| | | | KIAA0795 | | | | | | |
| G protein-coupled receptor 132 | GPR132 | NM_013345 | G2A // MGC99642 | 3581404 | chr14 | − | 104586779 | 104622593 | 1.04 |
| phosphatidylinositol transfer protein, beta | PITPNB | NM_012399 | PI-TP-beta // | 3956290 | chr22 | − | 26577440 | 26645487 | 1.04 |
| | | | PtdInsTP // VIB1B | | | | | | |
| Snf2-related CBP activator protein | SRCAP | NM_006662 | KIAA0309 | 3656418 | chr16 | + | 30616551 | 30663998 | 1.04 |
| engulfment and cell motility 1 | ELMO1 | NM_001039459 // | CED-12 // CED12 | 3046197 | chr7 | − | 36860492 | 37455389 | 1.04 |
| | | NM_014800 // | // ELMO-1 // | | | | | | |
| | | NM_130442 | KIAA0281 // | | | | | | |
| | | | MGC126406 | | | | | | |
| metal response element binding transcription | MTF2 | NM_007358 | M96 // PCL2 // | 2346934 | chr1 | + | 93147511 | 93377202 | 1.04 |
| factor 2 | | | RP5-976O13.1 // | | | | | | |
| | | | dJ976O13.2 | | | | | | |
| hemopoietic cell kinase | HCK | NM_002110 | JTK9 | 3881651 | chr20 | + | 30086433 | 30153306 | 1.04 |
| SIN3 homolog A, transcription regulator (yeast) | SIN3A | NM_015477 | DKFZP434K2235 // | 3633403 | chr15 | − | 73448786 | 73535167 | 1.04 |
| | | | FLJ90319 // | | | | | | |
| | | | KIAA0700 | | | | | | |
| eukaryotic translation initiation factor 3, subunit | EIF3S9 | NM_001037283 // | EIF3-ETA // EIF3- | 2987441 | chr7 | + | 2315929 | 2386896 | 1.04 |
| 9 eta, 116 kDa | | NM_003751 | P110 // EIF3-P116 | | | | | | |
| | | | // MGC104664 // | | | | | | |
| | | | MGC131875 // | | | | | | |
| | | | PRT1 // eIF3b | | | | | | |
| transmembrane 9 superfamily member 3 | TM9SF3 | NM_020123 | EP70-P-iso // | 3301857 | chr10 | − | 98267858 | 98337079 | 1.04 |
| | | | RP11-34E5.1 // | | | | | | |
| | | | SMBP | | | | | | |
| chromosome 1 open reading frame 50 | C1orf50 | NM_024097 | MGC955 | 2332767 | chr1 | + | 43005527 | 43036633 | 1.04 |
| chaperonin containing TCP1, subunit 6A (zeta | CCT6A | NM_001009186 // | CCT-zeta // CCT- | 3003193 | chr7 | + | 56086894 | 56099148 | 1.04 |
| 1) | | NM_001762 | zeta-1 // CCT6 // | | | | | | |
| | | | Cctz // HTR3 // | | | | | | |
| | | | MGC126214 // | | | | | | |
| | | | MGC126215 // | | | | | | |
| | | | MoDP-2 // TCP-1- | | | | | | |
| | | | zeta // TCP20 // | | | | | | |
| | | | TCPZ // TTCP20 | | | | | | |
| zinc finger protein 410 | ZNF410 | NM_021188 | APA-1 // APA1 | 3543884 | chr14 | + | 73423093 | 73468718 | 1.04 |
| KIAA1468 | KIAA1468 | NM_020854 | FLJ33841 // | 3791168 | chr18 | + | 58005478 | 58125326 | 1.04 |
| | | | HsT3308 // HsT885 | | | | | | |
| nucleolar protein NOP5/NOP58 | NOP5/NOP58 | NM_015934 | HSPC120 | 2523144 | chr2 | + | 202811346 | 202926967 | 1.04 |
| guanylate binding protein 5 | GBP5 | NM_052942 | GBP-5 | 2422035 | chr1 | − | 89496930 | 89511122 | 1.04 |
| 5′-3′ exoribonuclease 2 | XRN2 | NM_012255 | — | 3879467 | chr20 | + | 21219705 | 21322172 | 1.04 |
| fragile X mental retardation, autosomal homolog 1 | FXR1 | NM_001013438 // | — | 2654394 | chr3 | + | 182113040 | 182179459 | 1.04 |
| | | NM_001013439 // | | | | | | | |
| | | NM_005087 | | | | | | | |
| oxoglutarate (alpha-ketoglutarate) | OGDH | NM_001003941 // | AKGDH // E1k // | 2999948 | chr7 | + | 44612716 | 44715181 | 1.04 |
| dehydrogenase (lipoamide) | | NM_002541 | OGDC | | | | | | |
| KIAA1967 | KIAA1967 | NM_021174 // | DBC-1 // DBC1 | 3089597 | chr8 | + | 22518038 | 22534618 | 1.04 |
| | | NM_199205 | | | | | | | |
| C-type lectin domain family 5, member A | CLEC5A | NM_013252 | CLECSF5 // MDL- | 3076868 | chr7 | − | 141273626 | 141298905 | 1.04 |
| | | | 1 // MDL1 // | | | | | | |
| | | | MGC138304 | | | | | | |
| exportin 4 | XPO4 | NM_022459 | FLJ13046 // | 3504434 | chr13 | − | 20251014 | 20375187 | 1.04 |
| | | | KIAA1721 | | | | | | |
| RNA binding motif protein 6 | RBM6 | NM_005777 | 3G2 // DEF-3 // | 2622359 | chr3 | + | 49942372 | 50089683 | 1.04 |
| | | | DEF3 // | | | | | | |
| | | | DKFZp686B0877 // | | | | | | |
| | | | FLJ36517 // HLC- | | | | | | |
| | | | 11 // NY-LU-12 // | | | | | | |
| | | | g16 | | | | | | |
| nucleotide binding protein 1 (MinD homolog, E. coli) | NUBP1 | NM_002484 | MGC117406 // | 3647956 | chr16 | + | 10729448 | 10771777 | 1.04 |
| | | | MGC130052 // | | | | | | |
| | | | MGC130053 // NBP | | | | | | |
| | | | // NBP1 | | | | | | |
| Yip1 domain family, member 3 // chromosome | YIPF3 // | NM_015388 // | C6orf109 // | 2954646 | chr6 | − | 43587500 | 43592701 | 1.04 |
| 6 open reading frame 154 | C6orf154 | NM_001012974 | DKFZP566C243 // | | | | | | |
| | | | FinGER3 // KLIP1 | | | | | | |
| | | | // dJ337H4.3 // | | | | | | |
| | | | FLJ44836 // | | | | | | |
| | | | MGC131686 // | | | | | | |
| | | | dJ337H4.2 | | | | | | |
| chromosome 6 open reading frame 94 // LTV1 | C6orf94 // | XM_928657 // | dJ468K18.5 // | 2929036 | chr6 | + | 144194149 | 144227146 | 1.04 |
| homolog (S. cerevisiae) | LTV1 | XM_941265 // | C6orf93 // | | | | | | |
| | | NM_032860 | FLJ14909 // | | | | | | |
| | | | dJ468K18.4 | | | | | | |
| intercellular adhesion molecule 1 (CD54), human | ICAM1 | NM_000201 | BB2 // CD54 // | 3820443 | chr19 | + | 10242765 | 10258289 | 1.04 |
| rhinovirus receptor | | | P3.58 | | | | | | |
| KIAA0368 | KIAA0368 | XM_001129450 // | ECM29 // | 3220513 | chr9 | − | 113131941 | 113286475 | 1.04 |
| | | XM_001131778 // | FLJ22036 // | | | | | | |
| | | NM_001080398 | KIAA1962 // RP11- | | | | | | |
| CD3d molecule, delta (CD3-TCR complex) | CD3D | NM_000732 // | CD3-DELTA // | 3393744 | chr11 | − | 117698596 | 117718659 | 1.04 |
| | | NM_001040651 | T3D | | | | | | |
| translocase of outer mitochondrial membrane | TOMM70A | NM_014820 | FLJ90470 | 2686371 | chr3 | − | 101565001 | 101614635 | 1.04 |
| 70 homolog A (S. cerevisiae) | | | | | | | | | |
| natural killer-tumor recognition sequence | NKTR | NM_001012651 // | DKFZp686F1754 // | 2619344 | chr3 | + | 42617105 | 42678166 | 1.04 |
| | | NM_005385 | DKFZp686G0426 // | | | | | | |
| | | | DKFZp686J06106 | | | | | | |
| | | | // | | | | | | |
| | | | DKFZp686N24126 | | | | | | |
| nucleolar protein 9 | NOL9 | NM_024654 | FLJ23323 // | 2394784 | chr1 | − | 6504004 | 6537329 | 1.04 |
| | | | MGC131821 // | | | | | | |
| | | | MGC138483 | | | | | | |
| proteasome (prosome, macropain) 26S subunit, | PSMC4 | NM_006503 // | MGC13687 // | 3833291 | chr19 | + | 45168772 | 45179181 | 1.04 |
| ATPase, 4 | | NM_153001 | MGC23214 // | | | | | | |
| | | | MGC8570 // | | | | | | |
| | | | MIP224 // S6 // | | | | | | |

1. The method according to claim 18, wherein the antitumor agent is a compound represented by formula (I), a pharmaceutically acceptable salt thereof, or a solvate of them: 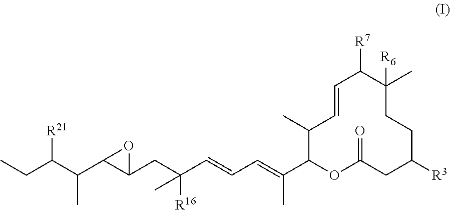
wherein R³, R⁶, R⁷ and R²¹, the same or different, each represents
1) a hydroxyl group or an oxo group formed together with the carbon atom to which it is bound, provided that R⁶ is limited to a hydroxyl group,
2) an optionally substituted C₁₋₂₂ alkoxy group,
3) an optionally substituted unsaturated C₂₋₂₂ alkoxy group,
4) an optionally substituted C₇₋₂₂ aralkyloxy group,
5) an optionally substituted 5- to 14-membered heteroaralkyloxy group,
6) RCO—O— wherein R represents
a) a hydrogen atom,
b) an optionally substituted C₁₋₂₂ alkyl group,
c) an optionally substituted unsaturated C₂₋₂₂ alkyl group,
d) an optionally substituted C₆₋₁₄ aryl group,
e) an optionally substituted 5- to 14-membered heteroaryl group,
f) an optionally substituted C₇₋₂₂ aralkyl group,
g) an optionally substituted 5- to 14-membered heteroaralkyl group,
h) an optionally substituted C₁₋₂₂ alkoxy group,
i) an optionally substituted unsaturated C₂₋₂₂ alkoxy group,
j) an optionally substituted C₆₋₁₄ aryloxy group or
k) an optionally substituted 5- to 14-membered heteroaryloxy group,
7) R^{S1}R^{S2}R^{S3}SiO— wherein R^{SI}, R^{S2}, and R^{S3}, the same or different, each represents
a) a C₁₋₆ alkyl group or
b) a C₆₋₁₄ aryl group,
8) a halogen atom,
9) R^{N1}R^{N2}N—R^{M}— wherein R^{M} represents
a) a single bond,
b) —CO—O—,
c) —SO₂—O—,
d) —CS—O— or
e) —CO—NR^{N3}— wherein R^{N3} represents a hydrogen atom or an optionally substituted C₁₋₆ alkyl group, provided that each of the leftmost bond in b) to e) is bound to the nitrogen atom; and R^{N1} and R^{N2}, the same or different from each other and each represents
a) a hydrogen atom,
b) an optionally substituted C₁₋₂₂ alkyl group,
c) an optionally substituted unsaturated C₂₋₂₂ alkyl group,
d) an optionally substituted aliphatic C₂₋₂₂ acyl group,
e) an optionally substituted aromatic C₇₋₁₅ acyl group,
f) an optionally substituted C₆₋₁₄ aryl group,
g) an optionally substituted 5- to 14-membered heteroaryl group,
h) an optionally substituted C₇₋₂₂ aralkyl group,
i) an optionally substituted C₁₋₂₂ alkylsulfonyl group,
j) an optionally substituted C₆₋₁₄ arylsulfonyl group,
k) an optionally substituted 3- to 14-membered non-aromatic heterocyclic group formed by R^{N1} and R^{N2} together with the nitrogen atom to which R^{N1} and R^{N2} are bound, and the non-aromatic heterocyclic group optionally has substituent(s),
l) an optionally substituted 5- to 14-membered heteroaralkyl group,
m) an optionally substituted C₃₋₁₄ cycloalkyl group or
n) an optionally substituted 3- to 14-membered non-aromatic heterocyclic group,
10) R^{N4} SO₂— wherein R^{N4} represents
a) an optionally substituted C₁₋₂₂ alkyl group,
b) an optionally substituted C₆₋₁₄ aryl group,
c) an optionally substituted C₁₋₂₂ alkoxy group,
d) an optionally substituted unsaturated C₂₋₂₂ alkoxy group,
e) an optionally substituted C₆₋₁₄ aryloxy group,
f) an optionally substituted 5- to 14-membered heteroaryloxy group,
g) an optionally substituted C₇₋₂₂ aralkyloxy group or
h) an optionally substituted 5- to 14-membered heteroaralkyloxy group,
11) (R^{N5}O)₂PO—O— wherein R^{N5} represents
a) an optionally substituted C₁₋₂₂ alkyl group,
b) an optionally substituted unsaturated C₂₋₂₂ alkyl group,
c) an optionally substituted C₆₋₁₄ aryl group,
d) an optionally substituted 5- to 14-membered heteroaryl group,
e) an optionally substituted C₇₋₂₂ aralkyl group or
f) an optionally substituted 5- to 14-membered heteroaralkyl group,
12) (R^{N1}R^{N2}N)₂PO—O— wherein R^{N1} and R^{N2} have the same meanings as defined above or
13) (R^{N1}R^{N2}N)(R^{N5}O)PO—O— wherein R^{N1}, R^{N2} and R^{N5} have the same meanings as defined above, provided that a compound in which R³, R⁶, R⁷ and R²¹ are all hydroxyl groups, and a compound in which R³, R⁶ and R²¹ are all hydroxyl groups and R⁷ is an acetoxy group are excluded,
R¹⁶ represents a hydrogen atom or hydroxyl group.

2. The method according to claim 1, wherein the antitumor agent is selected from the group consisting of:
(8E,12E,14E)-7-(N-(2-(N′,N′-Dimethylamino)ethyl)-N-methylcarbamoyloxy)-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-methylhomopiperazin-1-yl)carbonyl)oxy-18,19-epoxytricosa-8,12,14-trien-11-olide;
(8E,12E,14E)-3,6,16,21-Tetrahydroxy-6,10,12,16,20-pentamethyl-7-((4-methylpiperazin-1-yl)carbonyl)oxy-18,19-epoxytricosa-8,12,14-trien-11-olide;
(8E,12E,14E)-7-((4-Butylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Ethylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-propylpiperazin-1-yl)carbonyl)oxy-18,19-epoxytricosa-8,12,14-trien-11-olide;
(8E,12E,14E)-7-((4-Cyclohexylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-(Cyclopropylmethyl)piperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-6,10,12,16,20-pentamethyl-7-((4-propylhomopiperazin-1-yl)carbonyl)oxy-18,19-epoxytricosa-8,12,14-trien-11-olide; (8E,12E,14E)-7-((4-(Cyclopropylmethyl)homopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Cyclopentylpiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-7-((4-isopropylpiperazin-1-yl)carbonyl)oxy-6,10,12,16,20-pentamethyl-18,19-epoxytricosa-8,12,14-trien-11-olide;
(8E,12E,14E)-7-((4-Cycloheptylpiperazin-1-yl)carbonyl)oxy-3,6,16,21-Tetrahydroxy-6,10,12,16,20-pentamethyl-18,19-epoxytricosa-8,12,14-trien-11-olide;
(8E,12E,14E)-7-(N-(2-(N′,N′-Diethylamino)ethyl)-N-methylcarbamoyloxy)-3,6,16,21-tetrahydroxy-6,10,12,16,20-pentamethyl-18,19-epoxytricosa-8,12,14-trien-11-olide;
(8E,12E,14E)-3,6,16,21-Tetrahydroxy-7-((4-isobutylhomopiperazin-1-yl)carbonyl)oxy-6,10,12,16,20-pentamethyl-18,19-epoxytricosa-8,12,14-trien-11-olide;
(8E,12E,14E)-7-((4-Ethylhomopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-7-((4-Butylhomopiperazin-1-yl)carbonyl)oxy-3,6,16,21-tetrahydroxy-6,10,12,16,20-pentamethyl-18,19-epoxytricosa-8,12,14-trien-11-olide;
(8E,12E,14E)-3,16,21-Trihydroxy-6-methoxy-6,10,12,16,20-pentamethyl-7-((4-methylpiperazin-1-yl)carbonyl)oxy-18,19-epoxytricosa-8,12,14-trien-11-olide;
(8E,12E,14E)-3,16,21-Trihydroxy-6-methoxy-6,10,12,16,20-pentamethyl-7-((4-(piperidin-1-yl)piperidin-1-yl)carbonyl)oxy-18,19-epoxytricosa-8,12,14-trien-11-olide;
(8E,12E,14E)-3,6,7,21-Tetrahydroxy-6,10,12,16,20-pentamethyl-18,19-epoxytricosa-8,12,14-trien-11-olide;
(8E,12E,14E)-7-((4-(2,2-Dimethylpropyl)homopiperazin-1-yl)carbonyl)oxy-3,6,16,21-tetrahydroxy-6,10,12,16,20-pentamethyl-18,19-epoxytricosa-8,12,14-trien-11-olide; and
(8E,12E,14E)-3,6,16-Trihydroxy-21-methoxy-6,10,12,16,20-pentamethyl-7-((4-methylpiperazin-1-yl)carbonyl)oxy-18,19-epoxytricosa-8,12,14-trien-11-olide.

3. The method according to claim 1, wherein the detection of splicing defect comprises the steps of:
(a) measuring the expression level of pre-mRNA before and after administration of the antitumor agent to a mammal;
(b) comparing, based on the expression level measured in (a), the expression level of pre-mRNA before and after administration of the antitumor agent to determine that the antitumor agent exerts an action to the mammal when the expression level of pre-mRNA after the administration increases.

4. The method according to claim 3, wherein the pre-mRNA of which expression level is measured is pre-mRNA of at least one gene selected from the genes listed in Table 1, Table 2, Table 3, Table 4, and Table 5, or a homologous gene thereof.

5. The method according to claim 4, wherein the gene(s) are selected from DNAJB1, BZW1, NUP54, RIOK3, CDKN1B, STK17B, EIF4A1, and ID1.

6. The method according to claim 3, wherein in step (a), the expression level of pre-mRNA in samples obtained from a subject before and after administration of the antitumor agent is measured.

7. The method according to claim 6, wherein the samples obtained from the subject are selected from hemocytes in peripheral blood, plasma, and serum.

8. A probe or primer for assaying an action of a compound represented by formula (I), a pharmaceutically acceptable salt thereof, or a solvate of them to a mammal, which consists of a polynucleotide capable of hybridizing with a polynucleotide consisting of a nucleotide sequence of at least one gene selected from the genes listed in Table 1, Table 2, Table 3, Table 4, and Table 5, and a homologous gene thereof, or a complementary sequence thereof.

9. The probe or primer according to claim 8, which is capable of detecting a genomic intron region or a part thereof in a gene listed in Table 1, Table 2, Table 3, Table 4 and Table 5, or which is capable of detecting a polynucleotide lacking a part of a genomic exon region in a gene listed in Table 1, Table 2, Table 3, Table 4 and Table 5.

10. A reagent or kit for assaying an action of a compound represented by formula (I), a pharmaceutically acceptable salt thereof, or a solvate of them to a mammal, which comprises the probe or the primer according to claim 8.

11. The method according to claim 1, wherein the detection of splicing defect comprises the steps of:
(f) measuring the expression level of an abnormal protein before and after administration of the antitumor agent to a mammal;
(g) comparing, based on the expression level measured in (f), the expression level of the abnormal protein before and after administration of the antitumor agent to determine that the antitumor agent exerts an action to the mammal when the expression level of the abnormal protein after the administration increases.

12. The method according to claim 11, wherein the abnormal protein of which expression level is measured is a protein consisting of amino acids encoded by a polynucleotide of at least one gene selected from the genes listed in Table 1 and Table 3, or a homologous gene thereof where splicing defect has been caused in the polynucleotide, or a protein consisting of amino acids encoded by a polynucleotide of at least one gene selected from the genes listed in Table 2, Table 4 and Table 5 or a homologous gene thereof where splicing defect has been caused in the polynucleotide.

13. The method according to claim 11, wherein the abnormal protein of which expression level is measured is a protein consisting of amino acids encoded by a polynucleotide of at least one gene selected from DNAJB1, BZW1, NUP54, RIOK3, CDKN1B, STK17B, EIF4A1 and ID1 where splicing defect has been caused in the polynucleotide.

14. The method according to claim 11, wherein in step (f), the expression level of the abnormal protein in the samples obtained from a subject before and after administration of the antitumor agent is measured.

15. The method according to claim 14, wherein the samples obtained from the subject are selected from hemocytes in peripheral blood, plasma, and serum.

16. An antibody against an abnormal protein consisting of amino acids encoded by a polynucleotide of at least one gene selected from the genes listed in Table 1, Table 2, Table 3, Table 4 and Table 5 where splicing defect has been caused in the polynucleotide, or a fragment thereof.

17. A reagent or kit for assaying an action of a compound represented by formula (I), a pharmaceutically acceptable salt thereof, or a solvate of them to a mammal, comprising the antibody or the fragment thereof according to claim 16.

18. A method for assaying an action of an antitumor agent to a mammal, which comprises detecting splicing defect caused by the antitumor agent.
